(19) United States
(12) Patent Application Publication
(10) Pub. No.: US 2024/0376156 A1
(43) Pub. Date: Nov. 14, 2024

(54) MULTIPLE DE NOVO DESIGNED PROTEIN BINDING PROTEINS

(71) Applicant: UNIVERSITY OF WASHINGTON, Seattle, WA (US)

(72) Inventors: Lansing Joseph STEWART, Seattle, WA (US); David BAKER, Seattle, WA (US); Longxing CAO, Seattle, WA (US); Brian COVENTRY, Seattle, WA (US); Inna GORESHNIK, Seattle, WA (US); Buwei HUANG, Seattle, WA (US); Nathaniel BENNETT, Seattle, WA (US); Eva-Maria STRAUCH, Seattle, WA (US); Thomas SCHLICHTHAERLE, Seattle, WA (US)

(21) Appl. No.: 18/576,518

(22) PCT Filed: Jul. 11, 2022
(86) PCT No.: PCT/US2022/073590

§ 371 Date: Jan. 4, 2024

Related U.S. Application Data
(60) Provisional application No. 63/221,327, filed on Jul. 13, 2021.

(51) Int. Cl. C07K 14/00; A61K 38/00; C12N 15/70

(52) U.S. Cl. CPC C07K 14/00; C12N 15/70; A61K 38/00

(57) ABSTRACT

Designed polypeptides that bind specifically to a defined protein target are provided, as well as methods for their design and use.

### CROSS REFERENCE

This application claims priority to U.S. Provisional Patent Application Ser. No. 63/221,327 filed Jul. 13, 2021, incorporate by reference herein in its entirety.

### FEDERAL FUNDING STATEMENT

This invention was made with government support under Grant No. FA8750-17-C-0219, awarded by the Defense Advanced Research Projects Agency and Grant No. HDTRA1-16-C-0029, awarded by the Defense Threat Reduction Agency and Grant Nos. HHSN272201700059C and R01 AG063845, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Jul. 4, 2022 having the file name “21-0753-WO_SeqList.hml” and is 1,981 kb in size.

### BACKGROUND

Protein interactions play critical roles in biology, and general approaches to disrupt or modulate these with designed proteins would have huge impact. While empirical laboratory selection approaches starting from very large antibody, DARPIN or other protein scaffold libraries can generate binders to protein targets, it is difficult at the outset to target a specific region on a target protein surface, and to sample the full space of possible binding modes. Computational methods can target specific target surface locations and provide a more principled and potentially much faster approach to binder generation than random library selection methods, as well as insight into the fundamental properties of protein interfaces (which must be understood for design to be successful). Most current methods for designing proteins to bind to a target surface utilize information derived from native complex structures on specific sidechain interactions or protein backbone placements optimal for binding. For many target proteins, there are no obvious pockets or clefts on the protein surface into which a small number of privileged sidechains can be placed, and guidance by a small number of hotspot residues limits the approach to a small fraction of possible interaction modes.

### SUMMARY

In one aspect, the disclosure provides polypeptides comprising an amino acid sequence at least 35/u, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-1559 and 1561-1570, not including any functional domains added fused to the polypeptides (whether N-terminal, C-terminal, or internal), and wherein the 1, 2, 3, 4, or 5 N-terminal and/or C-terminal amino acid residues may be present or absent when considering the percent identity. In another embodiment, substitutions relative to the reference polypeptide are selected from the residues listed as “best” or “tolerable” at each position immediately below the reference polypeptide listed in Tables 13A-13HHH. In a further embodiment, substitutions relative to the reference polypeptide are selected from the residues listed as “best” or “tolerable” at each position immediately below the reference polypeptide listed in Tables 13A-13HHH. In one embodiment, 1, 2, 3, 4, 5, 6, 7, 8, 9, 10, 11, 12, 13, 14, 15, or all interface residues are as defined in the reference polypeptide listed in Tables 13A-13HHH. In another embodiment, protein core residues listed in Tables 13A-13HHH are substituted relative to the reference polypeptide only with conservative amino acid substitutions. In a further embodiment, insertion of amino acid residues relative to the reference polypeptide occurs at a residue indicated in the column “loop/insertion” column of Tables 13A-13HHH. In another embodiment, 1, 2, 3, 4, or 5 N-terminal and/or C-terminal amino acid residues are not included when determining the percent identity relative to the reference polypeptide. In one embodiment, all residues are included when determining the percent identity relative to the reference polypeptide.

In another embodiment, the disclosure provides fusion proteins comprising the polypeptide of any embodiment disclosed herein fused to a functional polypeptide. In a further embodiment, the disclosure provides fusion proteins comprising two or more copies of the polypeptide of any embodiment disclosed herein. In one embodiment, the two or more copies of the polypeptide are identical; in another embodiment, the two or more copies of the polypeptide are not all identical.

In further embodiments, the disclosure provides scaffold comprising 2, 3, 4, 5, 6, 7, 8, 9, 10, 11, 12, 13, 14, 15, 16, 17, 18, 19, 20, or more copies of the polypeptide or fusion protein of any embodiment disclosed herein; nucleic acids encoding the polypeptide or fusion protein of any embodiment disclosed herein; expression vectors comprising the nucleic acid of any embodiment disclosed herein operatively linked to a suitable control sequence: host cells comprising the polypeptide, fusion protein, scaffold, nucleic acid, and/or expression vector of any embodiment disclosed herein; pharmaceutical compositions comprising: (a) the polypeptide, fusion protein, scaffold, nucleic acid, expression vector, and/or host cell of any embodiment disclosed herein; and (b) a pharmaceutically acceptable carrier; and uses of or methods for using the polypeptide, fusion protein, scaffold, nucleic acid, expression vector, host, and/or pharmaceutical composition of any embodiment disclosed herein for any suitable use as disclosed herein.

### DESCRIPTION OF THE FIGURES

FIG. 1(a-b). Overview of the de novo protein binder design pipeline. (a) The two stage de novo binder design approach. In the global search stage, billions of disembodied amino acids are docked onto the selected targeting region and the positioning of the scaffolds is guided by the favorable sidechain interactions. The interface sequences are then designed to maximize interaction with the target. In the focused search stage, the interface motifs are extracted, clustered. The privileged motifs are then selected to guide another round of docking and design. Designs are then selected for experimental characterization based on computational metrics. (b) The distribution of the top 1% of binders based on Rosetta™ ddg and contact molecular surface after pooling equal-CPU-time dock-and-design trajectories for each of the 13 target sites and averaging per-target distributions. The top 30 PatchDock™ outputs for the 1,000 helical scaffolds tested were designed using the RosettaScripts™ protocol. RifDock™ seeded with PatchDock™ outputs generated 300 outputs per scaffold which were trimmed to a total of 19,500 docks with “The Predictor” and subsequently designed. The top 150 RifDock™ outputs per scaffold were trimmed to 9,750, designed, and 300 motifs were extracted. The motifs were grafted into the scaffold set to produce 150,000 docks, which were trimmed to 9,750, designed, and combined with the earlier 9,750.

FIG. 2(a-d). De novo design of miniprotein binders to 13 target sites. (a) the 12 natural protein targets are shown as surface and their associated interacting partners in the native crystal structures are shown as cartoons. The PDB ID codes for the crystal structures that are used to generate the figures (a) are 3ZTJ (H3), 2IFG (TrkA), IDJS (FGFR2), 1MOX (EGFR), 3MJG (PDGFR), 4OGA (Insulin®), 5U8R (IGFIR), 2GY7 (Tie2), 3DI3 (IL-7Rα), 1XIW (CD3δ), 3KFD (TGF-β) and 4O3V (VirB8). The same structures are used for design, except for H3 (PDB: 4FNK), TrkA (PDB: 1WWW), FGFR2 (PDB: IEV2), EGFR (PDB: 1MOX, 4UV7) and InsulinR (PDB: 4ZXB). (b) High-resolution sequence mapping of the de novo designed miniprotein binders. The scaffold core residues and the interface core residues are well conserved while the non-interface surface positions are more tolerant to mutations, which indicates that the design models are largely correct. (c) Binding of the optimized designs with the corresponding targets in the biolayer interferometry experiments. Two-fold serial dilutions were tested for each binder and the highest concentration is labeled. For H3, TrkA, FGFR2, EGFR, PDGFR, IL-7Rα, CD3δ, TGF-β and VirB8, the biotinylated target proteins were loaded onto the Streptavidin (SA) biosensors, and the miniprotein binders were tested as the analytes for association and dissociation. The binding affinities of the miniprotein binders for InsulinR, IGF1R and Tie2 are weak and different experimental setups were used. For IGF1R and Tie2, the biotinylated targets were loaded onto the SA biosensors and the MBP— (mannose binding protein) tagged miniprotein binders were used as the analytes. For InsulinR, the miniprotein binder was immobilized onto the Amine Reactive Second-Generation (AR2G) Biosensors and the insulin receptor was used as the analyte. (d) Circular dichroism spectra at different temperatures (25° C., 95° C., 25° C. after melt) and (insert) CD signal at 222-nm wavelength as a function of temperature for the optimized designs.

FIG. 3(a-b). Designed binders have high target specificity. (a) Biolayer interferometry assay was used to characterize the cross reactivity of each miniprotein binder with each target protein. The biotinylated target proteins were loaded onto SA sensors and allowed to equilibrate before setting the baseline to zero. The BLI tips were then placed into 100 nM of the binders for 300 seconds. The tips were then placed into the buffer solution and the dissociation was monitored for an additional 600 seconds. The maximum response signal for each binder-target pair was normalized by the maximum response signal of the designed binder-target pair. The normalized values were used to plot the heatmap. The binding signals for the other target-binder pairs were too low to be determined at 100 nM and they were not included in the cross-reactivity assay. (b) Surface shape and electrostatic properties of the designed binders. The electrostatic potential surface of the de novo designed binders. The interface regions are shown for each binder. The electrostatic potential surfaces were generated with the APBS Electrostatics plugin in Pymol™.

FIG. 4(a-e). Ability of Rosetta™ to predict mutational effects. These graphs shows the observed experimental effect of each mutation versus Rosetta™ expected effect. For each plotted point, the delta refers to the effect versus the parent SSM design; therefore a “Beneficial” mutation is one that would improve affinity relative to the original designed protein the SSM was based on. The ΔExperimental ddg is derived from FACS data using the SC₅₀ values (see Methods). Confidence intervals were collapsed to their center point to make this graph and “No effect” refers to mutations with less than a 1 kcal/mol change. Binder region definitions: (a) Interface Core: residue contacts target protein and has no SASA (Solvent Accessible Surface Area) in bound state; (b) Interface Boundary: residue contacts target protein, but does have SASA: (c) Monomer Core: residue has no SASA and does not contact target; (d) Monomer Boundary: residue has intermediate SASA and does not contact target; (e) Monomer Surface: residue has full SASA and does not contact target. See Methods SSM Validation for further explanation.

FIG. 5(a-f). Mutations observed in SSM experiments that improved affinity bind at least 1 kcal/mol graphed by relative frequency. Plotted is the #_times_Native_to_Mutant_improved_affinity/#_times_Native_to_Mutant_tested_in SSMs. A value of 0.10 with x-axis F and y-axis W could therefore represent that for 2 of 20 times W was substituted for Y, the affinity improved. Separated bars on each axis represent pooled data for the entire row/column. Only SSM designs with a validation score of 0.005 or better were considered. While some cells are clipped, none extended beyond 0.25. Binder region definitions: (a) Interface Core: residue contacts target protein and has no SASA in bound state; (b) Interface Boundary: residue contacts target protein, but does have SASA: (c) Monomer Core: residue has no SASA and does not contact target; (d) Monomer Boundary: residue has intermediate SASA and does not contact target: (e) Monomer Surface: residue has full SASA and does not contact target; (f) All ((a-e) combined).

FIG. 6(a-e). Competition experiments indicated the miniprotein binders bound to the targeted region. Yeast cells displaying the TrkA binder (a), InsulinR binder (b), IGF1R binder (c), PDGFR binder (d) and Tie2 binder (e) were incubated with the target protein in the presence or absence of the native ligand as the competitor, and target protein binding to cells (y axis) was monitored with flow cytometry.

FIG. 7(a-b) Experimental characterization of the influenza hemagglutinin (HA) binder. The FI6v3 antibody competes with the binder for binding to the influenza A H1 hemagglutinin (a) and influenza A H3 hemagglutinin (b). Yeast cells displaying the H3 binder were incubated with 10 nM H1 or H3 in the presence or absence of 2 μM F16v3 antibody, and hemagglutinin binding to cells (y axis) was monitored with flow cytometry.

FIG. 8. Target success rate versus hydrophobicity. The y-axis shows what percentage of tested binders against the indicated target showed SC₅₀ below 4 μM. The x-axis shows the hydrophobicity of the target region in SAP units. A greater Asap score indicates greater hydrophobicity. The trend is striking and can be used to estimate the difficulty of potential future targets. The Δsap_score can be calculated on the target structure alone by observing the SAP score of all residues a potential binder would cover.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn: N), aspartic acid (Asp; D), arginine (Arg: R), cysteine (Cys: C), glutamic acid (Glu: E), glutamine (Gln; Q), glycine (Gly: G), histidine (His; H), isoleucine (Ile: I), leucine (Leu; L), lysine (Lys; K), methionine (Met; M), phenylalanine (Phe; F), proline (Pro; P), serine (Ser; S), threonine (Thr; T), tryptophan (Trp: W), tyrosine (Tyr; Y), and valine (Val; V).

In all embodiments of polypeptides disclosed herein, any N-terminal methionine residues are optional (i.e.: the N-terminal methionine residue may be present or may be deleted).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense: that is to say, in the sense of “including, but not limited to”. Words using the singular or plural number also include the plural and singular number, respectively %. Additionally, the words “wherein,” “above,” and “below” and words of similar import, when used in this application, shall refer to this application as a whole and not to any particular portions of the application.

In a first aspect, the disclosure provides polypeptides comprising an amino acid sequence at least 35%, 40%, 45% 50%, 55%, 60%, 65%, 70%, 75%, 80%, 85%, 90%, 91%, 92%, 93%, 94%, 95%, 96%, 97%, 98%, 99%, or 100% identical to the amino acid sequence of any one of SEQ ID NO: 1-1559, not including any functional domains added fused to the polypeptides (whether N-terminal, C-terminal, or internal), and wherein the 1, 2, 3, 4, or 5 N-terminal and/or C-terminal amino acid residues may be present or absent when considering the percent identity.

The reference pol-peptide sequences are provided in Tables 1-12. The polypeptides of the disclosure bind specifically to a defined protein target, including binding proteins for a diverse set of different protein targets, as detailed herein. Biophysical characterization demonstrates that exemplary binders tested are hyperstable and bind their targets with nanomolar to picomolar affinities.

TABLE 1
CD3d binding polypeptides

| SEQ | |
|---|---|
| ID | |
| NO | Sequence of target binding polypeptide |
| 1 | NHIACEIHNPEAAKEIAKVANVRRVFVIKQP |
| | GNRYFVLLKDADPEGVKKVASKYNARCVIRE |
| 141 | NHIACEIHNPEAAKEIAKVANVRRVYFIKQP |
| | GNRYFVLLKNADPEGVKKVRSKYNVRCVIRE |

TABLE 2
EGFR binding polypeptides

| SEQ | |
|---|---|
| ID | Sequence of target binding |
| NO | polypeptide |
| 2 | SDELFSKVELKVTELSMIVMNAKTEDEKKTA |
| | LTKIKQIADKVQDEELSKFVKRALEHVKKEV |
| | G |
| 3 | SPDEAKKLLQEAEKLARKQNDRMELAYVEFL |
| | KHVLENAKRLNDKRAVESVRELARDALEELQ |
| | S |
| 142 | SLDEAKKLLQEAEKLARKINDRMELAYVEFL |
| | KHILETAKKQNDKRTIESVRDMARDALEELQ |
| | S |
| 4 | SLEEVKELIRKLVPDPRLVWALEDLLELLKK |
| | GDPLAEEVLRFYLASAREQGDKDETKATELV |
| | LKT |
| 5 | DDESLKLLLILVQIQLALERGEISNDQAKEL |
| | AKRVEEKARKLGDEQVQRNVENVKEVIERFG |
| 6 | DHWEEVERWALELLQEATEQNDPTKAKKILE |
| | EAHKLLRRELSEEEARAVVRYLKQLVDRELS |
| 143 | DHWEEVERWALEHLQEATQQNDPQKAKKILE |
| | EAHKWERRELSEEEARAVVRWLKQLVDRELS |
| 7 | DEAAEEVLRYLKKLGDPELAELIERLLERVR |
| | KKKDPDLERTLEIIAVAVLYGDPEIARQALR |
| | ALH |
| 8 | DELRELINDAQNKNDPELLKKVLELIEELIR |
| | RGDPSYQEIVLLLLFVADLLGDPEITRLAEE |
| | LLK |
| 9 | SELSEEALRLLEEGNPRDAEHVLIDLLFELE |
| | NRGDKELSELVLEILELLIRGDPDEARKLIK |
| | ELT |
| 10 | PLEEVKKVVEEALKDDPELVRAVKTIIEAVK |
| | KGENDPIFLELLLRLLIETFGDPRLREALEL |
| | VRK |
| 11 | SDEERRELEERLERAVREGDEHHLRFLSQRL |
| | ILLWWIAGKLDREEARKYVEELIKELKKRV |
| 12 | DLEEEAEKLVKEAEKYARSVDDPAAKMHVHL |
| | VKLRLKLAKERISDKEKILRFVKEQVDLVKE |
| | T |
| 13 | DRHVADLIQHLLERGVPPEEILELIEDYIRW |
| | TGNPREKKALELLEKLTRELGDPEEALRLFV |
| | ERG |
| 14 | SPEDAEKLIKTIKTIAKKRNDRMELAYVKFL |
| | ESSLKQAQRYNDEERIEDITELLEDALKEIQ |
| | S |
| 15 | DYTVDLAIKFVEEVVKELKNDPDPRSQKLAE |
| | ELERLIRKAKNAEDPNKKHTLVHKAFDIAVQ |
| | AM |
| 16 | DEEQETRLKLLLLLVQLQHLAKKDPDTAETE |
| | IDKIYKLAKKLGDETIRENAENAKRIVQELK |
| 17 | SEEALKIFLAILRIQLHYERGEISPETAIRL |
| | LQKLIKRAEELGDEDVKRLAKRLLEKIRKEK |
| 18 | DIDQLIETLIKLLKKLSKKYPEFKFHARYAI |
| | ERANRIRKSDPRNSRAAVSLLEQALERIQKQ |
| | A |
| 19 | DYFFHSTRTYRGDGAEERAEKEAKEIVRKIA |
| | ETIARKLNAKRVTLQVIVVRRGDLILISLLF |
| | LIR |
| 1561 | DHWEEVERNALEHLQEATQQNDPQKAKKILE |
| EGFRn | EAHKKERRELSEEEARSVVRWLKQLVDREKS |
| 1562 | SLDEAKKLLQEAEKLARKLNDRTELAYVEFL |
| EGFRC | KHSLETAKKQNDKRTIESVRDMARDALEELQ |
| | S |

TABLE 3
FGFR2 binding polypeptides

| SEQ | |
|---|---|
| ID | |
| NO | Sequence of target binding polypeptide |
| 20 | SEELIKKALELLRQGNPDRAAQVLLFLAFQTGDPR |
| | VRELFSLLVEAMQKNDPELLKRVRRLLEET |
| 21 | LEKLERVALLAVRLYMRIGDPEIAKIWFKVMELLH |
| | AYQAGHLDEEEAKRRADKLEKELRKEI |
| 22 | DPSKELDKVYRTAFKRITSIPDKEKQKEVVKEATE |
| | LLRRIAKDEEEKKLASLISLFLKTES |
| 144 | DRRKEMDKVYRTAFKRITSTPDKEKRKEVVKEATE |
| | QLRRIAKDEEEKKKAAYMILELKTLG |
| 23 | DKKELSDTAEKLLKDALDKNDDSKFISAVFFALKV |
| | AKDVNDERLERIVVKLYNEFMNRYH |
| 24 | DYERVISRAIELALKKGDEKALHILNEIFFAAHRG |
| | ELDPTEAERLARRIEKKERS |
| 25 | DLREKLQELWWRALEKGNEKAERLALRAFMGAWHG |
| | DTSEEEAKKIITEVERLIKS |
| 26 | SEIHKKLARLLSKAIQKGDRKANHIVIRAAMAYEK |
| | GLIDPDQARKLAEKAERILRS |
| 27 | DIEHLLARAIHRSAQLGDREAVELLTRIFFALEAG |
| | KVSEEWAERLARTI LEHQRN |
| 28 | SIVERVIEQVEKRGLPPEALVLTVWLALVKAGDPE |
| | KARLVEELAREVFEGKLPPEELKRLLREIE |
| 29 | DEVHHFLLRLFFKDPDDEDVERILALFLLLKKEGI |
| | PPEEVTEIVIKFAEKLGNPELAKELKKILK |
| 30 | SEEERIAKLIIELLKRGINPERAVARVMFEIMELG |
| | DPELNHLVLRFFFTLKRDPEKFEELLRKLG |
| 31 | SKEERLKELLRLIKEAIKRNLTEEVERLAREAAEI |
| | AQELRDEEVLQEALLLWERFEIRRR |
| 32 | DELFQRVMELAFRLVLAAREKNDKDAEERAQRLER |
| | SVAKALESGDQDTAKTLVDRIEKYVKKEG |
| 33 | DPEKIIRELVRLALEAARKLNDDRVATAVLILLYA |
| | VLRLEKRDPEKAKKLLKEVEEKVKKLIS |
| 34 | DEVEELSKKVLHIATRLAFSPNPEDQQLWFKAMRL |
| | LDKAESIPDRDKATKVLRKAEKLVSSS |
| 35 | TPEEVMRTAFELLSEAARNNDRDRLKEIVKWVEDL |
| | VRKDPRVKDALEQVRLTLREMTHLAS |
| 36 | SERTASEVIMHAMQLGDHHVMREANRVYFGIVRGE |
| | LRPEDAKKLLDTLRRQLE |
| 37 | SEDESLKESLKHASKFFDIPSFEVRNSNGRLSVTV |
| | DGDDEKARMELITAQFAAMLRGLKLDVRTK |
| 38 | DEREILRKLIEISEELLRKATDEELRELLELVIAL |
| | SKEALRENDEELLEAAIRYNEVLIRNA |
| 39 | DFDTHEFEELEKVLEEARRKNDFRLAERAARLAAE |
| | SLERYRDAELVREALQEALLLWLALYK |
| 40 | PELDIAVVVAQIMADKIVKKASNPEIQKKAKTVKK |
| | LLEEARQKKDKDTVQKLWWKAVTLWQLVV |
| 41 | SERKLAHLLMIAVQQGDHKANVMLLRAVELLHKGE |
| | ASEEQADKIADEAKKRLS |
| 42 | DWKEIEKTARDLLKKARENNDRRTAFLVAVALKLA |
| | EIARKIQDPEERKRIEEVAKAALADA |
| 43 | TEEKIIRLWLDAARKGDRKANTLALRILEGLKRGE |
| | IDESQADKLADKIKKSLS |
| 44 | SRLEEIIRLAEELGNPYVAETLKMFLRAVRNGQLS |
| | PEWALANARALADYFGDPELLRLIEELIKK |
| 45 | NPYEELLRLIERLFERLQKENDEEVEKALSFVNAL |
| | LMLIARTTDEETKKELLRLALEKAKKVL |
| 46 | DEEEEAREHIQRQLEELEKHDPRAAVIFAAYIIER |
| | ARENNDETARKIAEKLLKEIEKRYRS |
| 47 | SEELKEEAKRLIKLLKDLARKASDRRERFILAALA |
| | ASLEKALKNNDEELERKLIEVAKSAIRRIQ |
| 48 | SREEVLHILNHLYFIAYERNDEELQHILARAMYIV |
| | FELLNRGDEESAKKVAEKAKKIVDKK |
| 49 | DKEELIEEAIEKLAPSPEAERYLRELLKEFKELGN |
| | PEAFLFLLWLAAEREGNPEVKRLVEELLRR |
| 50 | DDKEKTVKTIVRKLLRRAAHVDDPEVRTALLTLAY |
| | ILERALENQDDEALKRASKLAEKVLKSG |
| 51 | HETRDLNIRIETKREEELEHAERAQRLYFEAYLDA |
| | LRNRGNKVDAEIRLHIDGDTSHSRAHFRET |
| 52 | SEEHEKAREIAKKLAKRVRSGKLDSRQAKKIVARR |
| | YHHASRSGDKELAAILILVLFLLDEILRS |
| 53 | DEAQELARIVFELIVRLHRSPDPKDRKAAHFALRL |
| | LMKALKAQDPNERRSLLKTAIKYVRNT |
| 54 | DITWHARYT FHRHDEQAKREAESLAFLEVYKLER |
| | RAEELGLTSRFRIRSHEHNNRITIHLEIHIH |
| 55 | DPEEIKQRAEEIKKEFQKKGVSPEIQFAIEQVIKY |
| | ALEVGLSPKDIRTTIELTVRFAEELEK |
| 56 | SPEHAERLAKMLETLVKHAVKNPEERKTAETLIKQ |
| | VKELIKKGDEDSAQELLAKVYKIVWKAQH |
| 57 | DKDEVVQLALRILFEAYRRGNEEYYRKAQHLAALA |
| | FEAAEKNDEELLKKIEKKLRTLWKKLQS |
| 58 | DDDKEKKKKVKKKLDSIVEQAKKDPKRAMSLVLSI |
| | WMEAHRKNDEIVAKIAFEAWLKIMRIYSK |
| 59 | DEHLRKLLEKVMTLATKAWENNDEKVVSLLAEAQF |
| | WIALVDQDPRKKEEAQKRVETLLKQAEST |

TABLE 3A
FGFR2 binding polypeptides

| | |
|---|---|
| 60 | SEKLFKDINSLVISAEMAGVISEHEASRIERRLFE |
| | AYDRGLTEEVKKIKKEVKKLIKKARKS |
| 61 | SLLSQLLMEALQKGDPELNHLLLAVIFLAHERGIS |
| | PEEIDKLLEKLERELGDPREAIKLLLELLR |
| 62 | GPLNLLELTLWLASRRNPVARVLLRILRELRKRID |
| | PEEVLKILEEVARRLGDPFLLRNIEFILRL |
| 63 | SEEEKLTELSERVQKFFGLEHVEVRLNNGTITVTV |
| | RGDDERADQAIFFVREFAQLAGIPVDYKKQ |
| 64 | SEVELATKMIKHLAKQFGVPSVSVSYSNGRVRVTF |
| | KGNDDRVQQARLTALFVAHLVGVPLEVQTR |
| 65 | DPDEILDRVAQTLLTLAFQHPDDPELLKAFRRVVE |
| | ISQKAKKLSDPTQKERLARLAQRILEEVS |
| 66 | SIEEEILRWQFLAFKLGDPTLARLALRASHAYIEG |
| | DKKKARELIKKARKWY |
| 67 | SFEEVAELLRRAGLSPTEIITILYRLLRAFRHDPE |
| | RAKELVEELVKKLGLPPEARELLKELLTKH |
| 68 | TKEILQKVIEIVLKLVKKGVDPEQAVKIIRKILSE |
| | LIRQGKISKEEANMATVTAMFVAHLAKG |
| 69 | DEKTLAQEIIEEAKRAWEKYKDDPDELREFLRELL |
| | REAYELRLEEAIHFIQFLFFRLQLEG |
| 70 | DEIVRELEKLAKLPDAHMRLTVFLIRLFIHDPNDE |
| | KVEKLLAVLLELLLRGVSPEEIIKILKRVE |
| 71 | SSEKLKRAVEEARRKGDFELLWRIQLIALELGDEE |
| | VQNLAARAVMEMAKR |
| 72 | DPKEEAKKVTRLLLKLAHNADDDEIRVELLTLAYA |
| | LRRALERNDEEALKRVISKARKFIKDVKS |
| 73 | SRDELVEENIRFLARSAGVPSVSVSVSNGRISVTV |
| | DGDDEAAQELSSQALLYAHLAGLDLKVTIR |
| 74 | DEEKEKLRELAHESIRVALKRARSDEDHLRIARLY |
| | LALEKAVENNDKDLVKHLLEITTELAKRL |
| 75 | DKSDAEKILREALRLVWELGDRELFHLVSRLYLAF |
| | VRADKNNDKDLLDEITRQAEELLKRIKS |
| 76 | NWEEAAALALESASRAGDRELNEEILRIFFAVRRG |
| | EISEEEAHEMLREIAEKSR |
| 77 | SELLEKELRELLKNGNLEQAVLLIWFALHEAGDPE |
| | RAEEVWKLLREALERGDPDLVRKIIEEALK |
| 78 | SWEELYEKAKKLVEQAKKENDEDKLNKATLLVYMA |
| | HLAASELGDPTAQTSVERLAAHVFHVAI |
| 79 | SEEIEELIKELFRKARNPEVRTVLATLWLALERAR |
| | SPEEREEIERLERLIEEDPRLAREILKKLN |
| 80 | DSKELKRLLKEAEKKGDLELANRVAFLAHELGDEE |
| | VLRLASEVLSRIMG |
| 81 | SEIHKKLSELMMQAMQKGDEKALHIVNHAYFAYER |
| | GEIDPDQARKLAEEAERLLRS |
| 82 | GEEVLEKVERLAGGDPRLIAYALLRLFREARRRGD |
| | KDLLKKIEEAVELLERKDPELARLIKNSLP |
| 83 | DEWDERLKETFKKLQELLKQGKEEEAEKILKELIH |
| | EAEERNDEEAVQKALLLWERLLTERRK |
| 84 | DHWKEVEKWADTLLDEATKQNDPQKALFILFMAFL |
| | LLRRELSEEEAQRVISKLIRKVHERLR |
| 85 | DEEEEIRELIEKAVPDPELRRLLKENLKRLEEDGV |
| | PPEEILLTLAFAAERYGDPEVARLLFELLR |
| 86 | SERELFQRNVEFLARTAGVPSVEVRYTNGKYHVHL |
| | HGDNEALKDAKRQVELFAMFANLDVKVTTH |
| 87 | DETTKTAETLIKQLAEKAKNVPDPINVLILLFAAM |
| | LLAKEANDPRLEKLVDKLVREVLQYV |
| 88 | SEEELRRLKELLKKVNSPEAQMVLVTLLYLEHLGV |
| | SPEMVLRLLEEMLKRINPEALKLVKELLRR |
| 89 | DRTEILTLAFAILLMAKEKQIPEIEKLARKVVHLF |
| | SAVQRGELDETEATKRARHLWSLASSY |
| 90 | DEIQRELNHVYFEAHERGDEELVQKVTRVLYALFA |
| | GKIDKKEARKLVEKLKKLLS |
| 91 | DEIRETHHLILRLFFWAEEKNDREAEKIAARLLEL |
| | QAHALARRDEELLKKISKEAAKIVKELLK |
| 92 | DPDHAARLVNVLAFLAVEKNDEEAQREVSELIMIV |
| | MRAKENNDEEAVKEVEKRAKELLSKE |
| 93 | SEIEELLVHLLRLARELGNREALHVVLRAIIAYKR |
| | GRADEKQIRKLIKKAKSLLS |
| 94 | EVDELQKIAKKVRELAKKQNDEAALRTVEIALRAL |
| | ERAKQKNDEREVEAAKLAIAAAREHVEG |
| 95 | DQETVKKLESKADELAKKITDPRKRFLASVVKVNA |
| | RRAKNAEDPNDQTELIELAEIILRHLG |
| 96 | DQEEAEKILQRLLRIAYEARDEEFFLEVSLLAFAH |
| | ELALEEGDHEALEESIRIAEELLKEA |
| 97 | DEELKTLEKIVARLFHHAARNNDRKSLELATRAFF |
| | ALVEAKNADEEQQKKLKTLVKQLIKKIK |
| 98 | DLEEEADHLLSRVLLVAFERNDEEVNHIVLQLVFL |
| | LQKAKEENDEDKLKTLLKKAIEIARKLLS |
| 99 | SESEILKHSVSQAAKFLGVPHFEIHDNNGKFTVTV |
| | RGPDHVSRMLKLTVRFIAHQLGLDVKVTTQ |
| 100 | SEEETFKRLVQEWARKAGVPRVFVTTYNNKVYVLI |
| | HGNDESAKRLTKIVKKLAKEIGIDVKVQTH |
| 101 | KVVEQIILFAKFLGNPEVAEIIEKLLRELKERGVS |
| | PEEIERIIEELLEELGNETLLRNFRYLLEQ |
| 102 | DDDKEKKKKVKKKLDSIVEQAKKNPNHAMVLVERI |
| | LEEAQKKNDEIVEKIAEEANHKILRIFFK |
| 103 | SEEDKEHVAMWVIFLATQAAHDEDLERKARKKLDK |
| | ILKDLKVPEAEEFKKMFDEIVKQVRSMLK |
| 104 | SEIAEKLERLAVKAYEKGDRKALRIIARAQIAYAK |
| | GEIDPDQARKLAEKAERILRS |
| 105 | GEELEEIVRELRKRGISPIQILFILMEILFEKGDP |
| | VAHLLSQLIFAVAQGDIDPEEALELIIRLK |
| 106 | TEEYRITIHNRNDSEEEILKKETKAITLIYRILRK |
| | AEERGLRAHTRLSRTHRNNTTTERVTITVQ |
| 107 | PELVELLIEFVKRYDPELAEKLEENLKRLEEQGVP |
| | PEVIIFTLLLAVRAKDEELARRLFELVRRG |
| 108 | GEKYKEIVKSASRHAKEASKKNDKHLLHMLITFIK |
| | TIADTVPDEDVTKYAKKVATKLEQNG |
| 109 | DDEEVKTWEKLAKEAINRIPDKDTRTEVEKMVRML |
| | IKTAHSPDPTEVALALSLVEQIIREFRG |
| 110 | TEREAVKESLERAAKLFGVPSVEIDTRNGKVTVTV |
| | QGNDEAAKQLSFFAKFLALLAGVDLQVRHR |
| 111 | DLQEEIKRALKEGDEELVEKLLRELSKRSGAPPAL |
| | VLNNILVQLEEEGVIPPEELRKILEKFAKK |
| 112 | DLEEQAQLTARFVAHLVGVESVRVTVRNGKVSVTV |
| | TGTDEKAQKASRMIKFALKSFGLDVKVQVR |
| 113 | SEVSELARWVAANARAVKRRLEDPTKRKKAEKAEK |
| | LAKKAGHDPDEELARRLLTHLMMMLIEL |
| 114 | SQIEKMLRELTALARHKGDDKARKIVERAIERYRK |
| | GKIDPDQARKLAVKAFEILQS |
| 115 | DEEEEKQKEVDRVESHARRIPDKEKAQRFVKKYIE |
| | EARRRNDPKLVEMLAMVLTMIKALSEI |
| 116 | SEELASDVLRLALEAGDMEAANRATMIAMALITGD |
| | LSDKEAKKLLKKLKKEISS |
| 117 | DEEEKRVEELLRDPELIERFLAELLEEAVERGDPE |
| | LNHLVLAVLFALRRGDKDLLKKLLEELIRK |
| 118 | SLHELVALLWIRVPDPELEREILRVFFAIERGELD |
| | PKELARLIEELAKKVGDEEVLELVRKLLEK |
| 119 | DVRNLLALARTLAEQAAREDSEHARKTIESLSRFV |
| | DKLLKNVRTEELKKLAKKVKKTIESLERK |
| 120 | DIVEEAHKLLSRAMSEAMENDDPDKLRRANELYFK |
| | LEEALKNNDPKTSQELAKEVVTWAQSD |
| 121 | DMVEKLTTEAERVATKLERSPDPRNKHVARVIKSA |
| | IKQALSDSDKDSATLILFFAVNLARTF |
| 122 | DEKKELRHLLKIVLELNRKSTDDEARTETQTLAFA |
| | AILAEEKGDLELAIRILRKAIKIAEESVK |
| 123 | DSKEIEELRDKIRKIAKEVQNPRVALAALEVHTLL |
| | RHVRAGELDETRATELARAAFRQASQS |
| 124 | SRLEEALRLLAEVAGLPPEEIRRIFEEYRKEAGDP |
| | RAAIFTLLLALRERGLIPPEVLREVIHLVE |
| 125 | DEELELRALLIIYRILRALEKIHDEEERKRFMEDM |
| | IRKFREIFHVEEMIRLFERVLRRAAERAKG |
| 126 | SEELKKRAEQLIRELKDKARKASRERERFILVALA |
| | ASLERALKENDEELERRIIELIEAQERHQ |
| 127 | DYRELMSEAVQLILKALSLPDPRAHQRAQRLAFIL |
| | AHANNVPDETTAKQLIETAWREIQKVLG |
| 128 | PEYEKIVETLTKQAEEAIRKNDEEKLSRVFELVVR |
| | FYEEADESGDPRLRDLAHKLNHRVLRLEF |
| 129 | EPEEKARTFEELVRKAYKKDPSKAIHLHNRLLWEL |
| | LEEARRKNDEKLYRLAVELYVKLYRYLE |
| 130 | TETERKAHRLAAELFKKARKANNEELAETALHLTT |
| | LLYALERARTEEERKRILEELERFLREWER |
| 131 | GSVDELSKLVHKLYRIAFEKVQNPEERRKVQFVAF |
| | QAHLALKSSDPEEQKKAEELVKTLKKLV |
| 132 | SLEEEVERLLKEAGVPPEVIELIKELIERLKERGF |
| | PPEAILFTIVELLEELGNPEAARLIFELLR |
| 133 | DENKDKLRKLKEKVDKASKNKDKDTLTKLWFEAFK |
| | LARDVGDPHVIRVVAELAVRIFALIHR |
| 134 | SEEEIKRAAKLLGVPPEELKRYERRLLEDAGDPEA |
| | VKFTLALAIEKAGLPPEAEELLKKVLEILT |
| 135 | PPDEYLKTLIRLVHELFTSGDPHKQHLASRVLTLV |
| | FQAHLAEDPNEKETLIRKAQKLIKKAG |
| 136 | SEEEVKQRVTELIELWKKSGDDRFLEEAERILVKF |
| | YHEAAKRQDKRLIRTASRLFFKLEEIKRK |
| 137 | SEEEAIEMAVVSINQLLSPEAREILKELLKRVKKG |
| | DPEATRIISTAFYLARRDPEEATEFIKKLR |
| 138 | SKEEETTRLYELLLKNPEEFKKEIEELIRKTGDPR |
| | LEELLRLIEEALKEGDPFKLIFLVTLLLET |
| 139 | SKEEEVRELLRLLEEGDPHAAMRLLFLVTEAAGGD |
| | PDLANEIYERLLRLLKKNEEEAVKLLVELR |
| 140 | SEMTRKAIEMILNAAKNTARDPRIQKKIDKVLTKE |
| | KEAVKKNDEDKLREVAEQAFELSYQAVT |
| 145 | SETDKVKKVVDTLLTKAKQQQDPELAQEASSFAIE |
| | VINKNPRDETVRELVVKANMVFLQLHS |
| 146 | DDSEVETAKKLVDEVKKHSNDPRVKFIATAVKLNL |
| | DEAKKNNDEERLKSAVEMARVLYKEAM |
| 147 | DEELVEKAREALRKNDPRAAEVIFRLQIKAMEEGD |
| | EEKLRLAVVALWLFAEGDPDKAEKVLSKVP |
| 148 | SYEEMLKALLKEARKAGVPKAQKIIKKALKLLQEG |
| | KLDMDQLTRLMWEAWHLIS |
| 149 | SSEDIKKTATKAYKKGDYEALFKASYIAWQEGDED |
| | AASYVLRLLWEAAS |
| 150 | TKELVRKLKELLEKGDPRWESLISKIMMLASELGD |
| | PELLHLLNLAYFLYEEGDPRVREVLKELEK |
| 151 | DEEEIEELLKRAGLLSPKQVESLKEFVRRMKELGI |
| | SPEEVLEQLLLTERFIGDEEAVKLLESLLK |
| 152 | SRRELFQRNVEKIAKSAGVPSVEVRYTNGKYHVHL |
| | HGDNRELKIAKNQVELFAMFANLDVKVTTH |
| 153 | DRHQEFIRLIWETLRISRENNNEESHHLAAEAFLI |
| | WARALQNNIEEELKKAEEIAEKSLRLAR |
| 154 | DAVEQAELFLREMNSPVAREILKVLKELKERGVSP |
| | EEILRLLTEVAEKLGDPQLARNLRFFLEID |
| 155 | SEHVLKNIKKVVEHETKEIPDSKQAREQAKLFIRF |
| | ALRHIEDPDERRKVEEEATKLVQKI |
| 156 | DEIYRELFRLAEELNDPELLMLLLTAAFMLSHGNP |
| | DVERLLKRVLEVLEKGDPEEAKKLLKKLLK |
| 157 | DEIEEKLARLLWRALELGDDEAARLALEAQRLVAR |
| | GEIDPSQAKKLAKKLEKLVRS |
| 158 | DPRKKLKELVQRALRLAKEVGDEEILTLVFALHLA |
| | AERAIKNNDEDTLKRVEELLKRAIKKLES |
| 159 | SEEEKKELEELVRRAEEALKRNDDERVKEVLQRLY |
| | ELAARLEDREILHFVNRIHEKLYEKE |
| 160 | DRELAHLLLEAAERNDPKLNQLVLVAFFLLERGVP |
| | PEEIERLLRELAEELGVPPELLERITRVVR |
| 161 | PLEEELEKALKEGDPDKLHRLLTRIMMEASRRGNK |
| | EELRLINIIYFAIEEGNPEMAEKAIKLLQK |
| 162 | DELINEIIQLAVKDPEKAKELIKEIVKELRKKDPR |
| | LVKEFLEQLLLTLRELGDPELVKLVEKLLR |
| 163 | SPEEVLLELARKHNIEQAIQLLWLAAARDEELYKH |
| | VEELVRRNLEHVIVEQAKELLDRAR |
| 164 | DKEAIENYIRLLKRETENISDEELARQIRKKLKKA |
| | EELLRRGLEEEANELLIRIYFELRRR |
| 165 | SFEELAKEYKRTGDPRYLALLFQLLFELQFETGDP |
| | RLTRLIWMLMEAMKRNDPDLVEKLARLAEK |
| 166 | TELLERVVRLVKEGDPEKAEELLARLYLRLAREGD |
| | PETLRFVIAVELALARGNPDEAEKVVRQLK |
| 167 | SVVEKAFRAAYLALNKGDEKAVREAQRALFLYYRG |
| | EADKNQLDKIIETIIRRLRS |
| 168 | DLEELVRELLERNPEFKRLLEELREAEEEGDEELV |
| | ILLRLTLALALAEFLNIPPEEARKALELLT |
| 169 | SLDRALAAALEPARELGDPRVNHIVLQIIFELRHG |
| | VNPEELERALVERLRQVNPEVARLVEELLK |
| 170 | TTEEISKEIYRKAWKLAESVPDPHLREKAERVILT |
| | LSLMVLLIPNPEEAKKLAKKLEEIVRKIV |
| 171 | SVEEILEELSKKLKDPRIEEVRERLEEIVRRLNVS |
| | PIIAAFILSFELFERGDPRASIVVTALFLA |
| 172 | PPSQEFFLRLVEKVVKKAKQIPDPRSQKIAKRLEE |
| | LVKRARQVEDPKEKLHLVAVAAFLAAKVV |
| 173 | SPEDEKLREELKRLYKIIKNVEDPKEATKIWFRAM |
| | EIFENVTDPTLQTLARLVNHLALRVFLR |
| 174 | SREEAIQKSVEHALKFLGVPSFSVSISNNTLKVTV |
| | KGDDSAARMAEFSVKSIAMLAGVPVEVRLK |
| 175 | DKEELIEEAIEKLAPSPEAERYLREALKEFKELGN |
| | PEAFLFLLWFAAKRLGNPEVERLVRELLRR |
| 176 | SRDELLRRNVEFLARVAGIPSVSVTVENGTVKVKV |
| | KGNDERAQFFSEQVKLFARFIGIPVQVDLR |
| 177 | DRTEAARKAIDRAAKWFGVPSWSVDVRNGTLHVKI |
| | DGTDYSVKQLSFFAKFLAQLVGLPLKVDVR |
| 178 | PEEEEIETRARKKATHVVEVAKKNPEKAKRLIARL |
| | LVEAMEKNDHILNHVVNHAYFLIEEVERS |
| 179 | DRTETARRSIDRAAKWFGVPSWSVDVRNGTLHVKI |
| | DGTDDSVKELSLFARELAHLVGLPLKVDVR |
| 180 | DEHKELKELYDHILKLLRNAKTEEEVQELARKSVQ |
| | LAELLNIEELVQESLLLWLRLHNRLK |
| 181 | SIEEEILRWIMLAHKLGDPTLQSLAIEASHAYWNG |
| | DKKKARELIKKARKWY |
| 182 | SEEYRKKLREAVELGDPELVAQLLLTLAFLTGDPR |
| | LQELFSMLSELIFRGDPSLEEILKLVKELG |
| 183 | DEEEEKEKEVHKVLQTALRIPDKEKAQRFVKKYIE |
| | EARRRNDPKLEHLLRYVLMVIKKESDT |
| 184 | SFLETLKHLAEYLGDPELRKLLERLEELLKRGVSP |
| | EELLRIALRVARELGNPMFALTVEYLLHVP |
| 185 | SDEKVKEALKEVEKAVKKAGLDPKVVEWAERAAKE |
| | MKRRGISDDQISLFLRFVVELIRKNSS |
| 186 | SEELVERAKRLAGGDPRRALYLLLLALARRLNLPP |
| | RELAKILQKLRREAGGDPELLLKLLAELVE |
| 187 | NYERVIAHAARLAAELGDREALRRLNEIFFGAREG |
| | RIPPELAERLARRIEKKLRS |
| 188 | SLLSKIQELLERGDPEKVEELLRRLVELLRRDPDN |
| | EHLAMLLMQAVTIVAGGDPERANELFKRER |
| 189 | DEDEQVRLTVLYVAHLAGVPSVEVDRSNGRFTVTF |
| | DGDDDSARTASKTVKRMLTKMGLEVDVKTR |
| 190 | DPKEEAKKVTRELLKLAVNADDDEIRVELVTLAYA |
| | LKRALERNDEEALKRVISKARKFIKDVKS |
| 191 | SKEEWFSLAVKVSARYAGVPDVKVRVSNGRFEVTI |
| | NGDTDSARLAQQMVRDLAEIMGVEVDVQVR |
| 192 | SEEEIREIIETWKKDPKKAHRLVNRLLFEAYERGD |
| | PEKQSVAIRAAYLLYEGNPEEAEEVLKEIQ |
| 193 | DLEELLEEALREAGEPPESIERAKRRVEELLRRDP |
| | REAAKFVALFLQFVGNPELLKKVREIFEKT |
| 194 | PEEADKILEHLQKLFREALENRDEEAIAFIATLYL |
| | AAARAKRNNDQEALERVKKLLKEFKERKK |
| 195 | GEEEALKILAEVLGRPPEEIRKLVEKIARELRVPP |
| | EHAIKALALTIAFRINDPRVLRLIAEALEK |
| 196 | SEEERIKTSIERAAKWFGVPSEDIQTRNNRIHVTI |
| | DGDDDSARQLKLFIRELAEVNGVQVHVTIK |
| 197 | SIVEAVIEQVEKRGFPPEVLVATVWLALEKAGDPE |
| | KARLVQELAREVERGELPPEELKRLLREIE |
| 198 | SDDMRVLALAVSLMMFAQKQNDEKVEKTAITLWVQ |
| | LMDARLRNDEETTDKISKLIKDLATKLKS |
| 199 | DVEQLLLRYAWRALELGLEEHAVFIFRLLALVAKG |
| | EIDEREAHKLLEESRKRLEN |
| 200 | SEEAIRMLQSLEKTDPEVQAQFLEVLIEHRVPDPE |
| | ERRRLKETLKEIVERNDEELFERLVEEVER |
| 201 | PKEEEIKKLLKKLIKLAKELSNTPEARRMVKALER |
| | TVKELVKKTSPEDALKFAALALVSIEYIS |
| 202 | NSEEIKKEIKKAEKKGDSQKLLQLLWLAIELGDEE |
| | AAVFALEAQSRVMS |
| 203 | DEELKKQAEKLARLLVRLAAEVPNEEIALELITLA |
| | YALERASRNDDDERLKKVIDTAKEVVSKG |
| 204 | REEKIERLVREAERRIREGKISAQEVFEVQHRVYE |
| | LLRKKGDPRVEKLLHKIMRLLWMLAK |
| 205 | DEIERKLLKLSIIAWELGDQKALNLAASALFAVVA |
| | GEIDPSQAKKLAKKLEKLVRS |
| 206 | DKEELIEEAIEKLAPSPEAERYLREELKEFKELGN |
| | PEAFLFLLWLAAERLGNPEVKELVRRLLRR |
| 207 | SEQDVVRKNAERILKLVGVPHYEVSKSNGTVTVTV |
| | KGDDERARLALNQVKLFAMFLGVNVQVRIK |
| 208 | PAEEILRELLHRDPRDKVVVLLLYALLRAREEGRE |
| | SPEELIELLVRLARRAGNPELAKLLERLLR |
| 209 | NEERRRAIERILTRAKKLARDPRIQKKIDKVLTKF |
| | KEAVKKNDEEKLEEVLILATTLWLRAHK |
| 210 | KDELTEHVLKQLRQVLEEIRDYDRVREAAKLLAEF |
| | FRELDPRAEEAAKKALKIFEEELRKKQG |
| 211 | SLEDINRLIFIAHKLGDDKALRVLSHALMAWEKGD |
| | EELARKLVKKARKLLS |
| 212 | TEEIKRAVKHSVKDMVKNGLDPEWAVRVIRTFLEE |
| | MIRQGKISKEEAKEATKVAEKVAKKAKG |
| 213 | NFFHVVWRAQRIAMKKGDEKALSILARIAMEAAQG |
| | KLDPTKAERLARRIEKKLRS |
| 214 | DHEEKAVRLLWAILEVAEKKDDEKLHALVAQLYEQ |
| | LVEAAERNDEDSLKEITKKIEELLKKASS |
| 215 | DDHKEVIQSMLKSALRELKQYARDDSIREKVERLA |
| | TEASRKNDHHLANRVLFLIVKLIKDS |
| 216 | STVSREVETIHTSNPEEVKKWKKEAQKRAEKIAKK |
| | YRKQGLDVKVHTFEVRRGDTVIIIVEVRIN |
| 217 | IEEAKKLAMLVSMAAQMLAEGRLDERKWKKIVKEA |
| | SKILKKLGISEEEIRNFQKSAKELKKQF |
| 218 | SEKEEFIIENVKKLGDPNQIELFIAFAVKLSGDPR |
| | LKELLEIVKELIERGVSPEEILRIIEEFLK |
| 219 | SDRVAQFLLFLAFQTGDPRLRILFSVVVRLEEEGV |
| | SPEEIKEILEKVAKELGNEEVIREIKKLLD |
| 220 | SEIANEIMRVLWEALERGDEELALKALHVSHLLWT |
| | GEIPDEEAKKLAEKLKKLLS |
| 221 | DEREHARQRIEFLSALAQQIRRDEALEKAIRLMEE |
| | LARWAGIEDIERLVEEAIRRSRRAVKRE |
| 222 | SEAERLLSHLARIAAEKGNEKALHVVNRAIIALIE |
| | GLADEKQIRKLLKKAKSLLS |
| 223 | DKEEKITEFSQKLKEKVEKASEEEAERLIAKAYHE |
| | AMQRNDPDLALVILRVMFEFREKRS |
| 224 | DEEEAERLLAKLLRKARSPEERRLLTAVFFALREG |
| | DPETARRIVEELLRRVGDPELKRVLELIEK |
| 225 | SSSQRYQIRVDSSDPKDTEQAHRDAKTIAQRYAEE |
| | LRRQGNPVSYDLNYHRLNNLWMVVVHVRTK |
| 226 | KEWRDKLKSRLETVVKKAQKDPSYEEEAERVISEA |
| | MSIAMEEHDPEALHEANKAYFELHEYM |
| 227 | PEEDEEFLKAAFRTAQEALKEGDKRRAEFIIAAAE |
| | LYARHLDVEELKELAKELSEQFKEEVKR |
| 228 | SEEQRKLAEKLLSKAQEALKKNDTETLEKVVRRVL |
| | EIALKYDENPYIHKVAIDASLLWLMAAER |
| 229 | SRDEQFKKLVKDVKTLAKRQNDEEVLKAVERATKL |
| | FREAKKKNDEEKALRAWFALAIALEKLS |
| 230 | DELREILREALEKGDPRLVLQLLALLAFLAHRTGD |
| | PRLVKVYFIVAEALDKKDPELLKKALKLLK |
| 231 | SPEKVLLELARKHNIHQAIQLLWLAAAKDEELYKH |
| | VEELVRRNLEHVIVEQAKELLDRAR |
| 232 | SEHEAATESIKQAAKTIGVPHVEVDTRNGTLKVRI |
| | KGDDDAARMLQMSVKSVARLANLKVDVRTE |
| 233 | DVEKLVKEVEKLARKRGDRRAAFIARAILAAFRNG |
| | KLSSEAAERALKKLAKRLKG |
| 234 | EELKKILTELKKKATKALRNNDQSKVQHVIMVLAE |
| | IIHKAQTPEVRRTAAKLAFTLMRLTIQ |
| 235 | DETIHVQSEARWTVEEERRRALRWFARVSTRARRV |
| | ADRLNVDVEFHVEHRFHGEKVVIRIVVHER |
| 236 | SEDDEKVSEIFLRAYEIALKKNDPDQLSELNRWAF |
| | LFVEAQQRNDEELVKRLAKKFEEWVSKAQ |
| 237 | DEEAKYVHLMVESLRYYAKQAKDPTLAKQIKELAD |
| | KAERDNDPETVREALKVVTKAQKKSH |
| 238 | DYQKVNKLLDKATEIARKQNNPDVVFILVALKLSI |
| | EWAVKENDEEALKQLEHIASRFAKKE |
| 239 | PELDRMVHMAQRIADKIVKKASNPEIQKKAKTVKK |
| | LLEEARQKKDDETVIRLLMIAELLFLSVE |
| 240 | DDLKEKAKTLLELVKEASKKNDQKTERELSLELVA |
| | VEMEAQDPNVRHLVEKAYFIAMELAFK |
| 241 | SKEFVDSLLKHVEKLAKEVPNPEAKKILTKIKRLL |
| | EEANRRNDEAKAYLLVFTAWHQIRQLK |
| 242 | SFMSVIQRAINLAMKNGDEKAMNILLRIQFLALQG |
| | KMSEEQAEKRARRIEKKLKS |
| 243 | SEEEHVRKVIETMAKRFGVPDVKVSVRNGTVHVKV |
| | RGTDDAARQARLTAMFFAHLLGVPVSVTIR |
| 244 | DEKVAVKVLVVAFRLWDKTGDDSARKAILYASRVF |
| | MAAMNGDLSEKDRKKLEKTLRKIEKKLRS |
| 245 | STIEFAISVAKFYLEELRKLGLISDEEAKRIKEEL |
| | DELLRRGDERRVSQILNTLVELVVRG |
| 246 | SEEKAKELVEALKRRVEEAGDEELKKRIEKLVQEA |
| | RKKGLSWEEIATIVETLILAFETKKK |
| 247 | DAVERLERQVEKLVKEVINSGNPNSERAQRLLKLL |
| | KELLKKATSKEEQALAALTIALAVANA |
| 248 | SLQEQLERIIKTIAKRAGVPQVKVRERNNKVEVTV |
| | DGANQAAALVHLAVVRVARRLGLDVHVRVK |
| 249 | TTQETLVKSMIKQLKDVADNRDDPTVKKTLKTLAK |
| | ILEKALKNNDMSAINRALFLAMEVLKSG |
| 250 | SSKELKKRIEKAEKTGDVHKVMELALIAFMRGDQE |
| | AARLANRALFRLYNR |
| 251 | SQDRREDAKEIIKELVKIGNEIDARSVIMFLAYEI |
| | SEEEGLDEDQASEVLQELHEVLERLL |
| 252 | NWEELVKRAQTLIKRAQDTGDKSYVNEAWSVANVA |
| | AFLAVELNDPEVQHIVSKLLEQIITVADS |
| 253 | SKEEVVERAVRLLEEGNPFEASILIQMELLRLGIS |
| | PHEIVELINREMELFKRDPEEAKRFLREVG |
| 254 | PHVEELVKTIVSKAEEAIRKNDQETLSRLIETLFE |
| | LQKKDPRERDAAAVALLLIAELQESN |
| 255 | DLREEFQKVVKKAQEAAKNKDHEKAIRLVWRALEI |
| | ARKIGDEELERQVARVLLQVHELLR |
| 256 | SREEAVELYHIAVEKGNEEAARIAAAVYLGLARGE |
| | LDEDEARKLLEKVKKLLS |
| 257 | DEELKRAAKSMLDQAKKLADQISNDDLRKKAQSLV |
| | EKARKIDDPTVALTVAFTVRTIYEEVQS |
| 258 | DYREALRLLMAILELSERRNDEESHHEAARAYNHL |
| | SRARERNDEEAIKEVVEQLREQLERIEG |
| 259 | PERLRELLEESLRLAEERGDEELVNRTILLAFAFE |
| | IAKRRGDEERLKHLARLIEELWHENRN |
| 260 | SEEDKIQTLIDEVRKAAKRLYPDDEEVKKIESNAQ |
| | ELVDQAQDQELAKLSERELKHLLDSE |
| 261 | DISQELSKIVKSLIDELKKSPDPKDKHAAELAESA |
| | WRQAEEAQDPHERALILFFAISVVRNT |
| 262 | SRDELLRRNVEHLARLAGIPSVSVTVENGTVKVKV |
| | KGNDERAQFFSDQVKLFARFAGIPVQVDLR |
| 263 | DDDKKEARKEAALFAQAVRQGRLDKRAAKEAVRRR |
| | IKKARKSGDERLAKILELVLKLLDKILRS |
| 264 | DEILHKLARLLWRALELGDEKALKLALEAQKAVAR |
| | GEIDPSQAKKLAKKLEKLVRS |
| 265 | DEEERFREHLRRQLHELLRTDLRAAWIFVAYALER |
| | AKENNDELARKIAEELMRETEELIKN |
| 266 | GPEEFIELYRRWLKLGLSPITIANVILFAAYERND |
| | PDLIRAAAIFMLLIQRFDPEEALRLLQELG |
| 267 | DIEEFIETFLRSHNEDLRRIGTWSEEELKELTKLL |
| | RKLAEELDDEREIKNLLILIAFRSQLK |
| 268 | DEQELVKKVKTILTEASKKNDKEKVREALSLATRV |
| | MLHAKDAKVFEQLFKLELYALHVIKS |
| 269 | TRDELTKKNIEKLAKIAGVPSVKVTRSNGRWKVTE |
| | SGDDEAARDASNQILLFARFANLEVELTLK |
| 270 | NLERKLVRLYNEAVKLGDKDLMSRAVAVFFLIEDG |
| | RISDDDIRKLVEEVERLVR |
| 271 | DEEAFKIIFKLTVTVNKLLRKNPEQAKEFVKRVLK |
| | EAKEKNDHRTYMAAKAILRLIKEHG |
| 272 | PEDDEVEHTLVRVLWKAAEDGNSEVEEKAFRLQEL |
| | FMEAKKKNDEETLKKVITKAKKVIKKQ |
| 273 | SEEEHRKKLETELRKIREALKREEEAKKTKKWLRR |
| | LFREAVEKRDEELARLAALLYLAFERLR |
| 274 | ARRYTLTVRVPTGDPTETKKAEKRAKKFATDVSEK |
| | WKRNGYTVRFRVDVLRNGDRISVIVIVIIK |
| 275 | DLKKLVSRVVENLAEEIKKQASPDEQKKLDTLLEQ |
| | LKKALKNDDESTASKLWMIISEIWVRER |
| 276 | DEILEEIEEALRRGDPDLVERLISKLMLLAAELGD |
| | PKLLHAANRLYFALKKSPELVKEILEEVRK |
| 277 | SKEEKMKEAALRLTLIAQRLGVDRKYMHTAVRIFV |
| | KLVKNGVDLDQARRIAEKWLKKVVKS |
| 278 | TITESNTIILNGNSERVQKWAEEWLEEWRRKLSEL |
| | AKRIGLEIKHRIHRERDGKFIVIRIRSRIK |
| 279 | DESKKILDHLKTLLEKAIKNNDTETLNHLVLHLFF |
| | LARELNDEEAEKVAVEAYEVVHRLLR |
| 280 | PIEEEVKELIKIVEEALKNNDRETVSKVLFRALRL |
| | AVEVNNEELISRVNRLAFIAFTVT |
| 281 | DEEIEKLLEEVIGDPRIAREVLELFKELRKRIDPK |
| | EVLMMIIVLLNRLGVSPEVINLVLKILLLP |
| 282 | DWREYSKKLKKALKRAAKDPHDAEAYHEIFRILLA |
| | LEEIVPDPRIAEKLAQEASKQAREEVR |
| 283 | SEETKRIKKWAKKLAKKAGLDEETIKQAQKNVEEL |
| | ARQGESPEEIKTVIKQLIWLAKKR |
| 284 | DEEVLLELLQRTGDPRVLFILFLLKLLKEKGIPPE |
| | EALKIVEELIKELGDPEAERLLEIVRRRLP |
| 285 | DPTHILLEISLLAQELLRRGIDPRKAVKIVKKILS |
| | KLIKEGKISPDEAKEALKMAKDVAKLLS |
| 286 | DSEKLKEKVKKALKKGDVQALFEVVELAMELGDQE |
| | LANLATKALMILAKR |
| 287 | DVRKLLKEIIEIARKQGDERAVEIAAALLAATEQG |
| | ESSEENSREAAERLFERLRN |
| 288 | DELEKKVEKLAREAMQAVDNGDKDKVNTLVTELKK |
| | VVKSQPDNPAAEFAALAITMLEYLIKH |
| 289 | IEEAKKLKDKVRKAAEKLSQGRLDERKWMKIVTEA |
| | MKILAKLGISEEEIMHFSMQAYELKKKA |
| 290 | SKKEEAKEAIKALRRMAKWLGVDEKYIRTASEIFK |
| | KLVKNGVDLEEALNFASIWLMQVLES |
| 291 | SEETKKLQKKVREILKKAKSGKIDPHKALSLVMEI |
| | LAKAMEKGDKKVLSEANRAAFKLIELESS |
| 292 | DVELMAETAIKIARKKGDEKAVRILEKIAKLERKG |
| | LIDEHEALRRINHIYERLMK |
| 293 | DEVEEELKKIAEKLSEAIRKNDEDKVEHLKEALIN |
| | AAFRASHKYDPTRVQRIYRKVMRLAEKLG |
| 294 | SIVEELEELLRRGEVDPERLVARFWELLVREGDPH |
| | KALKAISLFMALKRGEIPPDEAVELLREIE |
| 295 | TIRVTITWRVDAHNEDENRASRRLAEEVREEAEEI |
| | AHRLNRRLDLSITVVREGDKVVTIVEFTIQ |
| 296 | TREILNKVVEIVLKLVKKGVDPEQAVKIIRKILSE |
| | LIRQGKISKDEARMATLTARFVAHLAKG |
| 297 | LVIIVFYNVDPRLVKKFAKLIGAERITLFRFGQSV |
| | ILFVKDASPELVKKAKKIVKPNRVTVRH |
| 298 | SVEEVVEEAKRRGELPPEVLALAYLAIFRAKERGL |
| | PPEDVARYVEKTARELGDKDLEELAKKLKK |
| 299 | DPKKLDELRKKIETFLRKATDPDARMAAYFALNAA |
| | EKALKDQDPLAERAARAAYRQAKKAQ |
| 300 | DKDKKIKDIVKTLVSRAKKDPRQAEEALRIVALLY |
| | LRAQREGDEHAAKLLRKALRLIHEAR |
| 301 | SEEERKEEAIRIFIRYALKRAKSEEEAKEALEAIW |
| | RRAEKKNEEETLKLLKEALKRIEEKEKR |
| 302 | SAVEQAELFLREMNTPVAREILKVLKELKERGVSP |
| | EEILRLLTEVAEKLGDPLLAENLRFLLEHD |
| 303 | DRTHHATFTARTTDEREQEELRQRALLWLDRRFFE |
| | AKRLGLRSRFRLSLTWDGDDWRVEAHHEVR |
| 304 | SKKEEAKDAIRALRRMAKRLGVDEKYIRTASEIFK |
| | KLVKNGVDLEEALNFASIWLLNVLNS |
| 305 | DEEVEKLLRRAEKLLKEAKKKNDKEKLQEAARLVL |
| | KAARLTTKREDLQRATHLAWRIAKLIN |
| 306 | SDNLWTDIFELVMRALDEGVISEEEAKRIMARLNR |
| | AWERGLTEEVKKIKKEVKKLIKKARKS |
| 307 | DVEEALRLAIREARKQGDEKAVKKLEKLLKALRKG |
| | LTSEETALRRASEIMERLVN |
| 308 | DSLMIEALRNVIRKIAKEVQNPEVEKLAKKVDKLL |
| | KLVRKGELDEREANKRALHLFFLAVSS |
| 309 | GERYSLTIHVDQRDERLWREAQRLALLWAFRFIYE |
| | ARRRGLEVHHSNREDTRGNRATNHVELQID |
| 310 | TEIRKELEKIRKEVEKLQKKGRIDERLVEMIKRMI |
| | KQAKKTGDPFALMFARSLLEELKKLSG |
| 311 | TTRYSSFYTSVAASPKEQKEIEKKAEKLAQKERKE |
| | AEKNGLKVTVRVRTHRRNGVVIVVVEVTVQ |
| 312 | SLEEIVKEVVKQVATFAGAEDVKVRISNGKVTVTE |
| | NGTDESARLASRVIKNMLWLAGVDVKVKLK |
| 313 | DVEELLEELKKLLSPELRNLLKVLLSQVKRIPDPE |
| | KREKLLRLIKEAVERNDEQLLQRLMELAVR |
| 314 | DYEIIFKLTKLLHEDPRKFEELVERLLRELGVSPE |
| | EIEKILRKVREIIKRDPNKAVSFVFQLIQR |
| 315 | DEKVARTVLEVASELAIRTGDQRAMEAARFAAHVI |
| | ERARRGELSEKDRKKLEKTLRKIEKKLRS |
| 316 | DEKLRKIANWIMKEAAKAGVSRELIMEASLKLIDA |
| | YVKGKIDPKKLAKKLLKELRKEVKK |
| 317 | TEEIKEQVKRAVKAAVREGVDPEAAVRAIRLLLRL |
| | LIEEGKISKEEAKEATKVAEKVAKKAKG |
| 318 | SETDKVKKVVDTLLTKAKQQQDPNLARQAFSFAVK |
| | VSEKDPNDETVAKLVEKASIVWLQLHK |
| 319 | TRTFRWHWEFKGPDEETRKTIEQLERDAEKFAQEF |
| | AEKYNVRYNVVVEVVNTGDRIQVVILVIFI |
| 320 | GEHRAQRLLWLAVELGDQKVAVKILKVIQLWQEGK |
| | ISDEDLERLAKEAEEELKK |
| 321 | DESVHKLAQKLLRKWAEAARKKDKKKLEEVEREAK |
| | EIADRIDNDEIKQQLMFFHFYAQKLKKDG |
| 322 | SDETKRLQKIAKKALKLLRQGKLDIEEAKRIVQKV |
| | VREATELGDEEAVRVAATIALALERIEEG |
| 323 | DSLEKKERTIARRLLREAERRRDEELIQKILLLWL |
| | AFERALENNDEDALKDITKKLEETFRKLQK |
| 324 | SAKELAKRLVKLAKEVAKKLSDEEKSRKIKELAKK |
| | LEKAVKNNDERTVAQVQARLAILVLRAS |
| 325 | DEIARAAIEEILERAKKLARDPRIQKKIDKVLTKF |
| | KEAVKKNDEEKLAKVYHFASRLFAEAQS |
| 326 | SKEMQKVVTKVTSKAHKLGVSPEAIFEITLQIARL |
| | EDEGKLSPEQIEKWADKLAKKIAKKAS |
| 327 | SSEKAKKKIDKAIRNGDAQTAARALNEAYERGLIS |
| | PKEWYKLAERVVKARG |
| 328 | DEEEKVAKELRKVLKDAGATSFRVMILNGKIVVLE |
| | RGTSPEVKRASKVVTKYARSQGWKLQVNVL |
| 329 | SEEEQSRYLEMFLSHMFGVESVRVSVHNGTIHITV |
| | KGQTDAARAAQQYAKSYARVLNTKVDVKVK |
| 330 | LVIIVFAHVDPRLVKKFAKLIGAERIRVYREGDSV |
| | ILFVKDASPELVKKAKKIVKPNRVQVVH |
| 331 | DEENLKILIDQSGNPEDAVHQLSLFAKFKGISPEE |
| | IEKILEKVLRELGKVSPEEIEELKRLLKEK |
| 332 | QKHITITINLRNPHKDEEKEARERLRKFAKKVVEE |
| | ARKRGQKARIHETTVEFNGTRSVSVLVIIR |
| 333 | SMEQANVVLFVAAELLKKTGDDSARKAIRYALTVL |
| | HRAYEGKLSEKDRKKAEKTERKLEKKERS |
| 334 | DREELLRELNRLAFLVVRVIKDPKYVKTAARLFVK |
| | AYELANKGDEESAKKVQKSTETIITRYKS |
| 335 | SEEEKRKRAKKKVRKIARSLGLSEEIARTLEFIFD |
| | SLVELGVSIEMALKVIERFAKKLAKG |
| 336 | SSKELKKRIKKALKTGDVEKVIELSHIALQRGDEE |
| | AARLASRALFDEVNR |
| 337 | TQQVQTYVSYDPNDEHDEERAKEILEDRVRKVVKK |
| | HQKQGLEVRVRRRTINENGNRVLAVRIEIR |
| 338 | TDEREEVQELQKRLMDAAQKKDQEKLKKVVKEAKD |
| | IAKKVGDPMVENLLRYAITVAQKAVS |
| 339 | DEARKALVFVSLALAEYGVPHVKVNIRNGKITIRI |
| | EGDSPEAQTASEIAKKLVKQLGLPVDVQVR |
| 340 | LVIIVFENVDPRLVKKFAKLIGAERITVYRFGQIV |
| | LLFVKDASPELVKKAKKIVKPNRVRVVH |
| 341 | SKELEKVIKKVNKKAKKLGVSPEQIKRIEKEIREA |
| | YKRGERSPEEIERLAEFWAEFAARLAE |
| 342 | DERLEHLKRLSERLFREALERRDERAIRIASLTHL |
| | ALAKARNADKEEREEALRLVEHLLRLAE |
| 343 | DDIQKEARKVNHQLMEIFFRYPDNEKIVKLVTHAA |
| | SLALEAFDKNDEEILKKAKKLLEKVKKIC |
| 344 | DKLTKAAKELAKKVKSGKLSPEEAAMWLSMIATRL |
| | GIDREEIHKAIRKLLKLIKS |
| 345 | SEEVERRVKKLIEKAKEAVKKNDEKKVKDVLAILV |
| | SLMMEAQTEEERRIVITAWFIVMQLHH |
| 346 | EVIVLVTFTRNGKRTSVTFRVDDDPREVKKAEKLA |
| | KDLAKKLKDQGVPLVLRVRKGDQRKRIEFR |
| 347 | SDETKRLQKIAKKALKLLRQGKLDIEEAKRIVQKV |
| | VREAEELGDEEAVEVAASIYLALRKIEEG |
| 348 | DLKKIARKLLSIAFELGNHKIFTLVQRIFLAIKDG |
| | DEKTAKKLIKKAQSLAS |
| 349 | DEKEKVRRVQKYATKLARKAGVSEEAIKILRELVE |
| | ALIRNGVDASQAKDIARRFIEMTREL |
| 350 | SKELIEEALRLLEKGDPRVEKVIVKLENEAVRKND |
| | RDLNIEVIRVEMLLRNGNPEEARKLLRKLR |
| 351 | SLAEVVRKNVKRIAKMVGVPGVSVDVRNGTVTVTE |
| | DGNDEHARRASEQVKLFARFLGVTVKVTER |
| 352 | SIEELVKHVVKEVARVAGVPSVSVKIKDGKVSVTF |
| | KGTDEAAKEASMLVKLTAHLMGLDVDVKEK |
| 353 | SEEEKRKRAKKKVRKIARSIGLSEELAETLEFTFE |
| | SLVELGVSIEMALKIIERFAKKLAKG |
| 354 | DKTELVKRIVKEVAEVAGVTDFQVTVSNGKYHVRI |
| | NGNDESAQNVRSLIELSAFLAGIDVKVTVE |
| 355 | SEEERIEKSVKQAAKFAGVESVSVRVSNGTIEVHL |
| | QGDNDAARQVELFLRFVSHLSGIPVDVQIR |
| 356 | TEEEFLKTSIKELAKLVGVPSVRVRVSNGTVQVEV |
| | DGNDRAAKDFSQQLKLFARFAGIPLDVKIK |
| 357 | SEEVAKMIKKNVEKIAKMLGLSEKITRTAKKVVEK |
| | AYRKGVDPLEVTNLALQFLVKAAESK |
| 358 | SEEELIREAIELVAPDPNLKETLKLTLRFAKHLGA |
| | SPERIIFTLEKFLEDQGNPELAELFRKIVK |
| 359 | DYRKAVQYLIRVLREALRKNNEELVQLVNRAMFIL |
| | VEVSKSNDEEKLDKVASKLIKELQEASS |
| 360 | SSSTTWTYTHQPRDVQEAAEFLFKVLKEARERARR |
| | LRKQGIDVKLRVRREWRNGKVTIQVTVEEK |
| 361 | NLALIAVESLAKELYEKIGDPEIDKIRKKVEKLIR |
| | KYREGKLDESEAFNRAAKLQEELMKFI |
| 362 | SEEETRKRAKKSVRHFARWFGLSEEIARKLEKLFE |
| | KLVKSGISAEEAMNFISVIAVQISEG |
| 363 | SLLELLLRLLREGDPRVERVITRLLSLAIQRNDPK |
| | LLQVVNLIYFAIKNNDPDMVERAIERAKEL |
| 364 | SVTELVEEIVEKFKDDPEEVERLLQELAEKLKDPR |
| | LQQAVFTEKFALKLGASPEIIKETLRQFLK |
| 365 | SWIRLAWEALEIAERLGDEKAHRAVARILHLLARG |
| | RISEEEAEKALKRLIKKLK |
| 366 | SEEAIVRLYFKAARRGNRKVLRVLIRAVLALWRGD |
| | EETARKLLKLARKLD |
| 367 | SEEEKKIRKLVEKLKRKGKLSDREIANYVVKYWLK |
| | QGKLSEEQLLRATLIVQEVVG |
| 368 | TEEERHVEAVEVAARAAIQANDKEAIKKILEAAET |
| | VAKRISNPDLSKKVQKLIKKLRKALKS |
| 369 | SEISEKLLKLLEKAYEKGDEKALQIVTKALMLLSK |
| | GEIDPDQARKLAEKAERIERS |
| 370 | DIHSEYHIKIDPTDEQEEKRARLRAEALAYLAREK |
| | AEERGLENHNHIEIREHNGTIHVEIRINVH |
| 371 | SQMEKARRAQKYAEKLARKAGVSDDQIEQLKQAAR |
| | FLFAQGVDASQVKDILRKLIEKIKKS |
| 372 | SEETKRIKKWAKKLAKKAGLDEWAIKLAQELVERL |
| | ARNGIDPEVIKRMIELTIREMKKL |
| 373 | DEEEAKFAVAVVKQLAKELNDEETLKILEEAEREA |
| | RERNDWRIVEKVAVKLYALLSRER |
| 374 | DEIVELLFKLQYLALQLGDEKALRLAAKALMLLAS |
| | GNIDPSQAKKLAKKLEKLVRS |
| 375 | SVVVIVDDERLAKLVSKLVNVERFQISTHGNLVLL |
| | NLVGVDPELAKKAVKTANKDAVVRVH |
| 376 | DVIEEILRLLRELGRLSPSVERALEFLREMLKRGV |
| | DPKDVLNQLLLFARFIGDPDVEKLAEEFLK |
| 377 | DDRKKVEDTLRKKEKEAEKNNDEQALARVVWKALE |
| | LAEKEDDPELEKVAAELFETLARIQK |
| 378 | SEIEKKLARLLVKAFEKGDRKANEIVIKALFAYFH |
| | GLVDPDQARKLAEKAERILRS |
| 379 | DRDELIEENLRFFSEAANVPDVKFSVKDGRVKVTE |
| | NGDDEAAREAARAIRIFAWFAGVDVKVTTR |
| 380 | SRDELLRRNVEKLARIAGIPSVSVTVENGTVKVKV |
| | KGNDERAQFFSNQVHLFARESGIPVQVDLR |
| 381 | TEQEMASHTIKEAAFLAGVSDVKVDVRNGTLKVTV |
| | RGNNESAQVLKKNVERIAKHFGVRVEVQTR |
| 382 | DEEVKEILEELKKRGVDPEEVFKIAAKILEEKLNI |
| | SPTEAIFIAQYVALELGDPELLTVAAKLIL |
| 383 | DLWRLINRAAFLAVELGDQKVQQLALKAARAAMKG |
| | DEKTVKKLIKKAESLLS |
| 384 | SEEDKIQTLIDEVRKAAKRLYPDDEEVKKIESNAQ |
| | ELVDQASDQEEAKLTERELKHLLDSE |
| 385 | IEEVKKLMLLVSFAALRLANGKLDERKWKKIVKEA |
| | SKILKKLGISEEEIRDFQRIAKELKKQL |
| 386 | DHLDEILKRIFELERKAWEIGDEELERRLRLEQLT |
| | LLLRLAAHLDKEEREKLIKKWARLFQRAK |
| 387 | TTVRVYNFEARTDDEELLRKARELAKKLLKESKEW |
| | AERKGGKVKNRISFDRRGDKIWIIAVSSVE |
| 388 | SLKELLQEARERGDEDLLKVLSLLLFLIKLANIPP |
| | EEALKALERQLREHGDPDLLELLKELKELP |
| 389 | EERVLSLLQAAIFVAAEKGDSHRAEQAMKLAILLF |
| | LAEHSGDEELVKKISTKAEKLAKKVLS |
| 390 | DPLIFELLIRVQELADKFGLSPDEIKRELNIAARL |
| | IVKGVTPEEIERYIKEELEKFKRKQY |
| 391 | DEEKLERQARAALILAHQFDAEDEQARRFAKEAAR |
| | KLFEAIERNNDELVEEIIREIERHLKESRG |
| 392 | SKKEILEKVLELLKKNPDEAAQLILFLAFETGDPK |
| | VRRIFSVIAELIQTGDPELRKEAEKLLKRT |
| 393 | SWEELYEKAKKLVEQAKKENDEDKLRKARRLANRA |
| | AFAAVELGDPTAQRSAFHLYEIVHSVAE |
| 394 | DEHKAMLKTLIEELLRFAIELVDNPEERKKVTTLA |
| | KKIKKLLEKDPDTARRLALKLFREALKK |
| 395 | DVEVLMRVVEVANELLQRTGDDSANKAASWANRII |
| | FRAIRGELSEKDRKKAEKEARKIEKKLRS |
| 396 | DDILSEINRVVFEAHRRGDDEILRKASRVAMLLLS |
| | GKIDDKEAKKLAEKLKKLLS |
| 397 | DEHIRNTIDIHGTDKHARELVERYIVESERKAREE |
| | AERRGLKVHSEWETRERDGHVHVEHRVTLR |
| 398 | SEEEIRRVIELIKKKNEMLAISLAFLLELVKRGEF |
| | SPEEFLNSAKRLVRWYGDPDAEELLKELEK |
| 399 | DPTEWKRQVEMMAWINAEFGVDPRQVVKMVKKLLS |
| | EAQERGVVSPDEAKEALKVAKKVAKRLS |
| 400 | SRDETVERNVKFLARSAGVPSVDVKVSNGTVKVTI |
| | KGNDERAKFLTDQVRLFAMFAGLPFDVQVR |
| 401 | LRSEVIKYTIKGDSEEARKHAEELIRRERHRAREL |
| | AKRLHLRLRLFVKTTERGDTIHVDINILIR |
| 402 | DKEQEDKVKEVIKRAKELVKKNPEEALSLVFKLWK |
| | EARDKNDERLMRLANVAAFEVHRYIS |
| 403 | DIRKVTQLIFAIVEIAARKNDEELYKLAVKAYFEY |
| | EDARRKNDEKKSKEVTKKVEKLWKRAQS |
| 404 | SVREVLFELIKIVRELLERGVTPEKIKRIVEEILR |
| | RLLKKGKLTPQAVRFLRKSVERALEYLEG |
| 405 | SEMLATLVQNAAKKAGDKEATKIARKLVKLLREGK |
| | VSPSEVIEILFKLQIRLL |
| 406 | FELIVFQLDFHKDDSSARKKFKDTVKKLKKKLEDI |
| | ARKRNTTESVHTHERRNGDIVILLVNFVVR |
| 407 | SKEDKAIRALFRLSEIAMKLGVDMKYIRTASEIFV |
| | KLVKNGVDLDQARRIAEKWLKKVVKS |
| 408 | DPNEEWLHQQAEMVRELAENLRDPRQIQDLVTAAE |
| | KMANAAEDPRVSKEVKKKLEEIYKKAV |
| 409 | DMLEVLKAALLAYRLGLKEALREAQQAVERLADGK |
| | ASEEDADKELKKIRKKLNS |
| 410 | DELLKKVKKRLEKLEETAKRQNNPEALKVVQAVER |
| | DVKEALKRNDRERVHILLLFAEHWEKRLS |
| 411 | LLIIWFENVDPRLVKKFAKLIGAERIRLYRFGNSV |
| | LLFVKDASPELVKKAKKIVKPNRVTVKK |
| 412 | SRAAAKVMKVAIKAFEAGDPKAVRLANRILFALYR |
| | GEIDESQAKKLADEILKKIG |
| 413 | SEELKKLEKRLEEILRRLGVSPEQIKLLRKNLRKS |
| | YKISDSSAAKVEVEFVRQLFEKQRS |
| 414 | PIKDEAKKIAKELITKAVKHQNPELAFEATLVLVR |
| | LAEQYGLSLEWVNEVLKELVDKAKKS |
| 415 | PKWAEVNHILIRAILRAFEVGDEEVMRKAARLLFL |
| | LFEISDKNDEESLDKVEEKARKLLKKAI |
| 416 | DETVSHLLLLLLEAAYLALERNDPEGASRAQRALF |
| | IAMHLAQDPDPKAQKKAQDIAKTVYSEVS |
| 417 | DESEYETATRLAESAVKKVQDPEAEHLLRSIKKQW |
| | KELKSKYDDDEAVKYYHLMAYFLIQRFG |
| 418 | DEYEMISNQLTLFARFLGVGSFKVSIDNGRIKVHV |
| | KGNDHAAEDITEAAKFFSKLFGIPIDVQTH |
| 419 | DEEEEKEKEVRKVERTAIRIPDKEKAQREVKKYIE |
| | EARRRNDPKLEHLLRYVLMVIKMFSDT |
| 420 | DIHHTVQIDSDKLGEEQKEQILRALRFTAWLALRE |
| | FRERGIDVKVRITSETHGDRTHQRVHLRAT |
| 421 | SSTESAVRLIRETVKILNPPSVTVTSSNGRVHVKI |
| | DGDTRLARQLQFFALMNSFMVGVPLDVQVR |
| 422 | SVQDSVEFILRLLGVSPEIIERVVRRIEELLKEGD |
| | REEVIRLIKKIAEELGDPQLELFARFLKAL |
| 423 | SKKEKAKEAEKKLRKIAKKLGVDERMIREASRFFE |
| | INLMFGVDLDIARRMAEMYLKMMVKI |
| 424 | SEIVKLLVRLARLAVELGKEKALHIVARALYALAK |
| | GAIDPDQARKLAEKAERILRS |
| 425 | DLASEALRLAFEAFKRGDRKAMHLALKASILIFEG |
| | EISEKQAKKLIKRIKKLIKG |
| 426 | SSDESVTRAVKFLATMAGVKSVKIRKRNGRIEVTV |
| | HATTSAAEDVVNLIRLTAMRLNIELDVKIK |
| 427 | DETKKIQKEVKELADDARKSISDPRLRELVALART |
| | LAQVAARDPRFKEMVKTLKRIIKELKKQG |
| 428 | DEIQSKINRLAFLAFKLGDRKAQSLAIKASHLVMD |
| | GRIDPSQAKKLAKKLEKLVRS |
| 429 | SEEYEIRVTFDPNDREAEQEARLKVLFAVYRLLRE |
| | AEERGTQPEAEIREREDQDSITITIKVKFR |
| 430 | DHLKELFEHLLRLAFEASKTASEEQRHLLLTLLFA |
| | IRRALENNDEERAEKLLRSLEESIKRIR |
| 431 | DIEEEIRSLAKKLLEIIRKDPRHKEEAEKTLVRLL |
| | FEAMKKNDQKRAAAISMAFLFLHESQ |
| 432 | SEEEKKKLEKLFKKLIKILQNAPNPELRELARALE |
| | RLFREAKKNNDAQALSNLVLRAKLVVEKG |
| 433 | DEEEEIRELIEKAVPDPELRRLLKEQLKRLEEEGV |
| | PPEEILFTLIFAALRYGDPEVARLLFELAQ |
| 434 | DVEKQAKKAIKLARKKGDEKAVRLLEKIAKLYRKG |
| | LIDEREARTLILTIALALLL |
| 435 | DEEVKKLLTEATKLSKEARSKASDEMNRYFLENTY |
| | RYLISQVKNASEDQKRELAKMAYEVLKAG |
| 436 | DRRRRFRIAAALLSAKLLARDPRIQKKIDKVLTKF |
| | EEAVKKNDEDKLKEVEEQARKLFKQARK |
| 437 | SEELKKLEKRLEEILRRIGVDPRAIKRLRKFLREV |
| | YKDTTSEQAKLFVRQVRHLAEVHRS |
| 438 | DLRQANRAAFEAYRQGNREAQRLAVQLTSGLQDGN |
| | TSEEEAKDALRKLKEALSS |
| 439 | SSEKLKRLAKEALKKGDEKLLSRLMSIAMELGDKE |
| | ALREINRAYFEVAR |
| 440 | SDEKEKLKKWIDKLVKKEGLSPEQRRILELTLRFA |
| | WRLGASPDIIKEILRQAAEMMKKRG |
| 441 | DEELSEKIEEALKRGDERLVVQLVLRLATLAFEKG |
| | DEELARRLQVVLFLLSRDPEKAVRLVERLV |
| 442 | DYERVISRALELAAEKGDHKALRELNEIFFRAHEG |
| | KLDPTKAERLARRIEKKLRS |
| 443 | DELEKLHTEAERIATKLERSPDPENKTIAEAIKAA |
| | LRQALSDSDKESAKMVAYFAVNAARMV |
| 444 | DEREKERAEFIAAALSLLAASSDEREREEARRLAE |
| | AARRELSRKDSRLAEYFRHVFEESVRRLG |
| 445 | SREEVVRELLERFPDPREVLVLLYNILIRLGDPQL |
| | AALVAFLFHKLENGEIDPEEVRELVRRIVK |
| 446 | SREELLTRNVEFLAEQAGVPSFSVRVSNGKVKVTI |
| | EGTDEAAKFAVNQVLLFAMEMGVDVDIQVR |
| 447 | SDESKRHSFLTKLVALFMEARDPKFQHLLARAYDE |
| | FFRALDRNDKETLDKVIKKLDKIEKSY |
| 448 | NLEYDALRLIFLAFLKGDEKARTIARKLQAAIMKG |
| | DEDAIKKLATKLKRIL |
| 449 | SDETKRLQKIARKALKLLRQGKLDIEEALTIALRV |
| | VYIARELGASEAAEEAFRIQYALLEIEHG |
| 450 | PREEVLKILEELLKAPDPEKVIARLLLHASREGDE |
| | EKNRLVLAVIFLLREGVPPEEIREILRKLT |
| 451 | SEVARRLLELLKQGNEEEAQHLVVRALWEAAERGD |
| | SELALLLFRVQFLLMQGDPRVEEVLKRLEK |
| 452 | SSSQRYQINVDSSDPKDTEQAHRDAKTIAQRYAEE |
| | LRRQGNPVNYEVNYSELNNLWVVFVTVHTK |
| 453 | SEDEKIKKHVKTRIKQILSKNSPEARKEAANYANR |
| | VVFHLYHVPDPHKQKLANEIFHYWSRLR |
| 454 | LEREHAIKRIVEHLVKHFGLSRDIEKTLKKVVEKL |
| | LKKGVDVFRITLIVSQLADEWARRLK |
| 455 | SDESRRELEERLEEAVRRGDEEKLRLLASTLAYAF |
| | LRAGKLSSREALKYVEELIKELKKRV |
| 456 | SKEEVLSRMARLTFRFAGATDVKVTIRNGKVTVEV |
| | RGDTPSVRAASRMVTWLAKRQGVDLKIRVK |
| 457 | SRETLKRRVKEAYKRGDAHELFKILFEAMERGDDE |
| | VIRFVLRVQFKLARR |
| 458 | DHRIRITFHFTGDDEKTKKEIQKEIERIRKELREE |
| | AKRKGKDIRIFVQRRRHGSTLVIYVVVRRK |
| 459 | SEIMARAVIKLARKKGDEKAVKIAEKILKAAERGL |
| | YTHEALHKLNHLIFYLMRK |
| 460 | SEEETIEEIEKLLERAVRELRVEELRRLVEELLKE |
| | ARRINDLRFFEEAAQIALLAWLQDAL |
| 461 | SELREINRLMFLAVELGDPKLAQIALKAYRALRSG |
| | DEKTIKKLLKIAHKLVKS |
| 462 | NAERVVRLVKKLREELNVPPEEAIKILLEEALRRG |
| | DEELVFYLIFLLILVLGDPEKVREIFERLR |
| 463 | DLAREASHVLAQLAFSAEKTHNEELSRLQHRLAAL |
| | LAEAIIRNDEELLKHLIRLARELLKRVHN |
| 464 | PEDDEVQRTLQSVLEKATKDGNEEVIRKALKLNFL |
| | FFLAKRNNDEETLKKVITKAKKVIKKQ |
| 465 | SEDDKAHKVLQTVLELLSKDPKKAQDFVKTFLKKA |
| | QKNNNERLVHLARYILMQAQMER |
| 466 | TELVELAIELAKRLGDEMLVVSLMFILELVRRGEY |
| | SPEEAEHAVVRLARQFGNPVVEIIERLLKK |
| 467 | TERELIEENVKFLAKLANLSDVKVSFSNGKVTVNI |
| | NGTSEHADDLRRQLTLFARFANLRVDVQTK |
| 468 | SEEEIRELIEKLGDPRIAIFTLLYALERKGKVPPE |
| | ELHEALRRLLKLLKEGNEEEVRRLLTELID |
| 469 | SEEELKRIVERVVEGDPDARRIEVELYNELVKKGD |
| | PQKAIILAFILTLAEEGNPEDAKRLVELLR |
| 470 | TEERRWRWELRKPSRQEEDETRAEALRVAYKALRE |
| | ADERGGDAHSRVRVKHDGDTVTVETRVEIR |
| 471 | DRTETARRSIDRAAKFLGVPSWSVDVRNGTLHVKI |
| | DGTDDAVRQLSLFALFIAHLVGLPLKVDVR |
| 472 | SDRAFRKAVHIVEAAARRLNLEWAEELAKHLLRLW |
| | EEALRRNDEEALKWLFEELSRVMYRLAQ |
| 473 | DWRIAHRLLREANRIAYELGNERLWQLVARNFLAL |
| | AKAIKNNDEELLKRLIESSKRLLEEIKR |
| 474 | SSSTTWTYTHQPRDREEAFRFAAKVAKEARERARR |
| | LRKQGIDVKLRRRLEWHNGKLTIQVTVEEK |
| 475 | DEVRLAVTAFLLGDPEAASLINVLIFALVEGDPEK |
| | LREALKRAEEVAKRLNDPVLEKAVKIAKKA |
| 476 | PLEEELERLLKENPRAAVTTALFLAQERGDPDLFK |
| | ELLELLRLLEEDPNDEEVIRRVEKILRELQ |
| 477 | SLEQAIEFLLKTQGDPAARIVLEFVRELRERGVSP |
| | EEVLRLVEELLKKLNTPAARQLLLFIMELK |
| 478 | SSAHVIIEALKLARKKGDEKALRLLNRLAFRAVRG |
| | ELDPTKAERLARRIEKKLRS |
| 479 | DVEELVEEVLRKNDPRVLEKVLRLLREREGDPIRA |
| | FYILIELIAKILKVPPHEAAEILHRLLRLL |
| 480 | SAEDDVTSLVRLSAWMAGVPHVEVSFKDGRVSVTL |
| | KGTSPSVRSAKRVIQRVAKMMGVRIEVKTR |
| 481 | SIEEDVKMIVKLSAFLAGAEDVKVRISNGKVTVTE |
| | NGTDDAARSASRMIKHVLTMLGVDVKVKLK |
| 482 | DLEEAVERVIKRFKDDPENVAILLAFLLLLALEKD |
| | PRIVEEALRLIEELLGDPELVKWLERAVKQ |
| 483 | SETEILRVLFIAHRKGNREVAELALRASVAAFLGD |
| | PKTAEKLLRKAKKLLKS |
| 484 | DEDKELEEIVKKLAEDPTVQEIILKVASNSPYLRR |
| | TIIKLFREDPRQAHLFIRFLAHQLK |
| 485 | SIDELLEKNLRKIGDPNLLRLTAVFIVSEALNVDP |
| | EEARKLLEKLERELGDPEEALRLAVRLALK |
| 486 | NLIAEAVWKIARELYEKIGDPEIDKLRKYVEKLIR |
| | KYREGKLDEHELNERALRVLFKLYRFI |
| 487 | SYEEILVKAYFILAEQFFERPDDEHIRKALLYVLR |
| | LVMKAIKASDPKESHEIARQVEEFLKKVS |
| 488 | KDIEKEIVKVEHTAMERGDIELALKASLVFMALHR |
| | GEISDEQAKKLAEKLKKLLS |
| 489 | DELLTEIIHLAARDPEKAKELIKEIVKELRKKDPR |
| | LVKEFLSMLLITLRELGDPELVKLVEKLER |
| 490 | SRVEKILVRLIARTGDPKYTLIAFALILLRREGLP |
| | PEEILKIVEKIVREANDPDLLRILEEAIKR |
| 491 | DIVEKVRRLATVAIFVAHKQNNEEIAVTLMKLIHQ |
| | AHEAKLNNDEETLDKIRKIVETLYKRVV |
| 492 | SDETKRLQKIAKKALKLLRQGKLDWSEAIRIVLKV |
| | AKIAWELGDQKALNVANSIAFILVKIHKG |
| 493 | PEESEKFKKTVETRVKRAVDNRDMDLVNRVVWEAL |
| | EIARRLNDPELEKWAAKLWLKAVRNI |
| 494 | SEEFRRHIEFLNRLLHLAHHHRELAEEAIRLLREH |
| | AEHAKRINNEVLAELSERLARQLEEAIRH |
| 495 | DESERVRLAFWAAAHAYKHAQSEEEKKLAEAALRS |
| | AVKFAETKSDPKMQKLAKKLKEKLKKIV |
| 496 | SEKEAKKLLKIARKIGDRRAAFIAAAVLASLRRGQ |
| | LSEEAAKAALAVVKELLS |
| 497 | SKEELKREVAKLLGIPPEELEKLIELAERAAGGDP |
| | EIAELTLYLALARLGVPPEVIKRILELLKR |
| 498 | SEEAAHLVQVAAKRAGDKEATKIARKLVKLLREGK |
| | ISPEEVMRILNRLMERLV |
| 499 | DEDKKVSEKVKKLVRKAENANDPTQIFKLLRKALE |
| | LVMQVSDPTIHKEVATEAQRALFIAARR |
| 500 | TVRWEITVSFDRHNEEEERKARELLRRLLKKVHEW |
| | SRKRGLRNLYWRFETTRGDRVTIQVTSLSL |
| 501 | SLEEIFKVSFIAWRKGDEEVLKLAVRAIFAWLNGD |
| | PKTAEKLLRKAKKLLKS |
| 502 | TEQEKFKTTVKRLVDQALKENDPRLAVRVIWEALE |
| | EAEKKNDEDLARFVARLELKVSRWFA |
| 503 | PEEVAKMINKVAESQGDKKALKLARKILKLLREGK |
| | LSDEQAINELQRILFHLA |
| 504 | DASDIFRLLEIAMKLGDEKALRVLLRAQFALAKGD |
| | EELARKLVKKARKLES |
| 505 | PEVEIALFFAESVLKQAKRDNDSRSVRVVISILKR |
| | VIKKAQTEELRKKAKKLSEIAKKVEKS |
| 506 | DIQVAFTVQEVALQLLDETGDDSARKALNYATEVL |
| | FRAVEGELSEKDRKKLEKTLRKIEKKLRS |
| 507 | DDWTDHVEKLESKAMEAVKKNDEDTVKTIEKILKD |
| | LTKKAQDPKQRELAALAITEVQSLLKD |
| 508 | SLEEDLSRMARITFREAGATDVKVTIRNGKVTVEV |
| | RGDTPSVRAASRMVTHLAKHFGVDLKIRVK |
| 509 | TSRNRFTVEVNKKNEEERRLAEENLWILAAFARRR |
| | AREEGGRAKVRVEEHEDGDTTRFHNEVEVR |
| 510 | SEVEKILVRLENIAMKKGNREANRVVNHAYFAYKR |
| | GDIDEKQIRKLLKKAKSLLS |
| 511 | TRVFTWRFQAKNVHEQEIREAREHLKKLADHWLER |
| | IKRRGLRARVRTSSERRNNTHIIVVQLEVH |
| 512 | DEEHTKELFEEAKRIAEELNIEELKEFVAAALANQ |
| | ELARNIDDEEEARELTESAINILRLI |
| 513 | NSDELKKRLEKIYRLVEEGKLDPDKAEQIANEIWR |
| | EARKVGDDSVRQEALRVALYLHQFKK |
| 514 | SKDELITRNITELAKMAGVPHVEVRVSNGRVHVKL |
| | KGDDRHAKMAKRLVELTARFAGVDVKVTIR |
| 515 | DETLEKSLRKMLRLDPNMFELTVMFVLKLRGVSPE |
| | EIEKILRKVREIIKRDPEKAVEFVLRLIRK |
| 516 | TETHHFIWTIDGTNLRAAAKLTTLAAKEARREIKR |
| | AKRRGISVTVTITVRVNNGLAHVELILRLH |
| 517 | TEVEALSRIIEFVARVAGVPHVEVSWSNGKVTVTE |
| | KGTDHRVQEASPMALLTAMFLGVELKVQER |
| 518 | PTEEIQHLLVRILKKAAEDGDRRLLHAANHLYFEL |
| | EEAKKRNDEEKQKKILKRVRKLLKEAQR |
| 519 | SEIMELMTEAVDLGDPELLRLITLVAMAVAKGDPE |
| | LLKRVEKEVRELSRKNPLVRRLLKIIEKLK |
| 520 | SIQEEILRLQFIAFEQGDHKIARLALRAAEALING |
| | DEETARKLVKKAKKLV |
| 521 | DEKKAAKFLKKHLTERARRAGVSPKAIKKIQTIFD |
| | RLIKSGVDLLEATLIALKLIDKWRRS |
| 522 | DERVVMNASLLILALLNRGVSEEQIKKFVKRLLEK |
| | ERKSGKISEEAAESVKRLAKRLIKIARG |
| 523 | DLEKEAQRVLYLLMHLAMQIQDPKQRAKAQRIIWR |
| | LLEAVKKKDEDKIKKLVDLAKKLIKLV |
| 524 | SEIVKKLLKLAEKAVRQGKRKALRIVSTALFQLVS |
| | GDIDPDQARKLAEKAERILRS |
| 525 | TIEFENTIVLNGNSERVQKWAEEWLEEWRRKLSEL |
| | AKRLGLEIKHRIHRHRQGKFIVIHIHSRIK |
| 526 | PELDKIVHHALSLAAVIAFKATDPRIIRKAMKVSV |
| | LLMEAREEKDKDLVTKLLKKAQTLWKEVS |
| 527 | SVSILLLTAIFVAHEKGDEKALKKLQKLLRALQRG |
| | LTSEETAKRRAETILKRLRS |
| 528 | SLVEKIEKLLREGDPEEARQLALLLWLALHEKGDP |
| | RVELVRRLFRLLAKGDPEKAKKLAEELKRT |
| 529 | SIMEEIYKIQRLAEKLGDPKLEQIAMRAALAAASG |
| | DEKTIKKLRKIARKLIKS |
| 530 | SPQEAMELLFRAQYIARQKNDEELTHKLVRLLWLL |
| | VRAIKEGDKDTITKIERLAHEIYKKC |
| 531 | SLTETIQTSVTQAAKFAGVDKFSVTVANGSVKVRV |
| | RGTDDASNQLILFVRFLAHRANLPVEVEQE |
| 532 | NELEKKAQRLAHLLAEIIKKVPDPTIQHQAATLFI |
| | LLFRAIVKNDEEKLERLIKKAEKWIKSV |
| 533 | HRHYQFSVSVRGTDERAKKILKKFKEEVKKLTEKA |
| | RRDYNTKVTFRIRSLRFGDVVLVFVDIRVE |
| 534 | TEQKSEEIETDGTDEEAEKRAREFWERIRKQARRE |
| | ARNRGRKVFVFIFREEHNGHIRHSVYVVFR |
| 535 | DLSKELEQRVRKLEKKWQEGKIDTQEALTEVLTLM |
| | FKAEETGKLDPKYLEWLLRVFEEWLRRVY |
| 536 | TEMDVINRAIFLAHKNGDDKALRILIRITHLAMQG |
| | KMSEEQAEKRARRIEKKLKS |
| 537 | DTKKKSKELLEILERLAKRHNLEELKRNIEALRLA |
| | LRAAQQHNDEELIRKVDEIAEEAIRRG |
| 538 | SIRFKDYVQVSATDPHQVTRAKEIAKKFVKKVEKE |
| | ARKRGLSVSYDVFVRRYGNRVFIVVVIEVE |
| 539 | PETVELSIRLKDTDEEAEKRVEKWLETIIKKLEEK |
| | AKKLGLRVKVRKFKERRGSTLIIQVRIEIE |
| 540 | DRKEVQREVKERVDTALSNNDPRTAAQVVLDASRL |
| | AEELGDEELLRLVSQLMFKIAEFQG |
| 541 | SSEELKRLLDEARRRGDADLAAEVAWKAAEEGDRE |
| | AIEAAFRLQYELLN |
| 542 | DDWTEHVKKLVKDAEEAVKKNDKHTVVRIWLILFR |
| | LFIKAQDPKQRKLATRALALVLHLYKS |
| 543 | GPEEAKEILKKINSPIAREVLRLLEEAERKNDPRL |
| | LEEAILVALTAAFAGIVSPEVERLIVEFVE |
| 544 | NEQKMIETLIRSAIRDGNERQFRELVKVAKKHGLL |
| | SEDTLEQLKKEFKREKKY |
| 545 | TETRKITITVPGNDEKAKKILKDIVTKIKKDAETH |
| | AKKRNLRVDIRYNTLEHNGKITVTIVVIFK |
| 546 | DREWHAHIDVKGTDRRAKEIAEHWALTWAFRILLR |
| | TRRQGLESHVSIKVRRDGDTVRARVTVRAH |
| 547 | DVRTLATRVKQRLTELYKLDTPEARKEVIKVWFKL |
| | MEIYSRITTEEEAKIILEAVLVAISVLLG |
| 548 | SDLARLMKLSQRAFEAGVPKAERIILRAMFALHEG |
| | KLDEKQLRKLQKLAEKLIS |
| 549 | SPDEIATLVRKNVEKLRRTFDNDWKRQAKLTVRFA |
| | ILLAKKNNDERLVRLLKTILKQIEKLSQS |
| 550 | TELVEITLMFASRHNPLAKELLELLRELKEEGKFS |
| | EEELIKIIVKRAEELGNPLLARNLEHLERV |
| 551 | SSEEIKHLIALVAFIFHKKGDRRAVEVWNRLFRLL |
| | ELVRRGTLDPDEQKRLLKELKEEVKKL |
| 552 | SERYTFEVTEDKNDKEAEERARQKLLLFWARVAFR |
| | AERLNRSAEVRVRTSEHNGTIRIHVTIEIK |
| 553 | SEELLREVVERLGVPPEVLEVETKQLRRLEEDGIP |
| | PEVILFTLALAAERLGDPELAELLAHLAAR |
| 554 | SEEIEELIRELEKKARNPEVKRVLALLYLALKKAR |
| | SPEEREEIERLERLIEEDPRLAREILKKLD |
| 555 | SWKDLLAEINSAFMKGDEKKVKKLKKIAEKRGDER |
| | AAHMAESALRQLQRR |
| 556 | DELINEIIQLAVKDPEKAKELIKEIVKELRKKDPR |
| | EVKLFLETLLLTLRFLGDPELVKLVEKLLR |
| 557 | LDEEKRIKTTVERLVKHFGLSRDIEKTLKKVVEKL |
| | LKKGVDSEEIALMVSMLAQRWAERLH |
| 558 | SEIEKKLRKLKKKARELGNKEAALAVQQAIQAYRE |
| | GRISPEAAKRLAEQAERILRS |
| 559 | DEEVEKLLRRAEKLLKEAKKKNDKEKLREAAHLVL |
| | RAAALTQSDEDLKRAARLAMRIARLIN |
| 560 | DEHELQRLAWLALELGDEEIAREAFRVIMKVIEGK |
| | LSEKQAEKWAKKIRKKLSS |
| 561 | DSEHMVELTARFLAKRVGVPDVKIRKSNGRITVTV |
| | KGNDESAQIFSRVLKHMAKRLGVDVRVQTR |
| 562 | DKKYNELLRLLWKALELGDEKAARIAFRALVKLWE |
| | GKISDDDYKKVQKLITKALRE |
| 563 | DEEVEKLLRRAEKLLKEARKKNDKEKLKEAAKLVR |
| | KALRETTSEEDRIRATRLWLRIAELLK |
| 564 | TLDDVMLRAVESQARKLIKKSRDPNAQSKLKKAIK |
| | IIQKASQVSDPNIKKSLVQKALTVVMDAA |
| 565 | SEEVKKILKRIVKLLAEQLGLDEKAQKIAKKVAEK |
| | LADKGVDPLEATLILVRVLDTILRRKK |
| 566 | DEELKTIMNIVNRLMEEAWRNNDEKSIRLAWTALQ |
| | LLMRAHNADEEQQKKLKTLVKQLIKKIK |
| 567 | SEVQELVEEVLKRGNLEELIKEVLKLARELGDPYL |
| | EAAARDALELVKRDPERASFILVLVLEELK |
| 568 | DKEEIRQKIMNLAFAANLRVQDPRIKKTIHTVYRQ |
| | AMEALDKDDEDTERTLLKIVEKLLRDVHS |
| 569 | TEHYRWTVRVEGTDREAHQIIDEFIARILNEAFRI |
| | ALKDTSHIRITVHTKVKDGEIRVEVRAERR |
| 570 | SINEDVQVRDENNHESVLVQVEDPTEAEQQVLLFL |
| | MELGVTATVRVDPDTGSVEVQQ |
| 571 | DDEELQRFIQRHVEETVGAPDVQVSVSNGTVKVTI |
| | DGNNDAAQAVRFTAELAKQKAGRNVDVTTR |
| 572 | HEEIANKLSKKVAEILDQQNASDEQKSKAHEILLE |
| | ASLVLTNISDKDKAKSYADKVTKDISNM |
| 573 | DRLAYALTLSRSLLRDAKARNNEELIRLAEETSRL |
| | IEDARRRRDEELARRLVHKLLEAIRKA |
| 574 | TLREEAMKLAMEAMKRGDEKALRKAQRALFLMARG |
| | DISEKQAKKIIKEIKKLIKG |
| 575 | SETYKVTIDFDGKDEDTRERAELFARYFRLRAEEE |
| | ARKRNVEVRIEIRFDTRNDKLKTDVRAEFR |
| 576 | DEEVLELVEELLKQGDEEEVEKLLAHVAARLKEKG |
| | DPEKAILVINVWLRLKKNRERALEALKRVP |
| 577 | DRETEIFEFFAKTTIKRAVEENDEERLKELEHAIR |
| | REYKEAKKRNDEDKLKVAKRLLEWEKKEV |
| 578 | DLEEELEEALKRGDPELLKRIVEKIIKKLGLPPNA |
| | EYSLKWWLEFLLRTKDPRAREAVESQVRHG |
| 579 | IDDVRWAASNARAYWARLGDDTFDKERKKLEKVEK |
| | KVKEGKLDPEQARKFALEVERRLVKLV |
| 580 | VVIIFVDNEELAKQVEEVARRLGVEVRVSKQGDLV |
| | VVTLQVRDPTLAKTAASVVPEARVHEF |
| 581 | DEEKKELHKEIERLLKELLRHAKSEVEHLAAASLL |
| | QLHRKAWENNDDELWEESKEKLEDARHALR |
| 582 | SVVVVVEDERLAKLVSKLVNVQRESIFTFGNVVIL |
| | NLQGVDPELAKKAVKTANKDASVLVF |
| 583 | QEEVHRTLLNLFLMLSHNARDPNLQEKAQRLARKA |
| | LQAVDKNDDDTIKTLLKLLKREVDEVRS |
| 584 | SEKDVKRFIKKLLDEARKKGLISEALKESLKRVAK |
| | ELLKRGISTEEIKRILETTARFLAKENK |
| 585 | KDIEKEIKKVITTARRRGDELLVAQALAVAHLERE |
| | GLITDEEAKRLAEELKELLS |
| 586 | SEKDVKRFIKKLLDEARKKGLISESLKRQLKHLAE |
| | EALKRGADTSEIKKLLKIAAKVTAEFLK |
| 587 | SEEERKRLLEALERGEVSPYELALRLQRLAYENND |
| | PELLNRATKVVMMLAHGDPRAEELLKELLK |
| 588 | SLEEVVEEAAELLNDPALRELVELLKEARENNDPE |
| | LLTILVINVWLRLHELGNPELARRVAKLRP |
| 589 | DEEESAQRLVKRTAEMAGVPREDVKVSNNTLTLDV |
| | TGDDENVRDVTMNAHTTAHLVGLEFRVRTK |
| 590 | SIEEQARRVVEHVARRYNVKDERVSVSNGKVSVTF |
| | KGDDEAAKRASRLVELTAHLAGIDLKVTVR |
| 591 | IELEEKVEHLASRAMSVAMENNNREELRRVNELYF |
| | KAEELSSSPDPKESKEALDRVRKELEKIV |
| 592 | SREEELLELVVREKDDPKVILHLLEELLRLAQEKG |
| | DPDLEQLVAELFAILARDEERVVELAEKLR |
| 593 | DESLKKFKSDVEKKAREAKSNNDEKRLSEIVEEVL |
| | KFAQEAQNNNDQESASVAAVALFLVFKIQ |
| 594 | DEVLEASYLNKQLLELARRLNLEELVRLAEKSLRD |
| | LARAALNNASEEFKKTLVELSKELEELIK |
| 595 | DDEEAEEAKKLVDKVSKTNPRFREQAQLVKKNLTR |
| | AQRNKDKDDVEAAILLIISNYNVAS |
| 596 | DKREEVKTLYKILEEAAKKRDEETIDKVAARAYHL |
| | AVEIGDRELLLKIVSLWMHAQELLS |
| 597 | NEAQEKAAEIARTLAEKARRLGRPVDVSVRFKNQT |
| | LTVKVGPDPKEAEEAAIRIFAALFAKLEG |
| 598 | DLRFTFSVRDDSDDERSRRQSEDLAKAFVETAALA |
| | ATELGLELRFEIRRLRDGHSNEVRVEVRIT |
| 599 | SFAEMVLEYLEKAGNPEAAKAVRVLLELLKRTGDP |
| | RIRNLINQIAWAVMEGDPRREELIRRVLEP |
| 600 | SEFVIITVELRNVSPEQSKKAEKKLKDIAKKLTKT |
| | AEKRNLEVRFRIKEQSHRGTKVLIFNLVVR |
| 601 | SEIEEVLELIRRYDPRVVEMLLVSLMELKHMGASP |
| | EEILKYLLRLAKILGSPLATELLRRLLEKR |
| 602 | SEVRALTLALQVELLAKKVGDEEAKEAAKKABELF |
| | RRLREGKLDASQVEKLLKRITIKKLRKLG |
| 603 | RRHWEYRIRVDARDERQRREAKALASIIATRLWLR |
| | SKEQGQENHFHIREERHGEDLTFHIRNETR |
| 604 | SKDELIRRNVEFLARVAGVESVSVDVRNGRVSVTL |
| | KGDNEAAKDVKNQLELFAMFANLDVKVNIR |
| 605 | SKELEKVIKKVNKKAKKLGVSPTAIKDIERFIREM |
| | YKSGLASPEEIEKEADRLAKLSAFLAR |
| 606 | DQLEEAIRRLLELVPDPRDVVNQIELFLRFMNSPE |
| | AREALRLLEELRERGVSPEEIVEIVLKIAR |
| 607 | HLHLRFSFHSEGDSERSERAAEQFAEAVRFTAALA |
| | ARRRNLEVRVEIRIHIENGRTHVEIHVNIS |
| 608 | SETENVTFEIDGTDERAEEQIRRIVEKERRRAEKH |
| | AKKRNLRPHVHVLTIRINGKLLLIIVIVER |
| 609 | SEEAKRELEEAKKRGNLERHLRSLLFALAAFLGDP |
| | EEATKRLEEIARSINDPEAEKLVKKLSKET |
| 610 | TKEEKLKKAKKIVTTLAKLANVDPRQIKLLQSLVK |
| | MAWEKDDPFAKTMVEVIIRQLKHMVDE |
| 611 | DMRERSVEEMLRIIETVARRRDDPTAKTLVKRAKK |
| | ILEEARRKNDPKLLRKALRLANRAYFSLK |
| 612 | VEELKKAQKKLDKAIKILRKKNDEMAVKLAEAVKR |
| | ELKKASSDSDPRTIHRVIFVAVNAVRNAQ |
| 613 | TSQREDRNEFQPENEEAERLEKRAIRQAARAAVQR |
| | IRQQGIDVKVRERTETHNGRVTLSVTFRHR |
| 614 | DEKDDFSRLVKLTLWLAGAKSVDVKVKDGTLDVKV |
| | EATDPSVEFASKTLTKWAKDQGLRVSIKID |
| 615 | VEELKKAMKTVQRAWTILMDKNDIEAALEAVSVEM |
| | MLAKASSDSDPRTIKTVIDLAKQVYKKAT |
| 616 | TNEEVVEHLARFNPRNLELTLREMLELARREGNED |
| | LVRVLEIVERLLEEGVDPEEVVRVFKELLK |
| 617 | SDETKRLQKIAKKALKLLRQGKLDTHEALSIVMEV |
| | IRIALELGDRKAVQVANSIAFLLIKIEKG |
| 618 | DLRKVTRLIWAIAEIAKRKNDEELHKLAARAYAEY |
| | AKARRNNDEKKSKEVTKKVEKLWKRAQS |
| 619 | SKELEKVIKKVNKKAKKLGVSPESIKAIERNIRTL |
| | YKLGLLDPEEIEFFADMQAHMAAKRAS |
| 620 | DEEDKKFAEKVRRKALELIHKDTDEETRIHLINLW |
| | LRAEAAQKTTDSEARRLFKELVEELERRD |
| 621 | PEVEELLVMAEIMLKQAKKKNNHMLVRSVLRILKE |
| | VIKKAQTEELRKKAKKLSEIAKKVEKS |
| 622 | RIEIEHSVLIRGDSEEAKKRAEEHQERFRKFAEDL |
| | ARKLNRDVEITFFITELNGVIHSIATARIE |
| 623 | DETHKLYITVLSLARQAMSKNPEYHEEVARLYVKI |
| | FRLWEKGSPRVNEVLKKAEKILRSIIG |
| 624 | DSKTIAMLRVLIRIYAKRKQIPEIEKLAKKVDKLL |
| | KLVRKGLLDETEAEKRAARLWFLAVSY |
| 625 | HLAEMIKHLAKREITKAVQNNDEHKVELVVFFLKN |
| | AARSVDDPRLEEEIKRIVQTVLEQVL |
| 626 | DPDEILDKVANTLLTLAFQHPDDPELHKAFSMVAE |
| | ISQKAKKLSDPTQKERLARLAQRILEEVS |
| 627 | DEEVAMTVSKVASMLFLQTGDESAVKAMHFALQVI |
| | TRAEKGKLSEKDRKKLEKTLRKIEKKLRS |
| 628 | REQFFFFVRVNGNDPTQKKKAEDKAEXFRQKANTT |
| | AKRKGKKVRVTHSSSTQDGKYVVLVVIRVE |
| 629 | SEFIRELLRVAKELGDSVALMFLEQLLELLERGEL |
| | SPEDVLRLVLLTLRFIGNEELVRLAEEVLK |
| 630 | DLDKRAKIVLSLARHLRKKVQDPHIRKLLEHLAKI |
| | LEEAVKKKDEELIKKILVLVERVIVEAQE |
| 631 | DEEEKFRKTIEKIAKKLGVEQVFVKVFQGQFVVFI |
| | VGDTESARKVQKLAKEVAKKLGVRVRVRIF |
| 632 | DKELKKVINKVMKKASKLGVSPEAILHIAIQIQRL |
| | FREGKLSPEQIEKWADKLAKKIAKKAS |
| 633 | DMHEEAIKMLLRLLETARKEAPDPEKSKKIDKIIK |
| | KAKTWSSQVSLEESANYIFDLVVKVQRE |
| 634 | DEEERRKISERLKVDPKVAESLLRIKKWLERVLDP |
| | EFVRHSLLMFIFLLLKVDPREAERLLKLLE |
| 635 | SSTQRSEITVQARTEEEAREAEEELRHSAEALAYL |
| | ARRRGVDVKLEIRIERRGKRIRIRIRFELR |
| 636 | SSEELKKKIKKALKDGDRRAVTEIAMLAQELGDRE |
| | LARIALEAAEKLAN |
| 637 | DPTSKKLKKEIKKVLKLEKKGKIDPVEAWNLLERL |
| | IEKARRIGDEEAVQLAMHAIAIILHAMKS |
| 638 | DKDESFRRIVEWIARMLNVPDVKVSVNNGKFHVHL |
| | KGNDENAKDAERLVRLTALFMGVDVKIDLQ |
| 639 | PREEILHKINHLYFKAHEDGDKKVLKLLAKLLYKL |
| | MDAVRRNDEEKQKKIAKEVEKLEKIVQR |
| 640 | DERRTFTLEVKGTDEKAKEKLRRFALEVQFRVTTE |
| | ARKNNRTVRVHVRVERRGDKIRVKIDVTIR |
| 641 | DPSEEVTKTVQSILTEALKNKDKELVHKAIRIALE |
| | AARKINDPRIQEVITEAFTVALAVQLG |
| 642 | SEEELREALKELAERGDLHAAARVLNSLLFLLYEK |
| | NDPSMRKVAEVMIRANEDPRRLPELIKLVH |
| 643 | DEKLRKIMNWLMKEAAKAGVSRELIMRASMKLMMA |
| | YEKGKIDPKKLAKKLLKELRKEVKK |
| 1563 | GDRRKEMDKVYRTAYKRITSTPDKEKRKEVVKEAT |
| | EQLRRIAKDEEEKKKAAYMISFLKTLG |

TABLE 4
H3 binding polypeptides

| SEQ | |
|---|---|
| ID | |
| NO | Sequence |
| 644Z | SEHEEFLEWLLRKIEEAIKRGNKISAEFLIGLAKQ |
| | FLHVINDDEIRRRLERLERLLH |
| 645 | SQHEKFLEWMLRKIEEAIKRGNKISAEFLINLAKN |
| | FIHVLGDDEIRRRLERLERQLH |
| 646 | TEEEIEEVEELLKKATDPKLRHMLEFLLRRLREAL |
| | ERNNLLSARFLLGLARQFYKSI |
| 647 | TRMRISVKFLLGLARQFTDPRIREWLLRRAERLAR |
| | EGNDPELEELVRRVEEELK |
| 648 | SKVKLSVKELLGLARQFYRAGNPEAAEYLIKRAKE |
| | IAEKNNNEELRKEIEELEREIR |
| 649 | SERNEHLLRWLLEKIRKSTNEISLRFLLGLLEQFA |
| | RAAGTEEAREILREAEEIVK |
| 650 | DERSSAKISVEELLGLAKQFTDPRIREWLLKRAKR |
| | LAEEVGDDELKRLIEKVEEEL |
| 651 | TKERVEHLVKWLLERAKRMPSPISRKFLLGLAEQF |
| | ARAGNDPELERLVEKVKREL |
| 652 | DQAKRMVEWLLKKAREALKLGNEISVRELLGLAKQ |
| | FAHANNDEEAERLVREFEKEL |
| 653 | DLREKLKSMVKDLLRKAQESQDPYYARQAEILIET |
| | IRKLAKDPDVQRLAQEALESLRKYL |
| 654 | DDIQKEAKKVIEQLQRIAKTFPDNMNVRYLVMLAE |
| | RFAHAAQRMNDEEALKLAKRLLEKAKKAG |
| 655 | SLEMAVKWLLTVLKRLRDKAQDPTSRKLIEELYKK |
| | VEEAAKNKDPRAAHLAFYAVAQARMLV |
| 656 | DMITIAIKLATEVYRLLVEGNYDEASKLAKKAIEE |
| | ADKKNDEDLKKFAERLLERIKKLKKS |
| 657 | PEETYKRATKILTEAKKRAKDNPEIQRMIEMYQKM |
| | LEYAYKINSEWALREIAHLVENAVRRLLS |
| 658 | DRMQVALKVATAVYKALAHNDPDKAKKIVDDAERL |
| | AKELGDEMLERLVKKLKKRVEDEKG |
| 659 | DETVIKAVMHAYRAWMQAARENDEDKAKKIIKSLK |
| | DLADKLNDPYAQRAVKNLEDTLKKLKG |
| 660 | DEEIEKVLEEVLGNPWDARVLAQLVRALQKLINID |
| | PEEAIKILIESLERMGNPEQAEALKEVLRH |
| 661 | PEDEWVKQVETILRKAKKVPDPTERKKLLIEAQVR |
| | ARYAQQVATDPRVQKILEELIKIARKLYV |
| 662 | DENQYQRAYDWAAEWIRRVISASSEEESEKLIEKA |
| | KEEAKRRGDELALRLIERFQKLWRKNL |
| 663 | SQETVKDLVRKAKELADSLGDPISQEYVSTLEEAA |
| | RRATKNNDEHKLRILRISLEYELKQL |
| 664 | DLDERAKKLLKLLREVRKKAPDPDLRKKAEMASRM |
| | LEEALKKKDELYVQTLIRFAEVFVKLV |
| 665 | PEVKKKADHILKLLEEAMKKTKDPEVVQELMHLWF |
| | RLVWAAKEQNKEMLDRAAKHAKELIKKTS |
| 666 | PYQVTVHGRSETQTTTVSSYTEAWRWAYRAAKRLG |
| | LKITRVEFKDGTEHFHLE |
| 667 | SKELIYRFAIRYLLKEAGVPSFSVDVKNGTETVTV |
| | QGTDKLADYARKMVKEFADKAGVKVEIRTK |
| 668 | SEEELIEKVERVLGDPRLRELLRRLVEDAKKMGID |
| | PKMAVQVAARYIAQQEGNPEVEKVAKEVAK |
| 669 | SEDEKLEEKLKKIARIAGVPEFTISRSNGTLTVHE |
| | RGTDLAAQVLERYARQLAKSLGIPLRVEVR |
| 670 | SEELKKKLREALERGDRRAAAQLLYRIWAQARLRG |
| | NEEEARRHIRELLKEFGDPLLRYYLEYLEK |
| 671 | DESLKKWKSDVEKKAREAKSNNDMRALIRIATEVY |
| | RYYIHARQHNDEEAQKVAEEAYKLVRKII |
| 672 | TEEYDIQYRARAQARIAGVPSFSVRVSNGHLEVTL |
| | EGNDESVKTAKKAIERFARMRGIPVRVTTK |
| 673 | SEEEEKLIRIAAGGDPHAAFYILAQLRLLKKGDPS |
| | AKMLLERLKEELEKLGNPERAEALERLLRK |
| 674 | DLQKEIKTVIDLIKKIAKSKDNEMLKRAAKMLEEA |
| | WKKAAKLNSEYMLRQLLRMAEALLKRA |
| 675 | DETQKQALERYAEQVARYASRERASRALEMLIKIL |
| | EKQNNEELVKVARKLLEWVRKYV |
| 676 | DKEEAKKIIEEATRRWRNARSHWEQEMILIHAALL |
| | LLEAASDAREAKHWMKLLETVLKND |
| 678 | SEDAKDKFREYAERAHELGVDRETIKYSLEAILRI |
| | MVKYGFLSPEEAEKVRRWLEEYLDKY |
| 679 | DKYEKLLKILKKLAELALRNNNPEVARWVIHAVTV |
| | LYRAKVRDDPNLKEKAERLTRTAQKLV |
| 680 | TEKRKWIEKIRQLLHELMKLGLDPREILRVATHAY |
| | RLLIEGVSPSEVKKWVEKVVKEVKKRI |
| 681 | SEIKKKWEEVSKKVQKIAEQLSPTQRYLVELHARK |
| | ARKLLENASSEEEKEKVLKLFELVARIVI |
| 682 | DYQKLEKRVKTIVTLIKQLKKDEWEEKAAERILRL |
| | AEEASKKKDEEKLQTLIRLAELIVRAAKR |
| 683 | DTSEKLTKAIEQYAREVGAPSVRVSVSNGKVTLTV |
| | RGDDERALKVASYAYFAASQAGIRLEIRIE |
| 684 | DETELARRLVKWLLSMVKNASNKMNAEYLLRQAER |
| | AARSVNDPTLQEQITRLKKWVEKHER |
| 685 | DDDSHKKKLEDELESLIKKARDPEARKMVEWLARE |
| | AKKAADLGNKISAEFLLRLARQFAEASAR |
| 686 | DRKEQIKKELKMVKERLDKATSREQIEIIVQSAKR |
| | YAEQANDPEAKKVVEKLEKDAKSKG |
| 687 | DDRKKVEDTLRKAYKEAEKHNDVHKLFRIVWLAKY |
| | LAEKEDDPELRKVAKQLHDTERKWIK |
| 688 | DPRKEAKELVDRAKTLAKQVGDEHALKVIKKLEEL |
| | LKKAIDNNDEKTWVYAWTVVRIIILVLEK |
| 689 | SYLELLVELVKRVDPELAEEVERLIRELKKQGISP |
| | MLILYKLLLIATFLGNEELIRIIRKVLHEL |
| 690 | DRATKMVRDISKKAETAARSGDRQRAQALVRLLEI |
| | FARAYPESPVSEEAKRVIEKVKKLIDS |
| 691 | DEDLKLANIAYYLASQAGADHFEVRVRNGRLTLTV |
| | KGDDEKAQKVRKHVQTHEDKLGVDVKVTYK |
| 692 | PLEELIKTLQRLARELARKVPDPKNRILLWKVMTL |
| | LYLAQIISDPNQSKTAVDRAKEILDRVRS |
| 693 | NEVQKRAKRALQLLAELLKKLNVDPKMVRQNLQRY |
| | AKTLQRYIQNPENRKFVQTVAEELAKRVM |
| 694 | SPDDVKKLARIVQKLAKEKRDMVAWYYAHQVYRLA |
| | EVAKRENDKELADRLKEKLEKLVKKL |
| 695 | SELERLLELLKRVSPQLRLLVRMLEEALRRNDRMN |
| | ARYAFKLVVRVLRALGDPLREEAERAARKV |
| 696 | PEEARKLIEKLKKLMKLAKEKNDPELVHIAIRLAL |
| | LEWRAAQRNDEETLRKLEELIKEVEKRLS |
| 697 | DEYVKKIIEYLKKELRKIGDSEAEERAERHLRELE |
| | KRGVDALKIADWAYYWLFNARVRKG |
| 698 | DPTEIKRRVEKLAKELYKRGISPVLAALIVYRHLT |
| | QLVREGKISPDEATEAQKVANKVAKREL |
| 699 | SLEETVEELLKRLGVDPRLVYSVKAIIRIALRYGL |
| | SPEEILKLIIRYLRRAGDPEAAKLIEELLK |
| 700 | DLEEIARKAAKLAEEAYKKNDEYMRRLALMMLRSV |
| | IKYASRYDDPKYKEIRKLAETVYKKVKHG |
| 701 | DEVEKRIRSLLERIQKLLRHASPVARRYLLELARQ |
| | ILESLAHSPDDSVRKAAKEALERERREI |
| 702 | DPRKKLKELVDRARTLARQVGDLRAYRRVITLAYL |
| | AKMAIDNNDERKLKQAEEALKRIIKKLES |
| 703 | TEEIKYATKVYYAAVQAGVPHIEIRISNGTWKVTV |
| | EGDDESAQYVLKYAKTLADKLGVPINVRVR |
| 704 | SEEERKEKLRKVIERAARLVGISPEIIKRIQKEAD |
| | KIFDKLSLLWAAHWVYRAIFEYKRQL |
| 705 | ERVKKMVDTLLKQAEEAVKRNNEYLLEQIKRLLES |
| | IEKLARDPNQKKLARDALETIRRLE |
| 706 | NEELNAKIIERVAEGDPDAHYRLRAQIRYARELGN |
| | EEVLKILEEALELWEEGDPDKVRELAERLK |
| 707 | PEHEKIVQHAMELAKKIFEKASNPEIRRKALHVAL |
| | ALWRALMRNDKDLATKLLKKAQTLWKLVS |
| 708 | DEQEEEDVRHLAELLYRLATQIPDETKAKKLLDNA |
| | YRILKEAQDPREAKKLLKELYEKAKKKA |
| 709 | DKKEELLKILKKALELLKRSGASEEQIRTYAEQWY |
| | RAVAQNDEDQLRRLEKIARTVEKRL |
| 710 | SREEELKAYNTAYYLAAQAAEDEDAERKARKILDK |
| | HLKDLKVPNAEEFKKLHDRMVKMIRSDQS |
| 711 | SLSEKLKTLLEQAERAANIPDEDKSRTILKKAEEK |
| | VRKLLRHPDPSVYQLAYKILLILTELF |
| 1564 | SQHEKEAEWMLRKIEEAIKRGNKISAEFLINLAKN |
| | FTRDPELKRRLERLERQLH |
| 1565 | SQHEKFAEWMLRKIEEAIKRGNKISAEFLINLAKN |
| | QTRDPELKRRLERLERQLH |

TABLE 5
IGF1R binding polypeptides

| SEQ | |
|---|---|
| ID | |
| NO | Sequence |
| 712 | GSSTRNAEFIVLLAELCAKSQNDPSLQEYVKKVKK |
| | IVESLLSNGDEKSAEEVARKALEYCADGGS |
| 713 | STRNAEFIMLLLELCVKSKNDPQVQEYVKKVKKQV |
| | ERLVGNGDEKKAEEVARKALEYCADG |
| 714 | DKELLERVVELLRQGNPEEARKLLRELLRELAELG |
| | DPLKRLVFQLLVLLAAGDPEWKEILKKLEK |
| 715 | TAEELLEIAVKDGNPELLRAMLRDIARASALAGNL |
| | EELEKVLERLLKEVGDPIFEEAIRLVKELH |
| 716 | GSDETCKEAERNIEFLALLAKLLDDPEHAERLLKQ |
| | AEELAKKCNDPRLEKLVKKLSEEVRKYVGS |
| 717 | SEELEKKVKEAVEEGDPVIVELLLAELLAETGDPE |
| | VEKLERLLQEALRRDDPDLAREVLELVKKT |
| 718 | GSDEKCKEAERNIEFLALLAKLLDDPEAAEELLKQ |
| | AEELAKKCNDPRLEKLVKKLSEEVRKYVGS |
| 719 | DEEVRRLLKELLESGRFSCFAVVLLVSVDLIAKGI |
| | PPEEAAELVERAIKKLGDPCLEEALRELRT |
| 720 | SKIEKAIKLAKELGDPDAVRILELVKEFLEEGDPE |
| | RAKALLRFLILILRQFGDPELVKIVEEVLP |
| 721 | DIVEEVIRKAEELGNPHVARLIEFLLALVKLAGLP |
| | PEELIKMLEHALEIEGDPIIRELIELLRKK |
| 722 | SEDDKKCKSVEDKARELIKKARDPLVQRALKAAIE |
| | LIQKASQVSDPENKEFLCLLAKLLVESAIG |
| 723 | DEELEKAVELARRLGVDPEALKALLRAIKEALERG |
| | DKENLEFLLLLLELIVNDPEVRKLVRKVIK |
| 724 | DEEVRRLLKELLESGRFSPFAVVLLVSVDLIAKGI |
| | PPEEAAELVERAIKKLGDPDLEEALRELRT |
| 725 | SKVERLIELLKRLGDPNLRHLARALEEALRNNDEE |
| | NLEFILLLIELVARALGDPEVLKLVREVLK |
| 726 | SEDDKKLKSVEDKARELIKKSRDPEAQRALKAAIE |
| | LIQEASQVSDPENKEFLVLLAHLIVEAAAG |
| 727 | DDREFVAELILWLARENPENAEHLCEAIVKILGLS |
| | PELEELCRRIVELVERGDKDKVEELLRRAK |
| 728 | PLAEESVEIAKREGNLEEAIEFLLLLARLIGNEEA |
| | VRLLEKAKELLKEGDPDKAEELVRRVLEII |
| 729 | SDETAHRIIRRVLEICDKRGDEDCSQKVLRLAILA |
| | RSDAHHASSEEEASRIWQDAAKKVQKLESG |
| 730 | GSDLEDLKKEAKKLEKWVSNCRDPLLREAVSRLIE |
| | DVKKAADKGDKHNAEFAVLLAQLFVKSCGS |
| 731 | SDEQEEIRKKVQETLAELIKKCQKDPETAKKEVKK |
| | LRELAERLGDEELKPNVEFARLLCELLEGS |
| 732 | SREEEAVRESLREALEEGDPRTVSLALAICRIILG |
| | DPDLCREILREIAKELGNEEVVKEAEKLLP |
| 733 | GSDWRQAEHILRFIQHIARAANDEDALKRATTLLK |
| | QLQEATKKGDEDTVKRVIEEAEKLLRRAGS |
| 734 | SPLEEEVKKLVETAEKAAKDKNDPHAEEAVKSLDK |
| | LFKEAKKKNDEENLEFVSLLARLVLRAVIG |
| 735 | SEEAKKVLKFIKKAEKEARDANDPVAVELAHEALK |
| | LAEDAARRNDKEALRQAEFLAFLAAFIARS |
| 736 | SKVERLIELLKRLGDPNLRHLARALEEALRNNDEE |
| | NLEFILLLIELVCRALGDPECLKLVREVLK |
| 737 | SEEDELRKLVEELEEKARKVIRTEEQREAVESLID |
| | LAKKALKNGDKENVEFLALLVHLLIEAGGS |
| 738 | PEIEEKFEHIMRLLPPEDIQLLIFLLMVASEKGTE |
| | EEVIRYFVELVEKLGDPKEALKLLEEYVRR |
| 739 | PEIEEKFEHIMRLLPPEDIQLLIFLLMVASEKGTE |
| | EEVIRYFVECVEKLGDPKECLKLLEEYVRR |
| 740 | GSDWRQAEHILRFIQHIARAANDEDALKRATTLLK |
| | QLQECTKKGDEDCVKRVIEEAEKLLRRAGS |
| 741 | GSSTRNAEFIVLLAELLAKSQNDPSLQEYVKKCKK |
| | IVESLLSNGDEKSAEEVARKCLEYVADGGS |
| 742 | SDERERLREAVEELERLAREENDAFIAALAVLLAR |
| | SALEQDKDDELSRRLLELAEKVARELRSGS |
| 743 | SEALQILLRSARHHGDEEVVRVLERVIKLLEDGDP |
| | DSAEKLARELLEKTGDPIIRAALELLKRLK |
| 744 | SEELEKKVKEAVEEGDPVIVFLLLAELLAETGDPE |
| | VEKLERLLQECLRRDDPDLCREVLELVKKT |
| 745 | SDETAHRIIRRVLEIADKRGDEDLSQKVLRLAILA |
| | RSDAHHASSEEEASRIWQDAAKKVQKLESG |
| 746 | SCIEKAIKLAKELGDPDAVRILELVKEFLEEGDPE |
| | RAKALLRFLILILRQEGDPELVKIVEECLP |
| 747 | SPEDKEKARKILQSVLELVAHNQIDEKIASKLISE |
| | VKTLAKRVNDPQLLRNAEFVELLVRVLYRG |
| 748 | DEEVRRLLKELLESGRFSPFAVVLLVSVDCIAKGI |
| | PPEECAELVERAIKKEGDPDLEEALRELRT |
| 749 | GSSTRNAEFIVLLAELLAKSQNDPSLQEYVKKVKK |
| | IVESLLSNGDEKSAFEVARKALEYVADGGS |
| 750 | GSDLEDLKKEAKKLEKWVSNLRDPLLREAVSRLIE |
| | DVKKAADKGDKHNAEFAVLLAQLFVKSDGS |
| 751 | DEEVRRCLKELLESGRFSPFAVVLLCSVDLIAKGI |
| | PPEEAAELVERAIKKLGDPDLEEALRELRT |
| 752 | NEKEVIEELLRDGNCLIATALALTVEREINTPEAR |
| | ELLKLLREALERRDPELLCRLLELVLELLK |
| 753 | SDERERLREAVEELLRLAREENDAFIAALAVLLAR |
| | SCLEQDKDDELCRRLLELAEKVARELRSGS |
| 754 | GSDKELAKKAVKELLETALQQGDEENFEFAVLLAK |
| | LLARAVNDPDVERIIKELEKKEKDLKKSGS |
| 755 | DEEELKELAKRCGIPPEEVEKVLRKLEEHEGNPEA |
| | AKAMLFLALAACVYDPDSPTARVAREIIKK |
| 756 | SEEERILKLLEEAVRRGDPELLEELIRLLSELLGD |
| | PRNALFLAALYAIAAGNPEAEKIVEELRRR |
| 757 | SGSSKEDAEKLLEKLVEAARRNDDETVTKLQQLID |
| | EICKKEQDDNVCFLALLAEVLLRSLKRGSG |
| 758 | DAERLIEIIREIHKKNPEAARLIFLILFLLLLLKD |
| | PELRKLYELVREAVEKGDPELVRRLEKLVH |

TABLE 6
IL7Ra binding polypeptides

| SEQ | |
|---|---|
| ID | |
| NO | Sequence |
| 759 | HEVQELARKLWHATEKAEKRNDEKAVKLLEDASVL |
| | LVNAWLSNDEDLKKKANKYAKTVIKQAES |
| 760 | SVKKKVRKVEKKARKAGDELAVLLARRVLEALEKG |
| | LVSEEDADESADRIEEALKK |
| 761 | SVIEKLRKLEKQARKQGDEVLVMLARMVLEYLEKG |
| | WVSEEDADESADRIEEVLKK |
| 762 | DEVHKLAEKAARLALKKGDWDAHAKVVNVFLALKH |
| | GIISKEQAKKILKKIIEEILS |
| 763 | DDTKLARRKVKLALTIVKNASSKEEADRALHIAEI |
| | VADEVNEPTLQEQVTRTKKWVEKYES |
| 764 | DYREVIKKAAHLAAKKGDEDAHAILVNIFLAADNG |
| | ILDPTEAERLARRIEKKERS |
| 765 | SFEEVIRRAIKLAEKKGDELAVYLLRYILKAVRKG |
| | LASEEEAEKAARRIEKVLKS |
| 766 | DKEEKVKKLKDRANEVASKAKTRLAMLLKILVEEL |
| | EDALKKGDPDDYRLAYKKVEQAIKIIE |
| 767 | SEDSRLDQMKVKQAAKLAESGKVDPHAVKIIIKII |
| | LEAARERGDEDVIKVAERALEKVKKIESS |
| 768 | TIEEELKKLARKAWKKGDEDAHAVLVNAWRAYKLG |
| | DEKALKKLIKLAKELLDS |
| 769 | TKVQELIERLREHAERKGNAVAKVIADELEEALRR |
| | GDESDIRMNSRKAKQAIKA |
| 770 | DKRKKLEKIAERLAREGKLDEEEAEKLAREAWKAG |
| | DEELLAKIVNAWLHAKLS |
| 771 | SRVELLLEVLKRVARIDGNEKALRVVKKAIEAYKK |
| | GLADEKQIEKLARKAQRLLI |
| 772 | DYQKLEKQVKTLVTLIKQLKKDEEFEELAQILLEL |
| | AEIASKKKDEEELKLLVYALKWLVDEAKS |
| 773 | DDTRMARLKVTLALNIVKNASSKEEADSALHIAEI |
| | VAREVNEPTLQEKVTRTKKWVEKYES |
| 774 | PEVKKEAKKAEELLTQARKLAEDPRIRYIIEVLIE |
| | ELRKAVKNDNEEDLHIIIWRAKAIWKYVL |
| 775 | DKEEKVKKLKDRANEVASKAKTRAAIILKILVTEL |
| | EEALKKGDPDDYELARRKVELALKHIE |
| 776 | DPRKKLKELVDRAKTLAKQVGDRMALLVVKALEME |
| | AKEAIDNNDEEQLRMVEMKLRLAIKHLES |
| 777 | DPRKKLKELVDRAKTLAKQVGDRMALHVVKVLELE |
| | AKEAIDNNDEEELKLVERKLRLAIKILES |
| 778 | DQELAKIIATAVYEQAEKSGDPEDERLAILKLEMA |
| | IKHVTDPNIRKYLKKLLENLKRLQ |
| 779 | DEDEKLIRRKIELALKILSKIPDPTAQKVVETAKK |
| | LYEEATKKNDKELLEDVAAYAVIQAIRI |
| 780 | DVEKLVKEVQKLAKKRGDEKAEEAAKIILHAYRAG |
| | VTSSKTASIALKILAKRLKG |
| 781 | EDREMLKRKVEIARRQGKLSDAILEALYAQARDLG |
| | DEEVRRLIETFRREKE |
| 782 | DETAELILKLASIALTVLAEVNDKVTVDITENGYT |
| | VRVDLDRDPKKAQEAIRRALEYLEKANKK |
| 783 | PDEAVRLIADIVLEMLEEAQDPTDRILLTKKVEQA |
| | LKHAKSIPDPKAQKIVKDLKERLDKIG |

TABLE 7
InsulinR binding polypeptides

| SEQ | |
|---|---|
| ID | |
| NO | Sequence |
| 784 | LIREEVQRRLEEIKQKIKEGKLDPFEAMSLLAELY |
| | ELVEKLGDPELRKELEKVMEIVMRLG |
| 785 | SKEELKERAIRLLKEGDPFEAAMLLMHLAFLTGDP |
| | RLEEVIRLLEEAFELGDPELLKEIIKLLEE |
| 786 | DVMEEAFTVWFQLENLAFRVDDPEQRKTVDTLSKL |
| | LQEAADRGDRELLKKVIKTARDVISKVNS |
| 787 | SELLERIERLLKEGNPEEAREVARELLERTGDPRV |
| | EALVSLIEELLRTGDPFYELFVEMILRELR |
| 788 | DEELLELVYEAVEKNDPRLLFEAWMILASLLDKTG |
| | DPKIEELLRLLQLVDRGDPDARRRIKELFK |
| 789 | DEELMELVYEAVEKNDPELLFEAWMELASLLDETG |
| | DPKIEEALGLLQQVDGGNPDAGRRIKELEK |
| 790 | DPFTAMELVARVFELALEKNDEELQKEAETLMEIV |
| | IKAAQNNDEEKVKEVIKRAKELLSKS |
| 791 | DPFTAMELVARVFELALEKNDEELQKEAETLMEIV |
| | IKAAQNNDEEAVKEVIKRAKELLSKS |
| 792 | DPFTAMELVARVFELAVEKNDEELAKEAETLMEIV |
| | MKAAENNDEEAVKEVIKRAKELLSKS |
| 793 | DPFTAMELVARVFELAVEKNDEELAKEAETLMEIV |
| | TKAAQNNDEEAVKEVIKRAKELLSKS |
| 794 | DDHAQAQLVKKLLKLWQDQGDDFYALLAVEAAANE |
| | RVKKYLKKYYPEVYTLLEQVKRTLEKKRS |
| 795 | PEVKKEAFKAFMLLMDALFLAEDPNIRKTIEELIE |
| | RLEKADENDDEEKLKEIIKKAKTIWKQVL |
| 796 | SEEVEEAVELFKEDPKTALNLLFELFMRLVKEGDP |
| | EKAKLVQEVAELLMNGDPDKAREVARRVRK |
| 797 | DEEVDRAKELAEELGDPELAELIEELKELSKRGDP |
| | RAVEVVLRAMELANRLGDPEIQEVLIKLAE |
| 798 | DEESETIKKKLDDILRVVQSVNDPELESVVERAVK |
| | DAKKALDNGDKNTARFLLKMADLARQFAS |
| 799 | DKSERDIVEKIFKLVMEIMQYNPELAKKVHSIVEK |
| | AEHNQDPKLLKKALDVAEKALKTVSS |
| 800 | DEVFKAAMKVLHLAFKKGDEEAMKEVQKVSELLEN |
| | GQASKEQAKKILESLARRLKS |
| 801 | DDSQVKDVESKAREKKKKHPQVSFLAFFVIEVARF |
| | AKEFNDPLALKVAEELLKEVEKRAK |
| 802 | TEEEFRVMMLLMTLSPELNELVEELSEAMDRNDPE |
| | LEKRVLKRLLELARELGDEEVVRILEKLLK |
| 803 | SMEDTVREALKAAKKTGDEFYVSMAEMAAQLIGDE |
| | ELKQRVKEAERYVREY |
| 804 | DESEEVKKMLEDLLQKVEKATDPTDRQFAIFMIEL |
| | VLEAIPDPNQQKLAKTVLELARKLD |
| 805 | DLEEIVREYVERTKDPIAREALELLEEIKKEDPET |
| | AKVLAEAVVDAIERNDPESLKFVLLLIKIV |
| 806 | SELLEEALELVRRGDPRVFEVLFEIWMLAVQKGDP |
| | ELEELIRKVDELLQEGNPEEASKLLEKLLK |
| 807 | DPFTAMELVARVFELAMEKNDEELQKEAETLMEIV |
| | LEAAKNNDEEKVKEVIKRAKELLSKS |
| 808 | DEDAKMSIKLVLEIARDLGDEDVYKKVKSLLETAE |
| | KQSRNASSEEEKSRIWQDAARKVMKLFF |
| 809 | DPFTAMELVARVFELALEKNDEELQKEAETLMEIV |
| | IKAAENNDEEAVKEVIKRAKELLSKS |
| 810 | SEVEELVELVVELIKKTDPELARDVKETLEELLKR |
| | DPKEVLLYLEFLAHFLGDPELLEKVRRLLK |
| 811 | DKEVLEEVLELVEEGNPFEAFFLLLELAFKLKDPE |
| | IDEVAQLIVEAFDHGDPDLIRELVRKLLEK |
| 812 | DDLDEIFTLSMLAMELAYKVGDPSLETEVQQVVDT |
| | ASELVDKDPRFREEAKKILKKLVEKAKKT |
| 813 | NEEEKKKAQELVKQIEKILKQIDDPFYKEMVRMLL |
| | HIAKEMQDPEIIKEAYEAAKELLELAKS |
| 814 | DPESDAKRIATELATLYKKLDPEESRKVADTAFTI |
| | AMRLLSSPDPNDQKKAQIIAEVEDKLAT |
| 815 | DVKKLAKTVKEILKEAQKRHPTPEVKHEAEFISWF |
| | LDAATSVPDEKYATSILTELEKAARSLS |
| 816 | SELFELVMEAMEIANRLGDEDLMRLLVELVEKALE |
| | SPEVRELLEKLARLIIEDPEKARELVKKLK |
| 817 | SFEELLRELVRRAGISPDDIDFVLWFVKVALENGN |
| | PEIAKELVEELLRKAGVPEEEIEKVKRIVE |
| 818 | DPSEEAQKLVDKVKEDVKKKNDEDAEEQVKVABEL |
| | LKEAKNAQDPRDREFLVLLIKLIVKNLS |
| 819 | HLEELNRHLLHLAFEAYFKIDDEHEAERFHKISEE |
| | LQRLFENARSEEEKEKIVRLQEELIRRVI |
| 820 | DEFEKHMAQLAVEAIEEILKDIDDPHYKDLVRKLL |
| | ETAKKEQDPTKISKALEKAIKLAEKAKS |
| 821 | RQEKLRRLLEKVRKLVEKGKLDPFQIMSLAAEVIQ |
| | YAEKVGDETVRELAERLIEEVVKLAQ |
| 822 | DETKKVIDKLVSEAKKLADSIDDEFKRKLAQMAVE |
| | SLEFEVNNAKNPEQVKKLWKEIHKIITDL |
| 823 | SEKEREVKELLEEISRRDPRISFYLFFVLIALEAG |
| | NPELAEELIEDLIRRFGDPLLRELLELIRK |
| 824 | DELEFLVWFAQVALEMVQEDPTNEEAKKVATDIVR |
| | DLDELEKKSRDPRAQELARRIKELYKKAC |
| 825 | SAFKLALKAMHIANELGDEKAMKTVQDIVRLFDEG |
| | RISEEEAEKALKRLIKKLK |
| 826 | SEESEKVKRWVETELTKANKQNDVEKAMSIVATAL |
| | ELAEELNDEELEKWVRSLAEEVETKAV |
| 827 | SLLERIEEALRRNDPELVAMLIMNLAFMTGDPRVD |
| | EVERREVKAFEEGDPEKAKELVKELLKRVK |
| 828 | MEASESAKELIKIVKSLYSNGDHNAKFYASMLKML |
| | AEALELNAETEEIKKVVKEAKSVADSVLS |
| 829 | DEDKKTLDSLVSYVETVIQQANDPFLSFMLEVLLR |
| | FAQENNDPEAAKEAYKLAQDVKKNVD |
| 830 | DTEEIRNELFKIAMIALRVNNEELYEVAQKAAELL |
| | KEVSKDPRKREELKKLLKRARELSKRS |
| 831 | TELEFLEEMVRLVLENALENGLLSPDDVKKYEEKV |
| | KELRKRGDKDEVQKILEELLERLERW |
| 832 | GEFYIELVEMALDFVRMLSGDEEFIKRVRKRLKEV |
| | KERGDDSKLQEILEEVSEYIERVA |
| 833 | DPFTAMELVARVEELALKKNDEELQKEAETLMEIV |
| | MKAAQNNDEEKVKEVIKRAKELLSKS |
| 834 | SDDEFWRWMVKAAVDAAKELNPDPTVQTKADEILK |
| | KLEEALKKKDPTLVTRVLEEATRLAEEA |
| 835 | DEKEELEKRLREAEKLVRQIQNEFYKMLADMLTKT |
| | FKEALDNNDTDQLRMIVEVIEELARDALS |
| 836 | SDFLIHFAKEALEEARESGLLTPDEAEKWKKLLEE |
| | VRKRGDDNQIRKIYEEIIELVERING |
| 837 | DDEEVKTWEKLAKEAINRIPDKDVRTEVEKMVRDL |
| | IKAAHSPDPQEVRFALEMVRMIIETERG |
| 838 | NEESEKLSRHLLELVERLVDDEDVVKTARELAREA |
| | IELSNSSDHRDRERAEFLNLLLKLLT |
| 839 | SEEDKIRKKVEEYVKKAIKRGINPSEVADWVAKYL |
| | TKAVREGKLSPEAARKIVNLAFELALKLI |
| 840 | SEVEEIIEELRRLGNPEVAEILELALRVAKELNDP |
| | QTEDSITELARLLKEGDPFAKHMLELIARD |
| 841 | DDSEEITKLLDDLVTKAKKKIKDPRDQKIAEDLAK |
| | EAKKLASSPDPFDKHMAKLLVEALQQFV |
| 842 | DPSEEAQKLVDKVKEDVKKKNDEDAEEVVKIAEEL |
| | LKDAKNAQDPRNRSFLVLMIKMIVKNLS |
| 843 | PEEREKLEELIKKDPSEAVFLALSILARLFEETGD |
| | PRIEELFKLLFEALERGDKDLLERLLKRIG |
| 844 | KEEAKLNADFLVWMATVAARDSSPESRTIAKELIK |
| | EAKKLAKEANDPNVEKKVRSVEELIKKLG |
| 845 | SAFELALKAMEIANRLGDEKAEKTVKDIARLVSEG |
| | RISEEEAEKALKRLIKKLK |
| 846 | NEEEKHNAEFLLWFAKVMADYISDDDKRKKAQSLV |
| | EKARKLDIPEEAKKVYKIVSTILERDAK |
| 847 | DLIEKVLEALERGDPELLARLLFELVMKLLSLGDP |
| | RLLELAQKLANAFDDNDEELLRKLLELLEP |
| 848 | SPKEELDKWSKIVKEIARKKNDEELYRIAERIYKD |
| | AKEALEKNDETDVKFLIEMLKMLSENG |
| 849 | PAAETLEEAVEEGNERHVEFYLFFVLQILEHDPDS |
| | PEAKELRRVLEELRRKDPRVEELLKEAKRR |
| 850 | DAEEILRELAKQVGVPPEEVEEALEILTRVLGDPK |
| | EAAFELLAILAEAAEKKDPRLFRELVERVR |
| 851 | DEEEVRRVLEYAWFYLVALDTEEARKLAKTLDDLW |
| | KLAQSIPDPKQKKEVLDKLKEFAERVVS |
| 852 | DPSEEAQKLVDKVKEDVKKKNDEDAEEFVKVAEEL |
| | LKQAKNAQDPHERRFLVEMIKMIVDNLS |
| 853 | DEEEIKRILEEEKRALEEAGLDEEAVRELLEESKR |
| | EWDEVERRNREEEWEKLKEVQRFRLAIIK |
| 854 | DPELEEAFKLTMLLMHLSMQVPDPEQQKTVETLAK |
| | LLTEAADKNDKDTLRKVKKKAKDVYSKLS |
| 855 | SEAEKVVKELLEDAKKLVENKQASPEDIEFIEFEV |
| | NIAAEANGDEELLKKVKKVIKELKDKS |
| 856 | SVEELIKELRELGDPELLRLAEEVLKLARKLNDPF |
| | LEKSIEELIRRAKKGDPFAKEMLEMLVHEG |
| 857 | DKEIEDKAKEVSKRAKELVKKDPFEAMSLVAKLFE |
| | EAFRENDEELLRWAEKVWEEVRKYIE |
| 858 | DESLSKLVFTLLSLLAEAWKYDPDSAETAVELLIK |
| | AANNNDEEQLKKAVDYAKKVVDRATS |
| 859 | SLEEEARKLAEKLKRQGDEMAKELAKYLEDAVKNG |
| | DKNDVSFAIWMVRVFQE |
| 860 | SVREAVEIILKRVGVPPEFIRELKEELEEAEKEDP |
| | REAEELAEFIIAFALNVPPEEARRLLEELQ |
| 861 | DEEAVKELEKLAKELGDPELLEALKKLRERLEQGD |
| | PEANDLLERLWEAVVNNDPDLVKELVKRVP |
| 862 | SVEELLELLERLLKETGDPDVSFILWFVRVALESG |
| | NPRIATEIVLQAAEQLGDEELKKLAEELLR |
| 863 | DEHEKEFLKALAKMALDLLRNASDPNLAKELVSTV |
| | KDLADLVNDEDAKELKKQADEVSSKAS |
| 864 | TEKERWKDKIFQLIMELLQLGLDPEEILKVAEKAL |
| | ELEKKGVSPSEIKKWVEKVVKEVKKRI |
| 865 | TDELKKEIEEVEKKVNTSLSKNPFLLWFARMAIEQ |
| | AKEAVKKGDEEEAKSALTTVKELQKLVNS |
| 866 | SEEIERLVRRAAKGDEEAERLIEEIIEELKKRDEF |
| | RAAMVKMILEALKEVPDSEELLEDLTRMLE |
| 867 | DLEELLRREREELGDPRDAIFVLLILLMEEGRLTP |
| | ELLELADEADELIKRGDPRLEEVLKLLVKP |
| 868 | SEMKEVVRALVEEAEQRIREGKISPHEVREYLEMA |
| | RMAVEALGDPEAKKLVERLERLWRKIM |
| 869 | SEELREIVKRALEGDPEAEKLLEEILKELEERGDP |
| | RAFLLWFLIVAIREGNPEVVEDVLEDLLKR |
| 870 | DPELEEAFKLTMLLMHLTMQVPDPEQQKTVETLAR |
| | LLSEAADHNDKDTLRKVKKKAKDVYSKLS |
| 871 | PKEVAEVARLLVEDPNNKEALSLLMKLWFEAVERG |
| | DPELERLVRLVNELLQKDPREVRKLVERLR |
| 872 | GEFYIMLVEMALQAVRENSGDEEFIKRVRKRLKEV |
| | KERGDDSKLEEILREVTEYVQRVV |
| 873 | SEEEVEELLERLKKISPDVQFYLWFLEVALRSGNP |
| | EVAIEIAEDLLRLLGDPLVREIIKLLRKIR |
| 874 | DDEEVKTWEKLAKEAINRIPDKDVRTEVEKMVRTL |
| | IKAAHSPDPQEVEFALEMVRMIIETERG |
| 875 | SEEEEIEELVRKDPIQAYFLLLELANKLGNEELVE |
| | EVAERVQELYENGNPEEATKLLVELLERLR |
| 876 | DPESDAKRIATELATLAKKLDPEESKKVEDTAFTI |
| | AMRLLRSEDPNDQKKAQIIVEVEDKLAI |
| 877 | DDIQKEAKKVIEQLQRIKKTYPDNEKILFLVELAI |
| | EAAQFAIKENDEEALKVAKELLEAVKKIC |
| 878 | SKEEVEKLVFKVMEVAVRVGDPTAESIAKTIVEQA |
| | NEAALNNDDEKLKKVSKRLKSFIKTE |
| 879 | PDELVEEVVKKEGISPFIRFFLRIVVEQLKRGEID |
| | PEDAEKTLRDLLETVGISPEEIEEILRRLK |
| 880 | DESLAKLVFTLMSLLAEAWKYDPDSANTAFELLMK |
| | AAKNNDEEQLKKAVDYAKKVVDRVLS |
| 881 | TVMDEAFKVTFEVMELADKVPDEKVSRLLKTLAKL |
| | LTEAADRNDEELLRKLIKKVKEVRKQAKS |
| 882 | SEMKEVVRELVEDAEQRIREGKISPEEVEFLLLMA |
| | RMVVEELGDPEAKKLVERLERLWRKIM |
| 883 | RKEEAEDTAFKLFMLWLTLTNPEERKKVEKQIETL |
| | VKKLSNAEDPQEQKVAKTVKETLDLLRS |
| 884 | SAFELALKAMEIANKKGDEKAEKTVQDIVRLVMEG |
| | KAPEEYAEKALKRLIKKLK |
| 885 | DELEAVREYAKFLAEIAGVPSVSVRVSNGRIQVTL |
| | KGTDEAARAVQKILTEAVKHWGVDVDVQVR |
| 886 | SDDDVKKLLDELEKLADQIPDEFVSLMVKMIVDAA |
| | RKALKLGDTEEAKMIAKVAEELVSKG |
| 887 | SVEELLELLERLLKETGDPNVEFILWFVRVALENG |
| | NPRVAIEVVLEAAEQLGDEELKKLAEELLR |
| 888 | DVDEAVKAVEEAVRMVQKHPDDEALRWFARVVIEF |
| | VKELASSIEDEDQSKTAQKRATELERKLE |
| 889 | DTFTYLIKLLSKGDPFARHMLENLLEDAKERGDEE |
| | LYRLIKEAIELVEEGNPEEAEKLAKRVDEK |
| 890 | DAREIFRVLALAAETIEKASEEEVRRELEKLIEEL |
| | FQAAHRNDEEALKKVLKRLKELIKEQK |
| 891 | DEFELAFTVMSLAFQADLAEDEKELRTLQSLIEEI |
| | LRKAQSEFLRKLAEDLLKEVANNS |
| 892 | DEEETIRSVLKELKDDASNASSDEDREFLIWMVNV |
| | FAETVPDPTARKIAKEINETLSKLS |
| 893 | DALERAKELAERLGDPLVEVLLYFLEQALRQTGDP |
| | KYEKLAELILELLERGVPPEEVLHLVEELG |
| 894 | DIFELMFLIMHLLKDNPELSELFREVLERLWRGEI |
| | SPEEALRLLKELAREAGNPEVEELLKRVEK |
| 895 | DEEEVIEKVVRLYEEGNTFEVMALLAKLFEETGDP |
| | RYQELLDLVFEVIKKGLSPEELEKLLRKVP |
| 896 | SEEEELLKELKKRVSPEAREYVERLLELLERGDPN |
| | FLFVLLELLFLADELGDEELKRIVEKLLKK |
| 897 | SEAKEVVRALVEEAEQRIREGKISPEEVEFLLDMA |
| | RMAVENLGDPEAKKLVERLERLWRKIM |
| 898 | DEEEVEKLFFKLEEFAYRQGSEEAKKALKKEHELI |
| | EKAQHVPDPTERKKLLDSASKVAKSVIS |
| 899 | SLELLEELVELAKKGDPRVFELALEAMQLANERND |
| | PELSELVQRIVHLITNGNPEEAEKLVEKVK |
| 900 | DEVKEVAEKAVKTGDPKLIKEALELLTKLVLKTGD |
| | PELRRINTLLFEAVMVGDPELLKKILKEIR |
| 901 | DWRELTETLRRLVEKAKKSVEDPFVRALIEIALEA |
| | AEEASKRKDEELLHLLAEVVEELRHAS |
| 902 | SVFELSMKVMHLVAEVERNAEDPEVQKILKEVNEL |
| | VREAAEKNNEELLKRAEKLVKKAKELVS |
| 903 | DDEEAKKLLDLARKALKEVNKTASPEEQKQLTDAV |
| | REAEKLLHSPDPFQREMARMLLEMVVMLG |
| 904 | DDENVKKDVKTVKDIAKKLNDERILKTLQDAERKA |
| | KENNDSRIVRDVAFKLWFQLMNVE |
| 905 | DADSFYSWLLEEALEQAKELQDPEVARMAYESAKD |
| | ALEKYPDSELVRKVLKKAEELQRKLTS |
| 906 | DESLFEALLELMQVSPEAQILVEIISDLFVRGVPL |
| | EEIEKIIEEEAKKLGIPEEVLRLAKELLRK |
| 907 | SELSEEALRLLEEGNPREALKVLFELWFELVDRGD |
| | KELSELVQEIAELLIRGDPDEARKLIKELT |
| 908 | DEVEKLTTEAERVATKLERSPDPDNQFVAFMIKMA |
| | IEFALTISDKDAAKEALKSAVEMARTY |
| 909 | DEEELKELAEKLKEDPQVAVYIWALLAHLWYKTGD |
| | PEVEKVERRFEELVHRGDLETASELVKRIG |
| 910 | SLDEVEEELKKLVNDPEAERNIEFVVWMAKVANLS |
| | PEELLKLLEDSARASGDEELLRAVKELLRK |
| 911 | DDKTLTEVWQHASTLAKQKNDEFVLHFIEVMYTTA |
| | QELGDPDEKRNVEKVLRDLSKDLEG |
| 912 | SEELREELKRAVDRNDVFLVVMLLLDESLETGDPR |
| | YEELARRIVEVDMRGDPETERELLKLAKEP |
| 913 | EEDELRKLVEELEEKARKVITSDEEREIVERIIDD |
| | AKKALKNGDKTTVKFLAKMVEMFIRTG |
| 914 | DEEVKEILREALNLDPEEINDLIFREWFLAVQLGD |
| | PELERVLDEIAELLQKDPEKARKLIKELLK |
| 915 | PEELVKEVEFLILMLKVIATDPEIRKDIERLEKEI |
| | RTRIKKDPRFAEEAVRRLRKILKRLVEEI |
| 916 | DEYQEKANKLYKEAEKLVKTYRSNELVKELFEIAT |
| | RLLKEAREKQDPMLFAFVSMAIDALKENG |
| 917 | DETKIAKELVDSARDLRKQNTEFAKHMVKLLSEEA |
| | KQLKSNIDDPTERKVVSEAIEEIDKLSKG |
| 918 | DKEIAELLKLLLEDPNDKEVADRLERLLMELMHRG |
| | DPLLELVLEAAQLIEEGDPRVREVIKKVEK |
| 919 | PEEEEDVKRIIEDLLKLIQNASSEHEANFLYHMVE |
| | LVVEAVNSEYAKKAHELAKKVLKELRKR |
| 920 | DEEHYRMNIDFLIWMAQTAVSLDPDVLKKITKKAE |
| | KIKKEVPDPDQVVKLLEKLVQEAVELAN |
| 921 | DDEEVKTWEKLAKEAINRIPDKDVRTEVEKSVREL |
| | IKRAHSPDPREVEFALSFVRMIIENERG |
| 922 | DESKKIADEVKKTAEKLIKNGNVFEAMSLLATVLE |
| | KALKNNDEKLYKLVMTVAKEVESLAKS |
| 923 | DEEFATWFVRIALENGDVELAKELAEDALKQGEIS |
| | PDAADRLRKEIESVEKR |
| 924 | DSEEVKDLSKRLQEAAEKAQDPNMLDKAFKVWFQL |
| | VEAYNNNDEDKVKDASKKAKEVIKTIDS |
| 925 | DEREELAQEVAEEMIREAKHAESERHAEFYLFFAE |
| | RLARAINSEETLELIKEVAREIREKG |
| 926 | SSEDVVRELLKAAKKTGDEFYVLLAEMAARWIGDE |
| | ELKQRVKEAERYVREY |
| 927 | SEEEVREEVERTGDPDVKLALELIEELKRRGDPES |
| | AKIVTEMVLKLIRTGDPFLRALLRMMIENG |
| 928 | DEMKKVSEELKKDIKEALDKNDSHNLRFLIMIAKM |
| | VASTINDEDEKKEAEKAEKILRDRES |
| 929 | DHEEEIRSVLKDLKDRASNASSDEEREFLIWFVNV |
| | FAETVPDPTARKIAKEINETLSKLS |
| 930 | RSEEDLRFRLHMIELLLRAIGVSEDEIKKINKTLE |
| | ELIRKGDSETFKRIEDEVEKRVKKET |
| 931 | DDKTLTEVWQHASTLAKQKNDEFVLWMIEVAYEAA |
| | QIYGDPHVKKEVEKSLRDLSKKLEG |
| 932 | SDEDVRSLLERIDSELKNVSNEFVKFFVELAVREA |
| | KEALKQGDKEYAEMVLKAAQEVLSVE |
| 933 | DEEVVELLKKLVNDPELEKVLEILLKLIKEGDPDA |
| | QFLLFMLKLALEVGDPDTVRSILEDILRQT |
| 934 | DEAFEAAHILAESLFIAREIGDEELIRLAEHLLEL |
| | LSKAAEENDKDLVREILRLARESLKRSR |
| 935 | SEELREELKRAVDRNDVFLVVMLLMTLSFETGDPR |
| | YDELAQRILHVASEGDPETLRELLKLAKEP |
| 936 | SEELEEVLELLKRDPKKALELLFELWFEALRRGDP |
| | ELLRVLEELNHLILEGDLEEAKELLKELLK |
| 937 | SETEFVKFFVDVALESAKELQDPHVAEIAVRIAED |
| | VLKEDPTDETVRELQKKAETVRDQLKS |
| 938 | TVEELARKLLEEGRLSPEEIVEVLLRELRKLGIPP |
| | FEAALLLLKVANDLNDPELYKAVQELAERT |
| 939 | PQLDKMVKDAQDLADKIVKKASNPEIQKKAKTVKK |
| | LLEEARQKKDEQTVAELAFKAMELWEMVT |
| 940 | SADEATDSAAKAAEKGDSEYIERLLKKAKSDNIID |
| | EFHIWFIKMLLEFARE |
| 941 | SEETITRLYKDAKKKAEDKKNEELKKTVKIAKDLL |
| | DTAKNVDDRMTKEFLETMAELALRDV |
| 942 | DPELEEAFKLTFLLLHLLMQVPDPEQQKTVETLAK |
| | LLTEAADKNDKDTLRKVKKKAKDVYSKLS |
| 943 | DPQKELHKVYETAVKRIRSIPDKEKQKEVVKEATE |
| | LLRRIAKDEQEKLSAEELSWMLKVLS |
| 944 | DPEQELRTLEKIVTEAAKDPRRSEDFFKAASRLAE |
| | LIYKANDPKIQTIAREVLTKAFSIAN |
| 945 | TVEELEKLVEKLTEKVKRKDPRLSEKAQRILDQAK |
| | DLLSKAENPLLQEMIARFAVSQLQRLV |
| 946 | SMEDIVRELLKAAKKTGDEFYVMMAEMVAREIGDE |
| | ELKQRVKEAERYVREY |
| 947 | DKSDAQRILFEAWFLVVELGDEELEKEVDRLQKEF |
| | LEADKKNDKDLLDEITRQAEELLKRIKS |
| 948 | DLEKLKKDAKKALEILKKLNNEFLVWFLENLVKAL |
| | EMAIRKNDEHVVKEVSKAIQEIIERLQK |
| 949 | DEEELKKVAETAKTVAKQKNNEEVLKAIKEMATEA |
| | EKLIKSSNPRDQMNAEFIIWMLRVLIED |
| 950 | NEQEKRNASFLLEMAKMLADFISNDDLRKKAQSLV |
| | EKARKIDDPNVAKKVYKIVSTIFERVVK |
| 951 | FEDAEFILWFAKALVESVGDEDALEKLKEFEKKVT |
| | EAIKNNDEEKVRKLLSKAEELAERVLQ |
| 952 | DEHLKKLEKKVRTLLEKARKNNDEEVMFLLFFAEL |
| | SIEMVKMNPEAKEDAQKTVEDLLKQAEST |
| 953 | DEESSRAAKDLLKKAKEAVERNDSQTVSFLLWMVK |
| | AVAQNAGDEEVEKSVSELEKLWKKIS |
| 954 | DEVEKLLREANELAFKLLSDDWRAFDLLQTLARIA |
| | DEANLSGDKETASKLLKTIIELLKRG |
| 955 | DPSEEAQKLVDKVKEDVKKKNDEDAEEAVKIAEEL |
| | LKAAKNAQDPRDREFLVLIIKMMVENLS |
| 956 | SEEEYKKKIKELLRRGDAFEAALLAMEAFFRLGDP |
| | RFEEILRIIHELFERGISPEEIVEILERLE |
| 957 | DWRELTETLRRLVEKAKKSVEDPFVRWMIEALLDA |
| | AEFASKNKDEEMLKLIAEAVEDLRHLS |
| 958 | TEELIKLVKELAKRNPEEAKKVVEELVKLLGLDPL |
| | AERMLKELVEEYYRTGDPFYLFMFEVLVRN |
| 959 | DELEKVVKELSTDADKAVKNGDKDKVKFLVWFLKS |
| | VVEFDPDNPAAKLAKKVIEKLEKLIKS |
| 960 | SEEIKRIVELVKKGDPEALKRLRELLEELKKKGDP |
| | RAFLLWFVIVALEAGNPDIAKEVAEQIERR |
| 961 | DPSEEAQKLVDKVKEDVKKKNDEDAEEEVKIAEEL |
| | LKQAKNAQDPTEREFLVEMIKMLVKQLS |
| 962 | QEEYKRLLEELLRKAQKAVKNNDQHEVSFLAWFIT |
| | ISAENVEDEDVSKHFLELAKELHKFVNS |
| 963 | DRDEQLKKAIKDLLEQLKKASSRHEASFLIWFIRL |
| | LREDAQTPEVRTLATEADKIYKKET |
| 964 | DEWQELSKIVKSLIDELKKSPDPKDQFAAAMAEMA |
| | WEMAERAQDPNVRREALKTAIEMVRDT |
| 965 | DPFTAMSLVARVFKLALEKNDEELQKEAETLMEIV |
| | IKAAQNNDEEAVKEVIKRAKELLSKS |
| 966 | DDTKKLHDTVTKYAQEVLKRARDEDVKKVIEELLR |
| | DAQEVIKNHDEETAKFLYLMIQLASKQAI |
| 967 | TEDYEKVKRELEKVEEEAKRKNNLEALFLVFMAKL |
| | SLEAFNLDPDNKDQIISVAKDLLKLAKNS |
| 677 | SAEEALDSAVKAAEKGDSEYIERLLKKAKSDNIID |
| | EFHIHMIKLLLEEARR |
| 968 | SVFELALKAMEAANRKGDEDAMRKIIKIVQLASDG |
| | KIDESQASTRLRRILKKLRS |
| 969 | SALEEAERLARELGDEDLLFLLAMVKMARSTNPEV |
| | AKELLEEAVKLAEELGDPDLLKLLRLALKE |
| 970 | TEEERVKEALERNDPDLVKNIIFEAVMELLKRDTE |
| | KAQRVAELAVELLKEGDPDKLRELARRADK |
| 971 | DDETKELQNEAFKAVMIVMQLNDPRLEKVVRDVQT |
| | RFEEAQKHNDEETSKKVLKKLVEIVRKT |
| 972 | SEEKEVVRHLVEDAEQRIREGKISPEEVEFLLMLA |
| | RMVVENLGDPEAKKLVERLERLWRKIM |
| 973 | DEEEKKEKTLDKLLSEAKKKSSQNPEVQFLAWMLE |
| | MLVEQFKLVPDPEIRKEIYKAASDLYKQT |
| 974 | AELAFKAMSLAAQLAKALLDGDEDKVKTARSLLDK |
| | VEKEVKKQNDEEVLKFVQSLKKTAEDVT |
| 975 | NEDEKRNAEFLLWFAKVLADSISDDDGRKKAQSLV |
| | EKARKLDIPEVAKKVYKIVKTIFERDVK |
| 976 | DVFTVALKVIEILNRLINNAEDERIKKLLETVSRL |
| | VVEAAERNDEKLVRDLYKTLEKLVRELRS |
| 977 | DESEKFQTVVKELEDLVHSVEDPTRRKKARNIAFK |
| | LVMKLMN.AEDPKEFKETVDKAKEIVKKAA |
| 978 | PLEEQLRELLEKAFEAWAKIRDPEEAETFVEKFQT |
| | LEKQFRNASSEEEKKKVVEKIKKLIDSVL |
| 979 | SEIEEVLEKLKRLSPEISFFVWFIEVALRNGNPEV |
| | ALKLARDVLERFGDPLVKELLELIEKLVRK |
| 980 | DDKERLRKLVDDLIRKAKEVKDPETVRNLLFQAIM |
| | AAMLINDPELEKKVKEVFKEIKKKVS |
| 981 | DEEEKLQTVIKELEDLVKSVEDPTRRKKAHEIAFK |
| | LVMKLLQAEDPKEFKETVDKAKEIVKKAA |
| 982 | DIDEKLDSLVKKAKRYVDETNSDELKEEVSIAEEL |
| | VRRAKNASDPNDKREUTWMVEAIYELVI |
| 983 | DPKKLDELRKKIETLLRKVTDPFAKEAVKAALELA |
| | EEALKKQDPFLEFFARMAYEFAKEMQ |
| 984 | SEESEKVKRWVETELTKANKQNDYRKAINIVETAA |
| | MLAMQLNDEELEKWVKSLAKEVMSKKR |
| 985 | DPKEELERVTKTAQKTVTELNDEFALEMVNNLVEF |
| | AEEAVRNNDEELAKTVIRALQETFKNMM |
| 986 | GEFYIMMVELALQTVRELSGDEEFIKRVRKRLKEV |
| | KERGDDSKLEEISREVVEYIERVL |
| 987 | DLEKEAKTVVTLLKRLKKQVTNPDVKKLVDEVAKS |
| | VETAVKKKDEDKIKSLVFEAQILIFAI |
| 988 | DLHKEAFKVWFEAVELIFKVQDPHIRKLLEHLAKI |
| | LQEAANKNDEELIKKILKLVQKVIKEARS |
| 989 | SIEDIAHKIISLALELGDEDALSQAAEVLEKWLSG |
| | KISPEQAKKLADRIRKILE |
| 990 | DPFTAMELVARVMELAIEKNDEELQKEARTIMEIV |
| | LKAARNNDEEAVKEVIKRAKELLSKS |
| 991 | TEEEKKAKKWVEKLVEKAGLDDFYKEMAKLVFEDA |
| | VLQGVSTKEARKIVKKVIKEARNV |
| 992 | DKREEVHTLLEILAEAAEKRDEETIRKVASRAQKL |
| | AKEIGDEFLLSFIQMLFEYAQQLLS |
| 993 | GELAKRALELIERGDLDEVEKLWFEIVERANRTKD |
| | PELEELLRLIHLAAQGDPEAKKELEKIIRK |
| 994 | SELLEEAFEIWFKVVEIAYKLNDPDKQRLAEEVTR |
| | LIETAMNVPDPTQVKKLVTKAKEIFKKVK |
| 995 | DEEELLREVVERFKDDPFHVAFLLLEIAERRNDPE |
| | LAELISEVIEEVSQKGLPPEELLRLVLELK |
| 996 | DFEELLEEARREGDPRTLVFLIALLALQRGDPELV |
| | REIEEVEELIHRGELPPEELRRLAEELIKK |
| 997 | SVELLFEVWFLIAQLGNEEVLRLLEIFAEAAIRGD |
| | PDLLEKVERRIEELSKTDPAARLVLEAIRG |
| 998 | DEFLIMLAKMLLEFARNNGLLTPDEAEKWKKLLEE |
| | VRKRGDDNQIRKIYEEIVELVERLAG |
| 999 | SEETITRLYKDAKKKAEDKKDEELKKAVKDAKDLL |
| | DTAKNVDDEMEKKFLEWFAEVLLRAV |
| 1000 | DIDEKLDSLVKKAKRYVDETNSDELKEEVSVAEDL |
| | VRRAKNASDPNDKRELTEMVELIYELVI |
| 1001 | DLEEKAKEAYEEGDLELLVFLVLMILMEKGDPRLE |
| | ELLKLAEEVKKTNDPELRKELRRLAYELIP |
| 1002 | SADEASDSAAKAAEKGDSEYIERLLKKAKSDNIID |
| | EFHIWFIKMLLEFARE |
| 1003 | SDSDEISSAVETLLKQAKDAPDPTDAEMLLWFAKV |
| | LVDYLNNDELRKKVEKASKEVEKINRS |
| 1004 | DESKKIADEVKKTAEKLIKNGLVFEAMSLLATVLE |
| | KALKNNDEKLYKLVMTVAKEVESLAKS |
| 1005 | NEKEKMDAQFLLWFAKVMADNISDDDKRKKAQSLV |
| | EKARKLDIPEEAKKVYKIVATILERDQK |
| 1006 | DPESDAKRIATELATLAKKLDPEESRKVEDTAFTI |
| | AMRLLSSSDPNDQKKAQIIAEVENKLAT |
| 1007 | SSELRDRLKKTFQELLKRGVSPWQAFEIIEKLLEE |
| | LFRKGKLDVDVAFEVMHILAELMRKWE |
| 1008 | DRTEKDQTVIKELMDLVKSVEDPTKRKKANDIAFK |
| | LVMKLLKAEDPKEWKEIVDKAKEIVKKAG |
| 1009 | NEEVKKEIKSLEKTVSTLASKDPFALFMAQVLIEF |
| | AKEGLKKGDEEDVKEANSIVRDIIKRLQS |
| 1010 | DPKKLDELRKKIETLLRKVTDPDAKEIVKDALELA |
| | EEALKKQDPFYEWAARVLYELAKQLQ |
| 1011 | DDEEVKTWEKLAKEAINRIPDKDVRTEVEKMVRTL |
| | IKRAHSPDPNEVEFALLMVALIIETERG |
| 1012 | NEEEKKKAKKLLDEAKKLADQISNDDLRKDAQSNV |
| | ERARKIDDPRDAKFVYFFVKLIYEFVQS |
| 1013 | DETQKRQVEKYVRTIEKLLKDAKDPKEITELLEKA |
| | LTYLERVNSELAMEAMYRLMEVYFEAT |
| 1014 | NEELLTKEVLEFAQSLAQRASSQEEVDKISKEAQK |
| | ILKELNVDPRLVEKVSRLVQEYYKKS |
| 1015 | NEEEKRHAEFLLEFAKMLADNISDDDGRKKAQSLV |
| | EKARKLDIPEVAKKVYKIVTTIWHRDQK |
| 1016 | PKEELEKLVRTLEELVKKKVRDPFVKWMADVTVTF |
| | AREAVKSGDKDLATMVIEDLKELLKTI |
| 1017 | DIMSLAFEAMELANKNGDEDAMKTIQKLFELFDEG |
| | KIDPTQVQRILRKIAKRLKG |
| 1018 | SEVEELLEKAKRTGDPRLASRAIFLVLMELLKRDP |
| | EEVQRVQEELLRLLKEGDLEELERLLRKVQ |
| 1019 | SEVLKELKRLLEEGDPEEALRLAERLLKEAKEKGD |
| | EELAFILWEVIVALRQGNPEAIRVARELVR |
| 1020 | DKEELKEELEKLIGDPDAAELLLHYIEAVRRGEDP |
| | PERVEALVEFIVKIAGLPPEVEKRLRELAR |
| 1021 | PHVENLVFTLVMKAMEAINKNDEDTLKKLKDTLDE |
| | LVKKDPRETEAAKLAKKLIKEQEELT |
| 1022 | DPVEFLSWMAETAISLLEELGLLSKEELQKWKEIV |
| | RKLTERGDTENLEKVLDKITEIIEKLQKS |
| 1023 | KEEVNNQAFKLVMEAMELANSSDPNNRTKAKELIK |
| | EAKKLAKEVNNPEVEDTVREVEESIKKVG |
| 1024 | DEFTEIVKELVKLAEEAVKKNDEDSVKFIEAMLKM |
| | MKEAATDPKQRELADRAIKKVQKLLKS |
| 1025 | DEHLKKLEKKVRILLEKARKNNDMKVAFLLFMAEL |
| | DIELVKLNPEAKEEAQKSVEELLKQAEST |
| 1026 | DPFTAMELVARVLQLAIEKNDEELQKEAHTLMEIV |
| | IKAAKNNDEEAVKEVIKRAKELLSKS |
| 1027 | DEVERVVELLKRLGDPVARELAELIEEILKRNEEM |
| | ARSAASSAEMALKKNDPESLKFLITLLKMV |
| 1028 | DIDEKLDSLVKKAKRYVDETNSDELKHAVSSAEEL |
| | VRRAKNASDPNDKRELTAMVEMLYDFVI |
| 1029 | DDSEEITKLLDDLVTKAKKKIKDPQLQLKAEVLAW |
| | FAKVAASSPDPEAKKVAKELVKELQKLV |
| 1030 | DPKKLDELRKKIETLLRKVTDPDAKEAVKMALELA |
| | EEALKKQDPEYEWEARVAYEWAKELQ |
| 1031 | SDEDEIFTLAMLAMEAIFKAAKTEEEFERLMKKLQ |
| | DVIEKAQQLDPSQQKKLLKEVLDEVKKL |
| 1032 | NEVFELALKALHVAFKVGDEDALKLIQKIAEKVSD |
| | GKLDEKQASKLLKSLIKKLE |
| 1033 | DVDTEALRAAEDMIELLKNAKDPTEAKFAKTMVEL |
| | LVELVKDPEARKIIQKKLKEAEEYVNKL |
| 1034 | GEFYIEMVEMALDMVRMLSGDEEFIKRVRKRLKEV |
| | KERGDDSKLEEILREVTEYVERVL |
| 1035 | DAFDALSILAEAAELVRKLGDEELKKEVDRLLEEF |
| | TEAARNNDKDLLDEITRQAEELLKRIKS |
| 1036 | DDDSYKKKVEDTLESLIKKAKDPLARKVIEELARD |
| | AKKAVDNGDKEKVEFLVLLAKLVADNAAS |
| 1037 | TDAKEIIKDLKKRFEEAVRRGVDPRQAEFLVKMVE |
| | MLAEDVGDEEVKEVVKELRKRLKELKT |
| 1038 | DELEKMVKRISTEADKAVKNGDKHEVEFLVFFLKL |
| | VVEDDPSNPAAKLAKKVIEKLEKLIKS |
| 1039 | SVSELLSEAASKNDPRLLEEAVELLVKELGLSPEE |
| | EERLRRLVREALKRGTLFEVVMELMTLANR |
| 1040 | DPREAEELAKKVEEVASKRGDEVMLRAVEEIKKLI |
| | ETAKKSPDEFYRSFVDMASDELKEITS |
| 1041 | DELEKVVKRLSTEADKAVKNGDKHEVEFLVWMLKA |
| | VVDFDPDNPAAKLAKKVIEKLEKLIKS |
| 1042 | SEEKEVVRALVEEAEQRIREGKISPHEVEFYLWFA |
| | AVAVELLGDPEAKKLVERLERLWRKIM |
| 1043 | SLEEELKRLISERLGVPPEFVEEIVEFDSEELKVP |
| | PEEALLELAKAVARLERDPELEELIRRLEK |
| 1044 | NNEAQFLLWMVQMLVEAAKLDPDSAEELSEAIKDL |
| | LKRIRKLGDEDASKKATELLKEAEKVIS |
| 1045 | DEEEEKLEELIRRGNTEEAKKIVEKIVKELGLDPF |
| | EEWELKLLVENAKVDPLVLEVLKRALKELR |
| 1046 | SKDEKLHDKLFKLWFLLVNLHTEEAKKLSKKVHTA |
| | AEKASTLSPEERRRLLEKVAKEAEKLIG |
| 1047 | DESLAKLVFILMSLLAEAFKYDPDSAETAMELLMK |
| | AARNNDEEQLKKAVDYAKKVVDRVLS |

TABLE 8
PDGFR binding polypeptides

| SEQ | |
|---|---|
| ID | |
| NO | Sequence |
| 1048 | DVQQEAKFVIARLAMLALHVDDPEQRKTVFKLMLLLADAV |
| | ERGDRELLKKVIKTARDVISKVNS |
| 1049 | SEIEEVLELIRRYDPEVVKELEKVERALKRSGVSPEKILK |
| | YLLIVAHFLGSPLAVRLLFRLMTKR |
| 1050 | SLEELERLIRKLVKEGDPAARRALLVLESLKRRGVSPEEI |
| | VRRLIAFLFILGNPELIRLAIRLFL |
| 1051 | DLLEKAKKVAREILDPEVVRIFELAVEVIKKKGANPSSIL |
| | IAVLRLLRRRGVDPELLREVERRAR |
| 1052 | DEREELIELIERNLDDPEKVHELLKRLLELAEELGDRRLY |
| | RYVMLIFWALRRNPERAERLLRKLK |
| 1053 | PELIEEALELLKEGDPKKVFRVLARLVQLLREKGDPRYRL |
| | VLLILAYVREGNPEAARQLLRIVLK |
| 1054 | DELREILREALEKGDPELVKELLELLARLADETGDPKLRI |
| | VIAYVWFAKKKNDPRLLKTALQVLK |
| 1055 | DDERLARLAFRVLIKRAGVPDEDVKVINGKVRVTITGRDQ |
| | ASQEALQLVFALARRLGLQVQIDTR |
| 1056 | DDERLATLAFRALIKRAGVKNLDVKVTNGKVRVTITGRDQ |
| | ASFKALQLVFALARRLGLQVQIDTR |
| 1057 | SELAEEILELIEKGDPRVRKLILELLKELKERGDEKRFKL |
| | LVRVWELLRSGNPELAKQLLKKIKK |
| 1058 | DKAREFLRAAAKYGDPSAKRALKLLEELRKRGIPPEEALK |
| | IVVEFLRKAGNPRLARVVEEALKDE |
| 1059 | SLIEEILKEVRKKNPRVVYILERLLRRLKEDGVPPEKILR |
| | ILIAAAKRLGDPEASELLRRALKRK |
| 1060 | SVEELVKRILELLKEDEHRALRRLVVLLLNRGISPEEIAR |
| | IALRVLKLFKRDPEKARELIEKVLR |
| 1061 | SELFREAIELLEEGNPEEARRVLERLAIRLAQEGDPRRRA |
| | VALAYFYLKTGNPKEAAQLLEEARR |
| 1062 | SLKRIVVILLLEAGVSPRDIARLFRIIDELKERGVSPEEI |
| | LKILIKILEELGDPRAAKLLRKLLK |
| 1063 | DETEEKAKRVSQYARNLGIEDVSISFHDNRLRIRVGGDDE |
| | KARRARRQIVRIAIQLGFRVLVITK |
| 1064 | DLRDEFFRLLLTAIEIAKNVPDPRKASKIRILVAHVYFKA |
| | RTLSKEQRVKLARELVDKAKKE |
| 1065 | DLSETVSKLASEVAKKVGVPSWEVRVHNGKVIVTLKGQDR |
| | AARRAMLLIRRIAEKLGVPVRVLVL |
| 1066 | DIVELLTTHAERVATKLERSPDPRNKKVAKFIKTLIKKAK |
| | SVPDKDKSLRILAMAFFFAKRE |
| 1067 | NEELEKRVEELIKRIPNPKERKVLRLLLKRLKEGDPKAAA |
| | LLEILLAVFERGDPERLILILKRVK |
| 1068 | DKQHRAVALIRLVARRLKVPDVSVDVRNGRVTVRFGGTDE |
| | RVRQAFLLARLIAKHVGVPIDVKVK |
| 1069 | GPLERTARELAKELGIPPEDIERVLEVLERILGDPRRALW |
| | ELASLLTRAKKDPYVRELLKALIRD |
| 1070 | SEQETASRLIKEAADRLGVSDVKVDVRNGTLRVTVRGNNE |
| | SAQQLATEVLQIATQLGVRVLVLER |
| 1071 | DKERIFARALRALARRAGVPSVRVEVSNGKVTVSVKGKDR |
| | IARLFAQLVYNLARREGIDVDVQLR |
| 1072 | DDWRRILARALLLAKELGVPRVVVVRIDGRWVVLVKGDNE |
| | SAKKLITILKKWEEKKGDEVRVQIS |
| 1073 | DVTEKLKKLITEAAKRAGVPSVEVTELRGKVLVRERGRDR |
| | SVLRAALLARRLADKENVPLRVIFH |
| 1074 | DKLATRAIRLALQAHLQARQRGTPVRIHLSFKGFTIDVTV |
| | TEDERSLERAIRLLILFERRSSEN |
| 1075 | DPTSFRLVMEISLVLQLFKKGEIDPSQAKTLLKRLQEKAK |
| | KIGDRFAVVLAEFAIRVIKKAEKS |
| 1076 | SLERIVRAAIEKAARRLGVPDVKVSVRNGTVHVKVRGSDR |
| | AALAAKIFAWNIARRLGVPVSVTIR |
| 1077 | SREKAFASYVRQVAKRAGVPSVDVKVSNGKVTVILDGDDE |
| | RARQAELLIRWESRHLGVPVTVKKK |
| 1078 | SDDQEKLERIVHAIVRHFGLSPEWEKRLKKVVRELLKKGV |
| | DPEQIVKIVVKIAIRVHNQK |
| 1079 | SEELVKKVEELVKKTGDPLLKLLLEILKRLLKINTPLARV |
| | LIDLILLLIDAGDREKLERAIRRLR |
| 1080 | DKELLEEAIKLLKEGDPEKARKVLLELAREAHERNDPKLY |
| | RAIALVYFYLRSNPRLAIQLLELLT |
| 1081 | DVERLVRAAVRKAAKKLGLPHWHVSVSNNTVRVQVRGRDK |
| | AALALLIFAWNIARAFGLKIELRHQ |
| 1082 | PRRIEDALRSLINSPNPELRLLAKVAIELLKRGVSPRRIA |
| | LIIRRLADRLGLSPRVLRLVLIAVE |
| 1083 | SKDEKARRLVLRILLEVGLKREVVLVRNGRILVVIRGSDE |
| | RAQTATKLVRTLAKKLGLEVRVLIV |
| 1084 | DRRRRFISRVLTIAKQLGVPRVIIIVNDGRVTVRVRGDTE |
| | KARTVQKFAQKVAEELGLDVDVQVR |
| 1085 | DKLAVRAIRLALQAFLRARLRGTPVRIHLSEKGFTIDVTV |
| | TEDPRSLQRAVRLLLLFLRRSKEN |
| 1086 | SFEEVIHRALMVAFVEGDKKAFRILTKILELARQGKMSEE |
| | QAEKRARRIEEKLRK |
| 1087 | DERKKVDAFLRFVIVRVFFVKDPETKKDILRKLQEFAEQI |
| | SDEEAKKKAQKVWELLTRLS |
| 1088 | SVEERIEELKKEGDPKKALILELALELLKRIPPARVYALL |
| | LALARRLGVPPRDVARLLFIALQAK |
| 1089 | DRALEIARSVLRIANEAGVPRLRVFVSKNTVRVRVEGDDD |
| | AAKRAKKLVEEYAKKLGLKVTVTKK |
| 1090 | DYRTLFLQIWMVATTLAKEAKSEEDVKKAEKKLQEWLDKL |
| | NVNKLLQATARRILRRVYKQLS |
| 1091 | DKDLKELYKELRKKLSPEEVVKIVFKVIEEKFGDPRRAFA |
| | ALYLVERSLGIDPEEVRRLIRWAFI |
| 1092 | SEEELIEKVERVVGDPALRQMURQMIEWLKKQGVDPKEAV |
| | VLAAFALARHLGDPELRKVAREVLK |
| 1093 | DPTRHRLLLRIFMVLVLFKKGKIDPSQAKTLLKRLQEKAK |
| | KIGDEDAVFAAKQAIDFIKRAEKS |
| 1094 | SLKELAREARRRGNPDVAFLVMLARYARKLGLPPKTIKKL |
| | LERQAKRRGIPPESLEEAKEIVEKG |
| 1095 | SERVAKIALLAALLGDPRYERSVKERVKELVKRDPRVEEL |
| | VRELIKRLREAGNPRAARILEEALR |
| 1096 | SLEEEIERALKRAGVPPQLIRIIKKLIRELEKRGVPPERI |
| | KRLVFTFLLQYDRRLALRVFFLLRK |
| 1097 | SEDETFKKLVKKIAKEANVPSVRVTKVRGNELVEIKGRDK |
| | AALRAALLIRRVAERLGLKVRIIMR |
| 1098 | DELREILEKALKTGDPRLLKLVLELLMRLALKGDPKARRL |
| | LAFLQVLRRRHDPEEVVKILLRLLK |
| 1099 | SVLEELLRLARELGDKRLADIIRALIRAVRNGKISPEKAL |
| | RLILAYAHFLGNKELFRVVLRLEKK |
| 1100 | DKLTKAAKELAKKVKSGKLSPKQAMRLLFVIAFRIGIDRK |
| | EMVRAAQHLMLLITK |
| 1101 | SEKEYLERVLRLLEQGNRRRAIARLLQILQELKRRGDPRV |
| | EEFRKLVRLLARDEEKAKELVRRLR |
| 1102 | TEEEKLKTKIKELAKKVGVPRVKVRVRDGRVLVLVIGDDE |
| | NAQTFSKLLLRIALREGIPLFVFIQ |
| 1103 | DNKVEMRAYEIVQDIQKKYPDPREAKKVAKEVLKRIVDSI |
| | DDPDERFVVHAAARLLLRKIG |
| 1104 | SLEEALEIIEKELGNPKLVQALRQLIRLLKRRGESPELIL |
| | AALRLFLRRINTPEARLAYKILRET |
| 1105 | SEESKRAKELIEAAARRLGLDPSAVEKARKLFDKLVKRGI |
| | SPDEAFHIAIRFLMKLSTSK |
| 1106 | DARLRLIFWARFAARSAGARRVRVTEHNGTVRVEVDGNNE |
| | RVQQLLKSVKRLAEKLGVPLEVTTR |
| 1107 | IEEAKLLKRMVKAAAKRLSQGRLDEEKWRLIVIMASSRLF |
| | SLGISREEIRDFQRQAKELKKKA |
| 1108 | DERTKKKAEELFKRLIKILQNAPNPFLRQLAKELETKERR |
| | AKKNNDDDTLIKLVEFAIMVVRAG |
| 1109 | DERVRRIFKVLRELARISRNLSEEESERFFKKILEEAARR |
| | NDRILYLAAKLALRDSRRALHE |
| 1110 | SELDKIVKKLTDIAREAYKANDKETIEKALRWAALFARQY |
| | RDPKFRELFRIIFQLRQRVER |
| 1111 | DERKKVARAVWEIAKRAGVKGFFVFVKEGKVWVTLRGTDE |
| | SAQTAKKLIEEWSRKNNLDVTVRVR |
| 1112 | DEKRRIFMRILLAARRVGVRRVLVFERNGREKVFIQGNDE |
| | RAKTASKLVTKLARKLGLDVKVTVV |
| 1113 | SVDELVEEAIRRGLPPEELEKLLEELLKKTGDPRVRYVIV |
| | ALRILLRLGAPPQIVLWVVLRIARR |
| 1114 | DERREELVKRLKKSPDPSEKAIAAAYEYFLKMGLDPEMVL |
| | YQLKRFARKVGDPKLEELVKRLEET |
| 1115 | SRLLRFALLVRRLAEDANVPHVRVVVSNGRVRVLVRGNDE |
| | SAQKFIKLFKTVATKLGIDVRVHVV |
| 1116 | THREILKLILKIAKKDEELAKRIYLIVWALRRGAIDPTQV |
| | ERLLEKLIREFGDPLVRLVLELVRK |
| 1117 | SLELVEEFLKKENTPEARILLRALEFLKRRGWPPRLVVQI |
| | LYTLARKLGNPELAEEIRRLAREAP |
| 1118 | GPEVLKKFEELRKRDPREALLFVLRIVFELGDPRLKRLAV |
| | RAWELLKDGNPLVEQVIRRLRELLH |
| 1119 | SEEEKITKTIETLARRAGLDDVKVNVKNGKVIVQIRGHDR |
| | AARRLAFLVIRWADEQGLKVLVQIT |
| 1120 | SEDEKIRKTVEQIAREAGVESVRVNRINGKLHVILKGDNR |
| | AARRVAALVIRYADEVGIRVDILLL |
| 1121 | SSRRRVISRALQLAQKAGVKRITITEKNDRLRVTINGNSP |
| | SIQKARRLIEKLAKEAGVEVEVRTR |
| 1122 | SEEEKVAKVIETIAKEAGVPSVRVVRVRGKIVVIIRGTDE |
| | KARRALRLIFQFANKQNLPIYIRLR |
| 1123 | DRSEVRKVETLAFRVARLLASEDPNNRKRAKKEASTIKKE |
| | LSKKSPSLSSLFEFEVRRITSRLG |
| 1124 | DPVEEKFKRLEKELRKSIKKGKISPEQALRILIKEMMHLF |
| | VRGKLSRNVAEMAVKEMREVDERKR |
| 1125 | DVEKLVKEVQKLAKKRGDNRAHVLAFVILLMYRRGLISSE |
| | IASRVLESVAKRLKG |
| 1126 | SEERKTLARLLLIAKRAGVPRFIVLHVNGRLQIIFKGDSP |
| | SVQRARKLAEEFSKKSGIPLKVRVK |
| 1127 | DLDETVKKIAEDVAKEAGVPSFRVRVSNGKLTVRFKGNNE |
| | SARRAFMRIRLLLRRLGVPVIVQVV |
| 1128 | SRVKRLSVRIVMLLLFARKAGLIDPEEVERLYEEVNRLAR |
| | KGDWKQLQKLFRKILEEIRRR |
| 1129 | SLLEELLELLKRYVSPELVERVERLLRALLRHGFSPQLVI |
| | LSVIRLLEARGDPELARLLREILRR |
| 1130 | DQDRRILEIIHQIAEEANVPSFTSYVSDGIVFVRVWGNDE |
| | AAKTALTRIKKTAEKLGVQVRVRTL |
| 1131 | PELRRLTARLLFLVVQAIRRNDEDTVRTLLKIAKELADRN |
| | PKYKFVFLVLRSMIFRMRRRI |
| 1132 | DKSETFSKLAKEIAKKVGVPSVKVYNIRGRLLVTFRGQDR |
| | AARRAALLVRRLADKLGVEVRVLIQ |
| 1133 | SRHRLVSQLIRKAARRAGVPHVDVRVKDGTVSVTIDGTDV |
| | AARAVFAFVKYVSKLLNVRVSVTEK |
| 1134 | DEELIERALELIERGDPSVRKVVAELALLLARKGDRRRLS |
| | YVARVLRLLKRDPRAAKQALKILLS |
| 1135 | SRLLALSIFIWNIARAAGVPSVTVRVKNGKITVTVSGNTE |
| | SAKLARRAVEAFARRYNVDVDFQIR |
| 1136 | SELLRFRLRLRRVADRANVRRVTVFRINGKIFVLIKGNDD |
| | SAKIVEKFVRTVAKKRGIPVVVKLE |
| 1137 | DEKEKTVRSVIKRLQELSKKASDLRKRMRLIMLSKILTEA |
| | LKEDKDEAHKLAQRVKEMLDKD |
| 1138 | SELKEVVERVLKGELSPSEVLRVLAELYRKTGDPRYRNLL |
| | ARLYLARRRGDPELLKRILELIRRA |
| 1139 | SRERIARLMMIAFEKGDRRALVVVAQAWFALRRGDEKTAK |
| | RLIKLASKLLS |
| 1140 | DKETAFAALLRAMAKRAGVPSVRVEVSNGKVTVSVKGTDD |
| | AAELFRLLVMFLAKRLGIDVDVQIR |
| 1141 | DDRDKLARLVIKAMELADQIKDERWSLHVKTLIAHAWFRN |
| | DTKLAEEVLRLAKKIVESVQ |
| 1142 | SERKTLYQLVARLAQELGVPRVLVRLSEGEFLVFVFGRDE |
| | SAQTLSKILTTWAKKVGLKVEVILR |
| 1143 | DEEVSRAYRLAAKLGDPRLAALIRLLLKLVKRGDPNLRLV |
| | VAVARRLAKELGDPEILEVLEKLLK |
| 1144 | SSEEALKLIKELAPPEVRIAIEALHRHLKRQGLPPRLIVQ |
| | IIIKFLERRGVLPPEVAKEARELLR |
| 1145 | DRDEEFQKKVESAYRRLKERNASDKRILSYIARLLREAQK |
| | IGATKARKLAAEIALRLARRLD |
| 1146 | HEEIAKKLKKKVRTILDQLNASDEQKVRAFELLAFAERVL |
| | SRISDKRHAKREADEVTQMISNM |
| 1147 | SEELEEVLELLKRDPKKAFRLLAELAREAQRRGDPELFKV |
| | LMLLLIYIRLGNLKLARQLLEVLLK |
| 1148 | DKRQVLRVFEKAFHHMRHHPQVKKLAIKVMMLAVQAKKQN |
| | NPELLKQAEELLKEVEKRAK |
| 1149 | SAEEVLELIKELLPPDVRREVERFIRKLREDGVPPEKIVE |
| | LIRAALKYYGRLTPEIERLVRELRK |
| 1150 | DVVKLVIEVQKLAKKRGDLRAHILASRIIFAFMKGLMSSE |
| | EASKLLKTLAKRLKG |
| 1151 | SELEELLERIKRTGDPKLIARVARLLFELAEEKNDPKLAR |
| | LAALVYFYLRTGNPKAAKQILKRIE |
| 1152 | DERRRFIARLLQVAQEANVRRVLVMYANGSIRVEINGNDD |
| | SAKIVEKFVRTVAKKRGIPVSVTTR |
| 1153 | TEARRLARLIAEYARKAGVPHVVVVVLNNHVFVREDGNTD |
| | SVKKASEFAKEVARQLGIRLRVVTR |
| 1154 | SPETLKLALFIALVRRLFKNGEIDPSQAKTLLKRLQEKAK |
| | KIGDHFAVRLAEHAIRAIKKAEKS |
| 1155 | SDELSKKLEKKVKDALKKGDKRLAKFLVIKAILEAKRLND |
| | RKLEHVAARLALLIFKWS |
| 1156 | DLDEEVKKTLKKLYEIAKKDPSKKDEVEKLLVKVAAHAHF |
| | RIQNPEKRRKALELALTLMRRIG |
| 1157 | SFEEVIRRAIKLAEKNGDKHALVELKVILFLARQGKMSEE |
| | LAERVARTIEKRLKS |
| 1158 | DKEEAERILSKIHELARRKNDRKVQRLVMRLYFALQFADK |
| | KNNKKMVDDITRLAKELLKKVES |
| 1159 | TKEKLREIVRLLREGDPRARELLRRLLKELAERGDRRLFF |
| | LVFAILRLLKRNPEEATKLLAALAR |
| 1160 | SDESRKLQRRLALIFAALRQGFISNDEAKELAKRVEEKAR |
| | KLGDKHVRQLVKFVKDAIERAG |
| 1161 | SLEELEEILRRLARKTPAAAILLRILERLRKRGIPPRLVL |
| | RALLLFAKHLGDPELEKLIERVLRK |
| 1162 | TRELLLRVLILLWQLRTPIARAIIAFIYIAKKEGVSPEEI |
| | LKIILKLARRAGDEEVVRIIEELSP |
| 1163 | SLLEELEELLKKLNTPAARMLIALLRRLLEEGRNSPKLIL |
| | LFLWLLARRLRDPEVVKVIREILRK |
| 1164 | PVYVFISNVTPEAEKIVKKIARLGRVIVIKRPGNRIFVYV |
| | QNPELAKQLVELVRKAGARVLIER |
| 1165 | SELEELIERALKTKDPELLKKALLLAARLLDETGDPKLRI |
| | VVAYLWFAKARNNPKLIKRALKAAR |
| 1166 | SERRTVRRLLMEAALRLGVPFVFIRTVDGRVYVFVRGKDE |
| | SAQTLSKLAEELAKKLGVKLHVRRR |
| 1167 | PLEKLARHVVTKAKEIVKKAQDPRLSKIFKEIADKLETAL |
| | KNNDEKTVRYLIAKFFFLKRRLS |
| 1168 | SSEDMRKVFIMVLILAHEAAKDEDLERKARKKLDKILKDL |
| | KVPNAHFFKQMEDLLVKKVRSKLS |
| 1169 | DREEKITTLVKEAAKRAGVPHVDVNFSNGRIHVRVRGNDR |
| | ASRRLFAFARNLALAYGVQLQVLNR |
| 1170 | DPTRHKLMLNIFLVLVLFKKGKIDPSQAKTLLKRLQEKAK |
| | KIGDHDAVHVAELAIVRIKQAEKS |
| 1171 | NRDRIRAELLLWAIARRLERNNLDPRLVRTAIQVVARRLQ |
| | REIQNPENRKEVQTVAEELIKRVM |
| 1172 | DRRERQLKLLIDELVTRIKKYNPELAKKVESIVKKAEDNQ |
| | DPNLLVKALLVAIKALKTVSS |
| 1173 | DPKKLDELRKKIETLLRKVTDPTAYRLVMRALFLAIMALK |
| | QQNPLLEQLARELYEKAKKIQ |
| 1174 | PTEEVIRTVMLLVFMARRSQDPTRVEKLLTKAEKLAKNIN |
| | DPRLEELVKIAKKQARSILS |
| 1175 | DLKVISRALLIVLKIQKKYPDPREAKKVAKEVLKRIVDSI |
| | DDPKERVRVFFFAHMLLRMIG |
| 1176 | SELAELLEEALRRNDPELLRRIVRLLARLAEKREDPRLWR |
| | LAALLWFALRTGNPLLIKAVIKKLK |
| 1177 | PEELRKLLELIERGDKKKVFELVRKIAEKLKKKNPSLVRL |
| | LVAYVELALRGSPAARRLLRSLAEK |
| 1178 | SEEIFRKLMFAMFIAKRTGDPRFKKIAEKLNRILETIAKR |
| | DQDPDAKRLLRIFRQAAKRRKKS |
| 1179 | DINARIFLLILRAARKNDEKLARELLDKSKKDPQVESAVE |
| | RLRWEDPRVDKAYKILEKVVS |
| 118C | DKKLREELLRVALEAMKKIKDPDTQKSILKIAAQAFFRAR |
| | NIDDPTERDKVLKKWLKLLKDEIS |
| 1181 | DEKAEKLILKVKMFILMIKLAQSKEMKSKASEAAKRLLTE |
| | ISKIPDEKAQKIVKKLKEILDKLS |
| 1182 | SQLEELIERALKTKDPKLLRHAIIVAFLLLRRTGDPKLRE |
| | VLHLLAEAFFKKDPELIERALRVAR |
| 1183 | RYTEHVFFVWEATDEEARKRIRKRVEQIEREAKEEAKKRG |
| | REPQTYFVERTRDGYVSAVLIIIVE |
| 1184 | DDRRTELLSRLVKKLLEQARQISDPRQARKISLKLHMVKN |
| | LAQNPKVRTLADRAIKQVNSIVRS |
| 1185 | SKLEEVERLLRELAKKDPYAYILLRLLKKLKELGVPPERI |
| | LRLLVAAAKRVGNPELAKLIEEILR |
| 1186 | SVLFEVAMVALELGDPRLEILVIRAILLLKRGDPRVKEVL |
| | KKLVELLRKLGNPELAKKVEELLKK |
| 1187 | SKHEKARKALAMLLQIAVELGVDRRYMVRAMFIFLELVKN |
| | GVDLDQARRIAEKWLKKVVKS |
| 1188 | NRADQLIYEVRKIAKRLGAPDVKVDVRNGRVTVTIKGTDF |
| | RARAVAAAARIVARLLNLELDVQIR |
| 1189 | SYVERVVELLKRLGDPVARELAELIEEILKRNYMLAMFAA |
| | QMAEEALKINDPDMLDQLIRFLKHH |
| 1190 | SAAARVALLVRRVAEKLNVPNLRVEVLNGKVQVLIRGDDE |
| | SARTAYKIVKKISDTLGAPVRVQIH |
| 1191 | DKFAIVMHLLFLAKRASKRNDKLLVLFVLRLAKRLYDKHP |
| | DDEKIEKVLKIVEKIYRKVE |
| 1192 | DIEEVLERLKELIRRGDPRVRRLLARVAMRLRQRGDPRAR |
| | RFFLAFFRFAAGNPEHALSVIRKLP |
| 1193 | SELEEEFEEALERGTVEEFLRRLYRELAREGDPRSAALVL |
| | AAFRLVKEGDPEAAKLALRIIERRR |
| 1194 | DPTRRHLMSAIFMVLFLFKKGKIDPSQAKTLLKRLQEKAK |
| | KIGDREAVFLAELAIRIIKKAEKS |
| 1195 | DDRLRRELFLVVVRVARRIGLDRHAILVAALLAARLADKG |
| | VDPTQAERILEKVLRTIKKRKS |
| 1196 | DEEDKVRTTVTEIAKKAGVKGFHVTVRDGKVRVVLKGTDE |
| | SARRAFRLIFQARHKNNLKVFVLRF |
| 1197 | MEKYHRLLTKLSRLVIRAQQIPDKDKARSLVKTVAEEAIK |
| | SIDDPTLRIRFFFFVRFILENM |
| 1198 | TLKEVAQLLQEILQHVPDPRVRRLILIVYLALRKGNPELA |
| | ERALEEVIKRLGDPRLREALKLIRR |
| 1199 | DKDEVLELAQRILKEVQERGNEEYSRRILALVAAAFTASR |
| | KNDEDELKFILKVIRELYKKLQS |
| 1200 | NLSKHVAAAARIIARFVGVPSWSIDVKNGTVTVSLKGRDS |
| | RAQQAAYMIERFAKRLGVPVDVQVR |
| 1201 | SNSEELAKLIRQVAREANVPSFSVDVRNGTVHVTLKGDDE |
| | SFRRVFVIALQLAADLRVKVRVTVR |
| 1202 | GPEEVIEIIAKALGVPPEKVRRAVELMRKRLRIPPERALW |
| | LLLNLVKRLDPRFKEEVEEARRLLQ |
| 1203 | GEEEALKILAEVLGQSPEKIRAAVRHIRRRFNVSPEEAIL |
| | LLAKLIARHLNDPRVAEEIRKALEK |
| 1204 | SDRKIAARAIRIVARRAGVPSVEVTTINNTIRVTLQGRDE |
| | AVRLAAIFIWNISRRLNVPVEIRIR |
| 1205 | DELQKIATRLIKLAEKKGDKRLAMIARAVKFMIKRGELSK |
| | EEATRLLRQIEERAS |
| 1206 | SEFREVIRLLEELGNPRVARFVRLAWWLLRLGNPENAKAA |
| | LKAALRLAERLGDEEAKRLIEKLLK |
| 1207 | SARKKITRILLELADRGDPLRKHVVRVWFILRKDLDKAEK |
| | LVRQLIERFGDPLLREILELIKELK |
| 1208 | SSREELKKLVEKIAKKVGVPSVTVVERNGKILIVLKGHDE |
| | AARRAALLIRRVAEKLGVPVRVVIR |
| 1209 | DWRHAALLLAVIARIAKQDNDEDLLKRARTLFELLHRAAQ |
| | RGDEDTVKRVIEEAKKLAKDL |
| 1210 | PLESMARFVVKRAKEIVKKAQDPRLSKIFKEIADKLETAL |
| | KNNDERKVRILIAFFFVLRQHLS |
| 1211 | DDRTARLLSKVVKSLLEQARQIEDPQQARRIQVKLFMVKR |
| | FAQDPRVKTLADRAIKQVKSIIKS |
| 1212 | DQEELEDMIKAVEKFAEHLERNPDPKKKKAAKIVKDAVKN |
| | AKQIPNPRKAKKYLMIVVERLRKS |
| 1213 | NVEELAKKVQKLAKKKGDRRAFLIAFVLVRALRKGILSEE |
| | FAKRVLEFMEKRLLK |
| 1214 | SEETVRKAFQVATRRGDQKAARTLMRILEMLRMGVLDEEE |
| | QKKADKYAKSVIS |
| 1215 | DEFRRKAARLALRHLEILRNLSEEEAKKWARKILQEAAKK |
| | QDSALWIAARAALDFWRRALKD |
| 1216 | SERLELIVRTLAHLIRFADERLRKLIFKELLQLHEDELRK |
| | SSHFLKRASDEEIKKTAEEIIRRA |
| 1217 | PIEELIKELSKRVPPRLRQYLEAALRLKKRNPETARRLLA |
| | IVIYEILRLDPNDEIARKVLRLLMR |
| 1218 | SETSKKVREFLELVIQRGDERKEMMMIRFAKKRGLLSDEE |
| | LDRWRKRFEKVKK |
| 1219 | SIEEVVEKVLKEAREKGDQRVVLLAEALKRALERGDKRRA |
| | LALLIILRRLVGDPELRKAATLLVR |
| 1220 | LERDILTAFIIADELKQEFKDDPHIKSLVHRALIELMRAW |
| | RENDEDKIKEVIKKIKTIKKKADS |
| 1221 | SLLERIEEALRRNDPRLVLLLLAELLHLTGDPRVRFVLVY |
| | FYFALKEGDPEKAKELVKELLKRVK |
| 1222 | DQMERLRKLAAHIVMLAQKVGLSPEIIHRIARKAFQVFQD |
| | RDEDEARKWVEEVINRWKKKL |
| 1223 | TKEALRLILRLLDKRPDDSLLRRIMAYIFIAKRLGNPEFV |
| | ERIVRQLLKRTGDPRLKEVLELLKK |
| 1224 | SEELVERAVKFAGLSPHVEKILRAVVRVLERLGASPQQIK |
| | YILTRFLRRLIPDPEKLRRAEEILR |
| 1225 | DEELKKLLKIVVRLLIEADENNDEKSARLARRAYLLLWLA |
| | KRADEEHQRQLKTLVKQLIKKIK |
| 1226 | SLELVEEFLKKARTPRAFLLLRLLERAKKQGLPPEKVVEL |
| | LRAAAKALGNPELAEEIRRLFEEAP |
| 1227 | DIEEALELARRLGAPPRVVDYVEKLARRLKEKGLPPRVVL |
| | ALALYIARLLGDPLLEEAIKRLLKK |
| 1228 | TEIKKIAAAVERALREGTLEEFLRELLELYRKKGDPRILH |
| | RLLIAISIARTLSPEAREIVKRVRK |
| 1229 | DDRVKKARKIASTILRRLGVDEAEIRAAEAIVERLVKKGV |
| | DPKDAARVAVLAVRFLLRIRR |
| 1230 | DAIRRIAELAHRYGDPLLKLVVAAVWLLKRDPNDRAARHL |
| | LRALEQAAKKKGDPVLEEAIRLAKK |
| 1231 | PEIMVMMVEKLITLLKKLAQNPEMRKDIERLEKEIRTRIK |
| | KDPRFARKAFMILLQLYHFLKQQM |
| 1232 | PHREVERLLAELLERARDPRQRSLLRLILFALHERNEEML |
| | KQLLEALERVLGDPELVRKLEELIK |
| 1233 | PEARQITLRLSLEVARAIDRNDEDTVRTLLKIAKELARRN |
| | PQYREVFILLYNFIHRAKRAL |
| 1234 | DVEELLEELERLGLLDPELLELLKELIRRLKEEGDPEKLR |
| | KLHLLIAMARKDPLARFLLHFLVRK |
| 1235 | DEARSLALEVAIRAILKARQLNRPYDVTFTVGGQKYTIRV |
| | DTDPSKQRRALRRILLIFMRYAFK |
| 1236 | DKTEIIREMLTELAKKIKSVSDPTQKEKLKRHLFLLVFIL |
| | KNAPDPTEREAAKYAETLIKRILS |
| 1237 | DPRHEALRRVFSRLYLLLYRLTNPEEKKKAQKWAEKVLQE |
| | AGVDPQIARAAVEIVTREADRAR |
| 1238 | DEELKTIRSIVETLLEKARRNNDERSRRLARLALFLLERA |
| | RRADEEERKVLKRAVKALIKAIK |
| 1239 | SDELSKKLEKKVKDALKKGDKKRAHLLVVHALLEAKRLND |
| | RKLEKVAAKLEMLIQKWS |
| 1240 | SEAEEISRLLRLAAKALGVPSVEFDVDNGTVTVTVHGDDD |
| | AAQILWSLVRRVSREEGVPVRFEFK |
| 1241 | DETSKAVPMLAKVILLLKRNPNNEEAKKLYKEAVTIAQKV |
| | IKKLKDPKKQRHLHFLIHLATLES |
| 1242 | DEVERVLEILKRLGVDPRLLQAARQLFRLLRKKGLDPEQV |
| | RLLVAVELRRRDEDASRLFRKLLRK |
| 1243 | SERLFFSLRLRGDNRTSRELAEAWARRLAERAREEARRRG |
| | LEIRVHFRFTRENGKLTVTLRLHIR |
| 1244 | DVEKKAKEAIKLARKKGDKRAVLILHAIAVMYRRGLIDKN |
| | DADKLIEFIIRRLRS |
| 1245 | SAVEKAVKIIKKLAKDLNIPRVQVRYRDGRILVLIHATSP |
| | EAKTLRKLILEVARRLGVSVEVYLQ |
| 1246 | SLEELERLIRKLVKEGDPAARYALRVLRLLKKQGIPPDFI |
| | VAILLKELRRLGNPELIRLAEKLKN |
| 1247 | NYMRVIMRAVELALKKGDKKAVRILFHIAMAARIGTLDPT |
| | KAERLARRIEKKLRS |
| 1248 | SLEEEVERLLREAGVPPKIIRLLKAIIRLLLKRGIPPVAI |
| | LRILIKFLEKLGDPRAAKLLEELLK |
| 1249 | SEDEKIKKRVKEITKLAGVPSVEVRIKDGTLTVTVKGDDR |
| | AVRAALARLLNLALLLGIPIRIHVR |
| 1250 | DKEEEKILELLKQGNIEEAIKLARELARRLKDPRLAIAAA |
| | AIKHLLRRGAPPQIILYVVRRLLEP |
| 1251 | DEKEELEELVKKLGSERWERMVVYLIARRLGVPPEEVLKR |
| | IYELVKRLKVPPKSAALHLLQELNS |
| 1252 | DAEKIIRAAVRAFARLARIPSVTVRKSNGKFHVKINGRDA |
| | LAQQVAYAIKRIAKKLGLDVTVEYR |
| 1253 | SESDKLSKRIKELLKKAKEKQDKRKAMLAYLLALRVLRRY |
| | PDDEKIREVLKEAREVVRKIYK |
| 1254 | VPELERVKELVDRYAEKVGLSEEEKKKLRKLVDKLYKKGL |
| | SPMQVAIFVILFIRDLKKKL |
| 1255 | DVLEKVLKFVRKKNEQLARAVEALIRRLRERGVSPRTILI |
| | LLWTILRKAGNPEAVELVEELLKRE |
| 1256 | DELKKAAKELAKLVKKGLLSPLKAIALLIDIARKLGASKE |
| | EQRRALAALLYLIRH |
| 1257 | SEVREILREAARIPDPREVKRLVLKAAKLAIKLGDPKLAR |
| | LAALVYFYLRLGNPEAALHLLERIG |
| 1258 | TKLLRALAILYEKTGDPKERSLLLVVFIELRKGRRSPEEI |
| | LELIKKLLERLGNPELAEELERLFK |
| 1259 | DELEELLRELRKDPRLRAAVLLFRALLRQGYPPELVSIAV |
| | RHFLRRNGVPPELIEKLLKILRDLG |
| 1260 | SLQERFQRLARAAARYLGVKSVTVRVRNGTVHVTINAPDT |
| | RAYILLKIIKKVAEQEGVPVDVKIK |
| 1261 | DVERVRRRIFVVLWFAHQQKDPRYADLALKLAQTLVKLYP |
| | DNAHARAILREVKERVEKIKS |
| 1262 | DERRRLFVAIRNIALAVGVPSIRVVIHRGKFRVRVSGDDD |
| | NARKALKLIKEVAEKLGLEVTVEYR |
| 1263 | DVEKLVAEVALLALKRGDSRAMWKAIAILLNYRRGKLSSE |
| | EASKLLKTLAKRLKG |
| 1264 | GPEELEEIVRRELPPKVVRYVRALIRALARLGAPPQIINY |
| | AIILFLRKQGLVSPEELEELEKLLK |
| 1265 | VREEALRIIFKAILLLMNGQISRREAAKIILLAREASKKN |
| | DEELIKKVKSLLDSVEKKNQS |
| 1266 | DLKQRVRHLMIRILLLARKKQDPRLRRLAARLALLAIEAQ |
| | ERNDEEKLDKVRKEAKKVVDSG |
| 1267 | PPVLERLLELLKRLGNPLREAVRALLRALRRGEIPPEKVL |
| | KRLLFAAYRLGDKEFFKLVAELFRK |
| 1268 | TRREAMFVLEVAHFAYRKGDKDMVHAAYEVAKKLLEKEPE |
| | DERVRKVLEIVEELLKKVS |
| 1269 | SAAEEALKLLREVLPPEEREILERILKELRKRFPPAAVIF |
| | LLAKILLKLGLIPPQVAAKVLEILR |
| 1270 | DAQRTVEKVIRHAQEFVSKKGDERIKKQVDTLAKKAQTLL |
| | KQGDEDSALEVAALLMHLLWVVKR |
| 1271 | DSEEAEKIIRRVLIFAIFQNDKKIFRSAFELLKILEEATK |
| | KGNEELIKKVTKIARKLLSYV |
| 1272 | SREDLFAWLLRRVVKRLGVPSVDVKVSGGRLTVKVKGTNQ |
| | DARLAAAAAKVLARFFGIEVDVKVK |
| 1273 | DERRRFISRVLQIAQQLGVPRVLVLVKNGRFTVRVRGTDE |
| | RAQKAKEAIEKLAKRLGLEVELKDG |
| 1274 | GPLEELEEILKELAKTDPAAKILLAALKLLRRRFDPRLVL |
| | MILELVARRIGNPELLKIIREILRK |
| 1275 | SHLERLIERALKTKDPRKLRIAIAYAWFLKERTGDPKERR |
| | VVDLLARALVEKDPELIERALRVAR |
| 1276 | DEDTKAEKIAKDVLKDLVKKATTEREVLRALLHAIMVIRT |
| | LIPDQEKAKEVRRELAEIALKWI |
| 1277 | DREELKDLVSKVEELVRKVIKDPNYKSMATRLIKKAKELA |
| | KKGDEESAKRVIWMLIAIVFMYKR |
| 1278 | DVEELLEEAVKINDPRLVREALRLLFKLLDRTGDPKLKRI |
| | IAYLLFALKKNDPKLLRQVVELLKR |
| 1279 | STESVKKALKKALKSGDARKAFRIVMLAKVEGLISEKEAR |
| | KWRNLILKVSSI |
| 1280 | SELEEELKRAIEEGDRRLLARLLERLSRLLGDPRRAVRLL |
| | LRLIARVLNVPPEEAAKILRRLLKT |
| 1281 | DPTRHHLLKRIFMVLMLFKIGKIDPSQAKTLLKRLQEKAK |
| | KIGDKAAVSLAELAIVLIKQAEKS |
| 1282 | SREELLEELEKKARNPELRDLIRLLKELLRRGDPKVRRVL |
| | FLLFFRARLNGNPELARLVMKVEKT |
| 1283 | SEQHLREWIKKVEEQIRRGTITIRWAMIVHILIRQAQKLG |
| | DEEVEKRLRELLRRIAEVFH |
| 1284 | SIEELLEKLLRELGVPPRQIKIVKAAFRRLRREGLSPERA |
| | LEVLTILLRQRGNPELAKAVRLVLK |
| 1285 | SERHIKHALALLRRGDPKRALFVLYYLARRTGDPRVKELL |
| | RLLLEAFLKNDPELLKRVRRLLEET |
| 1286 | NEQDKKFAKRLLDQAKKLADQISNDRERKKAQSLVEKARK |
| | IDDPNVAMKVEVFVHLIYRHVQK |
| 1287 | SRKVIRELAMYVTRVAEKLGLDRRAFPRAFSLAYLLAERG |
| | VDPTQAKRILEKFLRTIKKRKS |
| 1288 | SLEEALEIIEKELGDPKLVKQLRAAIRVLKRFGADPFLIL |
| | RILRRILKDADTPEARLALKILEET |
| 1289 | SELEKILREVAKRVYKKKGDPRTIMKVIYQIVQRLIKTLG |
| | LDEEQQRELMKRAFMVWMDVV |
| 1290 | SEHLKRILKAAVRFIARLLGLDEKAQKIAKKVAEKLADKG |
| | VDPKEALRLLIRVLQQLYVRLR |
| 1291 | DEVLERLRKLLRKLGNPHLVRVVEAFIKYFRRLGLDPRMI |
| | IFFLLKFARRLGDPELTRVIEEAIK |
| 1292 | SLLEELEELLKKLNTPVARLLIRVLRALLKRGRESPKEIL |
| | QILWSLARRQNDPEVVKVIEEILKK |
| 1293 | DELLEELEELLKKNDPKVRRLVLELLRLAEELGDPELFKR |
| | LRYIAAAVELGNFKLALQVLKHILK |
| 1294 | PEKEILKTLYKLIQEALKKNDRRKVVRLYILLLRLMRRAQ |
| | TEEARKLAFKLLLLVIDLG |
| 1295 | TEELEKLLEELLEKTGDPRVRILLRALRVLLRRLDPKQVV |
| | QILWSVARRLGNPEAQRLIEEVARK |
| 1296 | SLEELVKELASEALGRPPEAVEKALRHISERFQIPPKRAI |
| | RLLVRALLHADGDPRLKELYKLVTK |
| 1297 | SELLRSARRLLAFIALALARGVHABEIIRWLEEWLKRARE |
| | NQNKQEIYSAQAALLHARKWLER |
| 1298 | SPEEKAKKLAEAAVKELVKLASEQIAKYNVSREVEMVAKD |
| | ENLDDSQTEKLQKYAQTLVHQLYS |
| 1299 | PYEEEIKKYADTLLTLIKNAKDPRQRRRLSKELFTTLLKL |
| | LKAPNPEKRAAAAQVIFKVFRVLK |
| 1300 | KEIAKKLADKLVTEATKLATSSDPNNRTKAHKLILRAMMF |
| | ARFANDPNVYRKVQSVYQLIRKLG |
| 1301 | SVELIEEALKLLGDPDARILVALIRYLRKLGVPPRIVEQI |
| | LEKAARQLGSEEIFRRAKELAERLP |
| 1302 | SKHEVDLLVAFVMVIARTRGDPTARKIAAKLALEAMKAKD |
| | NNDDEKLKKVSKRLKSFIKTE |
| 1303 | SDEEDRLLKRALAYLWALKAGKISVHLAKLAVRRVIEKAR |
| | RLGDEKAVKVAKKILEILKKIEKG |
| 1304 | PEELYKRATKILTEAKKRAKDNPEILRAIIRLEFLLRVAY |
| | KQNNEKTLKAVVKLVERKIRTLLS |
| 1305 | PEAKEVERTLLEVLHKAREDGNKEVVKLASALVAYFFFAR |
| | KRNDEELLKKVITLAKKVIKKQ |
| 1306 | SPVKEIINRALQILQKLLRLGLISPEEAQRLALFLLLAAK |
| | EGDEELARRLYKKIEKIRRKLS |
| 1307 | DKEYVKRLVEELIKRGDPHRALMVLFVELQHTGDPDIRRL |
| | ERLLARVIIRYPDPREVLKFFKKLK |
| 1308 | SKEEEAEKLLREVLPPRQRKLLEAILRILRKYAPPQLVIW |
| | LLAKILLRLGAIPPEVARRVEKILR |
| 1309 | SEDEQLSREVEKTIKKAGASHVLVRVKDGTLRVLVRRPSP |
| | EADQVLNKLRQKAKKLGLKERWFIE |
| 1310 | SEELKELVERFERGEIPPKELFRLILRLLQRTGDPKLKRI |
| | LAYLLFLIRRNVPPEEITRWLKAVT |
| 1311 | SELLRVMEVAHFLARRAGVPHVHVRVSNGRIQVTLKGTDE |
| | RARTVQKRLTQFVRAFGVDVDVQVR |
| 1312 | SDELSKKLEKKVKDALKKGDKRRAKLLVIEALVQAKHLND |
| | EKLHHVAVRLMFLIMKWS |
| 1313 | EEAREIIKLLFRAMQLASKKGNEKVVRIIKHIMARLFVAL |
| | RSNDEKMIEDASKKAKKVIKII |
| 1314 | PEEFLEEIKRLLEKLRTPAAWLILRVLRKLEELGTSPEEI |
| | LRILRALARRLGDPEVLRLVEEALH |
| 1315 | SKLEEVLELLKRVPDPRVHAVVAAILALLERGRYSPRKLL |
| | LLLHELLKRLGDKDALELLERLRRR |
| 1316 | TEHEKVRLVVEVAERLIRRGDQRRAQFVLWIAELLVRKLG |
| | DEKWLKIIEELKKELEKLKKK |
| 1317 | SVEEVVELARREGNPALAANLENIIRKLKRRGVPPRYILV |
| | ALLYVARLAGDPEVARFIHELAKSP |
| 1318 | SVAEELEELLKRGNAEEAEKFLRELVERHGDPRLRIALRL |
| | LERLKKSGVPPEKVIRFVVAALRRL |
| 1319 | SEVARRLLELLKQGNEEEARRLVLRAAIRAAREGDKKLAH |
| | LLALVYFYLKTGNPAVEDLLRELEK |
| 1320 | PKERIKELARRIKEGDPEAKKELAKLFLEAVRRGDKRLFL |
| | AIIFILALVLRIPPREAARLLIRLG |
| 1321 | DKDKLLEAAVRVVARVAGVPDVKVRKRNGKVEVTFRGTNH |
| | QVQQASEMVKKIAERLGVTVTVRFE |
| 1322 | DERRRFISRVLQIAQQLGVPKVLVRVKNGRFTVRVKGTDE |
| | RAQKAKELIEKAAKELGLEVELRDG |
| 1323 | SLRLKVAFLLVKAGVDPRLITRLLQYIHQLERRGVSPEEI |
| | LKILIKILEELGDPRAAKLLRELLK |
| 1324 | DKEEVIKTVTKLENEYTKRAEDPRKRLWAKAVVKMLQDLF |
| | KKDPDEAEHLARRISETVKKKLS |
| 1325 | SELKEVVERVLKGELSPEEVLRILLELAIRKGDPKYIYLW |
| | LWLRSALRRGNPELLRLILKQIRRA |
| 1326 | DRRRRFRLTQQILTTALELYKRDSRQAEKFLKTLKEEARR |
| | HNDYALWIATRLAEREFKSDTHD |
| 1327 | SELMEAVELARREGNPTVASFLMQLLFALKKGRISPEEVR |
| | RILEELLKKYGDPALELALELLRRK |
| 1328 | SRTDRVVKRLREILKEAKEKNDPKLAIKAAMFAHVELKED |
| | PTDEKIRKVEKEALKVALDLLR |
| 1329 | SEIEKKLRKLFKKAHEKGDLRALAYVLFAIKFLRRGKLDP |
| | RAAEQLARIAERRLRS |
| 1330 | SNSEELAKIIRQVARQARVPSFSVDVRNGRVHVTLHGDDA |
| | AVRHVLARAYMLARRLNVKVQVEIR |
| 1331 | SEDEKSKEISKTIQKLAGVDRVQVRIRKGRLLVFVRGSDE |
| | RAQTALRLALEIAKRLGVPVTRHVQ |
| 1332 | PMDTEMVELAKRLADEIKKLAQDPNLQKKADKLKKLVDEA |
| | YKKNDHITLREVVAMIILLWKRVL |
| 1333 | DQREILKVEREAWHTIRDASDRRVRAALRHLLVRLYEALR |
| | RKDEEALKKVLKRLKELIKEQK |
| 1334 | SVREAVEIILKRVGVSPEKINQLKANLWVLERRNPLAAEW |
| | LALKIIARVLNVSPKEAQRLLKELQ |
| 1335 | DKEEKIKKLAERLARRAGVPRVSITFHRGRVTITVDGPNS |
| | AVRRLAAYIFLFAEKLGIRLSVRSK |
| 1336 | NELFRLFEELVRRGISPEEILKLLVKLLREKGVDPQEIWR |
| | RLALILLQLGISPEEVARLVQKAVR |
| 1337 | DKDDEFLDKVRKTYESLQRNNVDPNQIVKILARLSMTAME |
| | LGLTRAMIFVIQLELKAKQQAS |
| 1338 | VREEAEKIVRKARTMLKKGLIDEKEARKIIFLALHAAKND |
| | DEELIRLVKRLLEQFEKKRQT |
| 1339 | DEEFRVRMLIRFLARLAGVPDVKVSVRNGTVHVKVRGTDR |
| | AARTALQVARIVALVLGVPVSVTIR |
| 1340 | SRDKAIRRLAELLGLSPEEVKKRLEELERKLGDPRRAVQV |
| | ELSELHRRGELTPRVRLFLLLIKRN |
| 1341 | SKKEILEKVLELLKKDPKKALRLIIELLRRTGDPRVKRIA |
| | AYILFLERKGDPAARDIAEKLLKET |
| 1342 | DEVLERLLKLLRKLGNPQLVIIVEALIKALRRQGLPPRLI |
| | IQVLAAFARKRGDPELTRVIEEAIK |
| 1343 | DFVIVFVFEGRRTRTFFFRSEEQAKELVKRLQEKNPRAKV |
| | VTRHRGDRVIIFVDV |
| 1344 | SKEDEISKTLEKIAKRAGVPDVKVTVRNDRVKVEIKGNDR |
| | RALAAYLLLLRVARKLNLKVSVLLK |
| 1345 | DEKQQKLRRIAIRIFAFANFVGDPNVRTEVERLAQHIMTV |
| | VQRDPDKAEELLRKEMTKVRRETR |
| 1566 | DDERLATDAFRSLIKRAGVKNLDVKVINGKVRVTITGRDQ |
| | ASQKALQKVFALARRLGLQVQIDTR |
| 1567 | SEQDKQVKKLTDIQREAYKHNDKETIEKALRTAASFARQH |
| | RDPKFRELFRIIHQLRQRVER |

TABLE 9
TGFb binding polypeptides

| SEQ | |
|---|---|
| ID | |
| NO | Sequence |
| 1346 | TCTIEVVGVDPEAVEAIAAAFGAEVREKDGKLEIHLDDPH |
| | GAESAAAAISVLANVRVRLQC |
| 1347 | HCTIEVVGVDPEKVEAIAAAYGAEVCEKDGKFEIHLDDPH |
| | SAESAAVAISVLTNRPVRLQC |
| 1348 | DEHERVRKKVEKVVKDAGVPDFDVEVEDGYVVVLIKGSDE |
| | SSQRARKAVIEWAKEQNIPLTVLEL |
| 1349 | DLHEKLKELAEFLAKLAGVPRVSVDVRNGTVSVTCHGRDA |
| | HCLALLLLLRLEARRQGLEVRVTVK |
| 1350 | DQITVIVHVDDPSLAEWVAKQCADQFLVNGKTVFLIVDGV |
| | DPTEAKRCVEELAKKANLEVELTV |
| 1351 | DEHERVRKKVEKVVKDAGVPDFDVECEDGYCVVLIKGSDE |
| | SSQRARKAVIEWAKEQNIPLTVLEL |
| 1352 | SLEEKLEEALKRGDEELVRRILEKILSEALDIPPEEVKEY |
| | LRALEERAGNPEAVIFLVLNLEQRR |
| 1353 | DEHERVRKKVEKVVKDAGVPDFDVEVEDGYVVVLIKGSDE |
| | SSQRARKAVIEWCKEQNIPCTVLED |
| 1354 | TDEELEKLAEYAAEAGDTEALLEIYSLISALRLGAISPEQ |
| | ADELARRIKERLDK |
| 1355 | DVEELVKFILELLKENREAAEFYLAHLLEKLGIPPEEIDE |
| | ILKRVAELFKRDPEKARELIEKVLR |
| 1356 | TVTIEVCGVDPEAVEAIAAAFGAEVREKDGCLEIHLDDPH |
| | GAESAAAAISVLANVRVRLQE |
| 1357 | KVIIIHVHVDDYKSAEECAQKANVYVNVNGKHVTIFLDDI |
| | DPSVARKVEKCLRELNADVKVFV |
| 1358 | SEELEKLLKCVKEGDPECFKRVAEIIEELKKRDPEKAQLV |
| | EAALFLADNGILDPELIKFVVELVQ |
| 1359 | TVTIECVGVDPEAVEAIAAAFGAEVREKDGKLEIHLDDPH |
| | GAESAAAAISVLANVRCRLQE |
| 1360 | TIEELLEELRELGLIPPEVVELVKRVEKELAEKGDPRAME |
| | LLILVSDAVALNDPRAVEKLEELVR |
| 1361 | SEVERDLQFFIEAGVSPEAIHFLLANLLERTGDPRIEELL |
| | RLLAECLKRGIDPEECLEILLKFIK |
| 1362 | PRVIVIVDDPTVAELIVRRCNVKLVVRFGDQFLITLDDYQ |
| | CAQTVARELQPDVLILTSE |
| 1363 | SKLEEILEEARRRNDPEIVELLEFLLELAKENPSEAVFLA |
| | ENLLELYGDPLLELVVRVLEELVRR |
| 1364 | SDEDLELLAEWTGDPEQAIEALLFLADNRGDEKLVKILKK |
| | AVELLRRGEVSPEELIELIKELLRR |
| 1365 | DTEETLQTIVKELAKKAGVPSVEVDVVKGFIIVELKGTDE |
| | NAKKAFQLISKTCAELGYPCVVSTK |
| 1366 | SEIEEEAERAEEIKKLAGVPDVYVVVLNGRFFIQIQGDDE |
| | TAKEAEKIIDKYCRDNNLECVVFIE |
| 1367 | PKLEEVLRKLLEEDPNNDCLRELLFVLERLRRLGVDPHQI |
| | EINLEFLVAANHDPECERLLKEAIK |
| 1368 | DELLEHLRELVERLGDPEQVAEALLFLADNKGTAEELLKL |
| | LEELLRKTGDPEVKKILEIVERLLR |
| 1369 | TEDEKLAREILQLARDLNLDDVLVFVNRGVVFVLINGNDD |
| | RAKKLKKLAETIAKKQGIDILILEL |
| 1370 | GPEELRKELEELGDPKCVEVLEDALALLEQGVDPESVKQF |
| | VELLADQRGCSPELVRKIKKAVEEK |
| 1371 | SESEKIEKDVTDIARKAGVPDVDVELYNGEVVVVIKGDTE |
| | SAQRAETLILRWCREQGVECTILIS |
| 1372 | SEVERDLQFFIEAGVSPEAIHFLLANLLERTGDPRIEELL |
| | RLLAELLKRGIDPEEVLEILLKFIK |
| 1373 | SLEEALEIIEKELGDPELVHQLRINIEFLKELGVSPEEIL |
| | DLLRLILRQCNTPECRLALKILEET |
| 1374 | DEAERLKKELEDLGDPKCVEVLEDAIALLEQGVDPESVKQ |
| | FVELLADQRGCDPELVERIKEVVEK |
| 1375 | SEELEKLLKLVKEGDPEAFKRVAEIIEELKKRDPEKAQLV |
| | EAALFLADNGILDPELIKFVVELVQ |
| 1376 | SEKVQEIAKELRERGNPAAHQAAINFEFLLDLGIDPEDVE |
| | FLVCRALEEDLGDPEEAKRICKLLK |
| 1377 | SELDKIVKKLTDICREAYKHNDAETIARAYETAKRLAHTY |
| | DDPKFRELAIRCFRLDHEVDR |
| 1378 | GVIIVEITNEEAKKCVSIARKVPLVQVFTSNNKVYFIIVR |
| | DERALRLIEECLKRGNVNEVTRH |
| 1379 | DRELTELLLDFYSAVAFLKQVPDPNAQTLAKKLEEKFRTA |
| | LENDDDESLEKIVKEAERLANEQ |
| 1380 | SEYRTIDQLLARLALELGVGSFFVNCNDGRCTVFVLGNDE |
| | AAKIITEAAKKLSKTYGIPIEVVLF |
| 1381 | DCVLKILEELSKEDPRVREILELLERLAESHNLDPEDAVE |
| | ALLFLLDNRGDPRVELLRKALELCK |
| 1382 | SNREAAEVVHLARKAGDPKALELAEELLQCLNEGDKECID |
| | TLAKRIKKVIS |
| 1383 | DAHALLEIYSVLHFAKREGDEDLYKKVKSLLETAHKRAER |
| | ASSEEEEHRIFQDAAKKVEKLVR |
| 1384 | GLEDVKKVIREVLSPEEVEELERLIEKLRELGISPRDIIR |
| | AILAELFILGNPEAFRLVLEVLHKE |
| 1385 | PRKELAKELAKELITKAVKHQDPEAERDAKQACDVLADAA |
| | GLSDEWCKEVVKELKDKAKKS |
| 1386 | SELDKIVKKLTDIAREAYKANDAETIARAYETAKRLAHTY |
| | DDPKFRELAIRIFRLDHEVDR |
| 1387 | TIEELLEECRELGLCPPEVVELVKRVFKELAEKGDPRAME |
| | LLILVSDAVALNDPRAVEKLEELVR |
| 1388 | SEVEEVLEEVAKYVSPRLETEILLLVYSAKSAGLDPEEVV |
| | RLLERFLRKLGDEEAVRVVEELIRK |
| 1389 | SESEKIEKDVTDIARKAGVPDVDVFLYNGEVVVVIKGDTE |
| | SAQRAETLILRWAREQGVEVTILIS |
| 1390 | SEEFEKLVEELLRRGDLEKVKEVLKEAYEKLGDPRYHQAL |
| | INLEFLEDNLSPEDVAFLVLRQLKD |
| 1391 | QILIEVHVSDPNLAESCARAVNATLLELPGGTELIIVDTE |
| | ELSKIVSTCLKRLGAEVHVVG |
| 1392 | PELLEELLKLLEELGDPELLQTIRFLLECVRRGECSPESA |
| | LFLLLNEAQLLGNPIKEEIERLIRK |
| 1393 | DYEFQRLVLLLAQRAHECAEKNDEECLKKIESTAKKLKKE |
| | AQDPRIRRALQHVILAVQRLS |
| 1394 | GPEELRKELEELGDPKAVEVLEDALALLEQGVDPESVKQF |
| | VELLADQRGVSPELVRKIKKAVEEK |
| 1395 | SVQVVIKGEDDLHAFTVDSPEQARRIVTEWKKLYPDAKVF |
| | ETEHDNVLILEVHN |
| 1396 | SFRELSRKALEIANELGDREAERRVFRINQALVEGEAPEE |
| | WAEKALKRLIKKLK |
| 1397 | DACKIASQILLKHPDPREVRELLKELIKRLEENGNPEEAE |
| | LVKRLLELAKRDPREAQYLVEKCLY |
| 1398 | SNREAAEVVHLARKAGDPKALELAEELLQALNEGDKEKID |
| | TLAKRIKKVIS |
| 1399 | DTFIVSHDKREAEWCARAANVKEVIFELEGGEILIIVEGV |
| | SEEARELLDECFKILNQDNLIFVN |
| 1400 | TRILVIVHVEDPKKVEKCAKLAGVLLNTVGKSLLIVLNDN |
| | DPNRLEKCLRKVGAEFELIEF |
| 1401 | KILLIVFTEDEEALREVIELARRIHAVECIRIGDQALCEL |
| | GDDEEAKKFLEFTKNERVKVTELEL |
| 1402 | SEEEHTEFLLRLLRENPLEFEFYIAHLIDRTGDPRLEELL |
| | RLIREALKEGDPELLEKLVRLLLKT |
| 1403 | SEEEYKKKIKELLRRGDLDRAYELAVEAYHRLGDPRFLEI |
| | AAEIFVLDAKGISPEEIVEILERLE |
| 1404 | DEAKRRAEILKKEAEKLIKNGNRTEALLLLYSALSFAERK |
| | NDEKLYKLVETVKKEVESLANS |
| 1405 | TLVVICRVDNVEEVEKFARNADAKVYVNGKNVFVVLKSEE |
| | AAKKLTKWAKKMNAKECVVHKI |
| 1406 | SDEDLELCAEWTGDPEQCIEALLELADNRGDEKLVKILKK |
| | AVELLRRGEVSPEELIELIKELLER |
| 1407 | DEEFKEEAKELIKEGNPEEAVKVIEELAERLNDPELHQCA |
| | INLEFLLDAGEDPEDILFILELCLK |
| 1408 | SQSELVETLVKEVSELLEIPSLEVSCSNGSCTVTVKGNDS |
| | LAAAAAILIDNAALRVGVPVSVTER |
| 1409 | SNCERASEAIRVALKAAGVPSFSVTVDNNSVTVTVRGNDE |
| | AVREALQNALFIADHFGCPVSLRTQ |
| 1410 | DIEELFRECRKKGLSPEECLKLIAELLWRSGDPELKSVAA |
| | LLLLLLDNGVDPEEVLRDADFFLRL |
| 1411 | DEVEKLKKTVEQIAREAGIPDFDVFISDGQVFVLIKVHDE |
| | RATKATKRILKVCKKLGIECSIVLA |
| 1412 | SEEYIREVIKLEREKGDPEAAEFVEELLRSLKDLSPEEVL |
| | HQIAAYLVAKGDPELLRIIFEALEK |
| 1413 | DVREVLEELLERGEVSPRVVTEILLEVYSARHAGISPEII |
| | KELVVRSLERLGNPEVARLVEKLLR |
| 1414 | SIEERLEELCEKKNDPECREAVELFKLLVERTGNPEEVER |
| | QIEEAIEVTGDPDERLALEILREAK |
| 1415 | PVVLFLIDAPPEAARWLYELVGVKRVISYEDGLYILEVDD |
| | EEAARKACELVARLFPIKVVCLK |
| 1416 | PRVIISCGDHGIEEAYKLVKSANAKVILLGDVVEVENVDP |
| | REAEKVVRYLQSLGIPCFVFVLE |
| 1417 | SERFEELVRCAAKGDERCERVILFLIDLLKLRDEERAREV |
| | KKILELLKKDPDNEELLERLTRLLA |
| 1418 | PEEVLELLLEALERGDKDLLEEVKRLLEELAKTDPEVHQL |
| | LINAEFLEDAGFSPEDIVELLKIQL |
| 1419 | PRIVCIVRVNSPYAEKVAKIAKKLGVHVLQHGNDIFLLDA |
| | DEEVQRLVEEAKKYGVEVLCVEFE |
| 1420 | DLLQLLAHLLEIARRLGNEKALEVIDRAIKAWLEGDLETA |
| | KKLTKLARKLL |
| 1421 | TVTIEVVGVDPEAVEAIAAAFGAEVREKDGKLEIHLDDPH |
| | GAESAAAAISVLANVRVRLQE |
| 1422 | PEEVLELLLEALERGDKDLLEEVKRLLEELCKTDPEVCQL |
| | LINAEFLEDAGFSPEDIVELLKIQL |
| 1423 | TIQEEVERIIREAQEFVSKKGDEDIKRKVDHLAELAQRLL |
| | KAGDERYAKIVAEALRAILLKVKQ |
| 1424 | TELLRELFRRALERGDNSQVLIDIWEICRIVAGGDPELTK |
| | RCFERIVQLLERDPEKARELLEKLG |
| 1425 | DEEKKELKERIDKLLRYAIEVVDNPEERKLFTELAKKIKK |
| | LLEKNPTEALLEAYSLAHYARLK |
| 1426 | TEDEKLAEEILQLCRDLNLDDCLVFVNRGVVFVLINGNDD |
| | RAKKLKKLAETIAKKQGIDILILEL |
| 1427 | DLKEEIEKLLRECGVSPEEIEELKIFFEELLKRASPEQVL |
| | EALIFLLDNKGDPCAAKLLLELLRK |
| 1428 | SEDEEIERAIREAAKRHGLTDVDCKVRNGTITCKVGGDDK |
| | DARAVLDFAELLADALGVPVNLRTG |
| 1429 | SEEHSRFVEELLKDNPAAAVFYAVNAVQNLRRRNEEEARR |
| | VEEELRKLLERLDPRLKEAFEYTLR |
| 1430 | DEEKKELKERIDKLLRYAIEVCDNPEECKLFTELAKKIKK |
| | LLEKNPTEALLEAYSLAHYARLK |
| 1431 | SESEKIEKDVTDIARKAGVPDVDVFCYNGECVVVIKGDTE |
| | SAQRAETLILRWAREQGVEVTILIS |
| 1432 | SERFEELVREAAKGDERAERVILFLIDLLKLRDEERAREV |
| | KKILELLKKDPDNEELLERLTRLLA |
| 1433 | HLLVEVKDERVAREVAKLLNAPCIELHLPGDKYLFFCRGV |
| | SKEQAKRVAKTVHKKYKNVQLIVL |
| 1434 | SESEKIEKDVTDIARKCGVPDVDVFLYNGEVVVVIKGDTE |
| | SAQCAETLILRWAREQGVEVTILIS |
| 1435 | KKCLVFVQDVDDDVVKKLSEVANVPVVKLPGNIVIERVHD |
| | DELAERILKVAAEVGARVFINCHE |
| 1436 | NEAATEELKRLIKRLPNREEIEKLIKEVSELAGVPPEEVE |
| | RQVAQAVDFLGLPPEEVLRALLYQF |
| 1437 | DASRRVSTEALLNAYAAGVPHVRVEVKDGRVKVTLRGDTE |
| | AVRAATRAIKRLAEKQGVKVDLKVQ |
| 1438 | PFCAVDVEDEEAAKIIARELDGVLVTFSSGEALVYISGYD |
| | WKEVVKRAREIAKKLNVNVRIFCSE |
| 1439 | SVTELLLALYADLHYFGDPRVEEILRRVEELLHKGDPRTA |
| | IELVLRAAEELGNEHLKKLAEELLR |
| 1440 | DRSDLFALVLTALLRLGIPPEEVRRIAAEAFRLCGGDPEC |
| | IIKIVKEILEREDPELARLFEENVK |
| 1441 | SEEEVEILKEEVKRIAKKACENNDEKTLHSLEAFAEIILD |
| | SNNNEEAKEILEYIRKVITDCSK |
| 1442 | DEVEKLKKTVEQIAREAGIPDFDVFISDGQVFVLIKVHDE |
| | RATKATKRILKVAKKLGIEVSIVLA |
| 1443 | DRSDLFALVLTALLRLGIPPEEVRRIAAEAFRLAGGDPEL |
| | IIKIVKEILEREDPELARLFEENVK |
| 1444 | SLEETVKKVVKKVATEAGAEDVDVFIYDGYVLVLFKGSDE |
| | SAKLASELIHEILRRLGVEVTLLIL |
| 1445 | SLELLEEALRCLGDPAIEDVLFLVRELLESGDPYSALFLI |
| | ANLLERLEEKGNPDCVKIIRKALEV |
| 1446 | SVVVLIFGEDSTHYFVVDSPEQARRIVTEWKKLYPDCEVR |
| | EVEQNNQLILFVKC |
| 1447 | SEEEEDRALEEFIRRAADVPDVKVRCKDGTCTVTENGNDE |
| | AAHQARINLEFLSDILGLDVKVRVK |
| 1448 | DRSDLFALVLTALLREGIPPEEVRRIAAECFRLAGGDPEL |
| | IIKCVKEILEREDPELARLFEENVK |

TABLE 10
Tie2 binding polypeptides

| SEQ | |
|---|---|
| ID | |
| NO | Sequence |
| 1449 | PEEFVREALRRLIPDPRLQKIVEEALELAKRLGIDPFEVL |
| | QILDFLLIYLGDPEEAAKILEELVR |
| 1450 | SIVEELERLLEEAGVDPELIEKLSAVILQLLIRGLDPKDV |
| | LRFLLEMLERDGNPLRRVVEELLKR |
| 1451 | SIDEELERLLEEAGVDPELIDDLYAVIYQLYIRGLSDKDV |
| | ERELLENLERDGTPERRVVEELLKR |
| 1452 | SVEEFLKKLVEEFGVPPEEVERALEIIARLLNVPPEAALF |
| | FLIAIILDYNVPPEEAVEIVRRNVT |
| 1453 | DESAAEQIARLLIQGDPAAKQTVRAFVQAAKRGNPAAKRA |
| | LEVIRKVLRKLGNPEVVKKVKKLIK |
| 1454 | TEEIARRIVEALERGDEHLYFDLLQKLSLLVVRGDPVARE |
| | ILEVIREAIRRNDPDLLRELLKRLG |
| 1455 | DEERRHAEKLLRKGDLKTLKKLVKRLIKRVGDPNLKLQLL |
| | QVEMVEKFGDPHVALLLEVFIVKEG |

TABLE 11
TrkA binding polypeptides

| SEQ ID | |
|---|---|
| NO | Sequence |
| 1456 | RDELKERIFKTIVRAVVTGDPELLKEAKKLLEKLKKLGRL |
| | DQSAKQLEKAVREVEKQLRS |
| 1457 | RDEIKERIFKAVVRAIVTGNPEQLKEAKKLLEKLKKLGRL |
| | DQDAKKFEKAIRQVEKRLRS |
| 1458 | DEIEKRLRTALEELVRADKNNDEEKVRKAWQKAVQIVIEA |
| | NDDNVSKLASKIYRELAKRVKS |
| 1459 | SPEEELKKLEKIAKKKGDTRALSVIQVAKKAYKKGDEWTE |
| | RIALHIAKQLL |
| 1460 | DEIEKRLRTALEELVRAVKNNDEEKVRKAWEKVVQIVIEA |
| | NDDNVAKLASKLYKEIEKRVKS |
| 1461 | DEIEKRERTALEELVRAVKNNDEEKIRKAWQKVVQIIIEA |
| | NDDNVAKLASKLWREIEKRVKS |
| 1462 | DEIEKRLRTALEELVRASKNNDEEKARKAFLKVVQIVIEA |
| | NDPNVSRLASKLYKEIEKRVKS |
| 1463 | DWKDQASELLDKLIKKARSNRDDELAKEVLKLLLRARWAS |
| | HLSDDERAKKLLKKIVEEARKM |
| 1464 | SDEKLKRLEKIAKKKGAWKALVAIHLAKLASKRGDEKTLR |
| | SAEKLAEKILS |
| 1465 | DEEEKRILKKLKEVLRSLSSIPDPTAQKVVETAWKLAVEA |
| | VIKNDKELLKKVAEYAERILKKA |
| 1466 | DESLKLLRKADKGAREKQISEKELKKAVEEARRRGLEEVA |
| | RYLELLLWVLRLL |
| 1568 | RDEIKERIKKAVVRARVTGNPEQLKEAKKLLEKLKKNGRD |
| | DQDAKKFEKAIRQVEKRLRS |
| 1569 | RDEIKERIKKAVVRARVTGNPEQLKEAKKLLEKLKKNGRL |
| | DQDYKKFEKAIRQVEKRLRS |
| 1570 | RDEIKERIKKAVVRARVTGNPEQLKEAKKLLEKLKKNGRD |
| | DQDYKKFEKAIRQVEKRLRS |

TABLE 12
VirB8 binding polypeptides

| SEQ | |
|---|---|
| ID | |
| NO | Sequence |
| 1467 | NAEEILEKATLIAIEAWMLAKDEEVKKLVRTLARQVRKLL |
| | SNNDDDSAKSVLDTLKSVLEDLKS |
| 1468 | NAEEITEKATLVGIEAWLLAKDEEQKKKVRTLNRQVKKLL |
| | QQNDLDQAKRVLDQLKSVLEDLKS |
| 1469 | TERDLAFLIVSNVSPEQQEKFKKTLKKIADELRKRAKDRG |
| | LSVSIDVKQIRINGTDIVLIDVQVD |
| 1470 | DELEKLLRELGVPPEVIEELKRLYEELRKKGIPEEEVERI |
| | LFRILLERGDPALAAFTLVAVLLYI |
| 1471 | DELEKCLRELGVPPEVIEELKRLYEELRKKGIPEEEVERI |
| | LFRILLERGDPALACFTLVAVLLYI |
| 1472 | DEEILRKIEEIIKRIGNEDLERVERELKEALERGDEEEYK |
| | ALLVLLAILAYILGDPDVARLVRKL |
| 1473 | DLIERLIKIARELGDEEAVRLLEKALEALKRGDYETVLKL |
| | LREVYKLLGDPEVTVVAVLLYIEAI |
| 1474 | SVEECLERLKREGVLPPELLKRIEEAIELAKRIGDEEALR |
| | VLELACRLGNEYAILAVIYVIELEV |
| 1475 | SLEEKIEEAIRRNDPELVLELLKKLAELRNDPYLRRVARA |
| | LELAVRLGNPVLVYAELILVELLVK |
| 1476 | DEERLRRLGVPPEVVELLKRLYRELEKQGIPPEAIVAYLY |
| | VIAVELNDPELEKALRLLAKILLTE |
| 1477 | DLSERVLKAIALAVKLGNEVLFKAQIILASITLGVPPEEV |
| | LKLAKELAKRIGLLSPEELELLERL |
| 1478 | DLSERVLKAIALAVKLGNEVLFKAQIILCSITLGVPPEEC |
| | LKLAKELAKRIGLLSPEELELLERL |
| 1479 | DVTITIIVFINGEFTIYRIRVGPDPREKKEAQKLIDEIST |
| | KAKKLGGSVWVWRQDGTDRQEYHYK |
| 1480 | DVNVYAAAISVVLAKDPQALEALKELAKKLGDEELLRAVE |
| | LFEEALRRGDEELLRKASELLLRLA |
| 1481 | DISYVTVERSSPGNEDEYRKWKKKVEKVQKDLKKKAEELG |
| | LTIKVRSNRVRQGDSTFYFVFIWVE |
| 1482 | SDNKVELFIFVNGNPEQYKEAEKRLKKIAKKLTDEARKRG |
| | QRVSYSIRKTSRGNVVLVVLEVQTD |
| 1483 | PKEVAEILEECARKAGDEDVEELIKELKEAIKRGDEEEIR |
| | ALLVLLAILLYILGLPECAEVARKA |
| 1484 | DEEELIAEAAAVSAAFIPDEEVAEKVVEAAVRLCLKLGVS |
| | EEECREICERAGVDPRLCEKILRKL |
| 1485 | SYWIVVQSDDGGRLEFIIVEVDDDPEDLKRAEELARRIAK |
| | KLTSRGLRVQVWVLDGSKWSTIKIE |
| 1486 | DLEEIIRLLKKVAKELGDEEALRLAEKAEKALKDGDPDEV |
| | KALLVLIAILAYIKGNPELAEVARS |
| 1487 | DLEEELKELGDPHILALVLLSAAYVEGDEELVRVVKEILK |
| | LLESGKISEEEALELLERALKEIK |
| 1488 | DERCAELLERIAEKLGDEELKELVKELKRALKEGDEEEFR |
| | ALLVLIAILAYILGDPEAAEVARKC |
| 1489 | SVEIVVVVTYNGKTERYRLHVGDDPEERKKAEKFVKKVTK |
| | RAKELNARVWVAIFDGHEVDESAYQ |
| 1490 | DQRVTVFIFYNGDRKIETYRYGTDEEAEKKLKKLIKDWKR |
| | KARKLGGIVIIAVRNGDKIREWWVW |
| 1491 | DDIEWYIIVVDSRDSKSQQEARDKLETIASKISKKVKKNN |
| | GRVSYKIRTYSENNRIAFILEVKWG |
| 1492 | DEELLERAIKLLREGNPDEAAEVLRKLYKILAEKGTPAAA |
| | DISLALVLLYIGNPKLARKVLEKLK |
| 1493 | DEDDLDLRVTVLLLAWMAVLDNRPYTYSVNVNGYNLTVTE |
| | DTDPSQAQKAAEDEVKKYEQLKRN |
| 1494 | TEHQIWRIRVKGTDEEAQKKVEKAVKKVKDTAKEQARKQG |
| | KSVEVVVRQFHFGDTIFVIIVVSIG |
| 1495 | DEELLRTLKRLSPEAREIAEILERLLRNGIIPPEEAEELL |
| | RAVKTCLDRGNPLACEAQKLLVEVN |
| 1496 | TEELREVYKILREVDPEAAAAVTILLVLLYLDPDDEEVRK |
| | LAELITEAVRRGDPELIEKLAREIN |
| 1497 | DEEAVECLKKLIPDPEVRKLLEELLERAKRGDPEAKEILE |
| | LLRELCKLGNTEAVHTILAVLLYIK |
| 1498 | SEWDEVLWEENKDDPDQRKKAEKKVKDFEKKAREKAKRKN |
| | VPITVKRRSFTKDGTVFIVVEVEVG |
| 1499 | DIILEAALIALEITGSPEFLKAAKKYAEKLGDEELVRLIE |
| | KLLEALERGDEELYRELLELVKRLL |
| 1500 | DELSKELEELIKNGDLETVLKIAEELVRKVGDPRAEKVLE |
| | AIKEAIERRNPVLVYAAVALLLHLT |
| 1501 | SELAEKLLEALERGDRAALTLLLIKLYIELLKKEPPKEVK |
| | KIVRTVLELLKRDPEKAKELLEKLR |
| 1502 | SEVDFYLILLDGNDEDAQKTADRLVKEISKTATEEAKKNG |
| | GSVKIHVTKWHSGELLIVIVEVNID |
| 1503 | SIKISIWVLYNGRTERYEVSVGDSPEEQKKAKKEVKKIVD |
| | TARKLGAIVVVENRNGEDLEFHEDV |
| 1504 | SVDEVAERILRTGDPELVKKLEELLEELCKTDVLACAALL |
| | VLKVALELNDKDLVELVLRALKRIF |
| 1505 | NVCLALELGNDVNLYAALALLLVEAKAKNDEELLRKLEEV |
| | ERLAKEALEKGDEELLKEVCELYRK |
| 1506 | TEACLVLLAILAYIKGNPEVAELVRELAKLALEGKVPPEE |
| | LTKLLEKLLRELGDPEVLKLLKECL |
| 1507 | DDFEFYVEKIDVKDEEDKKKAEKYAKKVKKDIEKHEKDRG |
| | LDVKVKLWYDTLNGEFFFVVEVRSR |
| 1508 | SFEELARLIEKIARELGDEELEKVARLLKECLKKGDTVCL |
| | IALLALLYVKAYEKRDEEAKKAARS |
| 1509 | DKEEIYIVLLENVSEEDVKKAHETLKKTAEKLKREAEKRG |
| | QKFTESIKVVEWGDIWFILLTVHKG |
| 1510 | DQKVTVFIAYNGDRKIEKYRYGTDEEAEKKLKKLIKDWKR |
| | KARKLGGNVVIVVRNGDDERVWIVE |
| 1511 | DVERVEKAIKLAVEEGNELLVRVALYLLAAYLESLGNPEE |
| | AKRVKELLELLERGEVPPEEILKLL |
| 1512 | DEEAIRLIEEALRLNIDPHILELAVIAVLAVLSLQLGDPE |
| | KAKEIIKRLLEELGDEEAKKLLEKA |
| 1513 | SSVDFINVELQGASPEQVKKVEDEVRTWAKKVKKTWKKKG |
| | KDVKVSVWVDTVNGAYAIYVFVQVD |
| 1514 | DEHYVVIVSEKDVSPEQQKKAEKIVQDFTKHLTEKAKKRG |
| | LDVKYVRWSWEENGKTVVIVIVTVR |
| 1515 | DDFDIFLYNIDSTDPEQIKHARKLAEKFAKDVTDDAKKRG |
| | QRVKVSILQINWGNQVNFAVFVHIK |
| 1516 | SVTVYVWVNDNGQEDTITVTLGDDPSEQEKAKQVARDLNK |
| | RAKELNLEVAVWVDYGDRVELWLVE |
| 1517 | SERRIYFIYLDGNDEDAQKTADRLVKEISKTATEEAKKNG |
| | GSVKIDVTKWHFGATLLVLVRVNID |
| 1518 | PEVLVYVVVEDWRKVSEVARRLGAKVWTFGDVVFVAFENV |
| | PPEVAKKLAETLSKLAQVSVWVIG |
| 1519 | LTIRIWEWKYTNISPEQQKKVEKKIRELAKKLQKEAKERG |
| | QRVTVVVFRFESNNRVLELLISIER |
| 1520 | DKEELTELAKEAVKKGDFELLERLLEEARRRGDEEAVRLI |
| | LKLLAKVALELKDPEILALLAYLYV |
| 1521 | CEELVKELGAPPEVEKVCRLVEKLLRERGVPPEEIRVLLY |
| | ALAIILLLLLELGDEETVKRIRERL |
| 1522 | SRVWISVTDEKAARVISKLVNVKRVLKRGDFVFLSVDLDW |
| | RKAKSIAEKVSKELQVPIFVFWVP |
| 1523 | DEIEDLLEKAKEKGTEEEVERRLIEEAEKEGDELLVLVLR |
| | ALLEAVKLGNEVLTRAALILASVTR |
| 1524 | STVAIFVDDWKAAEEVAKVVNAEVEVTRKNGVYFIEVAIS |
| | DEDKARKVVELLKKVGVEVVIILP |
| 1525 | DELLEELLERCDTPVCRELLKLFRECKERGVPPEACVAYL |
| | YVFAVELNDPEVEKVLREAAKLVLS |
| 1526 | DIALLALAYVLAVEVGDPELEEFVRRLAECLLRGECPPEE |
| | VRRLLEEFIEAVERNDEELLKRLVS |
| 1527 | GWIFVIISDWKKAYKYAKSLGVKVEVWTWDNRRYAVVLKD |
| | VSEEVSRSLKEWASRVGAHFIVFF |
| 1528 | GEYDFILVDVRGDDPNKEKKARDTLKEFARTLEEKAKKRG |
| | LDVKYSLFEIQLKGDIFFVLEVHVD |
| 1529 | DEFEEKVTKIIRKVAKEARKKNRPLRVRVTVNGTTIDILV |
| | PPDPDLATTMAKLAIMYVKIRQDI |
| 1530 | SENEVVFWRENKDDPDQRKKAEKKVKDFEKKAREKAKRKN |
| | VPIDVKKISFTINGRVIIFVVVTVG |
| 1531 | GHLSLSTYEFQTSDPRDAHRLVKELKKELEKEAKDLRRKG |
| | LDVKIWVWVNQIGDSVVVWVEKRTE |
| 1532 | TEILVYAELILVEVAIEAGDEDLARLAVEALKRLGDPRAK |
| | EIARLLEECIKRKDPELCKEIIKKA |
| 1533 | PYCKEAAKVLLEAIEAGDEYATAAALYVLKLLCGDPEEVK |
| | KLLEEVAKELGVPPEVVELAKKLLE |
| 1534 | SEVDVVIWAFNKDDPDQRKKAEKKVKDFEKKAREKAKRKN |
| | VPITVHKSTFTVNGLVFISVIVEVG |
| 1535 | SDFREWKYSVEGTDEEAKKEIKKFVKTATTRAKKLAKELN |
| | RSVTIRYFIHSDDNSIDVIVVVVVQ |
| 1536 | DDIDFFIETIDVKDEEDKKKAEKYAKKVKKDIEKHFKDRG |
| | LDVKVTLYYDTLNGTFWFVVWVRSR |
| 1537 | SEEEVVSWTFNKDDPDQRKKAEKKVKDFEKKAREKAKRKN |
| | VPIFVWKQWFTKDGTVSIVVWVVVG |
| 1538 | AEIVRVRFKYDKSDSKSEKKAKDTAKKISDEVKKKAEKKG |
| | LPVSVWSNTLEKDGKVKFFLVVFVG |
| 1539 | DQKVVVWIFYNGDTKRETYRYGTDEEAEKKLKKLIKDWKR |
| | KARKLGGSVVIFVRNGDHVRVWIVE |
| 1540 | DEVIFLVTWEDGDDDRAKKVVKKIKKEATKLAQKLSKEYN |
| | LSYSVETKKRTENGQVVVQIALRVK |
| 1541 | SIVDFYIYREDARDPDQRRSAKEILTRVRKRLTKEAKKQG |
| | LDVKVTLFSRQDGTTLNVVVAVWVD |
| 1542 | DKVYLYWYVVKGTDEEAKKIADKERKDAKKHAKKYAQSVG |
| | GSVRVRVRYYESGDIIVLVVVVTVR |
| 1543 | SDIILYVYTETGPDAEEKARKTATKVVKKVATKLRKKVGV |
| | DKFKYLILVRKKDGTVLVVIFVRVE |
| 1544 | DEFYIYLYEVKGTDEEAKKIADKERKDAKKHAKKYAQSVG |
| | GSVRVTVRYREDGDAIFLVVEVTVR |
| 1545 | SEELIETIKELLKRVPDPRVRELARKALKLAEELGDPELL |
| | KLLKTCLDRGNPLCVEAQALLIEVN |
| 1546 | PLEERAEEAIIKALKLGNPVNIHAQLALIAVALGVPPEEV |
| | LKLVLKKAKERGLLSPEELKLLKEL |
| 1547 | DVLFLVAVENNGSAEWFRVRVGPDPSEKKKAKKLVKEFVE |
| | RARRKGALVVVQTIDGDKQEHQFYK |
| 1548 | DVFAVYIYTVKGTDEEAKKIADKLRKDAKKHAKKYAQSVG |
| | GSVRVRVWFREDGDTILLVVLVRVR |
| 1559 | DQIVVVVIIYNGDVEVEYYRYGTDEEAEKKLKKLIKDWKR |
| | KARKLGGSVTIFVINGDKSRRWHVQ |
| 1549 | DWQAVWIYFVDGTDEEAKKIADKLRKDAKKHAKKYAQSVG |
| | GSVRVRVRFLEFGDIIVLYVEVTVR |
| 1550 | DVEEAERLAKELGDPDILEALKELKERLKKGDPEAEEIIK |
| | LLSELLKEGNKEAVHTIVAVLLYIK |
| 1551 | DLVEELAKLALELGDLELIALVAHLYVAILAGDPELLKEL |
| | LKELKEVARKLGDPRIERLVRLIEE |
| 1552 | DELCKVVLELLERGVSPEEVKEIAKELGFSEEQVEVLLTV |
| | EAVLKYLGVPPEEIEEACKLLIKLE |
| 1553 | DIQWIDVAFVDGTDEEAKKIADKLRKDAKKHAKKYAQSVG |
| | GSVRVRVHWREWGNLIALFVEVTVR |
| 1554 | SELEEELKRLLCEGDPKKVEKLIRELAKRLGRPPRAVLRK |
| | LYKELARRGDPCAAALTVLAVRLYV |
| 1555 | DQKVSVSIVYNGDLKIEHYRYGTDEEAEKKLKKLIKDWKR |
| | KARKLGGFVVIIVHNGDEVRIWLVE |
| 1556 | DKEEELIKELVRLIEKAGNPHAALALLYVKVLRRNDPELA |
| | RLLKKIAKKITKDPREVEELRELAR |
| 1557 | ADWRWWIIIIDTTDPKQQKKVEDKARKLAKKIKKDAERSG |
| | LDVKVQVRSWTANGRFTVVVQSAVQ |
| 1558 | GDIDVVTIIVDSTDPDSQKKAEDTLKKISKKAEKKARKLG |
| | KSVSYKINSDTENGLYVVQIWVEVE |

A number of the polypeptides were subjected to site-saturation mutagenesis (SSM), as described in the examples, to demonstrate the ability to make significant changes to the primary amino acid sequence while maintaining activity of the polypeptide. In one embodiment, substitutions relative to the reference polypeptide are selected from the residues listed as “best” or “tolerable” at each position immediately below the reference polypeptide listed in Tables 13A-13HHH.

In this embodiment, for example, where the reference polypeptide is Cd3_mb1 (SEQ ID NO: 759), residue 1 may be N, K, or V; residue 2 may be E, R, K, Q, H, S, W, V, T, M, A, I, Y, F, C and so forth.

In another embodiment, the substitutions relative to the reference polypeptide are selected from the residues listed as “best” ˜ at each position immediately below the reference polypeptide in Table 13. In this embodiment, for example, where the reference polypeptide is Cd3_mb1 (SEQ ID NO: 759), residue 1 may be N or K; residue 2 may be E, R, or K; and so forth, as detailed in Tables 13A-13HHH.

In another embodiment, 1, 2, 3, 4, 5, 6, 7, 8, 9, 10, 11, 12, 13, 14, 15, or all interface residues are as defined in the reference polypeptide in Tables 13A-13HHH (i.e.: position is denoted with an “X” in the “at interface” column). In this embodiment, for example, where the reference polypeptide is Cd3_mb1 (SEQ ID NO:759), residues 2, 4, 6, 26, 28, 30, 34, 36, 38, 40, 57, 59, and 61 are interface residues, as detailed in Table 13A.

In a further embodiment, protein core residues (core residue positions denoted with an “X” in the “protein core” column in Tables 13A-13HHH) are substituted relative to the reference polypeptide only with conservative amino acid substitutions. In this embodiment, for example, where the reference polypeptide is Cd3_mb1 (SEQ ID NO:759), residues 3, 5, 7, 12, 16, 20, 22, 25, 37, 39, 42, 47, 50, 54, 56, and 58 are core residues, as detailed in Table 13A.

In one embodiment, insertion of amino acid residues relative to the reference polypeptide occurs at a residue indicated in the column “loop/insertion” (i.e.: residues denoted with an “X” in the “loop/insertion” column of Tables 13A-13HHH). In this embodiment, for example, where the reference polypeptide is Cd3 mb1 (SEQ ID NO:759), residues 8, 9, 20-23, 31-33, 41-43, and 55-56 are loop/insertion residues, as detailed in Table 13A.

The polypeptides may incorporate any insertion relative to the reference polypeptide (i.e.: additional amino acids inserted into the sequence). In one embodiment, the insertions are made at loop regions in the polypeptides, as noted in the column “loop/insertion”). The insertion may be a single amino acid, a large functional domain, or any other amino acid insertion as suitable for an intended purpose.

Tables 13-A-13HHH provide details on interface, core, and loop residues and “best” and “tolerable” amino acid substitutions relative to specific binding proteins shown in Tables 1-12.

TABLE 13A

| | | | | | |
|---|---|---|---|---|---|
| | | | at_ | pro- | loop/ |
| posi- | | | inter- | tein_ | inser- |
| tion | best | tolerable | face | core | tion |

CD3d_mb1
NHIACEIHNPEAAKEIAKVANVRRVYFIKQPGNRYFVLLKNADPEGVKKVRSKYNVRCVIRE
(SEQ ID NO: 759)

| | | | | | |
|---|---|---|---|---|---|
| 1 | N, K | V | | | |
| 2 | E, R, K | Q, H, S, W, V, T, M, | | | |
| | | A, I, Y, F, C | X | | |
| 3 | I, F, V, Y | T, G, E, K | | X | |
| 4 | E, Q, M, V | A, K, F, I, G, T, L, | | | |
| | | R, N, S | X | | |
| 5 | C, P, M | G, K, R, D, I, Q, N | | X | |
| 6 | K, R, W, D, M | L, Y, I, E, S, H, A, F | X | | |
| | | , V, Q, G, T, C, N, P | | | |
| 7 | T, I, A, L | V, S, K, W, D, F, N, Q | | X | |
| 8 | H, Q, T, P, L, N, R | A, Y, W, G, K, M, F, | | | X |
| | | V, C, E, I | | | |
| 9 | S, N, Y, T, R | F, M, I, V, C, L, Q, | | | X |
| | | K, G, W, A, P | | | |
| 10 | K | M, L, I, F, R, W, P, V, | | | |
| | | Y, G, S, A, E, T, D, Q | | | |
| 11 | R, K | H, W, Y, S, F, Q, L, E, T, | | | |
| | | A, V, I, G, M, N, C | | | |
| 12 | A, M, T, L | V, G, C, R, Y, Q, I, H, F, N | | X | |
| 13 | R, L, I | V, A, F, M, W, C, S, D | | | |
| 14 | N | K, M, I, V, A, R, L, C, | | | |
| | | W, F, Y, Q, H, T, G | | | |
| 15 | K, R, F | H, E, M, Y, A, G, Q, W, | | | |
| | | L, I, S, V, N, T | | | |
| 16 | W, Y, L, F | I, C, V, D, M, S, R, P | | X | |
| 17 | F, I, A, R, | C, L, D, G, V, K, S, H, P, T | | | |
| | W, M, Y, N | | | | |
| 18 | K, S, G, V | T, R, C, I, W, A, F, L, E, H | | | |
| 19 | L, A, T, G | V, K, H, R, C, I, S, F, N | | | |
| 20 | G, Y, A | K, F, Q, R, N, S, L, E, P, | | X | X |
| | | T, H, D W, V | | | |
| 21 | P, H, A, W, L | N, R, C, S, K, V, T, F, | | | X |
| | | M, Y, Q, G, D | | | |
| 22 | R, Y, K, V, A, | N, G, W, P, C, F, D, L, I | | X | X |
| | S, E, H | | | | |
| 23 | Q | R, W, Y, C, L, F, N, V, P | | | X |
| 24 | N, P, D, C, E | R, V, Y, L, W, I, M, F, K, S | | | |
| 25 | V, T, S, A | L, C, H, K, M, R, F | | X | |
| 26 | Q, A | F, I, K, G, L, R, Y | X | | |
| 27 | F, I, H | R, V, Y, W, M, Q, L, S, K, N | | | |
| 28 | I, T, P, Y | R, M, W, L | X | | |
| 29 | I, K, L, T, M, V, | F, Y, A, G | | | |
| | C, W, P, S, R | | | | |
| 30 | S, K, R, Y, Q | W, A, I, L, P, M, V, | X | | |
| | | T, F, C, H | | | |
| 31 | L, P, Y, K | V, H, W, R, N, Q, M, F, T | | | X |
| 32 | G, R, V, C, E, D, E | W, S | | | |
| 33 | S, K | N, R, W, H, M, Y, F, | | | X |
| | | C, V, L, A, P, T | | | |
| 34 | R, S, T, Q, M, | H, P, K, I | X | | |
| 35 | Y, Q | L, F, I, K, C, R, S, W, A, V | | | |
| 36 | I, V | F, L, S, Q | X | | |
| 37 | V, C, M | F, Y, I, L, N, P | | X | |
| 38 | L, F, E, I | Y, T, M | X | | |
| 39 | L, V, F, W, T | C, A, M, G, D | | X | |
| 40 | K, G, E, V | R, N, I | X | | |
| 41 | N, D | T, L, G, K, R, S, C, A, | | | X |
| | | H, E, W, Y, P, V | | | |
| 42 | A, Q | G, P, D, K, C | | X | X |
| 43 | D, N, Y, Q | A, R, W, C, S, L | | | X |
| 44 | W, P, V, K | T, A, L, H, I, Y, Q, | | | |
| | | S, E, R, M, N, F | | | |
| 45 | E, K, Y, H, R, W, T | Q, M, I, F, A, V, N, G, C, L | | | |
| 46 | R, F, K, G, M, I, Y | H, P, A, W, S, C, D, L | | | |
| 47 | V, R, N, Y | P, L, I, T, A, D | | X | |
| 48 | M, F | K, I, Q, L, C, R, W, | | | |
| | | H, T, S, A, V, N | | | |
| 49 | H, Y, K, F, E | G, N, S, C, P, M, | | | |
| | | V, D, T, L, I | | | |
| 50 | V, Y, K, G, | R, W, C, E, S, A | | X | |
| | N, T, Q | | | | |
| 51 | E | A, V, P, Y, I, S, C, R | | | |
| 52 | N, G | M, S, F, R, Y, I, H, W, | | | |
| | | K, V, C, P, A, L, D | | | |
| 53 | V, K, R, M | F, Q, Y, W, T, C, D, A | | | |
| 54 | Y, W, V, R, C | L, A, H, G, I, F, S, T | | X | |
| 55 | K, N, G | Q, W, R, A, C, Y, H, T, P, | | | X |
| | | D, V, E, F, I, S, M | | | |
| 56 | V, A, M, K, S | I, F, E, L, W, Y, P, C, R, N | | X | X |
| 57 | R, F, N, K, V, H, L, | W, M, C, A, | X | | |
| 58 | C, F | N, E, Y, W, P, I | | | |
| 59 | H, V | Q, W, C, L, T, K, F | X | | |
| 60 | Q | I, F, E, P, R, K | | | |
| 61 | M, R, K | C, H, D | X | | |
| 62 | Y, E, S, R, C, I, K | F, M, L, T, V, H, | | | |
| | | W, G, N, Q, D, P | | | |

TABLE 13B
EGFR_mb1
SDELFSKVELKVTELSMIVMNAKTEDEKKTALTKIKQIADKVQDEELSKFVKRALEHVKKEVG
(SEQ ID NO: 2)

| | | | | | |
|---|---|---|---|---|---|
| 1 | N, A, S, P, D | R, Q, T, E, H, M | | | |
| 2 | D | S, K | X | | |
| 3 | E | A, S, Y | | | |
| 4 | E, L, M | I, Q, Y, C | | | |
| 5 | F,W | L, T | X | | |
| 6 | H, L, V, Y | S, M, I, T, F | X | | |
| 7 | Q, E, D, A, K | S, R, T, N, H, F, M | | | |
| 8 | V | T | | X | |
| 9 | Y | F | X | | |
| 10 | L, I | V, A | X | | |
| 11 | E, Q | D, T, K, R, C, H, Y, A, V, M, S, W | | | |
| 12 | V | T, I | X | | |
| 13 | T, K | G | X | | |
| 14 | R, E | D, N, A | | | |
| 15 | L | F, W | | X | |
| 16 | S, T | C, M | | | |
| 17 | R, F, Q, L, M | Y, I, K, C | X | | |
| 18 | E, Q, I | V, T, A, M, N, K, D, L | | | |
| 19 | A, V | L, W, M | | X | |
| 20 | R, K, M | L, T, V, I, H, E | X | | |
| 21 | Y, S, F, H, A, N, T | G, Q, R, M, V, I, W | X | | X |
| 22 | G, A | S, H, P | | X | X |
| 23 | P, R, K | H, A, Q, S, N, G, Y, E | | | X |
| 24 | N, S, T, D | K, A, Q, C, G, I, F | | | X |
| 25 | A, E, W, M, D, Q | C, V, T, L, H, N, I, F, K, R, S | | | |
| 26 | N, E, Q, A, D, V, S, I, T, | W, P, Y, C, F | | | |
| | H, K, M, R, L, G | | | | |
| 27 | E | K, A, L, D, Q, R | | | |
| 28 | R, K | Q, A, S | | | |
| 29 | M, Q, N, E, W, L, T, D, C, | G | | | |
| | I, V, F, A, R, K, H, S, Y | | | | |
| 30 | D, W, S, E, M, Q, A, I, G, | H, V, R, L, Y, F | | | |
| | N, T, C, K | | | | |
| 31 | A | D | | X | |
| 32 | L | M, F | | | |
| 33 | Y, Q, D, A, S, C, E, L, N, | I, V | | | |
| | G, F, W, H, M, R, T, K | | | | |
| 34 | A, Q, E, S, K, H, D | R, C, N, G, T, L, M | | | |
| 35 | M, I | L | | X | |
| 36 | Q, E, A, K, S | G, N, T, R, C, V, I | | | |
| 37 | M, E, D, A, Q, N, Y | I, C, R, H, L, S, F, V | | | |
| 38 | I | T | | X | |
| 39 | A, G | S | | | |
| 40 | D, E | C, A, G, L, F, R | | | |
| 41 | G, A, Q, D, H, T, S, E, N, | F, M | | | |
| | W, C, R, K, Y | | | | |
| 42 | V | I, C | | X | X |
| 43 | E, T, Q, Y | D, V, H, S, I, M, R, P, W, C | | | X |
| 44 | D | Q, G | | | X |
| 45 | P, E | D, T, V, K, S, C, I | | | |
| 46 | E | D | | | |
| 47 | V, I, T | L, C | | | |
| 48 | L, E, A, R, Q, K, Y, V, I, S | G, M, T, W | | | |
| 49 | D, E, Y, I, V, S, A, T, L, | K, R, H, C, G | | | |
| | Q, N, M, F,W | | | | |
| 50 | F | | X | | |
| 51 | Q, V | | | | X |
| 52 | E, I, N, R, Q, L, T, H, M, | G, S | | | |
| | F, W, Y, V, K, C, A | | | | |
| 53 | N, V, T, K, A, G, Q, L, D, | | X | | |
| | S, H, I, R, M | | | | |
| 54 | A | H, V | X | | |
| 55 | Y, L | W, F, M | | | |
| 56 | E | A, D, Y, K, H, W | | | |
| 57 | H | N | X | | |
| 58 | I, V | A, L | | X | |
| 59 | W, Q, T, I, L, M, V, C, E, | A | | | |
| | K, Y, S, R | | | | |
| 60 | A, K, N, M, S, Q | H, L, G, R, I, D, W | | | |
| 61 | K, R, M, Q, S, I, T, H, V, | A, Y, C, W | | | |
| | N, E, L | | | | |
| 62 | L, M, A, S, I, V | T, Y, H, F, W, R, K, G | | X | |
| 63 | F, W, E, Y, S, M, G, N, A | R, Q, K, D, L, I, C, V, P, T, H | | | |

TABLE 13C
EGFR_mb2
SLDEAKKLLQEAEKLARKINDRMELAYVEFLKHILETAKKQNDKRTIESVRDMARDALEELQS
SEQ ID NO: 142

| | | | | | |
|---|---|---|---|---|---|
| 1 | K, G, M, W, T, R, N, Y, A, I, C, V, S, F, P, E,H, Q, D, L | | | | |
| 2 | E, M, K, T, N, F, R, L, W, H, C, Y, G, D, P, I, S, A, Q, V | | | | |
| 3 | N, F, V, H, E, M, R, G, K, W, Y, A, L, T, T, C, D, Q, S, I, P | | | | |
| 4 | M, S, T, V, I, Q, A, D, G, C, E, L, P, N, Y, H, K, F, R | | | | |
| 5 | V, A, T, C, I, P, G, S | E | | X | |
| 6 | E, R, L, T, C, Y, G, F, K, H, D, M, N, I, V, Q, S, A, W, P | | | | |
| 7 | D, C, G, W, L, F, T, I, R, S, K, Q, H, Y, A, N, M, E | | | | |
| 8 | Y, F, M, I, L, W, V, D, T | C, A, H | | X | |
| 9 | L, I | | | X | |
| 10 | Y, N, G, F, H, T, S, R, V, D, A, M, Q, K, C, I, L, W, E | | | | |
| 11 | G | N | | | |
| 12 | Y, C, A | L, S, F, V | | X | |
| 13 | E, Y, A, D, G | H, Q, S, T, W, F, M, L, K | | | |
| 14 | H, W, E, C, S, T, G, R, Q, I, V, N, M, Y, K, F, A, D, L | | | | |
| 15 | M, E, I, L, W, Y, V, F, A, Q | C, H | | X | |
| 16 | A | | | X | |
| 17 | W, M, L, Q, V, A, I, R, K, F, H, T, C, Y, G, S, E, N | | | | |
| 18 | C, Y, L, E, D, T, M, W, R, I, V, N, A, H, Q, F, K, S, G | | | | |
| 19 | L, T, M, I, G, Y, F, F, W, V, C, Q, A, S, R, E | N, H, D | | | X |
| 20 | H, A, D, M, G, S, C, K, N, E, Q, R, T, F, P, Y | V, L, I | | | X |
| 21 | D | N, S, E, Q | | | X |
| 22 | T, P, Q, K, A, R, S, N, G, D, H, I, E, L, C | M, V | X | | |
| 23 | T, H, L, M, I | F | X | | |
| 24 | E | | X | | |
| 25 | I | V, M, T, L, C, Q, E, S, Y, | | | |
| | | W, A, F, F | | | |
| 26 | G, A, K, M | S | X | | |
| 27 | Y | | X | | |
| 28 | C, I, V, F, D, A | L | | X | |
| 29 | E, H, D, N, T, K, M, V | A, F, Q, S, R, G, I, L, | X | | |
| | | W, Y | | | |
| 30 | L, F, M | | X | | |
| 31 | L | | | X | |
| 32 | D, A, G, R, F, S, Y, W, Q, M, E, T, N, V, K, C, H, I | L | | | |
| 33 | S, E, G, A, Y, N, Q, H, D, T, W, M | V, R | X | | |
| 34 | Y, L, N, E, I, S, M | Q, W, A, V, D, F, H, T, C, | X | | |
| | | G, R | | | |
| 35 | F, Y, L, W | H | | X | |
| 36 | M, A, D, S, Q, N, F, C, E, K, H, Y, V, T, L, W, I | R, G | | | |
| 37 | D, Q, E, N, S, T | G, H | X | | |
| 38 | A, S, G | T | | X | |
| 39 | E, L, M, I, V, C, N, Q, T | A, Y, F, W, S, R, K, H, | | | |
| | | D, G | | | |
| 40 | W, H, Y, V, I, F, K, E, M, C, G, T, S, L, R, N, D, Q, A | | X | | |
| 41 | E, D, T, Q, Y, G, A, S, M | N, I, V, W, H, C, F, L, R | X | | X |
| 42 | Q, S, E, G, N, D, A, C, K, H | R, V, M, T | | | X |
| 43 | Y, D | | X | | X |
| 44 | E, D, H, A, T, V, I | S, L, M, Q, P, R, C, W, K, | | | |
| | | G, F, N, Y | | | |
| 45 | E, D, A, Q | N, S, V, P, G, M, H, T, K, | X | | |
| | | I, W, R, Y, L, F | | | |
| 46 | E, G, Q | S, A, D, T, W, Y, N | X | | |
| 47 | G, M, I, F, A, E, Q, C, Y, W, L, V, T | S, N | | | |
| 48 | D, E | Q, M, A, C, I, V | | | |
| 49 | E, D, Q | V, I, N, H, T, S, M, G, R, | X | | |
| | | A | | | |
| 50 | T, A, C, V, I | | X | | |
| 51 | T, V, Y, L, W | K, S, H, Q, G, F, M, N, E, | | | |
| | | R, A, I, C | | | |
| 52 | D, E | Q, A | | | |
| 53 | M, L | | X | | |
| 54 | A | S | | X | |
| 55 | W, H, V, L, C, M, S, I, E, A, Q, T, R, F, Y,, K, N, G | | | | |
| 56 | D | | X | | |
| 57 | E, N | A, Q, C, H, S, D, T | | X | |
| 58 | L, V, I, Y | F, C, W | | | |
| 59 | Q, L, T, W, H, V, C, Y, S, I, A, F, E, D, M, N, G, R, K | | | | |
| 60 | F | Y, A, D, S, E, C, V, H, W, | | | |
| | | Q | | | |
| 61 | L | | | X | |
| 62 | C, E, S, L, A, R, M, Q, K, H, T, V, Y, N, I, G | W, F | | | |
| 63 | Q, C, Y, D, V, F, I, W, E, T, G, K, H, N, M, S, L, A, R, P | | | | |

TABLE 13D
EGFR_mb3
SLEEVKELIRKLVPDPRLVWALEDLLELLKKGDPLAEEVLRFYLASAREQGDKDLTKAIELVLKT
(SEQ ID NO: 4)

| | | | | | |
|---|---|---|---|---|---|
| 1 | T, D, P, Q, H, S, R, N, V | G, I, A, L, C, K, Y | | | |
| 2 | M, D, V, T, I, L, A, H, Q, P, N, S, G, W | Y, F, E, R, K | | | |
| 3 | T, E, S, N, H, V, M, P, K, W, F, L, A | G, R, D, I | | | |
| 4 | S, C | D, N, E, P, T, K, Q, H, F, G, A, I, Y, L, | | | |
| | | V, W | | | |
| 5 | M, L, V, C, T, G | I, Q, S, A, E, P, Y W, N, R | | X | |
| 6 | R, T, K, Q | S, L, P, V, A, G, H, I | | | |
| 7 | C, V, D, S, E | N, Y, T, I, Q, H, K, R, A, L,P W, F,M | | | |
| 8 | C, A, M, I, S, F, K, V, Q, N, W, T, R, H | L, Y, G, D, E | | | |
| 9 | V, I | M | | X | |
| 10 | H, R, K | S, Y, E, N, I, W, Q | | | |
| 11 | C, A, F, I, E, L, Y, D, Q, K | W, H, R, G, S, M, N, V, T | | | |
| 12 | W, L | M, I, F, Y | | X | |
| 13 | V | I, C, R | | X | X |
| 14 | P, Q, D, L, N, A, T | F, M, S, K, H, I | | | X |
| 15 | D, E | H, F | | | X |
| 16 | E, A, D, K, P | S, G, T, Q | | | |
| 17 | N, Y, M, I, T, L, G, S, Q, V, A, H | P, F, K, R, D, W | X | | |
| 18 | M, V, F, L | Y, P, N | X | | |
| 19 | L, I, S, C | V, N, Q, W, R, A, Y, F, T H | | | |
| 20 | W, H | | X | | |
| 21 | T, A, W | I, L, N, V | X | | |
| 22 | I | L, V, P | X | | |
| 23 | E | C, I, V, T, L | | | |
| 24 | D | | X | | |
| 25 | M, L | | X | | |
| 26 | W, Y, G, A, M, F | S, L, Q, H, C, V, T, I | | X | |
| 27 | M, R, D, C, S, E, A, Q, H, K, G, T, N | Y, I, L, F, V | | | |
| 28 | E, H, Y, F, I | L, C, Q, P, W | X | | |
| 29 | Y, W, M, H, F | L, S, C, N, Q, R, T, K | | X | |
| 30 | Y, W, C, M, V, N, S, L, R, D, H, I, A, E, | Q, T, K, F | | | |
| | G | | | | |
| 31 | G, C, A, N, D, L, E, Q, Y | S, M, T, K, F, H, V, I, R, W | | | X |
| 32 | G, A, Y | E, R, Q, C, S | | | X |
| 33 | E | D, N, A | | | X |
| 34 | E, M, P | C, D, V, N, Q, L, A, T, G, S, H, R, F, K | | | |
| 35 | V, D, L, I | H | X | | |
| 36 | A | L | X | | |
| 37 | D, E | N | | | |
| 38 | E | | X | | |
| 39 | V | | X | | |
| 40 | L | F | | X | |
| 41 | F, C, H, E, Q, M, N, Y, W | G, R, L, D, I, A, V | X | | |
| 42 | Y, W | F | X | | |
| 43 | H | Y, F | X | | |
| 44 | W, L | E, Y, H, M | | | |
| 45 | M, N, L | A, R, Q, Y, S, T, K, D | X | | |
| 46 | T | L, S, C, G, I, Q | X | | |
| 47 | A, S | G | | X | |
| 48 | V, I, R, M, Q | K, E, H, L, F, Y, A | | | |
| 49 | Q, W, D, E | V, M, I, S, Y, A, L, G | X | | |
| 50 | Q | G | X | | |
| 51 | K, R, G, H | S, A, C, F | | | X |
| 52 | E | D, C, I | | | X |
| 53 | W, C, M, I, T, V, F, A, H, Q, K, R, D, E, | P, Y | | | |
| | N, G, S | | | | |
| 54 | D, P, E, N, L, Q | H, G, T, F, W, C | | | |
| 55 | Y, A | L, F, W, M, T, V, S | | | |
| 56 | E, A, G, Q, F, C, Y, V, T, H, S, M | D, R, W, I | | | |
| 57 | M, T, I, Q, V, L, K, Y | S, H, C, E, W, R, F, A, N, D | | | |
| 58 | A | C | | X | |
| 59 | C, V, I | L | | X | |
| 60 | Q, E, S, I, L, C, V, M, N | T, D, G | | | |
| 61 | E, I, F, M, H, A, Y, L, V | C, S, K, Q | | X | |
| 62 | M, I, C, V | A, T, L, S, Q | | | X |
| 63 | L | I | | | |
| 64 | M, C, A, G, Q, D, Y, K, T, N, L, S, I, F | E, V, W, H, R | | | |
| 65 | W, H, Y, F, C, I, L, Q, D, T, M, V, N, A | R, G, E, P, S | | | X |

TABLE 13E
EGFR mb4
DDESLKLLLILVQIQLALERGEISNDQAKELAKRVEEKARKLGDEQVQRNVENVKEVIERFG
(SEQ ID NO: 5)

| | | | | | |
|---|---|---|---|---|---|
| 1 | Q, G, A, H, N, S, T, L, D, P, E, F, | K, V, W | X | | |
| | I, M, Y | | | | |
| 2 | E, D, G, S, Q | H,N | X | | |
| 3 | A, Y, P, Q, E, D, K, I, V, T, H, L, | W, G | | | |
| | M, N, F, S, R | | | | |
| 4 | T, N, M, P, A, Q, S, D, R, V, I, E | K, G, C, H | | | |
| 5 | M, F, L | I, W | X | | |
| 6 | D, G, A, K, S, H, V, R, E, T, I, Q, | F, W, P | | | |
| | N, M, L, Y | | | | |
| 7 | Q, L, M, F, V | C | | X | |
| 8 | H,C, L, M | | X | | |
| 9 | W | Q, H, I, Y, F, L, M, R, V | X | | |
| 10 | S, G, W, H, Q, R, T, D, I, A, M, C, | | K | | |
| | L, E, N, Y, V, F, K | | | | |
| 11 | L, I | | | X | |
| 12 | V, M, G | I, P | X | | |
| 13 | R, N, H, Q, W, E, A, D, S, T, K, Y, | F | X | | |
| | G | | | | |
| 14 | V, M, I, L | C | | X | |
| 15 | K, Q | | X | | |
| 16 | Y, V, L, H, Q | M, R, T, W, K, I | X | | |
| 17 | E, G, Y, T, H, D, A, L, M, C, R, N, | V, P | | | |
| | K, Q, S, F | | | | |
| 18 | M, L, W, Q | F, I | | X | |
| 19 | D, G, R, E, S, N, Q, K, T, A, H, P | M | X | | |
| 20 | A, D, N, S, H, G, R, E, K, C, T, V, | W, M | X | | |
| | Q, Y, F, L, I, P | | | | |
| 21 | H, N, E, S, A, F, K, G, Q, M, D, Y, | W | | | X |
| | R, C, L, T | | | | |
| 22 | Q, T, D, S, E, N, R, H, A,V, L | K, C | X | | X |
| 23 | I, V, L, C | M | | | X |
| 24 | R, G, K, T, N, S, E, M, D, F, A, C, | Y | | | X |
| | H, Q, V, L, I | | | | |
| 25 | T, F, L, K, D, C, N, Q, M, Y, A, E, | | | | |
| | I, G, P, S, R, V, H | | | | |
| 26 | I, W, F, P, S, L, N, R, A, T, G, D, | | | | |
| | Q, E, K, H, C, V, M, Y | | | | |
| 27 | W, G, T, S, N, F, K, M, L, V, Y, C, | | | | |
| | D, Q, R, I, H, E, A | | | | |
| 28 | A, G | E | | X | |
| 29 | W, C, I, A, Q, V, R, Y, N, S, T, L, | D | | | |
| | K, H, F, G, M, E, P | | | | |
| 30 | S, W, G, Q, T, N, F, H, L, V, E, I, | | | | |
| | M, D, C, K, A, Y, R | | | | |
| 31 | W, T, D, F, H, R, L, E, I, Q, N, V, | | | | |
| | M, K, C, A, Y, G, S | | | | |
| 32 | T, N, A, V, L, C, S, P, E, K, G | I | | X | |
| 33 | R, G, F, D, W, H, S, I, L, V, M, K, | | | | |
| | Q, E, A, Y, C, N, T | | | | |
| 34 | V, K, W, F, Y, T, N, I, L, R, A, S, | P | | | |
| | Q, M, D, E, H, G, C | | | | |
| 35 | I, V | M, C | | X | |
| 36 | E, D, G, S, A, Q | N | | | |
| 37 | F, D, R, T, M, Y, V, Q, W, S, A, E, | | | | |
| | N, I, L, C, H, K, G | | | | |
| 38 | M, S, V, H, Q, D, C, N, A, K, E, T, | F | | | |
| | G, L, Y, R, I | | | | |
| 39 | G, A, N, S | | | X | |
| 40 | I, G, Y, M, K, W, Q, T, D, R, S, E, | | | | |
| | F, V, N, H, L, C, A | | | | |
| 41 | H, M, T, G, V, R, N, K, Y, L, D, F, | | | | |
| | Q, S, I, A, E, W, C | | | | |
| 42 | T, I, V, C, N, S, Y, L, A, R, M, W, | | | X | |
| | Q, H, K, D, F, E | | | | |
| 43 | K, R, C, D, N, G, E, Q, A, H, V | S, M | | | X |
| 44 | D, L, Y | S, A, N | | | X |
| 45 | W, Q, L, A, S, E, D, I, G, V, M, C, | R | | | |
| | P, K, N, F, Y, T, H | | | | |
| 46 | Q | E, V | X | | |
| 47 | V | | X | | |
| 48 | W, V, L, E, S, A, Q, F, T, R, I, M, | N, D | | | |
| | K, Y, H, G, C, P | | | | |
| 49 | M, Q, N, T, A, R, K, V, I, L, S, H, | | X | | |
| | G, F | | | | |
| 50 | N, C | | X | | |
| 51 | C, V, I, E, A, G | | | | |
| 52 | V, A, C, E, D, I, Q, R, N, S, M, L, | H, Y | | | |
| | W, K | | | | |
| 53 | N | | X | | |
| 54 | I, V, C, T | A | | X | |
| 55 | Y, F, I, H, R, V, N, K, W, L, M, Q | C, A, E | | | |
| 56 | Y, W, D, T, H, S, N, E, C, R, A, V, | | | | |
| | I, L, Q, M, F, G, K | | | | |
| 57 | I, V | | X | | |
| 58 | I | | | X | |
| 59 | L, T, G, C, Q, A, H, N, D, E, R, S, | V, I | | | |
| | M, Y, K, W, F | | | | |
| 60 | H, K, R, G, Q, L, A, Y, N, F | M, I, S, T, W | X | | |
| 61 | V, A, T, M, S, L, Y, H, F, I, Q, K, | G | | X | |
| | N, R, W, C, P | | | | |
| 62 | C, D, A, E, V, M, R, T, I, G, H, K, | Y, W | | | |
| | Q, S, N, P, L, | | | | |

TABLE 13F
EGER_mb5
DHWEEVERWALEHLQEATQQNDPQKAKKILEEAHKWLRRELSEEEARAVVRWLKQLVDRELS
(SEQ ID NO: 143)

| | | | | | |
|---|---|---|---|---|---|
| 1 | F, M, L, W, V, P, Y, T, A, C, I | E, H, G, R, K, Q, S, D, N | X | | |
| 2 | G, R, A, K, D, E, N, T, Q, H, S, Y | | | | |
| 3 | W | | X | | |
| 4 | D, E, T, S | | X | | |
| 5 | D, W, T, A, G, M, Q, S, E, N | F, H, K, Y | | | |
| 6 | T, V, I, A, E | | X | | |
| 7 | Y, F | | X | | |
| 8 | E, K, S, L, M, A, G, R, Q, C, H | | | | |
| 9 | Y, M, L, G, T, Q, V, A, H, E, S, R, D, | | | | |
| | I, K, C, W, N, F | | | | |
| 10 | S, G, H, A, T, N | | | X | |
| 11 | I, M, L, Q, P | | X | | |
| 12 | N, Q, G, S, Y, M, D, T, F, H, E, C, A, | | | | |
| | I, K, P | | | | |
| 13 | E, A, S, T, N, H, C, G, M, F, R | W, V, I, Q, D, K, L, Y | | | |
| 14 | Y, M, T, L, G | | | X | |
| 15 | S, P, N, V, F, W, T, G, R, C, D, H, E, | | X | | |
| | M, A, I, L, Q, K, Y | | | | |
| 16 | H, G, K, T, V, Q, A, S, D, N, E, I, C, | | | | |
| | R | | | | |
| 17 | P, S, T, Q, N, C, D, A, E, Y, K, V | | | X | |
| 18 | K, E, W, N, V, D, H, R, S, G, A, Y, P, | | | | |
| | T, Q, M, C, L, F | | | | |
| 19 | V, Q, P, G, K, A, L, N, R, H, D, Y, I, | | | | |
| | W, S, E, F, T, M | | | | |
| 20 | Y, P, V, A, L, T, I, S, E, H, D, M, F, | | | | X |
| | R, N, K, Q, G, C | | | | |
| 21 | C, R, K, A, H, Q, G, N, D, S, E, Y, V, | | | | X |
| | I | | | | |
| 22 | S, C, E, H, N, D, K, M, Q, Y, T, A | | | | X |
| 23 | E, M, H, K, I, W, A, L, D, R, T, V, N, | | | | |
| | Y, Q, G, P, S, F, C | | | | |
| 24 | I, N, A, Q, G, H, V, L, K, D, S, W, M, | | | | |
| | F, T, P, R, E, C, Y | | | | |
| 25 | N, I, V, E, H, G, W, F, M, L, R, S, A, | | | | |
| | Y, K, Q, C, T, C | | | | |
| 26 | K, L, P, C, R, G, T, V, Y, F, H, S, E, | | | | |
| | A, M, I, Q, N | | | | |
| 27 | Y, E, I, F, A, V, C, D, P, L, G, H, M, | | | | |
| | N, T, Q, R, K, S, W | | | | |
| 28 | W, R, L, M, S, V, D, Y, Q, E, F, I, G, | | | | |
| | T, A, N, K, H, C | | | | |
| 29 | T, V, I | | | X | |
| 30 | I, F, M, L, Y, A, T, V, Q | | | | |
| 31 | T, H, I, L, W, C, G, K, F, V, M, R, Q, | | | | |
| | E, N, Y, S, A, D | | | | |
| 32 | V, K, A, M, C, N, H, Q, I, T, D, W, S, | | | | |
| | R, E, Y, L, G, P | | | | |
| 33 | A, S, T | | | X | |
| 34 | K, T, Y, L, A, M, N, Q, E, G, F, D, S, | | | | |
| | H, R, V, P, C | | | | |
| 35 | Q, H, G, F, R, A, E, P, V, W, D, L, Y, | | | | |
| | N, I, C, K, T, S, M | | | | |
| 36 | R, K, W, A, I, D, C, F, Q, G, T, V, Y, | | | | |
| | S, L, H, N, M, E | | | | |
| 37 | F, I, L, M | | | X | |
| 38 | E, T, Q, G, S, K, M, I, V, L, A, R, C, | | | | |
| | H | | | | |
| 39 | Q, T, V, K, E, A, W, G, F, H, Y, S, M, | | | | |
| | N, I, C, L, R | | | | |
| 40 | S, T, Q, V, H | A, N, R, K, L, M, W, Y, G, | | | X |
| | | I, C, E, D, F | | | |
| 41 | L, M | | X | | |
| 42 | P, E, Q, M, D, T, A, S, H, N | | | | X |
| 43 | Q, D, K, A, E, N, G, V, Y | S, P | | | |
| 44 | G, M, N, T, L, Q, A, D, S, P, E, R, K, | | X | | |
| | V, H | | | | |
| 45 | E | | X | | |
| 46 | T, C, S, A, V | | | | |
| 47 | S, T, C, V, D, E, Y, I, L, W, H, G, K, | | | | |
| | R, A, F, Q, M, N | | | | |
| 48 | S | D, T, A | X | | |
| 49 | I, V | | X | | |
| 50 | L, T, G, S, A, C, I, V, Y, F, D, N | | | | |
| 51 | L, E, G, H, K, Y, Q, F, M, N, R, S, A, | | X | | |
| | W, C, D, V, T | | | | |
| 52 | W | H, Y | X | | |
| 53 | I, A, V, L, M | | | X | |
| 54 | L, Q, M, D, F, S, N, C, W, Y, P, E, H, | W, R, A, C, E, F, D, T, K, | | | |
| | R, I, T, K, G, V, A | N, M, L, I, Q, V, S | | | |
| 55 | Y, H | | X | | |
| 56 | L, M | | X | | |
| 57 | G, T, I, N, C, Y, M, S, A, F, H, V, W, | | | X | |
| | Q, L, E, P | | | | |
| 58 | K, F, R, A, S | M, I, Y, G, P, H, L, W, V, | | | |
| | | N, Q, E, T, D | | | |
| 59 | Q, D, E, G, C, N, F, Y, I, M, A, T, K, | | | | |
| | L, P, L, R, W, S, V | | | | |
| 60 | P, M, W, T, G, Q, S, K, D, H, R, E, N, | | X | | |
| | A | | | | |
| 61 | M, R, Y, C, E, Q, W, D, I, H, K, N, P, | | | | |
| | V, T, L, F, G, S, A | | | | |
| 62 | C, M, E, W, R, H, D, L, G, P, A, Q, T, | | | | |
| | F, V, S, I, Y, K, N | | | | |

TABLE 13G
EGFR_mb6
DEAAEEVLRYLKKLGDPELAELIERLLERVRKKKDPDLERTLEIIAVAVLYGDPEIARQALRALH
(SEQ ID NO: 7)

| | | | | | |
|---|---|---|---|---|---|
| 1 | D, E | N, V, Q, F | | | |
| 2 | W, Q, A, E, R | D, Y, H, C, K, M, V, L, I, G | | | |
| 3 | L, I, V, M, T, C | A, K, Q, D, E, P, S, H | X | | |
| 4 | A | | | X | |
| 5 | E, D | A, K, N, G | | | |
| 6 | K, A, T, L, F, I, Y, V, N, | E, G, D | | | |
| | M, S, Q, R, H, C, W | | | | |
| 7 | I, V | C, M, F, E, K, R | | X | |
| 8 | I | L, C | | | |
| 9 | K, R | T, V, I, N, S . L, C, Q, A, H, E, F, P, G | | | |
| 10 | Y, W | S, F, H, Q | | | |
| 11 | I | L, V | | X | |
| 12 | E, D, K | T, M, N, S, Q, A, C, L, Y, G, F | | | |
| 13 | K, R, C, N, H, A, S, D, G | Q, M, T, V, E, L | | | |
| 14 | A, V, T, C, M, I | L, S | | | |
| 15 | S, T, P, K, R, Q | G, E, N, H, D, A, C | | | X |
| 16 | W, D | P, A, G, V, Y, H, N, L, T | | | X |
| 17 | E, D, L, H, M, A, S, G, T, | I, R, W, P | | | |
| | N, V, Q, C, F, Y, K | | | | |
| 18 | E, G, Q, A | D, M, V | | | |
| 19 | I, V | L, R, C | | | |
| 20 | V, C, T | I, A, S | | | |
| 21 | D | E, N, L, M, H, F, Q, S, I, T, C | | | |
| 22 | L, I | V, H, T | | | |
| 23 | I, L | V, E | | X | |
| 24 | E | D, T | | | |
| 25 | K, C, A, E, V, S | R, N, T, H, Q, F, I, M | | | |
| 26 | I, V | L, F, M, T | | X | |
| 27 | L | I, A, N | | | |
| 28 | D, E | F, A, C, Y, I, V, N, S, W, H, T, L, G | | | |
| 29 | V, Q, L, C, A, K, S, I, N, | R, H, F | | | |
| | G, M, T, E | | | | |
| 30 | V, L | A, C, F | | | |
| 31 | A, E, Q, S, H | R, N, K, V, G, Y, T, D, W, M, L | | | |
| 32 | D, V, Q, M, A, E, C, L, I, | K, G | | | |
| | N, W, T, S, F, R, H, Y | | | | |
| 33 | S, Q, R, M, E, A, D, C | L, K, T, W, V, N, H, I, Y | | | X |
| 34 | N, Q, G, A, D, C | E, K, H, R, S, M, P | | | X |
| 35 | D | V, C, N, G | | | X |
| 36 | E, D, Q, S, T, N | A, Y, V, P, L, K, F, M, H, W, R, I | | | |
| 37 | D | E, G, N, Y, T, F, K, Q, R | X | | |
| 38 | L | E | | | |
| 39 | E | D, F, S, H | | | |
| 40 | K, A, R, Q, S | H, N, F | X | | |
| 41 | V, R, T | K, A, I | X | | |
| 42 | I, V | L, M, G, S | | X | |
| 43 | E | D, A | X | | |
| 44 | I | L, W | X | | |
| 45 | V, I | C, T, M, L, G, A | X | | |
| 46 | C,A | S, P, L, F | | X | |
| 47 | L,I | V | X | | |
| 48 | S | A, F, E, R | X | | |
| 49 | V | G, Y, I | | X | |
| 50 | H, L | I, D | | | |
| 51 | Y | | X | | |
| 52 | W, H, Q, N, G | K, A, D, R, C | | | X |
| 53 | D | Y, F | X | | |
| 54 | P | A, N, H | | | |
| 55 | D, E, N | G | | | |
| 56 | I,L | M | X | | |
| 57 | A, C, L | V, D | | | |
| 58 | R | | | | |
| 59 | T, D, N, E, S | Q, G, R, V. H, A, K, L | X | | |
| 60 | G | S, A, R, N, E, Q | X | | |
| 61 | L | F,Q | | X | |
| 62 | A, F, G, S, Y, K, H, W, Q, | R, E, L, N, C, D, V | | | |
| | M, T | | | | |
| 63 | E, D, Q | T, A, V, N, S, M, G, H | X | | |
| 64 | L, E, W | M, A, D, K, S, G, Y, I | X | | |
| 65 | H, L | | | | |

TABLE 13H
FGFR2_mb1
SEELIKKALELLRQGNPDRAAQVLLFLAFQTGDPRVRELFSLLVEAMQKNDPELLKRVRRLLEET
(SEQ ID NO: 20)

| | | | | | |
|---|---|---|---|---|---|
| 1 | K, A, Q, R, V, L, G | M, Y, T, S, I, F, D, N | F | F | F |
| 2 | Y, G, F, I, R, W, M, H, L, P, | E | | | |
| | V, K, T, Q, S, A, N | | | | |
| 3 | G, K, L, M, S, R, P, V, Y, H, A, I, | F, T, E | | | |
| | N, Q | | | | |
| 4 | N, S, C | G, L, K, T, Y, H, E, M, A | | | |
| 5 | K, R, P, F, M | I, Q, H, C, N, S | | | |
| 6 | W, R, K, C, A, M, S, E, P, I, H, V, | | | | |
| | G, L, Y, Q | | | | |
| 7 | F, G, E, Y, N, K, A | D, S, M, Q | | | |
| 8 | A, S, V, G, I, P, T, C | | X | | |
| 9 | H, I, Y, L, Q, T, R, V, F, W, E, C, | | | | |
| | G, D, N | | | | |
| 10 | R, A, Y, L, G, N, T, I | M, H, F, V, K, E, Q, S, D | | | |
| 11 | L, D, C, H, K, E, R, M, N, T, W, F, | | | | |
| | G, S, A, I, P, Y, V, Q | | | | |
| 12 | I, V, M, L, Y, C, H, E, F, A, Q | | X | | |
| 13 | Y, R, L, N, A, I, C, M, F, W, S, G, | | | | |
| | Q, T, D, V, H, E | | | | |
| 14 | L, V, S, M, Q, E, H | D, W, R, I, G, N | | | |
| 15 | N, D, K, H, G, A, Q, F, T, C, M, Y, | | | X | |
| | R, L, E, V | | | | |
| 16 | D, E, L | T, N, H, A, I, V, Q, F, R | | | |
| 17 | P, S, K, N, E, T, R, Q, V, A, M, H, | | | | |
| | I, Y, F, D, G, L, W | | | | |
| 18 | D, H, S, G, W, E, N | | X | | |
| 19 | E, G, N, Q, A, D, M, T, P, W, S, H, V | L, Y, F, R, C | | | |
| 20 | A, S, C, G, T | | | X | |
| 21 | A, C, V, G, S | | X | | |
| 22 | H, S, G | Q, T, A | X | | |
| 23 | Y, L, H, I, M, V | K, E, T, Q, R, W, F, D, | | X | |
| | | P, S, G | | | |
| 24 | M, L | | | X | |
| 25 | L, M | | X | | |
| 26 | T, F | | X | | |
| 27 | L, N, Y, H, T, S, W, A, I, K, R, M, | | | X | |
| | Q, C, F, V | | | | |
| 28 | A | | X | | |
| 29 | Y | F | X | | |
| 30 | W | Q, K, L, G | X | | |
| 31 | T, A, G, S | | | | |
| 32 | K, M, R, Q | G, L, H, S, N | | | X |
| 33 | N, R, K, L, W, I, Q, Y, V, S | M, F, D, H, G, T | | | X |
| 34 | E, K, D, P, H, I, N, S, T | Y, F, V, Q, A, W | | | |
| 35 | E, D | R, C, T, H, N, G, K | | | |
| 36 | L, I | Q, V, T, M, F, S, A | | X | |
| 37 | G, E, Q, A, T, S | R, K | | | |
| 38 | R, I, L, V, M, K, F | Q, T, E, H, S, W, N | | | |
| 39 | L, Q, W, E, K, D, H, V, S, I, M, N, | | | X | |
| | G, Y, C, R, F, A, T | | | | |
| 40 | F, T | | X | | |
| 41 | T, C, Q, M | K, S, I, G, V | X | | |
| 42 | S, M, W, L | C, Q, K, V, D, Y, N, E, | | | |
| | | H | | | |
| 43 | T, V | L, I, S, N | | X | |
| 44 | H, A, S, Y C, F | M, V, L, K | X | | |
| 45 | G, L, Y, S, D, M, H, E | F, A, T, R, N | X | | |
| 46 | G, S, A | F, T | | X | |
| 47 | Y, Q, E, C, T | H, G, M, V, A, F, D | | | |
| 48 | E | Q, H, Y, K, S, D, G, M, | X | | |
| | | R, V | | | |
| 49 | Y, W, F, D, H, L, K, N, E, V, Q, M, | | | | X |
| | S, T, P | | | | |
| 50 | H, N, D, P, C, A, E, Q, M, V, I, K, | | | | X |
| | W, R, T, F, G, S, Y, L | | | | |
| 51 | K, R | S, Q, T | | | X |
| 52 | N, L, F, I, R, D, V, A, E, T, M, Q, | K, S, C, W | | | |
| | Y, G, H, P | | | | |
| 53 | V, H, F, D, I, S, Q | W, E, P, R, N, Y, K, T, | | | |
| | | L | | | |
| 54 | I, M, Q, L | R, C, Y, N, V, P | | | |
| 55 | Y, H, V, Q, L, A, M, E | S, C, K, F, R, W | | | |
| 56 | S, L, M, K, T, Q, V, N, I, E, G, C, | | | | |
| | P, W, Y, R | | | | |
| 57 | K, R, Y | N, W, V, H, C, F, I, T, | | | |
| | | S, G | | | |
| 58 | C, V, I, A, F, S, Q, G, L, P, H, T, | | | X | |
| | Y, M | | | | |
| 59 | K, R, H, E, V, P, W, D, M, L, I, N, | | | | |
| | Y, G, T, S, Q, A | | | | |
| 60 | V, W, R, D | G, Q, N, M, F, C, I, A, | | | |
| | | E | | | |
| 61 | G, D, R, N, M, Y, S | T, E, H, L, A, I, W, K | | | |
| 62 | L, M, I, C, A, V, T, G, F, S | | X | | |
| 63 | A, K, E, V, T, Y, H, G, N, Q, M, I, | | | | |
| | F, R, W, C, D | | | | |
| 64 | Q, T, Y, F, S, W, H, I, G, K, E, M | P, A, R, L, C | | | |
| 65 | K, L, R, W, Y, F | T, A, H, M, V, G, I, N | | | |

TABLE 131
FGFR2_mb2
LEKLERVALLAVRLYMRIGDPEIAKIWFKVMELLHAYQAGHLDEEEAKRRADKLEKELRKFI
(SEQ ID NO: 21)

| | | | | | |
|---|---|---|---|---|---|
| 1 | V, K, F, T, Q, D, E, I, N, L, H, | A | | X | |
| | W, G, M, R | | | | |
| 2 | L, A, D, W, G, Q, P, E, R, F, N, | | | | |
| | S, V, Y, M, H, | | | | |
| 3 | H, D, A, Y, V, R, P, E, S, T, K, N | G, F | | | |
| 4 | T, W, I, L, Y | | | X | |
| 5 | Q, D, E | | X | | |
| 6 | E, G, Q, D, N, F | H, Y, A, V, M, I, L, P, W, | | | |
| | | R, S | | | |
| 7 | M, S, A, V, N, L, T | | | X | |
| 8 | Q, G, S, E, A, M, N | | | | |
| 9 | Y, L, V | | X | | |
| 10 | Y, H, Q, L, E | | | | |
| 11 | G, A, S | | | X | |
| 12 | V, I, L | | X | | |
| 13 | A, K, S, G, N | R, T, Q | X | | |
| 14 | M, G, I, V, L, A, K | | | X | |
| 15 | V, Y, W, F, N | | X | | |
| 16 | F, M, L | | X | | |
| 17 | G, K, A, H | V, M, Y, P, L, T, I, W, S, R, | | | |
| | | Q, F | | | |
| 18 | E, Y, K, N, F, D | G, W, Q, T, H, M, A, S, R, L, | | | |
| | | V, I | | | |
| 19 | Q, M | L, T, D, K, E, F, S, H, P, G, | | | X |
| | | V, N, R | | | |
| 20 | V, L, W, I, H, S, N, R, D, Q, F, M, | | | | X |
| | A | | | | |
| 21 | D, N, S, Q | V, M, K, H, R, P, E, A, G | | | |
| 22 | A, H, V, F, G, R, S, K, W, Q, N, I, | | | | |
| | E, T, Y, M, L | | | | |
| 23 | T, Q, P, S, V, A, I, H | | | X | |
| 24 | A, S, R | | X | | |
| 25 | A, E, T, L, M, V, W, Y, H, I, K, R, | | | | |
| | N, F, G, Q, D | | | | |
| 26 | D, I | G, H, V, S, A, L, R, Q, M, T, Y | | X | |
| 27 | W | | X | | |
| 28 | W | F | X | | |
| 29 | E, I, Q, D, T, V, Y, A, R, F, N, M, | W | | | |
| | G, S, K | | | | |
| 30 | I, M, S, L, D, E, V, A, T, Q | | | X | |
| 31 | H, M, K, R | | X | | |
| 32 | D, S, E, Q, T, N | | X | | |
| 33 | N, A, F, H, G, L, D, T, Q | R | | X | |
| 34 | R, D, A, S, I, V, Q, L, E | | | | |
| 35 | K, E, W, Y, L, S, V, N, G, R, T, M, | D | X | | |
| | H, Q | | | | |
| 36 | M, H, N, W, L, Y, Q, G, V, A, R, K, | | | | |
| | P | | | | |
| 37 | T, M, E, R, A, P, V, Y, Q, N, K, D, | | | | |
| | H | | | | |
| 38 | E, K, D, A, P, S, I, Q T, G | | X | | |
| 39 | N, I, L, M, Q, W, K, V, D, A, H, E, | | X | | |
| | R | | | | |
| 40 | H, A, V, I, D, S, E, F, K, Y, T, G, | L, N | | | X |
| | W, M, P, R | | | | |
| 41 | Y, V, G, L, A, R, P, N, I, F, S, H, | | | | X |
| | T, M, Q | | | | |
| 42 | Q, I, P, Y, E, K, V, H, D, L, A, T, | | | X | X |
| | G, M, N, F | | | | |
| 43 | K, M, S, W, F, P, G, L, Y, E, R, D, | | | | X |
| | Q, V, A,T H, N | | | | |
| 44 | K, D, I, Y, W, H, G, E, Q, T, R, F, | | | | |
| | L, P | | | | |
| 45 | P, H, S, A, F, I, Q, K, G, Y, R, E, | | | | |
| | W, V, M, T, L | | | | |
| 46 | K | I, L, Y, Q, H, R, A, G, M, P, E, | | | |
| | | D, W, V, F | | | |
| 47 | M, V, H, Q, S, G, T, A, N, P, D | | | | |
| 48 | H, W, A, D, Q, V, S, F, T, R, P, K, | I | | | |
| | L, M | | | | |
| 49 | F, K, N, D, T, A, E, I, Y, M, W, R, | | | | |
| | V, S, L | | | | |
| 50 | I, V | D, P, L, Y, K, N, G, R, H, Q | | | |
| 51 | Y, N, F, S, W, R, Q, K, H, A, G, P, | | | | |
| | V, E | | | | |
| 52 | W, K, Y, F, N, M, H, L, P, R, A, D, | | | | |
| | S, E, G, V, T, I | | | | |
| 53 | E | W, Y, Q, S, V, F, G, A, K, I, L, | | | |
| | | M, P | | | |
| 54 | H, Y | N, M, I, D, A, R, S, F, Q, K, V, | X | | |
| | | L, W, G, T, E | | | |
| 55 | N, L, K, F, G, W, H, T, E, S, M, Y, | | | | |
| | A, R | | | | |
| 56 | E, Y, W, G, H, S, F, Q, T, K, P, R | | | | |
| 57 | R, K | T, H, G, A, I, S, W, P, V, M, E, | | | |
| | | Y, L, F | | | |
| 58 | M, V, I, L, T, A, F | | X | | |
| 59 | E, Y, V, Q, A, H, L, F, R, G, N, M, | | | | |
| | S | | | | |
| 60 | H, V, R, D, E, F, L, W, P, T, G, M, | | | | |
| | K, A, N | | | | |
| 61 | Y, W, H, N, I, V, M, E, Q, R, F, K, | | | X | |
| | P | | | | |
| 62 | Y, G, N, S, R, I, H | A, L | | | |

TABLE 13J
EGFR2_mb3
DRRKEMDKVYRTAFKRITSTPDKEKRKEVVKEATEQLRRIAKDEEEKKKAAYMILFLKTLG
SEQ ID NO: 144

| | | | | | |
|---|---|---|---|---|---|
| 1 | N, K, Y, W, F, I, P, L, A, H, | | | | |
| | Q, V, S, E, R, D, T, G, M | | | | |
| 2 | F, N, R, W | V, G, D, E, I, H, A, Y, T, L, Q, | | | |
| | | K, P, M, S | | | |
| 3 | K, F, M, R, Y, Q, I | W, P, A, E, G, T, D, V, S, N, H, L | | | |
| 4 | S, T, N | I, E, V, G, W, Y, Q, A, P, M, D, | | | |
| | | F, H, R, K, L | | | |
| 5 | F, S, A, I, Q, M, D, L, N, | H | | | |
| | Y, E, K, W, V, T, R, P | | | | |
| 6 | M, F | I, W, L, Y | | X | |
| 7 | E, M, Q, I, L, Y | V, F, H, T, A, K, R, G, | X | | |
| | | S, W, N, D | | | |
| 8 | A, E, D, F, S, Q, W, L, Y, H, V, | | | | |
| | R, M, T, K, G, N, I | | | | |
| 9 | V, A | | | X | |
| 10 | Y | | X | | |
| 11 | V, K, I, Q, R, A, H | M | X | | |
| 12 | W, D | Y, S, Q, F, H, K, A, N, V, I, L, | | | |
| | | M, G, T | | | |
| 13 | Y, A, F, E | | | X | |
| 14 | Y, W, F | | X | | |
| 15 | F, A, E, Q, I, S, M, W, G, T, | | | | |
| | H, N, V, D, Y, L, K, R | | | | |
| 16 | L | Y, M, N, H, E, A, F, D, I, Q, V, | | | |
| | | K, S, R, T | | | |
| 17 | L, I, N, T | | | X | |
| 18 | M, E, I, H, S, K, L, N, Y, | R | | | |
| | V, F, Q, T, A | | | | |
| 19 | G, Q, D, R, T, A, M, H, L, | K | | | |
| | E, N, S, I | | | | |
| 20 | E, T, N | Q, D, S, A, M, Y, L, H, V, W, F, | | | X |
| | | I, G | | | |
| 21 | D, S, R, N, T, K, E, Q | H, I, F, A, M, L, W, Y, G, V, P | | | X |
| 22 | N, E, S, D, A, Y | | | | X |
| 23 | H, P, L, R, Y, T, W, G, S, Q, I, | | | | |
| | A, M, N, V, F, K, E | | | | |
| 24 | S, K, N, L, I, D, Q, H, M, V, R, | | | | |
| | T, Y, W, G, A, F, E | | | | |
| 25 | S, Y, I, A, T, M, V, L, R, G, E, | | | | |
| | Q, N, K, H, D, F, P | | | | |
| 26 | R | V, N, H, I, K, Q, S, A, M | | | |
| 27 | M, V, D, F, S, T, H, Y, Q, N, I, | | | | |
| | R, W, L, E, A, K, G | | | | |
| 28 | K, T, N, H, L, M, V, I, S, R, W, | | | | |
| | Q, E, A, F, Y, G, D | | | | |
| 29 | A, Y, V, I, T, L, D, G | | | X | |
| 30 | Y, W, L, I, V, A, S, G, F, R | | | | |
| 31 | A, F, R, W, S, I, H, L, G, Q, N, | | | | |
| | T, Y, V, M, K, D, E | | | | |
| 32 | D, V, T, K, H, Q, N, W, S, E, A, | | | | |
| | R, F | | | | |
| 33 | T, V, Y, A | | | X | |
| 34 | A, E, N, Y, M, V, H, D, R, Q, I, | | | | |
| | T, L, G, S, P | | | | |
| 35 | H, R, W, M, Q, D, F, I, Y, V, K, | | | | |
| | L, A, T, G, S, E, N | | | | |
| 36 | Q | K, S, A, E, D, Y, T, V, F, M, H, | | | |
| | | R, I, L, W, N | | | |
| 37 | M, L, V, I, R | | | X | |
| 38 | H, F, S, T, N, V, A, K, Q, M, Y, | | | | |
| | W, L, R, G, I, E, D | | | | |
| 39 | S, G, K, E, H, V, I, Y, N, Q, F, | | | | |
| | W, M, A, T, R, D, L | | | | |
| 40 | Y, L, F, V, M, I, A | | | X | |
| 41 | A, P | | | X | X |
| 42 | V, Y, G, H, Q, I, R, M, E, | A | | | X |
| | L, T, F, K, N, S | | | | |
| 43 | N, S, T | K, H, A, G, R, D, Q | | | X |
| 44 | P, N, V, K, A, Y, Q, T, D, | M, F, R | | | |
| | H, S, E, G | | | | |
| 45 | P, K, R, A, T, N, M, W, S, | L | | | |
| | G, H, Y, Q, E, V, D, F | | | | |
| 46 | D, H, A, M, Q, G, T, S, E, N, I | | | | |
| 47 | M, I, R, L, K, T, N, E | | | | |
| 48 | M, R, L, K, Y, N, Q | | X | | |
| 49 | K | T, R, I, V, Y, M, A, E, | X | | |
| | | W, F, H, L, Q, S | | | |
| 50 | V, A | | | | |
| 51 | A, G | Q, E, S, M, N, H | X | | |
| 52 | Y | F, M, L | X | | |
| 53 | M | Y, I | X | | |
| 54 | I | L, Q, T, R, M, A, G, E, V, S | | | |
| 55 | I, V | T, A, S, E, M, L, H, Q | X | | |
| 56 | F X | | | | |
| 57 | L | | | | |
| 58 | W, E, T, L | F, M, Y, H, D, G, Q, N, A, I, V, | | | |
| | | R, S, K | | | |
| 59 | E, S, Q, G, H, N, V, T, I, Y, L | | X | | |
| 60 | M, L, I, F, H | V | X | | |
| 61 | A,V, I | Q, L, W, H, T, Y, M, G, P, S, | | | |
| | | F, N | | | |

TABLE 13K
FGFR2_mb4
DKKELSDTAEKLLKDALDKNDDSKFISAVFFALKVAKDVNDERLERIVVKLYNEFMNRYH
(SEQ ID NO: 23)

| | | | | | |
|---|---|---|---|---|---|
| 1 | W, F, N, M, A, P, E, Q, V, | D, R | | | |
| | S, L, I, G, K, H | | | | |
| 2 | D, N, W, G, F, S, M, A, V, | E, R, K, C, L | | | |
| | H, Q, I | | | | |
| 3 | H, V, E, S, N, C, Y, P, D, L, | | | | |
| | W, K, T, F, R | | | | |
| 4 | R, P, H, F, W, L, A, S, I, | N, E, M, D, K, Y | | | |
| | G, C | | | | |
| 5 | T, C, K, F, S, I, M, Q, A, | W, L, G, N | | | |
| | E, D, V | | | | |
| 6 | N, P, L, H, W, T, E, R, A, | F, S | | | |
| | K, C | | | | |
| 7 | L, V, G, Q, H, I, K, C, R, | A, E, Y, P | | | |
| | M, T, W, S, D | | | | |
| 8 | R, I, Y, N, M, D, G, F, L, | H | | | |
| | Q, V, E, A, W, C, S, T | | | | |
| 9 | H, A, S | | | X | |
| 10 | K, V, A, Q, S, M, L, T, H | I, E, R, Y | | | |
| 11 | A | S, F, Q, H, G, C, T, W, Y, L, R, V, | | | |
| | | E, K, I, M | | | |
| 12 | H, I, K, N, T, F, A, W, S, L, | | | | |
| | Q, R, M, Y, V | | | | |
| 13 | I, M, L | | | X | |
| 14 | H, Q, Y, L, G, I, F, C, V, W, | | | | |
| | D, E, K, T, N | | | | |
| 15 | A, K, W, S, F, T, H, L, V, | E, D, N | | | |
| | I, Y, R, G, M, Q, C | | | | |
| 16 | L, F, T, S, W, Y, Q, G, H, | V, R, A, C, E | | X | |
| | N, M | | | | |
| 17 | A, F, W, N, V, T, G | I, S, L, H, K, Q, M, C | | | |
| 18 | I, S, A, F, L, Q, P, T | W, Y, V, G, D, C, H, R | | | |
| 19 | Y, D. H, T, V, E, G, L, N, | M, Q, I, C | | | X |
| | W, S, R, A, K | | | | |
| 20 | V, E, M, D, C, T, R, A, F, | L, K, S, Q, N, H | | | X |
| | P, Y | | | | |
| 21 | S, H, N, E, D | | | | X |
| 22 | M, I, E, F, Q, T, P, H, V, | W, R | X | | |
| | L, G, A, N, S, Y, D | | | | |
| 23 | D, N, Q, G, A, S, T, V, E | | X | | |
| 24 | G, N, L, H, V, Y, E, M, S, | Q, K | | | |
| | I | | | | |
| 25 | V, F, M, I | | | | |
| 26 | F, M, L, I, A | Y, V | X | | |
| 27 | G, A, P, S | | X | | |
| 28 | V, C, I, R, A, T | S, G | | X | |
| 29 | I, T, M, V | | X | | |
| 30 | Y, I, L, W, V | F | X | | |
| 31 | W, H, N, L | M, F, Y, G | | | |
| 32 | Y, G, S, C, A | | | X | |
| 33 | Q, Y, W, M, V, I, T, L, S | | X | | |
| 34 | G, D, N, M, Q, T, R, A, E, | H, S | | | |
| | K | | | | |
| 35 | R, H, M, L, A, C, T | V, Q | | X | |
| 36 | V, A, S, C | | | X | |
| 37 | E, D, S, T, R, Y, V, A, G, M, | | | | |
| | K, L, C, N, I, Q | | | | |
| 38 | W, I, A, E, M, G, L, R, S, | Q, D, V, N, Y, T | | | |
| | H, C, K, F | | | | |
| 39 | E, T, H, S, D, K, Y, N, W, | G, L, Q, I, V, A | | | X |
| | R | | | | |
| 40 | G, K, H, R, D, P, Y, E, F, | T, L, Q, N, A, C, I | | | X |
| | V | | | | |
| 41 | N, S, D | | | | X |
| 42 | T, N, K | C, M, G, R, I, S, Y, D, Q, L, F, E, | | | |
| | | A, P | | | |
| 43 | K, N, Q, I, V, F, L, G, C, | R, W | | | |
| | Y, D | | | | |
| 44 | L | | | X | |
| 45 | H, T, G, M, A | Q, E, D, Y | X | | |
| 46 | T, Y, E, K, G, M, F, A, S, | H, P | | | |
| | C, L, D, W, Q, N , R | | | | |
| 47 | T, N, A, V, E, Y, C, S, R, | | | | |
| | L, M, G | F, I, K, W | | | |
| 48 | C, I, T | V, A | | X | |
| 49 | I, V | T | X | | |
| 50 | N, M, H, Y, T, A, L, V, Q, | K, D, F, G | | | |
| | R, W, E, I, S | | | | |
| 51 | Y, M, I, R, N, F | C, T, S, V, K, H, D, G, E, L, A | | X | |
| 52 | Y, N | | X | | |
| 53 | D, C, S, W, G, Y, N, M, V | | X | | |
| 54 | T, I, M, R, A, H, G, K, N, | F, V | | | |
| | Q, L, S, C, Y, W, E | | | | |
| 55 | Q, K, W, G, E, H, A, F, R | | | X | |
| 56 | F, Y, W, N, I | H, S, G, C, M, T, V | X | | |
| 57 | M, C, S, R, I, W, F, H, L, | N, Y, V, Q, P | X | | |
| | G, K, A | | | | |
| 58 | F, D, E, V, Y, S, I, T | G, Q, H, R, M, C, A | | | |
| 59 | M, V, T, N, H, F, K, E, L, | D, I, R, Y, P, Q | X | | |
| | G, W | | | | |
| 60 | K, P, M, Q, W, Y, E, G, V, | S, A, H, T, I | X | | |
| | D, N, F, R, L | | | | |

TABLE 13L
FGFR2_mb5
DYERVISRAIELALKKGDEKALHILNEIFFAAHRGELDPTEAERLARRIEKKLRS
(SEQ ID NO: 24)

| | | | | | |
|---|---|---|---|---|---|
| 1 | K, R, N, D, M | T, Q, S, A, H, F, V, G, I, E, Y, L | | | |
| 2 | Y | G, W, I, L, F, P, K | | X | |
| 3 | D, E | P, S, N | X | | |
| 4 | I, P, R, V, K, Q, G | A, T, M, W, E, C, H, Y | X | | |
| 5 | P, I, V, L | W, K, Y, M, R, E, H, T, G, F, C, A | | X | |
| 6 | I | Y, C, S | X | | |
| 7 | S | | X | | |
| 8 | H, W, Q, N, A, R, M, C, V | G, S, L, F, K | | | |
| 9 | N, A, G, S, L | T, I | | X | |
| 10 | M, I | C | X | | |
| 11 | F, S, Y, G, E | A, D, P, K, T, N | X | | |
| 12 | E, Q, N, L, W, M, R, I, K, | C, A, T, D, S, V | | X | |
| | F, Y | | | | |
| 13 | S, C, A | T | | X | |
| 14 | I, L | M | X | | |
| 15 | R, E, Y, H, F, V, A, S, G, | N, L, C, M | | | |
| | I, W, K, T, D | | | | |
| 16 | L, E, V, H, E, N, K, Y, T | A, C, W, S, R, Q, G, D | | | |
| 17 | G, N, S, W | M, P, E, C, H, D, V | | | X |
| 18 | K, R, D | C, E, N, H, A, S | | | X |
| 19 | W, N, T, F, H, M, G, V, S, | R, A | | | |
| | I, Y, K, D, P, E, L, Q | | | | |
| 20 | H, S, P, K, V, Q | E, D, N, T, Y, I, L | | | |
| 21 | R, F, K, S, M, Q, C, H, L, | V, Y, T, I, D | | X | |
| | A, N, W | | | | |
| 22 | L | E, A, S, V, M | X | | |
| 23 | E, I, V, Q, L, P, T, H | K, M, S, R, N, C, A, G, | X | | |
| | | Y | | | |
| 24 | K, G, R, T, H, N, L, I, Q, | P | | | |
| | F, C, V, M, Y, S, A, W, | | | | |
| | E | | | | |
| 25 | A, C, L | V | | X | |
| 26 | D, N | M, Y, H | X | | |
| 27 | K, M, P, S, R, A, G, V, T, | H, D | X | | |
| | C, I, E, L | | | | |
| 28 | L, V, I, Y | T, F, Q | | X | |
| 29 | F | | X | | |
| 30 | F | | X | | |
| 31 | A, G | K, H, V | | | |
| 32 | A, C | T, G, D | | X | |
| 33 | F, I, H, Y | V, K, A, M, D, S, N, R | X | | |
| 34 | D, Q, W, R, N, C, A, Y, F, | G, E, H, L, V | | | X |
| | S | | | | |
| 35 | N, S, Q, G, T, W | H, F, L, Y, M, C, R, K, A | | | X |
| 36 | I, Y, R, G, K, M, F, P, N, | L | | | X |
| | T, H, V, S, E, W, A, C, | | | | |
| | Q, D | | | | |
| 37 | M, W, K, L, F | Y, I, N | | | X |
| 38 | P, Y, N, F, S, D, M, E, R, | L, W, A, I, H, Q, G, V, C | | | X |
| | K | | | | |
| 39 | T, L, P, R, Q | C, E, V, S, K, N, H, G, M, F, W, Y | | | |
| 40 | R, D, I, W, G, V, T, M, C, | K, E, Q, A, H, P | | | |
| | N, Y, F | | | | |
| 41 | Y, T, L, V, K, D, R, W, E, | A, H, I, N, S, M, G | | | |
| | Q, F | | | | |
| 42 | A | C, G, S, V | | X | |
| 43 | R, S, F, D, T, W, N, E, L, | H, C, M, A, K | | | |
| | Q, Y | | | | |
| 44 | I, L, Q, S, N, R, T, K | Y, H, V, A, G, C, F, D, M | | | |
| 45 | I, C, M, L, Y, H, F, W, T, | S, N, V, G, A | | | |
| | R, Q | | | | |
| 46 | A | S, G, V, K, M | | X | |
| 47 | K, L, F, D, T, R, Q, N, I, | A, M, H, S, W, C, Y | | | |
| | V | | | | |
| 48 | I, L, E, S, V, Q, K, A, M, | F, G, C, H, D, T, Y, W, P | | | |
| | R, N | | | | |
| 49 | M, I, Q, T | V, L, F, A, E | | X | |
| 50 | R, K, A, G, V, F, H, W, D, N, E, | | | | |
| | Y, S, C, T, M, L, Q, I | | | | |
| 51 | A, S, I, R, K, E, W, F | N, C, V, T, L, Q, H, M, G, D, Y | | | |
| 52 | N, Q, S, M, C, D, K, H, R, | Y, V, F, W, P, G | | | |
| | I, A, T | | | | |
| 53 | L, I, M, C | V, W | | X | |
| 54 | G, I, W, N, K, R, S, M | D, E, T, L, Q, H, F, C | | | |
| 55 | W, Y, M, F, H, E, S, L, V | D, R, C, G, I, P, A, T, Q, K | | | |

Z

TABLE 13M
FGER2_mb6
DLREKLQELWWRALEKGNEKAERLALRAFMGAWHGDTSEEEAKKIITEVERLIKS
(SEQ ID NO: 25)

| | | | | | |
|---|---|---|---|---|---|
| 1 | I, S, R, T, H, Q, M, W, Y | F, N, G, K, D, L, C, A, E | | | |
| 2 | Y, A, V, I, W, K, Q, F, S, | H, C, P, G, T | | | |
| | R, D, L, E, M | | | X | |
| 3 | T, A, S, I, N, G, D, V, F, P, | | | | |
| | Y, K, H, R, M, C, L, E, Q, W | | | | |
| 4 | P, R, Q, D, A, T, K, G, H, L, | | X | | |
| | E, N, S, I, V | | | | |
| 5 | R, A, T, V, L, K, W, I, C, | M, S, E, F, D | | | |
| | H, Q, N | | | | |
| 6 | Y, F, V, M, L | I | | X | |
| 7 | E, A, S, H, P, G, Q | | X | | |
| 8 | Y, K, M, C, L, A, W, N, S, | V | | | |
| | Q, G, E, T, R, H, F, I | | | | |
| 9 | I, V, Q, Y, L, M | | | X | |
| 10 | W | | X | | |
| 11 | Y, W, F | | X | | |
| 12 | E, Y, L, S, Q, I, H, M, G, | V | | | |
| | N, R, F, A, K, C | | | | |
| 13 | S, A | | | X | |
| 14 | Y, H, I, L, V | | X | | |
| 15 | I, R, G, Q, K, N, C, M, V, | T, D, S | | | |
| | E, H, L | | | | |
| 16 | Q, Y, R, L, H, I, D, S, A, | N, T, W, G, F, V, M, C, E | | | |
| | K | | | | |
| 17 | R, K, S, Q, E, G, D, H | N | | | X |
| 18 | I, Y, H, E, Q, N, D, C, A | S, V | | X | X |
| 19 | M, K, L, D, I, G, Q, S, H, | V, T, Y | | | |
| | P, R, N, E, A, F | | | | |
| 20 | P, S, E, N, Q, D, V, G, H, | F, Y, T, A, M, R, L | | | |
| | K | | | | |
| 21 | A | | | X | |
| 22 | N, E | M | X | | |
| 23 | H, D, W, F, T, L, V, Q, R, | Y | | | |
| | K, E, I, M, S, G | | | | |
| 24 | Y, F, L | | | | |
| 25 | A | S | | X | |
| 26 | L, I | | X | | |
| 27 | H, R, G | | X | | |
| 28 | C, A, L, V | | | X | |
| 29 | F, Y | | X | | |
| 30 | V, L, M, W | I | X | | |
| 31 | S, K, G, H, R, A | | X | | |
| 32 | N, Y, S, I, T, Q, R, L, G, | K, D | | X | |
| | V, F, A, W, M, H, E, C | | | | |
| 33 | Y, D, S, E, R, F, H, V, G, | M | X | | |
| | L, A, T, I, C, W, N | | | | |
| 34 | W, R, Y, A, N, K, Q, S, H, | E, G, L, T, M | X | | |
| | F | | | | |
| 35 | E, N, H, K, T, L, I, S, G, | V | | | X |
| | Y, E, Q, M, W, R, A | | | | |
| 36 | V, S, Y, M, R, I, F, T, A, | G | | | X |
| | H, N, K, Q, P, C, L, W, | | | | |
| | D, E | | | | |
| 37 | L, H, M, I, P, R, Q, E, C, | | | X | X |
| | N, V, T, Y, W, K, S, A, G | | | | |
| 38 | F, Y, K, P, H, T, M, W, G, | | | | X |
| | V, S, R, N, L, Q, C, A, I, | | | | |
| | E, D | | | | |
| 39 | I, D, P, M, C, T, V, A, L, | Q, R, Y | | | |
| | F, G, E, N, W, H | | | | |
| 40 | Y, C, Q, T, I, L, P, A, M, | N, G, W | | | |
| | V, E, D, R, S, K | | | | |
| 41 | Q, A, T, V, I, P, M, R, D, | N, W, K, Y | | | |
| | L, F, C, H, S, G, E | | | | |
| 42 | W, Q, T, F, M, Y, G, R, S, | C, N, E, H | | | |
| | I, V, A | | | | |
| 43 | L, C, T, V, Y, S, R, G | M, H, A, K, I, W, D, E, F, N | | | |
| 44 | T, V, I, H, A, W, K | P, R, L, N, F, M | | | |
| 45 | W, A, H, S, Y, V, I, Q, T, F, | | | X | |
| | L, G, K, R, C | | | | |
| 46 | V, C, I, L | E | | | |
| 47 | G, Y, R | D, S, N, P, V, K, I, W, F, L, T, H, | | | |
| | | A, Q, E | | | |
| 48 | N, T, W, Q, A, H, R, Y | M, K, L, E, D, I, C, V, F, S | | | |
| 49 | V, I, L, C | | | X | |
| 50 | S, N, T, R | M, V, I, Q, E, L, D, G, H, A, K, C, W | | | |
| 51 | V, T, I | H, W, E, A, R, E, K, Y, Q, C, L, M, G | | | |
| 52 | M | I, L, Q, S, E | | X | |
| 53 | V, F, I, M | T | | | |
| 54 | R, A, W, V, T, S, E, H, D, | N, Y, Q, C, I, M, F | | | |
| | K, L, G, P | | | | |
| 55 | Q, E, T, W, D, P, M, A, F, | C, V, L, I | | | |
| | N, R, K, Y, S, H, G | | | | |

TABLE 13N
FGFR2_mb7
SEIHKKLARLLSKAIQKGDRKANHIVIRAAMAYEKGLIDPDQARKLAEKAERILRS
(SEQ ID NO: 26)

| | | | | | |
|---|---|---|---|---|---|
| 1 | K, E, D, G, T, R, L, N, M, | C, F, V, P, I, W, H | | | |
| | S, Y, Q, A | | | | |
| 2 | D, S, G, E, Y, W | A, P, F, Q, N, R, L, C, M, H | | | |
| 3 | L, I | V, Q, F | | | |
| 4 | M, E, Q, S, R, T, Y, H, A, | L, V, N, F, W, D, P | X | | |
| | G, I, K | | | | |
| 5 | M, E, Q, S, A, K | Y, H, V, I, R, L, F, G, T, N, W, C | | | |
| 6 | Y, L, I, Q, V, E, T, H, M, | R, F, W, A, G, N, C, S, D | | | |
| | K | | | | |
| 7 | L, F | Y | X | | |
| 8 | A | G, S, Y, W | X | | |
| 9 | W, C, E, S, T, Q, L, F, A, M, | | X | | |
| | R, I, K, Y, H, D, G, V, N | | | | |
| 10 | R, L, I | V, A | | X | |
| 11 | L | M, W, Y | X | | |
| 12 | L, M, Q, E, A, D, F, H, R, Y, | | X | | |
| | N, V, S, G, W, K, I, T, C | | | | |
| 13 | W, L, E, M, I, Q, V, A | K, C, R, S, H, Y, T, D, F, N, G | | | |
| 14 | A | S, C, T, I | | X | |
| 15 | M, I, L | V, W, R, G, A, N, Y, S | X | | |
| 16 | A, R, L, K, E, Q, M, S | I, W, V, T, H, N, D, Y, | X | | |
| | | G | | | |
| 17 | M, A, K, L, V, Q | N, S, Y, I, R, F, C, W, G, D, E | | | |
| 18 | G, N | Q, K, H, C, D | | | X |
| 19 | N, D | S, C, Y, E | | | X |
| 20 | W, M, K, H, D, E, T | A, R, F, I, V, Y, N, L, | X | | |
| | | S, Q, P, G | | | |
| 21 | W, A, L, D, Y, V, Q, E, I, | T, N, C, H, S | | | |
| | R, K | | | | |
| 22 | A, V | L, M, I, S, D | | X | |
| 23 | T, L, I, Q, V, K, M, E, W, | H, S, G, C, R, A, Y | X | | |
| | N | | | | |
| 24 | M, I, A, C, N, E, L, S, Q | V, G | X | | |
| | F, D, T, R, H, K, Y, W | | | | |
| 25 | F, M, I | L, C, A, Y | | | |
| 26 | L, I, V | | | X | |
| 27 | V, I, L | M | X | | |
| 28 | Q, A, G, L, T, H, K, W, S, | V, Y, N, E | X | | |
| | F, R, M, I | | | | |
| 29 | I | A, T, V, L, F, M | | X | |
| 30 | F, Y, I, L, W, A, M, V | Q | X | | |
| 31 | I, Y, L, M, W, V | | X | | |
| 32 | A | G, M, S, W, F | X | | |
| 33 | L, M, Y, F | W, H, I | | X | |
| 34 | E, S, A, D, M, Q, H, I | Y, T, L, G, C, R, V, N | X | | |
| 35 | W, L, M, R, K, A | F, Y, N, V, I, Q, E | X | | |
| 36 | G | L, E, H | | | X |
| 37 | L, W, M | I, E, Y, T, R, K, A, S, | | | X |
| | | F, H, V | | | |
| 38 | I | | | | X |
| 39 | G, T, S, D, A, N | K, L, C, R, Q, E, P | | | X |
| 40 | A, I, D, E, Y, V, S, Q, L, | H, P | | | |
| | N, K, T, M, G, F, C, W, | | | | |
| | R | | | | |
| 41 | V, M, Q, W, I, D, L, F, A, | Y, K, T, E, S, R, N, P | | | |
| | G, H | | | | |
| 42 | Q, M, Y, A, H, E, F, T, V | R, K, L, C, I, D | | | |
| 43 | V, A | L, M, T, F | | | |
| 44 | W, I, M, L, A, E, Y, F, K, | D, N | | | |
| | T, R, Q, G, C, H, V, S | | | | |
| 45 | C, R, A, Y, I, E, D, M, N, | S, Q, F, W, H, G, T | | | |
| | K, V, L | | | | |
| 46 | W, L, E, F | Q, M, Y, N, I, H, C | | X | |
| 47 | I, M, C, L, A | V, F | | | |
| 48 | R, D, E, M, Q, L, A, G, H, | V, C, Y | | | |
| | N, I, W, S, F, K | | | | |
| 49 | L, I, M, Y, V, K, Q, F | T, E, R, H, D, W, A, C, N | | | |
| 50 | A, V | L, M | | X | |
| 51 | E, D, Y | Q, V, R, N | | | |
| 52 | N, A, K, E, Q, D, L, R, I, | S, M, T, C, Y, G, F, V | | | |
| | H, W | | | | |
| 53 | L, M, I | Y, R, Q, W, H, F, D, C | | X | |
| 54 | L, M | D, R | | | |
| 55 | F, M, N, Q, L, D, E, T, S, | V, W, C, G, K, I | | | |
| | A, Y, R | | | | |
| 56 | E, M, Y, Q, G, T, S, A, F, | D, W, I, L, V, P, R, K | | | |
| | C, N, H | | | | |

TABLE 13O
FGFR2_mb8
DIEHLLARAIHRSAQLGDREAVELLTRIFFALEAGKVSEEWAERLARTILEHQRN
(SEQ ID NO: 27)

| | | | | | |
|---|---|---|---|---|---|
| 1 | M, A, Q, I, H, K, F, P, R, E, L, | | | | |
| | D, W, Y, G, T, V | | | | |
| 2 | F, A, N, H, G, L, D, W, S, M, E, | | | | |
| | I, V, Y, R, K, P, T | | | | |
| 3 | S, Q, G, N, Y, T, D, E, M, | K | X | | |
| | A | | | | |
| 4 | A, G, I, K, V, R, P, M, Q, | T, S, E, L | X | | |
| | N, D, F, H | | | | |
| 5 | R, F, I, V, M, N, K, L, E | | | | |
| 6 | I, L, V, M | | X | | |
| 7 | P, Y, G, F, W, S, A | | X | | |
| 8 | K, T, S, L, I, W, A, E, N, V, | | X | | |
| | Q, R, H, F, Y, G, M, D, P | | | | |
| 9 | G, T, Q, I, N, K, F, L, W, R, V, | | | X | |
| | A, M, Y, H, S, E | | | | |
| 10 | M, W, L, Y, F, T, I, S, H | | X | | |
| 11 | M, L, I, W, A, P, D, N, S, | Y, R | X | | |
| | T, V, G, H | | | | |
| 12 | I, Q, A, L, T, H, F, E, N, G, W, | | | | |
| | R, D, V | | | | |
| 13 | M, N, H, F, T, R, Q, A, Y, G, | | | X | |
| | S, L | | | | |
| 14 | W, F, I, V, M, H, D, G, E, | Y | X | | |
| | L, A, R, Q | | | | |
| 15 | E, W, K, N, G, L, Y, A, T, H, D, | | | | |
| | R, M, Q, V, S, I | | | | |
| 16 | W, E, K, N, R, T, Q, A, D, H, G, | | | | |
| | F, L, S | | | | |
| 17 | H, N, D, V, K, Y, A, L, F, S, M, | | | | X |
| | Q, R, G, W, I | | | | |
| 18 | L, N, Q, K, A, R, D, Y | | | | X |
| 19 | F, N, I, H, A, T, V, S, D, | M | | | |
| | K, G, L, W, P, Q, R, E | | | | |
| 20 | G, R, Q, T, A, M, W, V, S, Y, | | | | |
| | I, E | | | | |
| 21 | D, N, T, R, E, V, H, A | S, K | | X | |
| 22 | L, I, M, Q, N, V | | X | | |
| 23 | H, L, F, I, D, S, K, N, T, | G | X | | |
| | E, V | | | | |
| 24 | Y, Q, E, H, N, A, I, S, K, | M | | | |
| | W, V, T, L | | | | |
| 25 | A, I, Y, T, S, V, F, G, L | | | X | |
| 26 | L, D, N, A, M, T, V, Y, S | | X | | |
| 27 | R, K | | X | | |
| 28 | V, G, L, S, I, A, W, M, T | | | X | |
| 29 | F, Y, V | | X | | |
| 30 | L, F | | X | | |
| 31 | G, S, A | | | | |
| 32 | A, M, K, V, H, T, S, Q, R, F, L, | | | | |
| | W, N, I | | | | |
| 33 | I, N, V, K, W, T, S, D, Q, R, E, | | | | |
| | G, Y, L, H | | | | |
| 34 | Y, E, H, G, D, W, N, T, M, R, F, | | X | | X |
| | A, K, S, L, Q | | | | |
| 35 | Q, H, R, E, D, K, T, G, S, N | | | | X |
| 36 | T, M, Q, N, K, A, R, S, H | | | | X |
| 37 | I, F, V, W | | | | X |
| 38 | T, H, P, R, A, S, Q, N, K, G | | | | X |
| 39 | Q, S, A, I, W, F, N, R, E, M, G, | | | | |
| | L, H, D | | | | |
| 40 | D, H, N, S, T, I, F, L, M, G, P, | | | | |
| | Q, K, R, E, V, W | | | | |
| 41 | Y, K, S, H, D, M, E, T, L, A, F, | | | | |
| | R, W, V, I | | | | |
| 42 | S, V, G, A | | | | |
| 43 | A, G, N, H, Q, R, D, E, T, F, Y, | | | | |
| | W, I, V | | | | |
| 44 | S, G, T, H, N, E, A, M, L, | P, K, W, I, R, Y | | | |
| | F | | | | |
| 45 | Q, S, A, N, I, M, R, E, H, | K | | | |
| | V, G, F, L, W | | | | |
| 46 | I, V, T, F, S, Y, L, G, A, H | | | X | |
| 47 | L, E, W, S, M, D, A, R, I, Q, Y, | | | | |
| | G, K, V, N | | | | |
| 48 | D, G, Q, E, K, S, L, T | R, H, V | | | |
| 49 | T, V, W, A, L, Q, N, G, S, R, H, | | | X | |
| | K, I, E, M, Y | | | | |
| 50 | S, F, E, N, I, P, D. H., G, V, M, | | | | |
| | W, Q, A, L, R, T, K, Y | | | | |
| 51 | D, V, W, I, T, H, Q, P, N, G, L, | | | | |
| | E, Y, R, F, M, K | | | | |
| 52 | Q, N, K, R, A, S, E, M, G, T, V, | | | | |
| | L, W, I, P, Y, F, H | | | | |
| 53 | G, N, S, E, K, P, I, Y, A, | D, F, L, T, V, Q, H | | X | |
| | M | | | | |
| 54 | D, E, Q, A, I, T, H, M, F, | L, S, Y, G, W, N, K | | | |
| | R | | | | |
| 55 | P, F, T, K, D, L, W, M, R, Y, I, | | | | |
| | A, V, N, G | | | | |

TABLE 13P
FGFR2_mb9
SLVERVIEQVEKRGLPPEALVLTVWLALVKAGDPEKARLVEELAREVFEGKLPPEELKRLLREIE
(SEQ ID NO: 28)

| | | | | | |
|---|---|---|---|---|---|
| 1 | T, R, G, H, S, Y | K, W, N, M, F, P, D, A | | | |
| 2 | Y, W, M, G, F | L, S, N, T, A, I | X | | |
| 3 | V, L | I, W | | X | |
| 4 | E | D, H, N, Q | | | |
| 5 | Y, W, N, F, H, S, M, D, E | G, A, Q, L, T, R, I, V | X | | |
| 6 | F, Y, L, T, M | I, V, D, A, R | | X | |
| 7 | I | S, L | | | |
| 8 | W, D, E, S | Q, N, F, H, Y, M, A, L | | | |
| 9 | E, Q, D | S, F | X | | |
| 10 | I, M, V | Q, L, G, H | | X | |
| 11 | E, D | W, A | | | |
| 12 | S, M, R, G, T, W, F, N, E, | D, Q, Y, L, A, K, I, V | | | |
| | H, P | | | | |
| 13 | M, E, D, A, N, Q, L, H, T, | V, F, R, Y, I | X | | |
| | G, K, S | | | | |
| 14 | G | D, H, E, Y | | | X |
| 15 | F, L | D, A, S, Q | X | | X |
| 16 | G | S, P, R, A, T, N, H | | | X |
| 17 | Y, P, I, G, M | S, A, H, Q, E, V | | | |
| 18 | A, Y, I, S, M, H | E, W, F, T, R, Q, G, K, | X | | |
| | | L | | | |
| 19 | S, T | A, H, Q | X | | |
| 20 | I, A | L, S, V | | | |
| 21 | V, F | N, P, W | | | X |
| 22 | Y, M | L, S, T, V, W, F, A | X | | |
| 23 | T | S | X | | |
| 24 | I, V | Y, M | | X | |
| 25 | Y, F, W, D | | X | | |
| 26 | Y, F | L, V | X | | |
| 27 | G | A, V, W, T | X | | |
| 28 | Y, H, W | F, L, M, N, I, A, S | | X | |
| 29 | T | V, K, Y, H, E, L, A, I, | X | | |
| | | F, M | | | |
| 30 | K | H, E, N, R, L, M | X | | |
| 31 | G, Y, H, F, W, S | K, A, M, V, N, D, R, I, Q, L | | | |
| 32 | G, S | N, D, V | | | X |
| 33 | H, W, Y, M, Q, L, I, R | K, N, F, D, S, A, E, T | | | X |
| 34 | N, D, E, A, G, Q, F, H, P | S, L, V, K, M, T, I, R, W | | | |
| 35 | Y, V, Q, R, E, W | H, N, M, A, I, D, T, L, S, K, F | | | |
| 36 | N, R, K | D, T, I, G, Y, M | | | |
| 37 | A, V | K, P, M, T | | | |
| 38 | D, Y, Q, N, A, M, G, S, F | H, W, T, R, E, L, I, V, K | | | |
| 39 | K, V, E, D, W, H, R, Q, Y, | L, I, S, N, M, T | | | |
| | A, F | | | | |
| 40 | V | A | | X | |
| 41 | E | S, N, D | X | | |
| 42 | D, Q, L | G, N, E, M, I, S, T, K, Y, A, P | | | |
| 43 | W, Y | L, M, I, T, S, V | | X | |
| 44 | S, F, W, Y | H, A, G | X | | |
| 45 | R, K, I | Q, G, W, H, N | X | | |
| 46 | L, Q, K, R, M, S, N | A, E, H, G, F, Y, W | | | |
| 47 | V | T, L, W, H, Y | | X | |
| 48 | F | | X | | |
| 49 | M, K, R, A, V, S, Q, L, T | G, N, I, E, D, Y, F, W | X | | |
| 50 | G | Y, V, H, N, T, D | | | X |
| 51 | T, R, N, K | Q, A, M, I, H, S, E | | | X |
| 52 | I, L | M, V, W, H, K, G | X | X | |
| 53 | A, Q, G, L, E, H, R, S | W, P, V, K, D, N, Y, T, I, M, F | | X | |
| 54 | M, W, Y, N, F, L, Q, T, I, | P, V, A, E, D, S, K | | | |
| | H, G | | | | |
| 55 | D, A, E, L, W, N, P | H, Y, Q, S, M, I | | | |
| 56 | R, E, Q, T | V, K, N, H, I, G, A, D, L | | | |
| 57 | L, M | S, A, I, H | | X | |
| 58 | K | I, L, S, Y, R, N, Q, W | | | |
| 59 | R, S, Y, F, V, Q, A | N, K, H, M, L | | | |
| 60 | S, K, M, A, R, E, L, I, V | N, Q, T, H, W | | | |
| 61 | L | M, T, A, F | | X | |
| 62 | R, N | Q, L, Y, S, T, H, F, V | | | |
| 63 | G, L, E, M | K, S, R, D, T, I, V, N, H | | | |
| 64 | L, F | M, I, Q, V, T, E, A, D | | X | |
| 65 | Q, T, I, L, F, K, M, V, H | N, P, A, R, S, E, D, G, Y | | | |

TABLE 13Q
FGFR2_mb10
DEVHHFLLRLFFKDPDDEDVERILALFLLLKKEGIPPEEVTEIVIKFAEKLGNPELAKELKKILK
(SEQ ID NO: 29)

| | | | | | |
|---|---|---|---|---|---|
| 1 | A, M, D, Q, S, G, W, E, P, | I | | | |
| | L | | | | |
| 2 | N, S, P, Q, Y, E, H, F, G, M, V, I, | | | | |
| | R, T, D, W, K, L | | | | |
| 3 | V, I, M, F, L, T, A, G | P, S | | X | |
| 4 | W, H, A, S, L, M, Y, E, N, | P, V | X | | |
| | Q | | | | |
| 5 | Y, H, T, F, N, M, V, I, S | R | | | |
| 6 | F, Y, H, W, L | M | | | |
| 7 | L | | | X | |
| 8 | L, I | V | X | | |
| 9 | R | G | X | | |
| 10 | L, M, K, V, Y, W | A, N | | | |
| 11 | F | | X | | |
| 12 | F | V | X | | |
| 13 | Y, K, M, W, A, H, S | | | | |
| 14 | D, A, N, E, K, Q, T | H | | | X |
| 15 | P, T, V | | | | X |
| 16 | F, N, Q, R, W, D, M, A, K, | H | | | X |
| | L, G, E | | | | |
| 17 | A, D, E, N, Q | | | | X |
| 18 | D, I, H, R, E, Y, Y, L, N, V, | M | | | |
| | K, G, P, A, F, S | | | | |
| 19 | G, N, S, D, T, Q, E, L, M, | F, K | | | |
| | Y, V, H, A, R, I | | | | |
| 20 | M, V, H, I, N, T, A | L, Y, K | | | |
| 21 | E | | X | | |
| 22 | A, D, G, K, Q, S, R, M, N, | E | | | |
| | Y, H | | | | |
| 23 | M, I, L, V, A, G, S | T | | X | |
| 24 | L, F | | X | | |
| 25 | A, N, S, V | | X | | |
| 26 | L, A, W, G, Q, D, R, M, T, H, I, | | | | |
| | E, F, Y | | | | |
| 27 | A, N, T, F, M, V, G, D, I, | | | X | |
| | S, H, L | | | | |
| 28 | Y, L, M, F, W | | X | | |
| 29 | M, L, Q, N, V, G, E, W, I, | F, A, D, R | X | | |
| | T | | | | |
| 30 | G, L, N, E, I, V, W | D, T, F | | X | |
| 31 | H, N, W, Y, K, M, F, G, P, | E, D | | | |
| | Q, V, R, T, S | | | | |
| 32 | G, F, H, M, N, Q, W, K, Y, | S | X | | |
| | R, I, T, E, V, L | | | | |
| 33 | P, E, R, D | Q | | | X |
| 34 | D, F, Q, S, R, G, P, A, K, | N | X | | X |
| | L, I, Y, M, T | | | | |
| 35 | G, I, Q, W , K, H, A, R, N, T, | | | | X |
| | L, P | | | | |
| 36 | A, T, R, N, E, P, D, S, F, | M | | | X |
| | Y, V, I, H, Q, W, L, G | | | | |
| 37 | A, G, H, Q, T, Y, P, K, M, | I | | | |
| | V, F, L, W, E | | | | |
| 38 | H, M, L, Q, R, W, E, T, F, | A, N | | | |
| | V, S, D, I, P, K, G | | | | |
| 39 | E, Y, D, L | G, W, I, N, M | | | |
| 40 | N, V, Q, R, H, L, W, Y, E, | F, A, M | | | |
| | K | | | | |
| 41 | M, L, R, T, F, E, E, D, A, | Q, N, S, G | | | |
| | H, Y, K, W | | | | |
| 42 | I, M, E, N, G, Q, S, D, A, | H, W | | | |
| | Y, F, R, P, L | | | | |
| 43 | K, L, I, M, N, W, Y, V, E, | G, F, A, D | | | |
| | P, Q | | | | |
| 44 | T, V, G, L | I, S | | X | |
| 45 | A, K, L, V, I, H, W, S, G, | M, T, F | | | |
| | R | | | | |
| 46 | A, F, H, M, R, Y, K, D, S, | G | | | |
| | Q, I, P, L, N | | | | |
| 47 | H, L, F, S, Y, K, R, Q, V, | W | | | |
| | T, I, A, N, M | | | | |
| 48 | T, A, I, V, G, L | E, S | | | |
| 49 | A, T, R, M, N, I, E, L, V, | H | | | |
| | K, S, Y, W, D, Q, F, G | | | | |
| 50 | G, K, D, H, R, L, M, A, Y, | | | | |
| | P, Q, S, E, T, N | | | | |
| 51 | F, N, L, D, V, I, Q, K, M, | G, S, W | | X | |
| | Y, H, E | | | | |
| 52 | P, S, T, W, Y, G, V, A, L, | F | | | X |
| | K, E, M, N, H | | | | |
| 53 | F, H, K, Y, N, W, P, V, L, | | | | X |
| | R, I, M, T, S, E | | | | |
| 54 | P, D, Q, L, I, S, Y, W, A, | M | | | |
| | T, R, K, E, G, F, V, N | | | | |
| 55 | G, M, T, V, E, Q, R, L, Y, | F, H | | | |
| | K, D, W, N, P, A | | | | |
| 56 | I, L, R, P, A, Y, H, V, M | K, T, W | | X | |
| 57 | G, L, Q, R, A, S, M, T, N, | P, V, D, I | | | |
| | F, K, Y | | | | |
| 58 | G, N, T, K, A, F, I, V, R, | Q, H, Y, S, W | | | |
| | L, M, E | | | | |
| 59 | M, E, Y, Q, S, K, F, H, G, | T | | | |
| | I, N, P, D, R, W, A | | | | |
| 60 | L, I, M, V | | | X | |
| 61 | G, Y, K, I, W, T, E, L, Q, | D | | | |
| | S, F, A, N, R, V | | | | |
| 62 | A, E, F, R, V, K, W, L, I, | M, Y | | | |
| | S, G, D, N, T | | | | |
| 63 | L, R, P, Q, Y, K, I, F, G, | W | | | |
| | E, H, M, A, D, V, N, T | | | | |
| 64 | F, Y, L, G, A, E, T, M, K, | H, W | | | |
| | Q, D, V, R, S, I | | | | |
| 65 | D, K, S, V, M, Y, H, E, W, | T, N | | | |
| | G, P, L | | | | |

TABLE 13R
H3_mb1
SQHEKFLEWMLRKIEEAIKRGNKISAEFLINLAKNFIHVLGDDEIRRRLERLERQLH
SEQ ID NO: 645

| | | | | | |
|---|---|---|---|---|---|
| 1 | S, T, N, R, P, K, H, G, L, | D | | | |
| | I, V, Q | | | | |
| 2 | Q, R, A, P, K, G, S, E, N, | H, F | X | | |
| | T, M, D | | | | |
| 3 | R, Q, H, K | F | X | | |
| 4 | K, A, R, F, H, Y, N, E, D, | L | | | |
| | S, G, T, Q, V , M | | | | |
| 5 | K, Q, A, L, N, M, R, I, H, | G | | | |
| | E, V, T | | | | |
| 6 | F | H, K, P, A | X | | |
| 7 | M, L, R, F, K, H, N | Q, S, D, T | | | |
| 8 | I, W, Y, R, F, M, N, G, Q, | P | | | |
| | V, K, H, E, L, S, A, T, | | | | |
| | C, D | | | | |
| 9 | W | | X | | |
| 10 | L, M, I, H | N, F | X | | |
| 11 | L, I, M, H, V, N, Q | A, C, R | | | |
| 12 | R, K, S, N, H | G, Y, C | | | |
| 13 | K | P, H, T | X | | |
| 14 | I, T, L, V, A, G, C | | | X | |
| 15 | F, K, R, Q, I, L, Y, N, A, V, E, | | | | |
| | T, S, H, D, W, C, G, M | | | | |
| 16 | A, V, M, S, I, L, T, N, E, | H, C, R, W | | | |
| | Q, D, G, F, Y | | | | |
| 17 | A, N | S, R, T, Q | | X | |
| 18 | F, Q, I, G, M, V, R, Y, N, L, | | | | |
| | H, S, C, W, E, K, T, D, A | | | | |
| 19 | R, K, N, V, Q, T, A, Y, G, | L, W, M | | | |
| | I, E | | | | |
| 20 | R, K | | X | | X |
| 21 | N, K, R, G, T, Q, S | M, H, A, E, P, D, F | | | X |
| 22 | N, R, Q | H, S, K, F, V, C | X | | X |
| 23 | R, K | A, H, E, P, Q | X | | |
| 24 | L, I, M | K, F | X | | |
| 25 | S | | X | | |
| 26 | A, G, Y, S, T, Q | | | X | |
| 27 | D, N, E | | | | |
| 28 | W, F, T | Y, S | X | | |
| 29 | L | Y, V | X | | |
| 30 | I, V, Q, T, A, S, C, G | L, M, Y | | | |
| 31 | G, N, T, P | C | | | |
| 32 | N, Q, L | T, S, C, W, P | X | | |
| 33 | Y, A, S, F, W, H, C | T, G | | X | |
| 34 | F, I, M, V, W, A, G, S, T, R, K, | | | | |
| | H, N, P, Q, D, E, Y, C | | | | |
| 35 | R, K, G, N, Q | S, T, D | X | | |
| 36 | F | | X | | |
| 37 | S, I, N, L, M, V, A | T, G, F, C, Q, H | | X | |
| 38 | R, N, S, G, H, T, Q, I, K | L, W | | | |
| 39 | P, Q, G, V, S, R, A, N, T | K, H, E | X | | |
| 40 | T, K, P, Q, E, N, H, L, D, | C, W, S | | | X |
| | R, V, G, I, A | | | | |
| 41 | K, Q, L, D, H, R, G, S, N, | F | | | X |
| | T, A, E, V, W, Y, C | | | | |
| 42 | H, Y, F, I, D, N, Q, E, K, R, | | | | X |
| | S, T, M, A, L, G | | | | |
| 43 | Q, Y, T, I, A, N, K, M, D, V, | | | | |
| | S, G, R, F, C, L, W, H, E | | | | |
| 44 | W, R, Q, H, G, Y, K, M, P, E, | | | | |
| | V, F, A, D, N, L, I, T, C, S | | | | |
| 45 | K, I, H, M, V, P, R, Y | T, D | | X | |
| 46 | K, A, R, S, E, H, T, M, I, | F, D, C | | | |
| | N, Q, G, L, Y, W, P, V | | | | |
| 47 | K, T, A, Y, R, N, L, W, I, | S, G, D, C, Q, E | | | |
| | V, M, F | | | | |
| 48 | R, Y, F, K, S, W, H, Q, N | | | | |
| 49 | L, T, A, V, F | G, Q | | X | |
| 50 | I, N, E, W, G, S, A, H, Y, | P | | | |
| | K, T, F, V, L, D, M, R, | | | | |
| | Q, C | | | | |
| 51 | I, R, E, K, G, H, W, S, A, | C, Y, V, D | | | |
| | L | | | | |
| 52 | M, S, L, F, Y, W, H, A, Q, | D | | X | |
| | N, V, C | | | | |
| 53 | E, Q, G, I, D, V, T, N, H | S, M, L, R | | | |
| 54 | H, R, S, I, D, K, P, M, E, W, A, | | | | |
| | T, V, Y, G, F, N, L | | | | |
| 55 | W, M, V, I, Y, K, N, R, F, L, | | | | |
| | S, A, T, Q, G, H, P, E, D | | | | |
| 56 | F, V, R, P, L, G, M, Y, K, | D | | | |
| | S, Q, W, C, I, T, A, N, | | | | |
| | E, H | | | | |
| 57 | G, W, P, H, E, D, Q, A, F, V, | | | | |
| | N, R, T, Y, S, M, L | | | | |

TABLE 13S
H3_mb2
TEEEIEEVEELLKKATDPKLRHMLEFLLRRLREALERNNLLSARFLLGLARQFYKSI
(SEQ ID NO: 646)

| | | | | | |
|---|---|---|---|---|---|
| 1 | H, K, S, R, A, G, Q, T, N, E, V | P, Y, D, C, I, W, L | | | |
| 2 | N, R, Y, G, H, A, S, Q, K, P, F, W, L, | | | | |
| | I, T, C, V, E, D, M | | | | |
| 3 | P, H, G, K, V, S, T, I, D, A, R, E, Q, | L, C | | | |
| | N, W, M, Y, F | | | | |
| 4 | F, M, P, Y, A, H, W, Q, N, T, C, S, V, | G | | X | |
| | R, E, I, K, L, D | | | | |
| 5 | L, I, S, A, V, T | Y, C | | | |
| 6 | L, R, I, Q, K, M, E, V, H, Y, F, G, A, | | | | |
| | C, T, S, N, D, W | | | | |
| 7 | E | | | | |
| 8 | V | | | X | |
| 9 | K, E, V, S, T, R, L, N, A, Q, M | I, C | | | |
| 10 | G, R, M, A, T, Q, E, H, C, S, D, N, L, | W, K, F | | | |
| | I, V, Y | | | | |
| 11 | M, K, S, N, F, L, Y, Q, W | R, H, A | | X | |
| 12 | L, Y, H | | | | |
| 13 | R, K, H, E, I, S, N, Q, A, Y, F, G, M, | W, V, T, C | | | |
| | L, D | | | | |
| 14 | T, N, K, R, S, Q, H, M, A, G, C | D | | | |
| 15 | A | | | X | X |
| 16 | R, T, P, N, H, K, V, E, Y, Q, M, S, A | F, I, D, G, W | | | X |
| 17 | R, Q, T, N, H, S, A, K, M, E, Y, D, V | P, I | | | X |
| 18 | R, S, Y, P, N, I, H, T, V, L, W, Q, A, | | | | |
| | K, G, E, M, F, D | | | | |
| 19 | Y, W, K, R, Q, H, P, N | T,F | X | | |
| 20 | K, Q, R, L, M | V | | | |
| 21 | I, R, K, V, Q | T, M | | | |
| 22 | N, A, E, S, D, G, Q, H, T, R, K, L | I, M, V | X | | |
| 23 | M | L | X | | |
| 24 | L | | | X | |
| 25 | E, S, M, T, I, Q, H, A, G, R, N, V | D, K, L, Y | | | |
| 26 | F | | X | | |
| 27 | L | | | X | |
| 28 | L, K, H | R, Q, C | | | |
| 29 | K, R, M, V, T, I | S, L, Q | | | |
| 30 | R | | X | | |
| 31 | L | | | X | |
| 32 | R, K | H | | | |
| 33 | T, Q, K, S, A, V, I, R, L, G, M, E, N, | D, P | | | |
| | W, F, H, Y | | | | |
| 34 | A | S | | X | |
| 35 | K, H, F, M, N, L, R, V, Y, S, Q, I, C | A, G, T | | | |
| 36 | R, A, K, S, F, Y, Q, V, G, N, T, M, E, | C | | | |
| | L, H, I, D, W | | | | |
| 37 | R, K | | | | X |
| 38 | K, H, R, N G, Q | S | | | X |
| 39 | R, N, K, S | H | X | | X |
| 40 | K, R, M, Q, L, Y, H, T, A, V, P | S, I | X | | |
| 41 | L | | X | | |
| 42 | S | | X | | |
| 43 | A, I, F, V, M, H, Q, L | T, S | | | |
| 44 | R, N, Q | H, S | X | | |
| 45 | F | W | X | | |
| 46 | L | | X | | |
| 47 | L, Y, G, I, S, T, V | M | | | |
| 48 | G | N | | | |
| 49 | L | | X | | |
| 50 | A | | | X | |
| 51 | R, H, Q | | | | |
| 52 | N, Q, H, S, T, G | R | X | | |
| 53 | F | | X | | |
| 54 | A, T, G, S, N, Y, E, V, C, | Q | | | |
| 55 | W, G, S, K, Q, N, Y, R, T, F, A, M, H | | | | |
| 56 | N, A, G, S | R, K, Q | X | | |
| 57 | R, Q, N, S, A, M, K, T, H, G, V, I, E, | P, L | | | |

D

TABLE 13T
H3_mb3
TRMRISVKFLLGLARQFTDPRIREWLLRRAERLAREGNDPELEELVRRVEEELK
(SEQ ID NO: 647)

| | | | | | |
|---|---|---|---|---|---|
| 1 | Y, N, E, D, S, R, Q, K, G, A, T, H, W, I, M, P, | L | X | | |
| | V, F | | | | |
| 2 | K, M, L, R, T, H, N, S, F, I, V | Q, A | X | | |
| 3 | V, I, T, Y, R, L, M, H, A, K, W, Q, F, N, E, S | D, G | | | |
| 4 | A, W, H, T, K, F, M, R, Y, Q, E, L, G, S, D, I, | V | X | | |
| | N | | | | |
| 5 | N, S, K, A, R, T, M, Q, D, P, E, H, G, I, L, V | Y, F | X | | |
| 6 | K, Q, M, F, A, R, S, L, V | I, H, P, Y, T | X | | |
| 7 | I, L, V | M | | X | |
| 8 | E, N, Q, D, K, S, G, R | H, T, M | | | |
| 9 | F, W | | X | | |
| 10 | L, T, I | | X | | |
| 11 | L, I, M | | | | |
| 12 | Q, H, K, R, I, S, G, T, L, D, N, Y, M, A, V | | | | |
| 13 | M, L, I, V | | X | | |
| 14 | T, C, A, V | S, Y | | X | |
| 15 | K, R, S, G, Q, H, A, N | | | | |
| 16 | K, R, N, S, Q | T, G, A | | | |
| 17 | F | | X | | X |
| 18 | R, L, K, V, T, I, M, Q, H, A, S, F, G, N, W, E | | X | | X |
| 19 | D | | X | | X |
| 20 | L, W, A, Q, K, V, R, T, P, N, D, I, H, G, F, Y, | C | | | |
| | M, S, E | | | | |
| 21 | I, V, A, L, H, R, P, N, M, T, K, Q | D, S | X | | |
| 22 | N, S,T, Q, I | D, V | X | | |
| 23 | Q, R, K, S, V, A, I, T | | | | |
| 24 | Y, W, H, I, L, M, T, N, V, E, D, F, A, S, Q, G, | | | | |
| | R, K | | | | |
| 25 | W | | X | | |
| 26 | L, I | | X | | |
| 27 | F, I, V, Y, C, M, L | W | | | |
| 28 | R, K, L, H, M, T, I, N, V, G, S, Q, A | | | | |
| 29 | R | | X | | |
| 30 | A | | | X | |
| 31 | W, F, M, Y, I, D, L, C, A, Q, H, E, R, T, S, G, | N, K | | | |
| | V | | | | |
| 32 | A, R, N, M, S, K, L, H, V, T, G | | | | |
| 33 | L, V, I, M, K, R, E, C, T | | | X | |
| 34 | A, T, V, C, L, S, I | | | X | |
| 35 | H, Q, W, S, T, M, R, C, F, V, I, N, G, Y, L, K, | D | | | |
| | A, E | | | | |
| 36 | D, S, T, G, A, E, Q, N, L, M, V, H, I | K, F, Y, R | | | |
| 37 | A, S, G, F, V, I, H, C, T, N, Y, W | | | | X |
| 38 | H, M, G, Q, R, N, S, A, P, L, F, K, E, V, C, D, | | | | X |
| | I, Y, T, W | | | | |
| 39 | E, M, S, C, D, A, Y, H, I, R, V, Q, T, K, F, W, | | | | X |
| | N, L | | | | |
| 40 | R, H, N, P, S, K, W, A, Q, T, Y, G | V, C | | | |
| 41 | W, V, K, D, E, A, G, Q, I, N, C, H, T, L, P, S, | | | | |
| | Y, R, M, F | | | | |
| 42 | C, Y, T, A, S, V, F, M, L, I | W, H | | X | |
| 43 | Y, L, T, M, V, W, F, S, C, D, N, A, H, E, I, G, | | | | |
| | R, P, Q, K | | | | |
| 44 | A, C, S, N, G, L, E, T, I, M, Y, D, F, K, W, H, | | | | |
| | R, Q, V | | | | |
| 45 | D, A, M, H, E, W, G, C, Y, Q, V, K, N, L, T, R, | | | | |
| | F, S, I | | | | |
| 46 | I, V, T, C | | | X | |
| 47 | K, M, S, T, L, W, A, R, V, Y, G, N, H, F, C, Q, | D | | | |
| | I, E | | | | |
| 48 | Q, H, S, R, D, A, G, M, N, K, C, L, V, I, F, T, | | | | |
| | Y | | | | |
| 49 | R, K, Q, N, Y, E, W, S, C, A, H, M, V, D, T, F | I, L | | X | |
| 50 | R, A, K, H, E, G, Y, W, M, D, N, V, T, S, F, Q, | | | | |
| | C | | | | |
| 51 | K, V, T, A, F, M, N, Q, W, G, E, I, R, Y, C, D, | | | | |
| | L, H, S | | | | |
| 52 | A, N, D, T, S, K, Q, E, G, H, R, V | | | | |
| 53 | L, M, Y, N, W, I, T, V, F, H, E, Q, D | | | X | |
| 54 | L, I, T, Q, R, F, H, M, K, S, D, V, A, G, E, W, | | | | |
| | N, C, Y | | | | |

TABLE 13U
H3_mb4
SKVKLSVKFLLGLARQFYRAGNPEAAEYLIKRAKEIAEKNNNEELRKEIEELEREIR
(SEQ ID NO: 648)

| | | | | | |
|---|---|---|---|---|---|
| 1 | P, Y, N, T, A, I, G, V, E, Q, M, S, R, L, W, | | X | | |
| | K, H, F, D | | | | |
| 2 | L, Q, R, K, M, F | H | X | | |
| 3 | V, T, E, H, D, Q, P, M | W | | | |
| 4 | S, R, K, M, I, W, L, G, T, E, N, H, V, Y, Q, | | X | | |
| | F, D, A, P, C | | | | |
| 5 | A, R, K, M, L, V, G, Q, S | T, I | X | | |
| 6 | K, Q, R, M, S | T, H, I, A | X | | |
| 7 | V | | | X | |
| 8 | H, K, Q, V, N, I , D, R | T, E | | | |
| 9 | F, I | | X | | |
| 10 | L | | X | | |
| 11 | M, C, L, I, T, N | Q | | | |
| 12 | M, Q, G, A, Y, T, R, K, L, N, P | S, I | | | |
| 13 | M, L | V | X | | |
| 14 | A | H, G, V | | X | |
| 15 | G, R, K, M, N, A | L, Q, S, H | | | |
| 16 | R, Q | A, S, G | X | | |
| 17 | F | | X | | |
| 18 | K, A, Q, E, S, E, Y, C, H, N, V, W, D, M, I | L | | | |
| 19 | R, K, I, S | F | | | |
| 20 | K, A | G, R, V | X | | X |
| 21 | M, N, S, R, K, G, Q, L, H, A, C, E, D, Y, I, | | | | X |
| | P | | | | |
| 22 | S, Q, N, A | | X | | X |
| 23 | P, Q | G, F, M | | | |
| 24 | P, F, Y, I, V, W, H, A, E | M, Q, C | | | |
| 25 | N, S, A | D, L | X | | |
| 26 | F, A, E, T, S, K, V, G, C, H, Y | W | | | |
| 27 | I, M, G, L, V, E, Q, H, W, R, S, C, A, F, T, | | | | |
| | N | | | | |
| 28 | W, Y | | X | | |
| 29 | L | | X | | |
| 30 | A, I, T, S, C, L, V, W, Y, R, M, G | Q, F | | X | |
| 31 | K, S, L, Y, N, A, G | M, D, T, V, I, W | | | |
| 32 | R | | X | | |
| 33 | A | G | | X | |
| 34 | A, W, K, L, I, V, M, R, N, C, Y, G, F, S, E, | | | | |
| | H, D, Q | | | | |
| 35 | N, Q, D, V, M, T, K, E, S, R, H, L | I, F | | | |
| 36 | C, L, I, M, V, E, A, W, N, S, R, K | | | X | |
| 37 | I, V, T, A, E, C | L | | X | |
| 38 | V, W, D, F, T, C, E, N, H, L, M, A, I, R, S | Q, G | | | |
| 39 | V, I, K, R, H, Q, T, N, G, F, Y, D, A, L, M, | C | | | |
| | W, S, E, P | | | | |
| 40 | A, F, N, H, S, V, Y | Q, D, T | | | X |
| 41 | A, D, R, V, Q, N, S, L, E, T, H, K, Y, C, I, | | | | X |
| | F, G, M | | | | |
| 42 | D, N, H, I, L, R, S, T, V, F, M, A, C, Y, G, | | | | X |
| | K | | | | |
| 43 | S, W, A, C, I, T, P, G, Q, E, M, H, N, L, K, | | | | |
| | V, R, Y, F, D | | | | |
| 44 | Q, S, E, P, G, A, N, D, F, Y, I, M, R, V, H | C, W, L | | | |
| 45 | M, L, C, I, E, D, Q | F | | X | |
| 46 | S, L, T, Q, V, R, I, Y, M, C, K, G, A | | | | |
| 47 | I, M, V, K, Y, G, W, A, D, S, F, C, Q, T, N | | | | |
| 48 | R, V, M, P, I, D, Y, N, C, E, K, Q, S, L, F, | | | | |
| | W, H, A, G, T | | | | |
| 49 | S, C, T, I, V | A, Y | | X | |
| 50 | N, Q, Y, F, M, E, G, H, I, L, K, W, C, V, D | | | | |
| 51 | H, A, Q, K, M, S, R, L, P, E, Y, W, I, F, V, | | | | |
| | N, G, T | | | | |
| 52 | V, A, R, Q, F, L, N, T, W, K | D, E, I | | X | |
| 53 | R, Y, Q, I, V, C, E, L, P, N, F, M, K, G, A, | | | | |
| | S, W, H, T | | | | |
| 54 | F, A, W, L, R, Q, H, P, E, G, M, Y, D, S, T, | | | | |
| | N, K, I | | | | |
| 55 | S, R, G, K, M, N, E, V, I, T, L, D, Y, C, W, | | | | |
| | F, Q | | | | |
| 56 | K, Y, L, N, Q, F, I, S, R, A, P, W, M, T | E, V | | X | |
| 57 | L, S, I, D, F, N, R, M, G, T, K, C, Q, W | E | | | |

TABLE 13V
H3_mb5
SERNEHLLRWLLEKIRKSTNEISLRFLLGLLEQFARAAGTEEAREILREAEEIVK
(SEQ ID NO: 649)

| | | | | | |
|---|---|---|---|---|---|
| 1 | R, V, K, T, S, A, I, H, G, E, Y, N, W, Q, M, D, P, L | | | | |
| 2 | A, G, N, T, W, I, V, E, R, M, P, Q, F, H, K, Y, L, D, S | | | | |
| 3 | R, H, A, N, Y, Q, G, P, I, E, S, K, W, T, M | | X | | |
| 4 | G, R, K, N, D, S, V, E, Q, H, L, P, T, Y, A, I | | X | | |
| 5 | A, G, H, F, T, L, S, N, E, Y, I, V, K, Q, R, W, D, P, M | K, W | | | |
| 6 | M, R, Q, G, I, H, L, T, D, P, N, E, A, V, S, F, Y | | X | | |
| 7 | H, M, F, N, L, Q, G, S, P | | X | | |
| 8 | L, C, M, A, H, T, S, Q, I, W, P, F, Y, G | | | X | |
| 9 | N, A, V, L, R, Y, I, H, K, F, E, M, T, G, W, S, C, D, Q | | | | |
| 10 | H, Y, W, F | | X | | |
| 11 | L, V, I, M | | X | | |
| 12 | K, L, Q, V, R, M, A, T | | | | |
| 13 | W, I, L, S, R, E, M, K, V, T, C, A, H, N, F, D, Q, Y, G | | | | |
| 14 | K, R, N, H, S, G, A | | X | | |
| 15 | I, L, M | | | X | |
| 16 | R, V, I, S, T, M, Y, L, K, A, Q, E | | | | |
| 17 | R, K, G, Y, H, F, I, Q, N, V, M, S, A, L, W, T, D | | | | |
| 18 | A, S, T, Q, K, V, R, H, G | | | X | X |
| 19 | A, T, V, P, Y, I, M, N, K, Q, S, E, R, H, D | | | | X |
| 20 | A, N, R, K, G, T, S, H, L, E, Q | | X | | X |
| 21 | I, K, Q, H, R, V, E, M, F, D, P, G, A, W, Y, N, L, T, S | | X | | |
| 22 | I, G, M, V, T, S, A, L, Q, N | | X | | |
| 23 | A, G, S, Q, N, T, P | | X | | |
| 24 | K, Q, R, H, F, S, L, G, V, I, C, T, A, M, Y, N | | | | |
| 25 | K, R, H, Y, S, L, F, I, N, M | | X | | |
| 26 | Y, W, F | | X | | |
| 27 | Q, N, L, V, C, M | | X | | |
| 28 | R, S, A, K, L, Q, I, G, F, W, M, T, N, H, C, Y, V | | | | |
| 29 | N, R, G, T, H, K, A, S | | | | |
| 30 | R, N, S, Q, H, T, W, L, I, V, A, M, P, E, C | | X | | |
| 31 | C, L, T, M, V, A, I, Q | | | X | |
| 32 | T, H, Q, D, K, G, E, V, R, M, S, F, L, N, I, W, A, Y, C | | | | |
| 33 | R, G, K, N, Q, P, T, S | | X | | |
| 34 | F, Y, W | | X | | |
| 35 | A, G, C, S, Q, V, N, T, H, I | | | | |
| 36 | R, G, V, I, D, M, Y, T, C, N, E, Q, K, A, S, W, H, F, L | | | | |
| 37 | T, R, A, S, N, G, V, P, Q, K | | X | | |
| 38 | H, P, Q, R, K, A, S, T, M, W, D, G, N, F, Y, C, E, I | V | | X | |
| 39 | P, A, W, K, R, I, Y, S, Q, N, H, L, T, G, E, V, D, F, | | | | X |
| | M, C | | | | |
| 40 | K, H, A, R, Q, T, N, E, G, D, P, V | | | | X |
| 41 | R, F, M, T, C, L, P, Y, E, G, V, W, H, Q, A, D, S, K, | | | | |
| | N, I | | | | |
| 42 | I, V, K, F, L, M, A, W, H, C, R, P, S, T, Y, N, G, E, | | | | |
| | D, Q | | | | |
| 43 | G, A, T, R, S, N, V, H, K, Y, C | | | X | |
| 44 | Y, G, R, H, N, E, S, D, L, T, P, C, K, F, V, I, Q, M, | | | | |
| | W | | | | |
| 45 | Y, W, N, K, F, E, D, V, L, T, C, M, I, H, A, R, S, Q, | | | | |
| | P, G | | | | |
| 46 | K, T, Y, H, R, I, S, L, A, C, F, V | | | X | |
| 47 | M, I, Q, N, C, H, F, R, V, K, L, S, A, Y, T, E, W, G | | | | |
| 48 | N, D, Q, F, M, E, Y, C, L, R, G, T, A, W, H, K, S, I, | | | | |
| | V | | | | |
| 49 | R, K, Y, S, C, L, E, I, N, H, Q, T, A, G, D, V, M | F | | | |
| 50 | S, A, T, C, G, K | | | X | |
| 51 | K, F, Y, I, R, G, H, L, A, E, W, T, S, D, M, Q, N, V, C | | | | |
| 52 | F, I, S, N, Y, L, E, R, M, D, V, P, Q, T, W, A, G, C, | | | | |
| | K, H | | | | |
| 53 | K, D, E, S, R, I, G, H, W, T, A, Y, C, V, N, P, F, L, | | | X | |
| | M | | | | |
| 54 | R, P, S, A, K, T, Y, E, Q, V, G, H, N, L, D, M, C, I, | | | X | |
| | F | | | | |
| 55 | H, D, C, K, V, P, Q, N, W, L, G, T, R, I, Y, S, A, M, | | | | |
| | E, F | | | | |

TABLE 13W
H3_mb6
DERSSAKISVEFLLGLAKQFTDPRIREWLLKRAKRLAEEVGDDELKRLIEKVEEEL
(SEQ ID NO: 650)

| | | | | | |
|---|---|---|---|---|---|
| 1 | R, G, H, Q, P, I, A, V, W, E, F, M, Y, L, K, D, T, N, S | | X | | |
| 2 | R, M, N, T, P, A, Q, K, S, I, G, E, V, D, H, Y, L | | | | |
| 3 | T, N, P, R, H, A, F, M, Q, G, V, S, E, W, L, I, K, Y, D | | | | |
| 4 | H, E, P, A, N, G, R, I, L, M, S, D, F, Q, Y, V, K, W, T | | X | | |
| 5 | G, K, H, R, N, Q, P, S, M, T, D, E, Y, A | F | X | | |
| 6 | P, R, H, S, A, V, Q, G, K, E, L, Y, D, N | | | X | |
| 7 | T, Y, E, K, Q, N, V, R, W, S, M, L, F, G, D, I, A, H | | X | | |
| 8 | K, P, R, T, V, Q, H, A, L, G, N, S, M, D, I, Y | E | X | | |
| 9 | R, Q, M, A, K, S, P, Y, T, N, H, F | | X | | |
| 10 | I, V, T, C | | | X | |
| 11 | K, S, Q, H, R, A, T, E, M, G, D, N | | | | |
| 12 | F, W | | X | | |
| 13 | L, I | | X | | |
| 14 | L, V, I, M, C, A | | | | |
| 15 | K, R, N, H, I, Y, S, V, G, T, A, L, M, Q | | | | |
| 16 | L, M, K, I | | X | | |
| 17 | A, S, C, R, T, L, Y, H, I, M, V, N | | | X | |
| 18 | R, H, A, K, G, N, S, Q, T, Y | V, I | | | |
| 19 | R, N, S, Q, T, A | G, K | | | |
| 20 | F | | X | | X |
| 21 | K, R, N, G, Q, H, T, S, M, L, F, I, V, E, Y, A, D, W | | | | X |
| 22 | D | | | | X |
| 23 | K, A, L, I, M, T, F, R, P, N, S, V, H, D, E, Q, G, Y, C | | | | |
| 24 | N, H, L, A, V, I, R, T, D, Q, S, K, M, G, P, E | | X | | |
| 25 | S, N, T, I, D | | X | | |
| 26 | V, K, Q, I, R, C, T, E, S | | | | |
| 27 | T, A, V, R, L, M, S, I, E, H, K, Q, N, W, C, D, G | | | | |
| 28 | W | | X | | |
| 29 | L, I | | X | | |
| 30 | I, V, Y, L, C, F, T, M, H, A | | | | |
| 31 | R, V, A, K, T, H, I, Q, M, F, N, S, D, L | | | | |
| 32 | R | | X | | |
| 33 | A, S | | | X | |
| 34 | M, L, R, S, V, G, H, E, K, Q, T, I, C, W, A, F, N, Y, D | P | | | |
| 35 | K, S, N, A, L, R, G, I, H, D, T, V, Q, M | | | | |
| 36 | K, L, R, V, I, H, M, A, Q, C | | X | | |
| 37 | N, V, E, Q, S, A, T, G, I, C, Y, L, H, M | W | X | | |
| 38 | R, W, L, D, A, T, V, S, C, H, I, Y, G, E, M, F, K, N, Q | | | | |
| 39 | R, S, K, N, Q, T, G, M, I, V, L, E, Y, D, H, A, F, P | | | | |
| 40 | E, K, A, Y, R, D, V, C, I, W, P, S, Q, N, T, G, M, H, L, | | X | | |
| | F | | | | |
| 41 | N, A, R, S, T, K, E, H, G, P, I, V, M, Q, C, Y, F, W, L, | | | X | |
| | D | | | | |
| 42 | V, Y, E, D, T, S, K, Q, G, W, M, N, I, H, C, R, A, F, L | | X | X | |
| 43 | M, Y, R, I, Q, L, W, V, E, S, F, A, G, D, N, H, T, P, C, | | | | |
| | K | | | | |
| 44 | W, H, G, Y, D, M, R, K, V, E, F, T, C, S, Q, L, N, P, I, | | | | |
| | A | | | | |
| 45 | D, C, T, L, S, A, V, I, M, Y, Q | | X | | |
| 46 | Y, I, M, S, L, N, Q, R, C, E, V, K, T, H, G, W, P, F, A, | | | | |
| | D | | | | |
| 47 | N, H, M, D, A, W, K, L, G, C, R, Q, F, I, E, V, S, P, Y, | | | | |
| | T | | | | |
| 48 | R, K, V, Q, G, W, D, M, I, P, T, E, S, L, A, N, H, Y, F, | | | | |
| | C | | | | |
| 49 | I, V, A, C, Q, T, M, S, Y, H | | X | | |
| 50 | N, T, R, S, G, H, W, C, V, F, K, M, E, Y, L, I, A, D, Q, | | | | |
| | P | | | | |
| 51 | G, M, P, R, D, K, C, N, I, F, E, H, Q, V, S, T, A, L, Y | | | | |
| 52 | R, K, Y, F, C, Q, D, N, V, H, W, M, T, A, G, S, I, L | P, E | X | | |
| 53 | A, R, K, N, Y, S, V, H, E, G, D, C, W, T, Q, M, I, F | | | | |
| 54 | L, N, A, I, V, C, Q, R, H, S, F, T, G, W, E, M, D, K, P, | | | | |
| | Y | | | | |
| 55 | D, G, S, H, T, A, K, R, W, N, V, Q, Y, E, F, L, M, P, I | | | | |
| 56 | T, E, A, Q, I, M, L, R, V, P, K, C, H, Y, S, F, N, W, D, | | X | | |
| | G | | | | |

TABLE 13X
H3_mb7
TKERVEHLVKWLLERAKRMPSPISRKFLLGLAEQFARAGNDPELERLVEKVKREL
(SEQ ID NO: 651)

| | | | | | |
|---|---|---|---|---|---|
| 1 | K, A, R, S, Q, T, P, H, N, G, E | | X | | |
| 2 | K, R, G, S, H, Q, A, N, T | Y | | | |
| 3 | A, Q, P, H, S, G, K, T, N, D, E, R, Y, M | V | | | |
| 4 | R | | X | | |
| 5 | L,A, R, N, V, S, Q, I, G | K, C | X | | |
| 6 | F, W, N, V, A, Y, D, T, K, S, M, E, C, G, R, Q, I, H, L | | | | |
| 7 | K, M, R, I, V, Q, L, T, H, S, A, G, P, N, E | D, Y | X | | |
| 8 | M, L | | X | | |
| 9 | V | | | X | |
| 10 | F, G, M, V, Q, K, L, N, H, R, T, Y, C, I, W, A, S | D | | | |
| 11 | W | | X | | |
| 12 | L, C | | X | | |
| 13 | L, I, C, V | | | | |
| 14 | Q, M, G, D, E, L, A, S, T, H, K, W, F, R, C, Y, N | | | | |
| 15 | K, R | | X | | |
| 16 | A, S | C | | X | |
| 17 | R, K, V, S, Q, T, N, L, H,A, Y | C | | | |
| 18 | S, R, V, K, L, Q, H, M, T, A, G | I, D | | | |
| 19 | R, A, S, N, H, Y, M, G, F, Q, T, K | | X | | X |
| 20 | Y, R, A, W, K, T, G, H, N, P, Q, V, S, I, M | E | | | X |
| 21 | T, K, R, H, A, N, S, L, F, Q, V, Y | | X | | X |
| 22 | D, V, Q, M, H, R, L, I, T, N, E, K, P, F, A, W, S, Y, G | | X | | |
| 23 | M, L, I | | X | | |
| 24 | S | | X | | |
| 25 | R, K, Q | | | | |
| 26 | I, S, G, N, R, K, T, E, Q, A, D, H, M, V | | | | |
| 27 | F | W | X | | |
| 28 | Q, L | N | X | | |
| 29 | L, I, M, F | | | | |
| 30 | G | | | | |
| 31 | L | | X | | |
| 32 | A | | | X | |
| 33 | E, S, T, A | Q | | | |
| 34 | N, S, T, Q, G | R | X | | |
| 35 | Y, F | | X | | |
| 36 | A | S | | X | |
| 37 | H, N, R, T, L, S, K | M | | | |
| 38 | K, R, N, Q, S, A, G, D | | X | | |
| 39 | S, G, A, N, R, C, Q, I | | | X | X |
| 40 | S, Q, M, R, N, H, G, A, T, L, D, K, P, V, E | | | X | |
| 41 | H, R, L, M, Y, D, A, Q, F, N, S, T, K, V, C, I | | | X | |
| 42 | W, K, A, P, E, M, T, I, N, R, G, Q, H, L, F, Y, V, S, | | | | |
| | D, C | | | | |
| 43 | K, Q, D, A, I, N, S, T, E, Y, P, V, W, C, F, L, M, R, | | | | |
| | G, H | | | | |
| 44 | L, M | | X | | |
| 45 | M, C, Q, N, S, L, Y, I, H, E, V, G, F, W, R, T, A, K, | | | | |
| | D, P | | | | |
| 46 | S, Y, K, F, R, L, V, Q, H, A, C, M, E, D, N, I, T, W, | | | | |
| | G | | | | |
| 47 | I, L, W, M, T, Q, E, F, Y, V | R, D, A, C | | | |
| 48 | V | I | X | | |
| 49 | D, W, A, T, E, M, L, R, Q, S, N, H, I | V, G | | | |
| 50 | M, I, N, K, H, T, R, G, A, L, D, Q, S, V, Y, C, F, E | | | | |
| 51 | S, W, T, V, N, Y, L, C, H, F, G, Q, M, A, I, E, R | | X | | |
| 52 | Q, A, K, S, T, D, N, G, R, M, E | H, V | | | |
| 53 | Y, G, S, R, Q, E, F, L, K, C, T, V, W, A, I, N, M, H | D | | | |
| 54 | R, Q, T, L, G, K, N, E, H, S, M, D, I, V, Y | F | | | |
| 55 | L, M, I, H, V | F, C | X | | |

TABLE 13Y
H3_mb8
DQAKRMVEWLLKKAREALKLGNEISVRFLLGLAKQFAHANNDEEAERLVREFEKEL
(SEQ ID NO: 652)

| | | | | | |
|---|---|---|---|---|---|
| 1 | T, N, G, S, K, Q, H, R, D | F, I | | | |
| 2 | A, T, N, K, S, Q | E, V | X | | |
| 3 | L, A, G | Y, S, P, R | | X | |
| 4 | L, R, Y, K, Q, N, I, M, F | V, E | | | |
| 5 | P, A, Q, K, R, T | V, E, L, H, W, G | | | |
| 6 | M | W, V, P, T, C, R, Y, S, G | X | | |
| 7 | V | M, L, C, W, A, P, T, I | | X | |
| 8 | I, V, E, R, K, Y, Q | W, P, N, G, M, L, C, H, A | | | |
| 9 | W | P, I, F, G, H, Q | X | | |
| 10 | L | A, C | X | | |
| 11 | L | F, A, S, P | | | |
| 12 | P, K, R | I, S | | | |
| 13 | K, R | Y, L, C, W, V, G, A | X | | |
| 14 | A | E, L, T, D, M, Y | | X | |
| 15 | V, R, H | L, P, A, S, T, C, G, N | | | |
| 16 | K, T, R, W, Q, I, V, H, Y, A, N, | | | | |
| | L, F, P, S, G, M, D, C, E | | | | |
| 17 | S, N, G, A, E | L, Q, I, T, C, W | | X | |
| 18 | K, V, T, A, R, L, Y, I, H, D, S, | E, C, P | | | |
| | M, F, N, Q, W | | | | |
| 19 | K | R, M, C, I, L, A, G, T | | | |
| 20 | R, K, H, L | G, E, I, F, W | | | X |
| 21 | R, K, G, H, N | F, D, W, C, V, Q | | | X |
| 22 | R, H, K, N, P | S, M, C, A, Y, L | X | | X |
| 23 | Q, T, M, A, W, K, R, H, S, V, I, | P, N | X | | |
| | E, G, Y, F, C | | | | |
| 24 | M, L, I | F, Y, S, T, P, K | X | | |
| 25 | S | L, Q, R, V, E, H, K | X | | |
| 26 | L, I, W, V | P, D, Y, S | | | |
| 27 | Q, D, N, R, H, S, T, E, M, V, K, | Y, P, F | X | | |
| | G | | | | |
| 28 | F | G, E, I, P, M, D, H | X | | |
| 29 | L | A, S, M, W, E, H, T, F, D | X | | |
| 30 | Y, H, F, L, W | Q, A, N | | | |
| 31 | G | T, Y, H, R, L, K | | | |
| 32 | N, L, T | S, P, C, R | X | | |
| 33 | A | Q, T, Y, M, E, H | | X | |
| 34 | Q, N, K | H, I, C, T, Y, M, S, R, P | | | |
| 35 | N, R, G, Q | K, S, Y, P, L, C, H, V, W, | X | | |
| | | I | | | |
| 36 | F | A, D, Y, V, W | X | | |
| 37 | A | S, W, M, D, P, R | | X | |
| 38 | F, Y, G, S, H, W, A, M, C, T, P | Q, L, I | | | |
| 39 | R, K, A, N | H, D, V, C, T | X | | |
| 40 | S, A, G, R, K, N, T | I, D | | | X |
| 41 | H, K, R, N, M, G | P, Y, I, F, Q, S, V | | | X |
| 42 | T, G, N, Q, D, I, R, L, E, H | C, F, V | | X | X |
| 43 | G, R, P, H, K, I, E, S, M, L, F, | N, Y, D, W | | | |
| | V, Q, A | | | | |
| 44 | Y, S, H, E, T, W, D, L, C, Q, R, | A, I, F, V, M, G | | | |
| | K | | | | |
| 45 | A | T, Y, V, Q, I, H, N, P, S | X | | |
| 46 | I, K, R, M, V, Q, T, S, E, N, G, | A, Y | | | |
| | D, P, C | | | | |
| 47 | R, A, H | T, Y, C, M, K, L, F | | | |
| 48 | M, W, L, Q, H, D, A, E, N, G | S, F, T, C, I | X | | |
| 49 | V | D, T, A, R | X | | |
| 50 | S, R, V, A, L | K, P, C, E, T | | | |
| 51 | I, E, L, D, N, G, V, T, H, Q | A, M, K, Y | | | |
| 52 | F | T, K, P, L | X | | |
| 53 | E | K, Q, A, C, F | | | |
| 54 | G, H, W, F, K, V, T, I, L, Q, Y, | A, E, N, D, M | | | |
| | R, S, C | | | | |
| 55 | P, V, E, M, R, L, Y, C, T, D, G, | W | | | |
| | N, A, I, H, K, Q, S, F | | | | |
| 56 | K, A, M, R, G, T, Q, S, Y, L, C, | | X | | |
| | W, I, P, N, H, E, D, F | | | | |

TABLE 13Z
H3_mb9
DLREKLKSMVKDLLRKAQESQDPYYAHQAEILIETIRKLAKDPDVQRLAQEALESLRKYL
(SEQ ID NO: 653)

| | | | | | |
|---|---|---|---|---|---|
| 1 | D, N | S, P, G | | | |
| 2 | L, I, M | P, F, V | X | | |
| 3 | K, R | | | | |
| 4 | E, G, Q | S, A, D, L, F, K, T, M, Y, V, W, | | | |
| | | H, N, I | | | |
| 5 | N, R, S | K, E, D, A, G | | | |
| 6 | I, L | | | | |
| 7 | K | M, S | | | |
| 8 | Q, N, F, T, S, K, R, L, W, | G, E, C, I, V | | | |
| | D, H, M, Y, A | | | | |
| 9 | R, T, H, K, V, S, I, A, N, | M, L, Y, F, D, W | X | | |
| | Q | | | | |
| 10 | I, V | F, M, L | | X | |
| 11 | R, K | A, L, S, M, T, Q, C, N, V, I, G, W, H | | | |
| 12 | M, L, T, K, H, F, Q, E, R, | S, D, A, G, W | | | |
| | I, V, N, Y, C | | | | |
| 13 | L | I, M, V | X | | |
| 14 | W, A, Y, H, F | L, T, S, V, Q, M, R, C | | | |
| 15 | R, K, Q, F, H, L, Y, W, M, | N, V, A, G, I, C, E, D | | | |
| | T, S | | | | |
| 16 | K, E | R, Q, M | | | |
| 17 | A | G, V, S, F | | X | |
| 18 | M, I, L, V, F, Y, K, Q | W, H, T, C, R, E, N, S, G | | | |
| 19 | E, A, M, L, Q, D, N, K | R, S, H, I, V, T, G, Y, F | | | |
| 20 | K, R, S | A, N, M, V, Q, G, H | | X | X |
| 21 | N, G, Q, K, H | D, R, E, A, S, T, M, P, Y, L, V, C | | | X |
| 22 | N, S, D | K, Q, T, R, E, A, H, M | | | X |
| 23 | P, K, V | R, A, I, H, T, S, Q, Y, E, M, C, F, | | | |
| | | W, G, L | | | |
| 24 | N, K, G, R, H, A, S, D, Q, | F, C | X | | |
| | E, L, M, V, T, P, Y, I | | | | |
| 25 | L, I, Y | F, M, K, V, W, A | X | | |
| 26 | M, C, I, V | A, Q, G, R, S, K, T | | | |
| 27 | R, K, G, A, P, H | M, S, Q, T, N, C, E, D | | | |
| 28 | Q | | X | | |
| 29 | C, V, T, I, A | Y, S, H, F | | X | |
| 30 | M, G, Y, A, S, C, R, E, K, | T, Q, V, H, W | | | |
| | I, F | | | | |
| 31 | L, I | | X | | |
| 32 | L, F | | X | | |
| 33 | I | | | X | |
| 34 | N, M, R, K, A, E, Q, D, G, | T, H, F, L, I, C, V, Y | | | |
| | S | | | | |
| 35 | L, K | T, V, M, I, G | X | | |
| 36 | I | V | X | | |
| 37 | K, R, A | Q, H, Y, M, G, C, W, S, F, N, L | | | |
| 38 | K | R | X | | |
| 39 | F, L | W, H | X | | |
| 40 | V, A | C, M, I | | X | X |
| 41 | I, M, A, V, S, L, P, N, G, | K, R, C | X | | X |
| | D, E, Q, H, T, F, W, Y | | | X | |
| 42 | D | A | | | X |
| 43 | P, K | R, T, S, H, V, A, Q, I, C, N, E, G, | | | |
| | | L, Y | | | |
| 44 | G, S, E, Q | D, A, T, H, I, M, N | | | |
| 45 | I, V | L | | | |
| 46 | V, T, M, I | E, Q, S, A, R, N | | | |
| 47 | R, K, Q, A, S | N, M, L, T, H, I, F, V, Y, C, E, G, | | | |
| | | D | | | |
| 48 | C, V, T, A, I, E, H | Q, L, Y, S, F, M, K, D, N | | | |
| 49 | C, A | T | | X | |
| 50 | T, N, S, G, A, E, Q, C | K, D, V, H, L, I, R, M, F, Y, W | | | |
| 51 | S, C, E, A, Q, N, D, Y, F, | R, V, L, I | | | |
| | W, H, G, T, K, M | | | | |
| 52 | A | S | | X | |
| 53 | I, L | V, E | | | |
| 54 | V, T, N, A, R, S, E, D, W, | G | | | |
| | L, Y, F, M, Q, K, C, I, H | | | | |
| 55 | W, Q, T, S, K, H | A, E, I, Y, D, V, R, N, G, M, F, C, | | | |
| | | L | | | |
| 56 | L, I | | | X | |
| 57 | R, K, A, Q | H, G, S, T, N, P | | | |
| 58 | N, V, A, S, G, I, K, T, C, | | | | |
| | H, L, F, Q, M, R, D, E, W, | | | | |
| | Y, P | | | | |
| 59 | W, M, F, Q, T, S, D, I, L, | N, G, Y, K, E, A, P, R, C | | | |
| | V, H | | | | |
| 60 | Y, F, T, W, H, P, I, R | L, Q, V, A, M, S, N, K, D, C, E, G | | | |

TABLE 13A A
H3_mb1
DDIQKEAKKVIEQLQRIAKTFPDNMNVRYLVMLAERFAHAAQRMNDEEALKLAKRLLEKAKKAG
(SEQ ID NO: 654)

| | | | | | |
|---|---|---|---|---|---|
| 1 | P, S, M, I, V, A, K, H, N, D, | | | | |
| | Y, R, W, F, L, G, E, T, Q, C | | | | |
| 2 | S, I, N, W, L, Y, D, A, G, V, | C | | | |
| | H, F, T, P, K, R, E, M, Q | | | | |
| 3 | I, V, M, L, T, H, F | N, P, K, Q, R, A, C, Y | | | |
| 4 | H, Q, D, T, F, M, A, W, Y, N, | I, C | | | |
| | E, K, S, L, V, R, P, G | | | | |
| 5 | W, Y, I, E, D, G, K, Q, T, R, | | | | |
| | M, N, V, L, S, P, A, F, H | | | | |
| 6 | R, K, G, S, N, A, E, Q, D, P | H, M, T, C, V, L | | | |
| 7 | C, V, T, A, S, I, G | Y, Q | | X | |
| 8 | V, A, I, F, N, L, D, K, G, E, | W | | | |
| | H, S, Q, Y, R, M, T, P, C | | | | |
| 9 | H, R, K, Y, L, F, V, A, S, I, | W, G, C | | | |
| | N, M, Q, T, E, D | | | | |
| 10 | V, T, I, L, C | A, M | | X | |
| 11 | M, T, E, L, G, H, V, I, Q, S, | R | | | |
| | C, F, Y, D, N, K, W, A | | | | |
| 12 | G, R, W, M, N, Q, K, L, Y, C, | | | | |
| | T, D, E, F, S, V, I, A, H | | | | |
| 13 | R, A, L, N, Q, E, K, D, T, Y, | W, G, F | | | |
| | H, C, V, M, S, I | | | | |
| 14 | L, M | C, I | | X | |
| 15 | R, H, W, Q, M, Y, D, A, T, K, | C, I | | | |
| | E, S, G, N, L, V, F | | | | |
| 16 | F, R, I, G, S, H, W, A, D, M, | C, E | | | |
| | V, K, T, Y, Q, L, N | | | | |
| 17 | L, C, T, M | A, I, V, Q, Y, F, W | | X | |
| 18 | F, M, Y, D, N, H, L, I, Q, A | S, W, V, G, T, C, R, E, K, P | | | |
| 19 | R, K, L, D, T, W, H, G, Y, A, | I, N, P | | | |
| | V, F, S, E, C, M, Q | | | | |
| 20 | R, M, L, Y, F, K, W, H, T, Q, | | | | |
| | I, G, N, A, D, C, V, S, E | | | | |
| 21 | H, F, Y, W, Q, C, N, A, L | V, E, R | | X | X |
| 22 | W, G, P, A, K | Y, T, S, H, M, F, N, D, V, R, | | | X |
| | | I, L, Q, E, C | | | |
| 23 | F, K, R, L, T, A, H, Q, Y, S, | E, W, C | X | | X |
| | I, G, V, N, D, M | | | | |
| 24 | N, D | E | | | X |
| 25 | W, M | | X | | |
| 26 | P, E | N, V, T, A, S | X | | |
| 27 | V, K | | | X | |
| 28 | Y, F, I, V | H, K, W, Q, T, M, R, L, C | X | | |
| 29 | L | Y, P | X | | |
| 30 | R, I | Y, L, V, W, M, T | X | | |
| 31 | G, A, Q, S | T, V, Y, W, E, C, I, M | | X | |
| 32 | R, L, I, Q, A, K, V, N, S, G, | T, D, M, H | | | |
| | P, E | | | | |
| 33 | L | | X | | |
| 34 | A, G | | | X | |
| 35 | I, V, E | H, F, Q, L, Y, M, C | | | |
| 36 | R, K | | X | | |
| 37 | F | | X | | |
| 38 | V | C, A, I, T, G | | X | |
| 39 | E, D, Q, A, N, G, H, V, K | M, S, L, T, F, W, I, R, Y, C | | | |
| 40 | Y, H, W, F | R, Q, A, V, N, T, L, S | X | | |
| 41 | A, S, G | | | X | |
| 42 | C, I, Q, V, M, A, T, E, D, S | G, H, L, K, N, R, W, F | | | |
| 43 | S, K, R, Q, N, E, T, G, A | D, H, V, M, I | | | |
| 44 | R, K, T | N, S, G, M, A, Q, H, P, L, V | X | | |
| 45 | L, N, D, V, T | G, K, Q, P, E, S, A, R, M, Y, C, | | | X |
| | | H, W, F, I | | | |
| 46 | N, H, S, G, T, Q, K | L, M, D, A, E, R, V, C, I | | | X |
| 47 | A, R, S, K, W, D, P, Q, T, F, | I | | | |
| | H, E, Y, G, M, V, L, N, C | | | | |
| 48 | L, V, I, T, M | Q,E,N | X | | |
| 49 | A | P | X | | |
| 50 | I, L, V, P | M | | | |
| 51 | M, R, G, Y, S, H, L, F, N, V, | E, A | | | |
| | K, T, C, I, Q | | | | |
| 52 | I,V | L | X | | |
| 53 | A, G, S, C | T | | X | |
| 54 | G, V, K, Y, W, H, R, F, A, N, | S, M, C, I, L, T, D | | | |
| | Q, E | | | | |
| 55 | V, K, L, I, N | T, R, Y, M, H, S | X | | |
| 56 | L | | X | | |
| 57 | R, Y, L, A, F, Q, G | W, V, M, C, K, E, I, T, S | | | |
| 58 | Y, F, W | H, G, M, D, E, V, Q, T, L, A, S, | | | |
| | | I, N, R, K, C | | | |
| 59 | F, M | K, Y, N, W, S | X | | |
| 60 | A, S, V | C,T, G | | X | |
| 61 | M, L, C, A, K, E, Q, I, S, G, | F, R | | | |
| | T, N, Y, H, V, W | | | | |
| 62 | K, R, T, A | D, N, V, S, F, Y, Q, H, M | | | |
| 63 | W, Y, F | H, A, N, S, Q | | X | |
| 64 | A, G | S,T | | | |

TABLE 13BB
IGF1R_mb1
STRNAEFIMLLLELCVKSKNDPQVQEYVKKVKKQVERLVGNGDEKKAEEVARKALEYCADG
SEQ ID NO: 713

| | | | | | |
|---|---|---|---|---|---|
| 1 | T, D, M, G, K, A, V, I, F, N, Q, R, | | | | |
| | Y, S, P, H, L, E | | | | |
| 2 | R, N, Q, A, F, H, P, G, S, D, T, W, | | | | |
| | M, E, Y, I, V, K, L | | | | |
| 3 | Q, S, P, A, M, V, K, Y, D, T, G, E, | | X | | |
| | R, N, I, H, L, F | | | | |
| 4 | H, D, S, N, E, Q, T, G | | X | | |
| 5 | A, S, W, G, T, I, P, L, V, C, Y, F | | X | | |
| 6 | D, H, Q, E, R, G, F, N, Y, P, M, K, | | X | | |
| | S, W, I, C, T, V, L | | | | |
| 7 | F | | X | | |
| 8 | Y, F, M, L, I, H, W, V, T, N | | X | | |
| 9 | M, C, L, F, V, W, T, Y, I | | | X | |
| 10 | G, A, K, R, L, T, M, Q, E, S, C, V, F | | X | | |
| 11 | M, V, L, I | | X | | |
| 12 | L, V, F, M, I, T, S, A | | | X | |
| 13 | Q, E, D, H, V, M, S, I, T, N | | | | |
| 14 | V, L, I, T | | X | | |
| 15 | C, A, I, V, L | | X | | |
| 16 | V, C, I, M, L, A, F, S, T, G, W | | | X | |
| 17 | R, Q, S, D, K, G, E, T, N, V, M, | | | | |
| | A, H, P | | | | |
| 18 | G, Q, P, D, T, H, S, N, R, E, K | | X | | |
| 19 | N, S, T, G, E, K, H, P, D, Q, C, V, | | X | | X |
| | A, Y, I, F | | | | |
| 20 | W, F, V, I, R, P, S, D, E, Q, K, L, | | | | X |
| | H, T, N, Y, G, A | | | | |
| 21 | F, K, H, E, Y, T, A, N, R, | P, W, L | | | X |
| | I, V, G, S, D | | | | |
| 22 | S, L, Q, M, G, A, V, I, N, F, T, K, | | | | |
| | E, H, R, D, Y, P, C, W | | | | |
| 23 | I, R, Y, F, E, K, A, L, N, D, Q, W, | | | | |
| | G, S, T, V, M, P, H, C | | | | |
| 24 | K, F, T, S, R, Y, A, V, Q, | I | | X | |
| | W, G, E, M, D, P, L, H | | | | |
| 25 | P, H, W, Y, A, M, R, L, G, S, D, V, | | | | |
| | N, K, Q, I, E, T, F | | | | |
| 26 | T, S, K, V, N, A, Q, R, M, D, G, P, | | | | |
| | E, H, W, Y, L, I, F | | | | |
| 27 | K, P, Q, R, H, N, E, S, T, G, M, Y | | | | |
| 28 | Y, M, I, F, T, Q, N, P, L, V, W, H, | | | X | |
| | G, A | | | | |
| 29 | W, G, M, N, H, Y, S, D, Q, L, T, K, | | | | |
| | R, F, E, I, A, V, P | | | | |
| 30 | Y, D, Q, M, R, T, E, S, K, N, V, P, | | | | |
| | A, G, F | | | | |
| 31 | N, I, G, A, M, Y, K, T, L, | Q, V, R, C, F, W | | X | |
| | D, P, E, H, S | | | | |
| 32 | R, P, D, K, S, Q, G, E, H, N, A, | | | | |
| | T, V | | | | |
| 33 | F, T, V, Q, S, G, Y, D, W, N, A, L, | | | | |
| | K, R, M | | | | |
| 34 | N, E, H, Q, V, D, C, G, M, R, L, K, | | | | |
| | S, A, I, Y, F, W, T, P | | | | |
| 35 | T, V, I, C, A, S, M | | | X | |
| 36 | K, D, N, L, H, T, I, R, S, A, V, Q, | | | | |
| | P, E, F, Y, W, M | | | | |
| 37 | L, T, H, I, A, V, M, G, P, F, K, E, | | | | |
| | S, N, Q, C, Y, R, D, W | | | | |
| 38 | N, C, F, E, M, D, R, S, Q, K, T, H, | | | X | |
| | L, W, Y, G, P, V, A, I | | | | |
| 39 | I, H, V, C, T, R, E, S, A, | L | | | |
| | N | | | | |
| 40 | V, Y, C, Q, I, R, G, H, L, N, M, K, | | | | |
| | S, A, D, F, E, T | | | | |
| 41 | Y, W, M, H, V, K, A, E, D, T, R, L, | | | | |
| | N, S, F, C, G, Q | | | | |
| 42 | E, A, Q, D, R, Y, G, S, N, K, T, C, | | | | X |
| | M, H | | | | |
| 43 | G, Q, W, N, R, Y, H, L, D, K, M, C, | | | | |
| | S, E, A, T, F, V | | | | |
| 44 | W, N, G, Y, A, I, E, P, M, S, T, H, | | | | |
| | Q, K, F, D, V, C, L, R | | | | |
| 45 | L, I, G, S, A, H, F, D, Y, P, T, R, | | | | |
| | V, M, E, K, Q, N, W, C | | | | |
| 46 | Q, T, I, H, F, W, P, E, R, | A, K, S, G, D | | | |
| | Y, M, N, V | | | | |
| 47 | G, A, S | | | X | |
| 48 | D, E, N | | | | |
| 49 | L, A, V, Q, R, T, D, S, I, N, E, K, | | | | |
| | G, F, H, M, P, Y | | | | |
| 50 | I, M, T, R, H, Q, A, W, S, V, K, L, | | | | |
| | C, F, Y, E | | | | |
| 51 | S, A, G | | | X | |
| 52 | N, Q, S, Y, A, L, D, R, F, H, I, T, | | X | | |
| | G, E, K, M, V | | | | |
| 53 | G, E, F, M, D, T, R, Q, H, N, K, S, | | | | |
| | Y, V, A, I, L, W, P | | | | |
| 54 | G, V, M, I, A, T, L, C, S, N, W | | | X | |
| 55 | M, L, V | | X | | |
| 56 | T, D, Q, N, E | | X | | |
| 57 | V, S, L, G, T, N, E, K, M, | C | | | |
| | F, I, A, W, R, H, Y, Q | | | | |
| 58 | C, A, V, T | | | X | |
| 59 | D, S, P, A, C | | X | | |
| 60 | E, L, P, G, D, S, N, A, V, T, R, Q, | | X | | |
| | H, I, M, K | | | | |
| 61 | D, L, E, H, M, C, Y, S, G, A, N, K, | | | X | |
| | T, I, V, F, Q, R, P, W | | | | |

TABLE 13CC
IL7Ra_mb1
HEVQELARKLWHATEKAEKRNDEKAVKLLEDASVLLVNAWLSNDEDLKKKANKYAKTVIKQAES
(SEQ ID NO: 759)

| | | | | | |
|---|---|---|---|---|---|
| 1 | E | Y, H, W, L, M, Q, S, R | | | |
| 2 | A | E, R, L, D, V, I | | | |
| 3 | M, A | V, F, I | X | | |
| 4 | Q | | X | | |
| 5 | E | | | | |
| 6 | L, M, I | | | X | |
| 7 | A | | X | | |
| 8 | R, E | | X | | |
| 9 | R, K, Q, L, I, A | | | | |
| 10 | L, I, M, V | | | X | |
| 11 | H, W | | X | | |
| 12 | H, N, K, Q, R | | X | | |
| 13 | A, M, V, H, Q, I, L, S, R | | | X | |
| 14 | T, R, S, H, M, L | | | | |
| 15 | R | E, H, V, K | X | | |
| 16 | A | N, K, L, T, I, E | | | |
| 17 | A, M | | | X | |
| 18 | E | | | | |
| 19 | K | | | | |
| 20 | M, E, D, R, K, A | N | | | X |
| 21 | N | | | | X |
| 22 | D, N, L, E, M, K | | | | X |
| 23 | E | | | | |
| 24 | K | | | | |
| 25 | M, A, N, L | I, R, V | | | |
| 26 | V, K, I, A, F, M, R | | | | |
| 27 | K | | | | |
| 28 | I, L, K, M, A, R, H | | | | |
| 29 | L, I, M | | | X | |
| 30 | E, N, T, V, Q | | X | | |
| 31 | D, E, S, N | | X | | |
| 32 | A, S, V | | | X | |
| 33 | T, Y, I, N | S, A, K, L | X | | |
| 34 | V | | X | | |
| 35 | E, L, M, I, H, A, R | | | | |
| 36 | L | | X | | |
| 37 | V | | X | | |
| 38 | Y, E, H, T | N, V | X | | |
| 39 | A, S, T | | | X | |
| 40 | W, Y, H | | X | | |
| 41 | L, A, I, M | | X | | |
| 42 | S, T | | | | X |
| 43 | N | | | | X |
| 44 | D, E, N, I, K | | | | X |
| 45 | E | | | | |
| 46 | D | | | | |
| 47 | M, L, Q, I, A, E, K | | | | |
| 48 | M, L, E | K | | | |
| 49 | K | | | | |
| 50 | K | | | | |
| 51 | A, S, V, I | | | X | |
| 52 | N | | | | |
| 53 | K | | | | |
| 54 | V, Y, H, F, M, E, K | | | | |
| 55 | A, V | | | X | |
| 56 | K | | | | |
| 57 | T | | | | |
| 58 | V, M, R, L, A, I, Q | | | X | |
| 59 | L, M | I, W, F, Y | | | |
| 60 | K | | | | |
| 61 | L, Q, I, M, R, F, E | | | | |
| 62 | T, I, A, S, Q, V, L | | | X | |
| 63 | E | | | | |
| 64 | S | | | | |

TABLE 13DD
IL7Ra_mb2
SVIEKLRKLEKQARKQGDEVLVMLARMVLEYLEKGWVSEEDADESADRIEEVLKK Optimized
SEQ ID NO: 760

| | | | | | |
|---|---|---|---|---|---|
| 1 | D, E, Y, F, I, H, S, V, Q, | P | | | |
| | G, M, A, L, T, C, K, N, | | | | |
| | R, W | | | | |
| 2 | A, T, L, V, I, S, G, P, F, | M | | X | |
| | D | | | | |
| 3 | I, V, Y, L, W, M, F, E, A | D, N, K, R, C, Q, S, T, H, G | | | |
| 4 | D, E, A, Q, Y, I | N, M, R, S, W, L, T, K, G, H, F, | | | |
| | | V, C | | | |
| 5 | H, K, R, N, T, C, E | | | | |
| 6 | L, V, M | I, C | | X | |
| 7 | E, Q, L, M, I, R, W, V | D, T, G, C, K, H, A, Y, N, F, S | | | |
| 8 | Q, A, M, E, D, L, N, W, I, | V, G, K, S, H, F | | | |
| | C,T, R, Y | | | | |
| 9 | I, M, V, T, L | A | | X | |
| 10 | L | V, Q, E, I, Y, C, A, M, R, F, W, | | | |
| | | K, G, H | | | |
| 11 | E, M, Q, A, Y, W, L, I, R, | G, T, H, S, D, N, V, F, C | | | |
| | K | | | | |
| 12 | M, A, W, D, H, N, F, T, R, Y, K, E, | | | | |
| | I, L, V, Q, S, C | | | | |
| 13 | A | | | X | |
| 14 | L, I, M, A, E | Q, C, Y, H, F, V, R, T, S, K, W, | | | |
| | | N, D, G | | | |
| 15 | L, E, W, C, F, M, H, K, T, | I, R, Y, G, A, D | | | |
| | N, S, V, Q | | | | |
| 16 | Q, M, N, A, K, H, F, V, Y, | W, T | | | |
| | I, E, C, L, S, R | | | | |
| 17 | K, G, N, H, R, Q, S, A, C, | | | | X |
| | D, T, V | | | | |
| 18 | D | | X | | |
| 19 | K, M, T, Q, G, Y, S, L, E, F, D, I, | | X | | |
| | P, V, R, A, H, C, N, W | | | | |
| 20 | V | I, L, T, M | X | | |
| 21 | L, M, C | G, A, S, Y | | X | |
| 22 | L, S, Q, V, Y, I, W, F, T, E, M, | | | | |
| | C, A, G, R, N, D, P | | | | |
| 23 | F, M | W, I, L, Y, T, R, V, A, | X | | |
| | | H, S, E, N, Q, D, C | | | |
| 24 | L, I, M, W | | X | | |
| 25 | S, A, C, I, V, G, P, E | T, M, L | | X | |
| 26 | E, C, N, Q, M, V, R, H, K, | A, Y, F, D, W | | | |
| | S, G, I, T, L | | | | |
| 27 | M | Y, C, S, I, R, H, K, N, | X | | |
| | | E, W, T, A, L, G, Q, | | | |
| | | V, D | | | |
| 28 | I, V, L, T, D, G | | X | | |
| 29 | M, W, F, I, L, Y, A, V, P | R, G | | | |
| 30 | W, C, Y, F | M, D, H, T, E, S, K, A, N, I, | | | |
| | | V, R, L, Q, G | | | |
| 31 | Y, F | M, A, L, C, S, G, H | X | | |
| 32 | M, F, L, Y, I, V, H, G, P, R | | | X | |
| 33 | E, S, D, Q, R, N, A, T | K, C, G | | | |
| 34 | M, L, H, T, D, E, S, K, I, A, G, Q, | | | | |
| | F, W, N, R, V, C, Y | | | | |
| 35 | N, G, H, D, K, E, S, A, R, C, V, I, W | | | | X |
| 36 | W | V, M, Y, E, L, Q, T, D, | X | | X |
| | | F, I, A, H, N, S | | | |
| 37 | C | V, I | X | | X |
| 38 | S, T, N | D | | | X |
| 39 | D, F, W, C, S, E, T, Y, N, | V, I, P, K, M, Q, H, G, R | | | |
| | A, L | | | | |
| 40 | A, T, S, L, D, Q, E, V, M, | Y | | | |
| | I, C, N, K, H, R, F, G, | | | | |
| | W | | | | |
| 41 | D, E | V | X | | |
| 42 | A | | X | | |
| 43 | W, C, F, T, D, S, N, A, V, G, Y, E, | | | | |
| | R, Q, K, L, I, M, H, P | | | | |
| 44 | S, D, E, Y, Q, A, H, L, W, | I | X | | |
| | M, N, C, R, K, V, G | | | | |
| 45 | A, C | S, T, V, I, F | X | | |
| 46 | A, M, V, C, I, L | S | | X | |
| 47 | R, A, D, M, E, S, I, T, H, | W | | | |
| | C, K, V, G, Q, N, L, F, | | | | |
| | Y | | | | |
| 48 | R | | X | | |
| 49 | L, I | | X | | |
| 50 | N, V, W, F, A, L, C, E, Y, | H | | | |
| | T, M, I, S, D, Q, G, R, | | | | |
| | K | | | | |
| 51 | D, T, I, K, W, N, E, F, V, H, L, Q, | | | | |
| | S, Y, R, A, C, M, G | | | | |
| 52 | V, I, A, C, M, L | T | X | | |
| 53 | V, L, I, M, F, H, P | | | | |
| 54 | T, W, H, I, R, F, Q, C, D, K, G, E, | | | | |
| | N, Y, M, L, S, V, A, P | | | | |
| 55 | D, Y, W, K, S, R, N, L, V, T, Q, M, | | | | |
| | H, F, A, G, P, I, E, C | | | | |

TABLE 13EE
IL7Ra_mb3
DEVHKLAEKAARLALKKGDWDAHAKVVNVFLALKHGIISKEQAKKILKKIIEEILS
(SEQ ID NO: 762)

| | | | | | |
|---|---|---|---|---|---|
| 1 | A, K, D, T, S, I | | | | |
| 2 | E | | | | |
| 3 | M | A, F, L, V | | | |
| 4 | D, V, R | N, H | X | | |
| 5 | K | | | | |
| 6 | M, H, I, L, E, W, F | | | X | |
| 7 | S, A, E | | X | | |
| 8 | V, E, T | | X | | |
| 9 | K | | | | |
| 10 | V, I, A | | | X | |
| 11 | A | | X | | |
| 12 | V, R, K, H, S | | X | | |
| 13 | M, Y, H, F, L, N, I | | | | |
| 14 | V, A | | | X | |
| 15 | I, V | A, L | X | | |
| 16 | K | | | | |
| 17 | M, T, N, R, K, V, Q, E | | | | |
| 18 | E, D | Q, N, G | | | X |
| 19 | N, K, I, S, D, A | | | | X |
| 20 | I, L, R, K | W | X | | |
| 21 | H | E, D | X | | |
| 22 | K, M | L, Q, S, A | | X | |
| 23 | H | | X | | |
| 24 | V | A | X | | |
| 25 | T, N, R | K | | | |
| 26 | I, V | | X | | |
| 27 | V | | X | | |
| 28 | K, R | N | X | | |
| 29 | K, R | L,V | | X | |
| 30 | W, Y, H | F | X | | |
| 31 | L | | X | | |
| 32 | R | M, H, I, A | X | | |
| 33 | N, V, M, F | Y, L | | X | |
| 34 | R, Q | M, E, K | | | |
| 35 | A | H | X | | X |
| 36 | G | | | | X |
| 37 | D, E, F, K, Q, R, T | I | X | | X |
| 38 | R, K, L | M, I | | | X |
| 39 | S | | | | X |
| 40 | K | | | | |
| 41 | E | | | | |
| 42 | K | E, A, Q | | | |
| 43 | F | V, M, I, L, Y, A | | | |
| 44 | K | | | | |
| 45 | K | | | | |
| 46 | K, E, R | I | | | |
| 47 | F, I, H, Q, L, M | | | X | |
| 48 | K | | | | |
| 49 | K | | | | |
| 50 | Q | F, M, I | | X | |
| 51 | M, E, V | I | | | |
| 52 | E | | | | |
| 53 | H, A, N, T | L, I, E | | | |
| 54 | M, V, I, D, Q, L | | | X | |
| 55 | L | | | | |
| 56 | S | | | | |

TABLE 13FF
IL7Ra_mb4
DDTKLARRKVKLALTIVKNASSKEEADRALHIAEIVADEVNEPTLQEQVTRTKKWVEKYFS
(SEQ ID NO: 763)

| | | | | | |
|---|---|---|---|---|---|
| 1 | E, D, H, V, C, S, G, I, A, | Q, T, Y, M, P, F, W | | | |
| | N, R, K, L | | | | |
| 2 | D, C, N, T | E, G, A, S | X | | |
| 3 | R, Q, L, V, M, I, D, A, E, | G | | | |
| | T, K, Y, C, W, S, N, F, | | | | |
| | H | | | | |
| 4 | E, W | H, M, N, V, I, A, F, L, R, S, | | | |
| | | T, K, D, G, Y, Q, C, P | | | |
| 5 | L, M | I | X | | |
| 6 | A, E | L, M, G, S, N, Q | | | |
| 7 | Y, W, I, R, Q, M, F, E, H, | L | | | |
| | V, K, T, A, C, D, N, G, | | | | |
| | S | | | | |
| 8 | A, I, S, T, Q, V, K, E | M, L, C, N, R, H, F, W, | X | | |
| | | G, D, Y | | | |
| 9 | K, H | | X | | |
| 10 | L | V, I, C, F | | X | |
| 11 | E, S, Y, H, I, M, D, A, K, | W, G, N, R, C | | | |
| | F, L, Q, T, V | | | | |
| 12 | M, L, V | T, Y, E, Q, I, R, S | X | | |
| 13 | A, L | I | X | | |
| 14 | L, S | I, M | | | |
| 15 | P, H, E, N, S, Q, A, G, T, | V, Y, K | | | |
| | M, D, R, L, I, C, F | | | | |
| 16 | I, M, A, C, H, F, E | Y, V, L | X | | |
| 17 | A, V, I, S | C, T | | X | |
| 18 | R, K, H, Y, E, G, S, F, N, | Q, A, M, C | | | |
| | D | | | | |
| 19 | N, Q, A, H, E | K, G, S, D, C, R, T, V | | | X |
| 20 | A, S | T, C | | X | X |
| 21 | H, Q, G, K, T, L, R, S, A, | C, D, F | | | X |
| | M, V, I, N, E, Y, P, W | | | | |
| 22 | T, S, C, N, Y, R, D, G, L, | H, K, V, F | | | X |
| | I, A, M, Q | | | | |
| 23 | R, K, F, A, E, T | I, Q, Y, W, L, M, H, V | | | |
| 24 | Y, W, E, Q, A, D, L, M, F, | H, K, P | | | |
| | S, R, T, I, V, N, G, C | | | | |
| 25 | E, I, D, L | V, A, M, T, Q, K, C, F, S | | | |
| 26 | A | M | | | |
| 27 | Q, N, M, K, E, W, L, Y, D, V, I, | | | | |
| | C, F, T, R, A, S, H, G | | | | |
| 28 | R, K, Y | H | X | | |
| 29 | A | | X | | |
| 30 | L, M, I | V | | | |
| 31 | F, Y | W, H, V, T, M, I, C, R | X | | |
| | | K | | | |
| 32 | I, R | M | X | | |
| 33 | A | L | | X | |
| 34 | E, S, A, Q, T, M, G, F | K, R, D, W, N, H, Y, C, L, V | | | |
| 35 | I, V | L | X | | |
| 36 | V, E, L, Y, M, N, S, K | I, C, F, A | | X | |
| 37 | A, S, C, N | L, V, M, I | | X | |
| 38 | S, D, E, G, A, Q, N, T, H, | C, L, F, V, K | | | |
| | W, M, Y, I | | | | |
| 39 | E, N | D, G, S, A, T, Q | X | | |
| 40 | A, S, C, V, T, I, Y | L, M, Q, G, H, F, E, N | | | X |
| 41 | D, G, N, E, A, S, Q, M, H, | P, F, W, T | | | X |
| | C, L, Y, K, R, V, I | | | | |
| 42 | E, D, C, S, I | N, M, V, L | | | X |
| 43 | P, K, R, Q, A, S, T, V, I, | W, Y, C, F | | | |
| | E, L, H, G, N, M, D | | | | |
| 44 | D, E, H, G, N, Q, S, A, Y, T, M, K, | | | | |
| | L, V, F, C, I, R, W, P | | | | |
| 45 | L, I, D | M, V, W, F | | X | |
| 46 | T, I, L, K, M, R, W, V, Y, | C, G | | | |
| | Q, S, F, N, H, A, D, E | | | | |
| 47 | D, E, A, Q, M, L, R, C, K, | H, Y, S, V, G, F | | | |
| | N, W, I, T | | | | |
| 48 | E, Q, D, M, A, L, I, V, S, | R, C, N, Y, H | | | |
| | T, F, W, K | | | | |
| 49 | V, I, C | L | | X | |
| 50 | Y, K, N, A, W, R, I, E, T, M, | | | | |
| | F, L, Q, H, V, C, S, D, G | | | | |
| 51 | N, K, R, M, A, L, S, Q, E, | I, F | | | |
| | V, D, T, W, H, Y, C, G, | | | | |
| | P | | | | |
| 52 | C, T, A, M, Y, V, S | I, H, Q, W, F, L, G | | X | |
| 53 | R, K, S, Q, W, I | A, H, M, Y, G | | | |
| 54 | Q, M, L, A, E, K, H, I, V, | | | | |
| | D, F, W, Y, N, S, T, C, G | | | | |
| 55 | W, D | E, F, Y, A, L, Q, C | | | |
| 56 | A, V, T, C, M, S, K, W, E, | I, L, F | | X | |
| | H | | | | |
| 57 | E, Q, D, N, A, M, S, K, H, | L, R, Y, V, G, C, F | | | |
| | T, I, W | | | | |
| 58 | Q, R, M, W, I, T, K, E, V, C, | | | | |
| | A, S, L, Y, N, F, H, D, G | | | | |
| 59 | H, K, F, Y, R, V, Q, M, A, | I, L, E | | | |
| | W, N, T, S, C | | | | |
| 60 | W, F, Y, M, L | | | X | |
| 61 | A, T, G, R, N, K, Q, S, H, C, | | | | |
| | V, I, M, L, D, E, Y, W, E, P | | | | |

TABLE 13GG
InsulinR_mb1
LIREEVQRRLEEIKQKIKEGKLDPFEAMSLLAELYELVEKLGDPELRKELEKVMEIVMRLG
(SEQ ID NO: 784)

| | | | | |
|---|---|---|---|---|
| 1 | L, E, W, T, I, K, N, P, S, Q, F, Y, | | | |
| | H, C, A, D, G, R, M | | | |
| 2 | E, G, M, L, Q, T, W, V, I, K, A, D, | | | |
| | P, H, N, R | | | |
| 3 | A, C, E, G, H, K, T, Y, R, M, Q, L, | | | |
| | V, F, D, N | | | |
| 4 | A, T, R, P, Q, M, N, F, G, D, C, E, | | | |
| | H, Y, S, V, W, L, K | | | |
| 5 | L, T, P, Q, N, M, E, G, D, K, V, H, | | | |
| | I, S, A, F, Y, R | | | |
| 6 | C, G, I, K, M, N, R, T, W, V, Y, A, | | X | |
| | F, S, Q, L | | | |
| 7 | Q, C, V, D, T, F, P, Y, L, S, A, H, | | | |
| | I, E, W, G, M, N | | | |
| 8 | D, I, K, M, L, T, R, H, N, V, C, Q, | | | |
| | A, W, G | | | |
| 9 | C, W, K, L, R, S, D, G, I, N, P, M, | | | |
| | Q, E, T, F, Y | | | |
| 10 | F, N, Q, P, R, Y, L, T, K, G, V, | | | |
| | E, M, A, C, H | | | |
| 11 | L, T, R, N, M, E, D, C, G, Q, A, S, | | | |
| | K, F, I, Y, V | | | |
| 12 | E, V, S, P, Q, I, H, L, A, G, R, W, | | | |
| | C, D, Y, F, T, N | | | |
| 13 | I, W, E, T, S, N, H, Y, A, L, M, G, | | X | |
| | Q, D, F, R, C, K | | | |
| 14 | A, V, S, N, H, L, F, E, K, C, Y, R, | | | |
| | W, M, I, D, P, T | | | |
| 15 | A, V, L, K, H, Q, I, G, Y, R, N, W, | | | |
| | C, M, D, S, E | | | |
| 16 | A, R, Q, I, N, E, C, G, K, P, Y, D, | | | |
| | M, S, W, L, T | | | |
| 17 | M, W, R, S, Q, N, L, I, H, F, G, E, | | X | |
| | K, D, T, Y, C, A | | | |
| 18 | F, I, H, L, Q, R, K, G, A, D, M, Y, | | | |
| | N, W, P | | | |
| 19 | N, W, T, S, P, Q, Y, L, E, K, I, G, | | | |
| | M, F, V, A, C, H, R | | | |
| 20 | A, W, T, N, K, H, M, F, E, C, G, L, | | | X |
| | S, P, V, I, D | | | |
| 21 | A, V, W, R, N, H, L, G, E, C, K, F, | | | X |
| | Y, D, I, Q, S, M, P, T | | | |
| 22 | N, W, R, S, P, I, L, D, E, F, T, G, | | X | X |
| | K, V, Q, H, C, A, Y | | | |
| 23 | G, M, N, P, S, R, D, W, H, E, L, Q, | X | | X |
| | A, Y, K, F | | | |
| 24 | A, G, P, M, V, T, L | X | | |
| 25 | F, W | X | | |
| 26 | E, D, R, S, H, K, M, L, Q, N, G, I, | | | |
| | T, W, A, Y, V, P | | | |
| 27 | G, M, V, Y, A, L, N, R, T, W, K, C, | | X | |
| | H, I, S, E | | | |
| 28 | M, L | X | | |
| 29 | G, H, S, T | X | | |
| 30 | I, H, N, Q, T, L, Y, A, D, K, W, F, | | | |
| | C, R, G, M | | | |
| 31 | I, V, L, C, M | | X | |
| 32 | S, A, G, R | X | | |
| 33 | A, D, G, H, S, E, N, R, V, I, Y, Q, | | | |
| | W, T, P | | | |
| 34 | A, V, W, S, Y, H, L, F, G, D, E, I, | | X | |
| | K, N, Q, C, T, R, M | | | |
| 35 | F, W, Y, I, M, H, L | X | | |
| 36 | E, D, T, I, M, Y, W, L, N, P, Q, H, | X | | |
| | F, A, V, R, G | | | |
| 37 | A, P, M, L, D, I, H, W, C, G, T, S, | | | |
| | N, F, R, V, Q, E | | | |
| 38 | A, C, E, G, M, L, N, Q, P, S, T, W, | | | |
| | V, D, F, Y | | | |
| 39 | P, E, D, L, I, W, F, H, V, S, M, A | | | |
| 40 | N, W, T, Q, Y, L, K, I, P, V, H, S, | | | |
| | R, A, G, C, F, D | | | |
| 41 | A, W, T, R, Q, N, G, E, L, Y, I, K, | | | |
| | M, H, P, S, D, V | | | |
| 42 | G, C, E, V, P, Q, Y, A, S, T, W, D, | | | X |
| | M, K, L, F, N, R, H | | | |
| 43 | N, V, T, S, P, Q, L, D, K, H, E, M, | | | X |
| | A, C, F, R, W, Y, I | | | |
| 44 | A, V, T, R, Y, K, L, F, H, P, E, M, | | | |
| | N, D, W, S, I, Q, C | | | |
| 45 | E, C, V, F, I, H, M, Y, S, R, T, P, | | | |
| | Q, W, G, A, K, N, D | | | |
| 46 | L, C, E, V, G, W, R, H, Y, S, Q, A, | X | | |
| | I, T, K, D, F, N | | | |
| 47 | N, V, P, I, R, D, E, C, G, W, A, S, | | | |
| | H, Q, F, Y, T | | | |
| 48 | A, V, W, P, N, Y, L, E, C, K, G, I, | | | |
| | S, Q, M, D, F, T | | | |
| 49 | A, V, W, R, S, P, Q, E, F, G, M, H, | | | |
| | C, D, N, K, T, Y | | | |
| 50 | A, G, N, P, L, C, H, W, T, F, Q, E | | X | |
| 51 | A, N, E, D, S, G, T, V | | X | |
| 52 | C, F, H, M, Q, P, S, K, L, R, W, E, | | | |
| | I, N, D, A | | | |
| 53 | A, Q, R, T, W, V, C, Y, K, L, N, P, | | | X |
| | F, I | | | |
| 54 | L, W, M, I, T, Y, C | | X | |
| 55 | A, F, H, M, N, S, E, T, Y, D, G, L, | | X | |
| | W, Q | | | |
| 56 | A, E, G, K, L, Q, S, R, T, Y, I, P, | | | |
| | V, N, D, M | | | |
| 57 | A, C, E, N, P, S, V, H, L, Y, F, I, | | | X |
| | M, T, G | | | |
| 58 | M, F, W, L | | X | |
| 59 | G, F, M, L, N, R, K, Q, P, S, Y, | | | |
| | W, A | | | |
| 60 | A, W, T, P, Q, Y, M, L, I, D, K, E, | | F | X |
| | R, V, S, N, G, C, | | | |
| 61 | D, N, G, E, P, H, S, Y, A, K | | X | |

TABLE 13HH
InsulinR_mb2
SKEELKERAIRLLKEGDPFEAAMLLMHLAFLTGDPRLEEVIRLLEEAFELGDPELLKEIIKLLEE
(SEQ ID NO: 785)

| | | | | | |
|---|---|---|---|---|---|
| 1 | A, E, I, G, D, Q, M, K | P, N, R, H, S, Y, L, T, V, F | | | |
| 2 | R, Q, S, G, K, P, T, N, E | H, A, V, D, F, L | | | |
| 3 | M, L, V, S, T, W, D, K, R, | G, P, I, C | | | |
| | A, H, Q, Y, E, N, F | | | | |
| 4 | G, L, F, K, M, V, Q, A | R, T, H, E, I, D, P, Y, N, C, | | | |
| | | W, S | | | |
| 5 | D, E, V, G, T, Y, K, M, L, | N, S, W, C, R | | | |
| | A, Q, F, P, I | | | | |
| 6 | R, L, H, M, T, K, C | N, A, I, Q, S, F, V | | | |
| 7 | T, S, W, G, N, L, R, I | F, D, E, A, K, M, H, Y, C, P, | | | |
| | | V, Q | | | |
| 8 | A, G, N, S, T, H, Y | D, M, Q, R, W, I, L, E | | | |
| 9 | V, I, A, S, G, T | C | | X | |
| 10 | I, T, L, V | | | | |
| 11 | D, E, Q, I, K, S, T, C, R, | F, W, G, V, H, N, L, Y | | | |
| | A, M | | | | |
| 12 | E, F, N, S, T, W, V, Y, A, | D, L, I | | | |
| | C, M, Q, R, K, H | | | | |
| 13 | M, L | | | | |
| 14 | W, Q, Y, K, T, F, R, I, S | M, H, A, V | | | |
| 15 | D, A, M, E, K, S, T, Q | N, I, L, V | | | |
| 16 | N, E, G | | | | X |
| 17 | D, N | | X | | X |
| 18 | Q, A, E, H | N, P, V, S, R, T, I, L, | X | | |
| | | C | | | |
| 19 | F, M | | X | | |
| 20 | T, S, E, G, N, A, H, Q | | X | | |
| 21 | S, A, G, C | | | X | |
| 22 | S, T, I, C | A, E, G, N, M | X | | |
| 23 | L, M | I | X | | |
| 24 | A, E, D, N, Q, S, C, M, T, | G, W, V | | X | |
| | Y, H, L, K | | | | |
| 25 | L, V, T, I | | | X | |
| 26 | T, Y, M, W, H | | X | | |
| 27 | E, D, R, Q | H, N | X | | |
| 28 | V, E, H, A, L, Y, S, T, W, | C, G | | X | |
| | Q, E, M, I | | | | |
| 29 | E, D, G, Q, N, H, S | V, A, L, C, T | | | |
| 30 | E, N, W, D, A, H, F | T, Y, G, S, M, L | X | | |
| 31 | E, L, Q, M, K, R, D | | | | |
| 32 | T, S, V, D, L, A, E | | | | |
| 33 | H, P, G, N, L, E, M, S, C, | | | | X |
| | D, A, Y, V | | | | |
| 34 | E, D, N | | | | X |
| 35 | T, V, A, S, Q, N, I, M, D, | Y, P, W, H, K, F | | | |
| | G, C, L, R, E | | | | |
| 36 | G, Y, W, Q, R, E | T, K, N, F, C, A, H | | | |
| 37 | Y, M, V, H, E, W, D, F, I, | P | | X | |
| | T, N, L | | | | |
| 38 | P, H, E, W, D | V, Y, F | | | |
| 39 | Y, F, V, D, N, H, I, E, W, | T, M, S, C, Q | | | |
| | L | | | | |
| 40 | S, T, D, H, Q, I, A, G, L, | K, R, P | | X | |
| | N, C, Y, E, M, V | | | | |
| 41 | P, S, A, N, G, Q, V, E, T, | I, L, C, F, W | | | |
| | H | | | | |
| 42 | F, I, Q, G, V, T, S, C, N, | M, R | | | |
| | P, E, Y, D, H, L, W | | | | |
| 43 | M, T, D, R, S, Q, N, W, A, | Y | | | |
| | E, H, P, F, G, K, V, C, | | | | |
| | L | | | | |
| 44 | N, T, S, C, I, V, A | M, Q, L, W | X | | |
| 45 | M, D, E, T, Q, I, N, H, C | | | | |
| 46 | W, I, L, E, V, Y | F, M, K, D, H, T | | | |
| 47 | N, E, S, G, Q | H, A, V, D, M, T | | X | |
| 48 | H, W, Y, A, G, D, P, T, V, | | | | |
| | C, E, F, N | | | | |
| 49 | L, M, I, G, P | S, V, E, W, A, Y, F, Q | X | | |
| | | T | | | |
| 50 | S, V, Y, M, H, D, G, Q, I, | K, L, C | | | |
| | F, R, P, T, N, A, E | | | | |
| 51 | G, P, Q, N | | | | X |
| 52 | Q, T, D, E, N, S, H, A, K, R, V | | | | X |
| 53 | A, D, H, M, S, R, T, Y, E, | F, P, K, L, I, V, Q | | | |
| | N | | | | |
| 54 | T, A, M, G, V, K, E, H, L, N, | | | | |
| | Y, F, D, W, I, Q, R, S, P | | | | |
| 55 | A, E, G, N, Q, S, M, D, Y, | I, L, K, H, V, W | | | |
| | P, R, F, T, C | | | | |
| 56 | E, D, M, Y, F, N | P, L, V, S, I, T | | | |
| 57 | F, Q, N, P, M, E, L, Y, I, | H, T, K, A, W, C | | | |
| | D, R, G, V, S | | | | |
| 58 | F, H, V, L, N, E, W, P, S | Y, A, D, G, I, T, C, K, R, M | | | |
| 59 | A, C, T, S, P, V, G, I | | | X | |
| 60 | E, A, S, I, G, D, F, H | Q, P, T, V, M, N | | | |
| 61 | I, N, T, L, S, V, Y, Q, E, | F, A, D | | | |
| | H, M, P, W, G, K, C, R | | | | |
| 62 | E, F, Y, M, D, S, G, T, Q | N, H, W, K, R, V | | | |
| 63 | L, M | | | X | |
| 64 | D, E, N | G | | | |
| 65 | C, D, Q, T, E, S, R, Y, A, N, | | | | |
| | M, K, L, H, F, I, V | | | | |

TABLE 13II
InsulinR_mb3
DVMEEAFTVWFQLENLAFRVDDPEQRKTVDTLSKLLQEAADRGDRELLKKVIKTARDVISKVNS
(SEQ ID NO: 786

| | | | | | |
|---|---|---|---|---|---|
| 1 | H, P, M, F, D, A, I, V, T, | S | | | |
| | L, E, N, K, Y, W | | | | |
| 2 | P, T, D, V | R, L, Y, E, M, F, Q, A | | | |
| 3 | T, I, L, N, M, F, S, H, G, Q | | X | | |
| 4 | S, Q, D, L, F, N, E, T, G | H, A | | | |
| 5 | T, M, A, D, V, K, H, E, W, | C, Y, Q, R | | | |
| | S, L | | | | |
| 6 | G, A | S | X | | |
| 7 | F, I, Y | | X | | |
| 8 | H, Y, F, A, L, N, Q, M, K | V, T, R, G, S | | | |
| 9 | L, Y, I, M, R, F | T, V, A | | X | |
| 10 | W | | X | | |
| 11 | F, I, M | | X | | |
| 12 | D, M, L, K, A, I, E, H, T, Y, | | | | |
| | S, Q, F, N, V, W | | | | |
| 13 | L, I, M | | | X | |
| 14 | N, V, T, I, E, C, Q, M, A, G | | X | | |
| 15 | G, M, Y, F, A, H, Q, E, L, | I, P, K | X | | |
| | T, S, N, D | | | | |
| 16 | I, A, M, E, T, Q, L, F, Y | S, V | | X | |
| 17 | G, W, M, R, A, H, L | | | | |
| 18 | V, D, H, M, S, W, E, F | N, I | X | | |
| 19 | C, N, Q, T, W, V, M, F, I, | D, K | | | |
| | E, H, Y, S, L, R | | | | |
| 20 | C, T, I, Q, H, M, S, L, V | Y, E | | X | X |
| 21 | G, W, S, Y, Q, T, F, I, L, | C | | | X |
| | A, V, N, R, H, E, M, K, | | | | |
| | P, D | | | | |
| 22 | T, R, K, V, H, E, L, G, D, I, | | | | X |
| | N, Y, M, Q, A | | | | |
| 23 | E, D, L, G, N, V, M, A | S, P | | | |
| 24 | S, R, P, G, K, T, I, M, N, | W, C | | | |
| | H, V, D, Y, E, A | | | | |
| 25 | M, N, F, A, S, T, V, D, P, C, | | | | |
| | W, L, K, G, H, Y, Q, E | | | | |
| 26 | G, Y, F, K, E, A, T, N, W, | D | | | |
| | I, V, S, C, H, R, M | | | | |
| 27 | R, G, I, S, N, T, M, K, H, | C | | | |
| | V, A, E, L, W, P, D | | | | |
| 28 | A, L, Q, S, K, I, V, H, R, | M, W, T | | | |
| | D, F, P, Y, G, N, E | | | | |
| 29 | C, F, M, L, I, H, V, T | Q, W | | X | |
| 30 | G, M, L, V, S, H, Y, R, W, | A, D, C, I, Q, F | X | | |
| | N, T, E, K | | | | |
| 31 | N, A, W, V, G, K, M, D, L, | H | | | |
| | E, T, F, Q, I | | | | |
| 32 | V, Q, A, W, L, Y | M, H | | X | |
| 33 | N, E, P, E, T, S, I, G, A, | V, D | | | |
| | C, L, M, R | | | | |
| 34 | N, A, E, W, V, S, Y, Q, I, | H, R | | | |
| | M, C, K, G, F | | | | |
| 35 | M, R, S, D, A, K, T, Y, V, G, I, | | | | |
| | L, Q, C, N | | | | |
| 36 | C, M | L | X | | |
| 37 | E, M, L, E, K, N, T, A, H, R, S, | | X | | |
| | Q, G, W | | | | |
| 38 | D, F, R, T, S, N, H, G, K, E, M, | | X | | |
| | V, L, C | | | | |
| 39 | S, C, M, T, A, L, Y, V, H, | E, F, Q, K | | X | |
| | N, G | | | | |
| 40 | N, S, A, D, Y | G | X | | |
| 41 | E, D, H, S, T, A | | X | | |
| 42 | W, H, N, I, A, V, K, T, M, | Y, S, F, E | | | |
| | L, D, Q, R, C | | | | |
| 43 | Q, K, L, A, P, N, D, E, M, G, S, | | | | X |
| | W, R, T, F, I, Y | | | | |
| 44 | N, E, S, L, D, K | R | | | |
| 45 | Q, I, Y, L, E, D, W, V, F, | S, A | | | |
| | H, M, R, G, P | | | | |
| 46 | Q, V, W, P, D, Y, F, L, T, C, R, | | | | |
| | I, S, E, A, G, K | | | | |
| 47 | N, I, W, F, Y, A, R, V, T, | M, Q, L, H | | | |
| | D, G, C | | | | |
| 48 | I, A, C, L | | X | | |
| 49 | A, Y, D, S, W, R, Q, H, E, M, F, | | | | |
| | K, T, G | | | | |
| 50 | M, L, T, D, H, N, Q, R, K, | V | | | |
| | E, Y, A, W, G, I | | | | |
| 51 | A, R, C, T, Q, I, L, E, F, Y, M, | | | X | |
| | K, V, N, W, S, G | | | | |
| 52 | M, W, L, S, V, C, K, I, T, A | | | | |
| 53 | D, S, F, N, Y, I, H, C, R, | L, W, M, E, V | | | |
| | K, A, G | | | | |
| 54 | D, W, C, L, I, H, Y, F, Q, M, R, | | | | |
| | T, V, S, N, A, K | | | | |
| 55 | V, S, G, T, C, A, I | | | | X |
| 56 | E, K, V, G, H, F, I, L, W, | Y | | | |
| | M, Q, R, T, D | | | | |
| 57 | S, E, R, V, E, N, I, W, Q, | G, L | | | |
| | K, T, A, M, D, Y | | | | |
| 58 | M, Q, K, Y, N, F, C, H, L, | W, R | | X | |
| | V, E, A | | | | |
| 59 | F, Y, C, M, I, Q, L, T, W, V, E, | | | | |
| | H, S | | | | |
| 60 | D, H, N, T, W, V, I, G, P, | S, K | | | |
| | L, R, Q, E, M, Y | | | | |
| 61 | H, M, F, Q, L, S, C, Y, G, T, N, | | | | |
| | K, E, W, R, D, I, A, P, V | | | | |
| 62 | C, T, I, W, A, L, G, K, M, | F, Q, H | | X | |
| | N, S, E, D, V | | | | |
| 63 | I, M, Q, S, P, R, K, W, L, | E, F, N, H, C | | | |
| | D, G, T | | | | |
| 64 | A, K, N, R, E, M, F, I, Q, | H, L, W, C, G, T | | | |
| | Y, V, S | | | | |

TABLE 13JJ
InsulinR_mb4
SELLERILRLLKEGNPEEAEEVARELLERTGDPRVEALVSLIEELLRTGDPFYELFVEMILRELR
(SEQ ID NO: 787)

| | | | | | |
|---|---|---|---|---|---|
| 1 | M, F, D, R, K, G, S, T, N, E, W, Q, | | | | |
| | H, Y, L | | | | |
| 2 | S, Y, T, D, G, M, E, A, K, F, I, R, | | | | |
| | H, Q, W, L, N, C | | | | |
| 3 | P, K, H, F, I, E, N, G, L, | A, R, D, W, Y | | | |
| | T, M, S, V, Q | | | | |
| 4 | P, V, A, L, C, M, F, I, W, | E, G, Q, T, D | | | |
| | S | | | | |
| 5 | T, N, R, E, L, D, Y, A, Q, | C, W, G | | | |
| | V, S, H, P, F, M | | | | |
| 6 | V, N, I, Q, K, D, G, R, M, | W | | | |
| | L, S, A, H, E, Y, C, F, | | | | |
| | T, P | | | | |
| 7 | I, C, L, M, T, V, A, P, Y | | | X | |
| 8 | T, A, E | G, F, V, D, N, C, M, R, H, I, | | | |
| | | L, S, P, W, K, Y, Q | | | |
| 9 | L, M, D, W, R, V, F, E, C, Q, I, Y, | | | | |
| | H, K, T, A, P | | | | |
| 10 | A, S, F, M, L, Y, H, C, N, K, E, W | | | X | |
| 11 | W, C | F, M, V, L, T, I | | X | |
| 12 | E, N, T, G, Q, K, M, D, Y, | A, W, S, V, I, C | | | |
| | R, H, L | | | | |
| 13 | Q, K, A, E, S, T, V, M, G, D, N, Y, | | | | |
| | L, F, R | | | | |
| 14 | G, N, H, K, E, D, S, L | | | | X |
| 15 | C, I, A, D, F, L, N, E, S, M, Q, Y, | | | | X |
| | V, R, W, K | | | | |
| 16 | W | M, K, G, F, A, L, Q, P, I, V, | | | |
| | | N, Y, T, H, S | | | |
| 17 | Y, L, R, K, E, V, M, G, F, W, I, S, | | | | |
| | N, H, D, Q, A, C, T, P | | | | |
| 18 | G, S, D, M, K, E, H, L, Q, | N | | | |
| | R, V, A, C, F, T, P | | | | |
| 19 | A, C | | | X | |
| 20 | A, T, G, Q, H, E, S, V, N, R, D, M, | | | | |
| | I, L, C, Y, F, K, W | | | | |
| 21 | A, N, C, I, R, M, P, Q, E, | T, G, S, W, K | | | |
| | F, D, H, L, Y | | | | |
| 22 | L, S, V, R, C, W, E, Y, M, | T, P, D | | X | |
| | A, I, G | | | | |
| 23 | A, S, G, C, K | | | X | |
| 24 | R, V, Q, F, L, C, K, A, S, I, E, N, | | | | |
| | Y, M, W | | | | |
| 25 | K, T, I, A, S, Q, E, L, R, | W | | | |
| | H, N, V, F, Y, C, G, D | | | | |
| 26 | W, L, M, F, H, V, Y | | | X | |
| 27 | W, F, Q, Y, H, L, A, S | M, N, K | | | |
| 28 | K, G, A, Q, W, N, E, Y, V, | D, C | | | |
| | S, H, R, T, F, L, I | | | | |
| 29 | N, L, A, R, H, K, C, M, E, | E, V, T | | | |
| | Q, W, D, G | | | | |
| 30 | D, T, C, N, S, G, E, Y, I, M | | | | |
| 31 | Q, N, H, A, T, G, R, E, F, S, K, Y, | | | | X |
| | W, C, M, P, I, D, V, L | | | | |
| 32 | D, S, N, E | | | | X |
| 33 | Y, A, Q, E, M, F, L, I, V, | T | X | | |
| | P, H, K, N, S, W, G, R | | | | |
| 34 | T, E, S, N, H, A, R, K, M, | Q, V, W | X | | |
| | Y, G | | | | |
| 35 | F, V, A, S, H, Y, I, M, W, G | | | X | |
| 36 | E, H, D, R, Q, S, F, N | | | | |
| 37 | A, G, N, H, D, S, R, I, M, Q | | X | | |
| 38 | M, T, L, A, S, G, C | | | X | |
| 39 | H, C, V, A | Y, K, G | | | |
| 40 | E, D, T, N, G | S, A | X | | |
| 41 | N, S, E, Q, A, T, C | D, M, L, H, Y, V, I, R | X | | |
| 42 | F, L, M, I, R | | | X | |
| 43 | N, D, Y, E | S, Q, G, K, T, H, R | | | |
| 44 | D, E, T, Q, N, A, S, V | | X | | |
| 45 | E, H, Y, W, G, K | I, A, M, L, D, F, V, N, S, P, Q | | X | |
| 46 | N, D, V, H, Y, C, G, W | F, A, S, P, T, I, L, R, K, M, E | | | |
| 47 | N, S, A, H | T, V, E, R, K, L, Q, G, I, M, | | | |
| | | F, W, Y, D | | | |
| 48 | S, N, G, T, A, Q, V, M, K, F, H, R, C | | | | |
| 49 | S, A, Q, N, G, M, E, L, T, | R, H | | | X |
| | D, K, V | | | | |
| 50 | D, G | | X | | X |
| 51 | M, P, K, T, L, D, G, N, Q, | R | X | | |
| | E, S | | | | |
| 52 | F, Y | | X | | |
| 53 | Y, H | | X | | |
| 54 | N, M, L, G, W, H, E, Q, T, | K, D, A | | | |
| | S, F, Y, R | | | | |
| 55 | Y, H, F, E, P, S, A, L, N, | G, T, R, K | X | | |
| | Q, V, D | | | | |
| 56 | L, M, F | | X | | |
| 57 | V, I, C, L, M, T, Q | | | X | |
| 58 | V, M, H, Y, T, L, D, A, F, C, W, E, | | | | |
| | G, N, S, Q, I, R | | | | |
| 59 | E, D, M | K, L, N | X | | |
| 60 | T, S | V, A, I, L, M, C, W | X | | |
| 61 | F, L, C, I, G, M, W, A, D, V, E, S | | | | |
| 62 | E, F, L, Y, V, P, M, G, T, | A, W, R, N, S, D | | | |
| | K, I, Q | | | | |
| 63 | D, E, N | G | X | | |
| 64 | L, F, W, Y, M, H, D | | | X | |
| 65 | P, Y, H, L, E, R, T, I, W, | C | | | |
| | K, F, V, D, M, Q, S, G, | | | | |
| | N, A | | | | |

TABLE 13KK
InsulinR_mb5
DEELMELVYEAVEKNDPELLFEAWMELASLLDETGDPKIEEALGLLQQVDGGNPDAGRRIKELFK
Optimized SEQ ID NO: 788

| | | | | | |
|---|---|---|---|---|---|
| 1 | K, T, E, D, Q, H, L, P, S, | | | | |
| | N, R, V, F, G, C, W, M, A, I | | | | |
| 2 | W, A, R, P, L, K, H, E, T, | G, V | | | |
| | D, C, Y | | | | |
| 3 | P, A, Y, E, M, L, C, S, I, | | | | |
| | F, K, Q, G, T, R, N, W, D, H, V | | | | |
| 4 | Q, D, M, E, K, R, G, N, H, | | | | |
| | S, Y, L, I, V, A, F, C | | | | |
| 5 | S, R, P, M, K, Q, E, G, D, | L | | | |
| | W, T, A, H, N, I, V, F | | | | |
| 6 | Q, V, D, R, I, P, G, A, E, F, | | | | |
| | N, Y, M, S, T, H, W, L, K | | | | |
| 7 | W, I, S, F, A, R, V, H, T, | C, L, Y, G, E, N | | | |
| | Q, K | | | | |
| 8 | A, V, T, C, M, I, L, F, S | | | X | |
| 9 | M, A, I, T, Q, L, K, F, S, | D, C, H, G | | | |
| | N, E, W, Y, R, V | | | | |
| 10 | S, Y, P, D, R, E, A, F, W, K, | | | | |
| | V, H, G | | | | |
| 11 | G, S, A, P, V, R, M, Y, K, | | | X | |
| | H, T, N, C, F, I, D, Q, L | | | | |
| 12 | I, V, Y, M, C, N, S, T, F, | | | X | |
| | A, Q, L | | | | |
| 13 | I, T, E, D, R, V, W, Q, N, | | | | |
| | L, K, A, Y, C, G, M, H, F, S | | | | |
| 14 | I, C, M, K, Y, R, H, L, Q, | | | | X |
| | E, T, N, W, P, A, D, S, F, G | | | | |
| 15 | G, A, S, N, E, V, Q, H, D, | | | | X |
| | P, R, T, M, K, L, C | | | | |
| 16 | E, V, D, N, S, H, I, A, K, | | | | X |
| | Q, M, T, R | | | | |
| 17 | S, T, H, K, F | N, P, A, Q | X | | |
| 18 | M, L, F, Y | V, P, S, I, R, E, N, Q, T, D | | | |
| 19 | Y, M, A, F, E, V, L, T, D, | | | | |
| | I, G, N, P, R, S | | | | |
| 20 | M, Q | L, G | X | | |
| 21 | F, V, M | | X | | |
| 22 | D | N, R, A, E, P, Q, V, G, | X | | |
| | | H, M | | | |
| 23 | C, L, A, V, K, Y, S, M, I, | | | | X |
| | G, F, W, R, T, Q | | | | |
| 24 | W, Y | | X | | |
| 25 | I, M, V, A, F, L, C, T | | X | | |
| 26 | A, G, S, D, N, K, M, E, F, | T | | | |
| | Q, Y, C, R, W, H, L, I, | | | | |
| | V | | | | |
| 27 | G, A, F, S, C, M | T, L, W, Y, H | | X | |
| 28 | C, A, T, V, W, Q, M, S, E, | | X | | |
| | I, G | | | | |
| 29 | G, P, L | A, S, C, V, D, R, M, E | X | | |
| 30 | C, R, G, I, Y, K, S, F, Q, | | | | |
| | L, D, W, M, T, E, A, V, N | | | | |
| 31 | A, G, H, S, Q, N, L, D, T, | | X | | |
| | I, Y, M, W, V | | | | |
| 32 | P, D, Y, N, A, G, M, T, S, | | X | | |
| | E, V, Q, I | | | | |
| 33 | D, E, G, P, A, N, H, F, V, | C | | | |
| | T, Q, Y, M, S, R, I, K, | | | | |
| | W | | | | |
| 34 | D, Y, N, E, G, H, Q, A, T | K, I, S | | | |
| 35 | D, Q, S, T, P, E, G, N, I, | H | | | X |
| | W | | | | |
| 36 | D, S, P, K, E, Q, T, A, Y, | | | | X |
| | H, N, G, I, R | | | | |
| 37 | E, P, R, K, A, I, G, M, H, | | | | |
| | Q, C, Y, T, D, V, L, S, W, F, N | | | | |
| 38 | V, R, Y, A, I, H, Q, G, K, | | | | |
| | E, T, D, W, M, C, F, L | | | | |
| 39 | I, N, Q, R, M, T, F, C, L, | | | X | |
| | Y, V, W, H, K, D, E | | | | |
| 40 | E, M, S, R, Y, T, P, F, D, | | | X | |
| | V, N, L, G, A, Q, K, H, I, | | | | |
| | W | | | | |
| 41 | D, E, M, S, K, H, T, N, L, | | | | |
| | I, P, Y, R, V, A, G, W, F, Q, C | | | | |
| 42 | G, A, M, I, T, L | E, C | | X | |
| 43 | M, Q, L, T, I, V, A, S, F | | | | |
| 44 | W, I, V, S, K, F, L, D, N, | Q, P, C, G | | | |
| | A, E, M, H, R, T, Y | | | | |
| 45 | D, L, W, Y, A, I, F, E, H, | | | | |
| | N, S, Q, C, M, V | | | | |
| 46 | M, L, I, T, V, K, C, Y, F | | X | | |
| 47 | V, E, P, N, Q, M, T | R, I, A | X | | |
| 48 | E, Q, L, D, A, G, W, K, V, | | | | |
| | H, I , C, R, S, N, T, M, P, Y | | | | |
| 49 | M, L, V, E, F, T, H, I, Q, | | | X | |
| | N, D, W | | | | |
| 50 | D, Y, E | | X | | |
| 51 | E, D | G, A, H, W, N, R, L, Q, S | | | X |
| 52 | G, Y, P, K, H, A, Q, R, W, | | | | X |
| | D, E, S, M, L, N, F, V, C | | | | |
| 53 | N, D, V, Y, C, S, K, A, H, I, | | | | X |
| | E, R, F, Q, G, W, L, M, P | | | | |
| 54 | E, D, T, L, S, C, Y, P, G, Q, | | | | |
| | K, I, N, V, H, A, F, M, R, W | | | | |
| 55 | I, M, Y, F, C, D, E, T, A, S, | | | | |
| | R, Q, H, L, K, W, V, G | | | | |
| 56 | A, T, I, S, N, G, P, V, E, D, | | | X | |
| | Q, L, M | | | | |
| 57 | S, H, G, E, I, P, A, T, F, | Y, Q, R, M, C, N, L | | | |
| | K | | | | |
| 58 | G, W, V, F, H, R, T, L, P, | | | | |
| | Y, I, D, E, A, C, N, S, Q | | | | |
| 59 | D, M, R, I, G, N, F, C, S, | | | | |
| | W, Y, A, T, V, K, Q, H, E, L | | | | |
| 60 | V, I, A, Y, W, L, S, T, C, F, | | | X | |
| | M, G | | | | |
| 61 | F, D, H, I, M, Y, V, K, W, N, | | | | |
| | E, T, R, G, Q, P, L | | | | |
| 62 | Y, V, N, L, W, S, I, G, H, | | | | |
| | E, Q, T, K, R, C, F, D, M, A, P | | | | |
| 63 | G, V, N, E, I, L, H, D, C, | | | | |
| | T, Y, Q, F, W, A, K, S, P, R, M | | | | |
| 64 | L, V, Q, I, C, R, S, F, K, | E | | | |
| | Y, H, T, N | | | | |
| 65 | G, P, Y, V, S | Q, A, N, K, T, D, W, I, E, F, L | | | |

TABLE 13LL
InsulinR_mb6
DPFTAMELVARVFELALEKNDEELQKEAETLMEIVIKAAQNNDEEKVKEVIKRAKELLSKS
(SEQ ID NO: 790)

| | | | | | |
|---|---|---|---|---|---|
| 1 | D, E, I, L, Y, G, S, P, N | R | | | |
| 2 | P, G, A | S | X | | |
| 3 | F, W | | X | | |
| 4 | E, H, N, S, T, Q, W, R, V, F, | | | | |
| | L, C, M, A, K | | | | |
| 5 | A, C, S, V, G, L, T | I | | X | |
| 6 | L, M, V | | X | | |
| 7 | E, P, G, Q | | X | | |
| 8 | L, E, Y, D, N, W, I, G, M, T, | | | | |
| | Q, H, A, F, S, V, C | | | | |
| 9 | M, V, A, I, T, G, C, S, L, F, Q | | | X | |
| 10 | G, A | S | X | | |
| 11 | D, L, R, N, P, M, S, B, C, V, | | | | X |
| | H, K, W, T | | | | |
| 12 | G, M, L, N, T, V, Q, A, F, | D | | X | |
| | Y, C, I, S | | | | |
| 13 | F, L, I, A, Q, E, T | | X | | |
| 14 | E, D | | X | | |
| 15 | D, M, L, I, Y, N, P, A, S, | G | | | |
| | K, V, C, R, W, F, H | | | | |
| 16 | E, A, Q, G, M, L, W, S, N | V | | X | |
| 17 | D, L, V, E, I, M, Y, Q, G, F, | | X | | |
| | A, H, P, N, T | | | | |
| 18 | E, P, D, G, K, T, V, Y, A, Q, | | X | | |
| | W, S | | | | |
| 19 | L, T, R, Y, K, D, P, M, H, S, | | | | X |
| | C, N, F, W, Q, V, E, I, A, G | | | | |
| 20 | E, I, L, N, S, A, V, Y, M, Q, | | | | X |
| | H, W, D, P, G | | | | |
| 21 | E, F, Q, D, R, G, M, S, T, | | | | X |
| | K, P, C, I, H | | | | |
| 22 | A, D, E, N, W, V, Y, F, G, | T | | | |
| | L, S, I, Q, R | | | | |
| 23 | G, F, E, W, H, R, Y, Q, M, D, | | | | |
| | S, V | | | | |
| 24 | C, I, W, Y, L, V, M, A, S, F, | | | | |
| | H, E, D, P, N, T | | | | |
| 25 | E, D, N, Q, H, P, A, M, S, G, | | | | |
| | L, Y, C, I, W, T | | | | |
| 26 | L, V, W, T, S, P, N, M, K, | Q | | | |
| | Y, D, C, R, E, A, H, I, | | | | |
| | F, G | | | | |
| 27 | A, H, R, T, E, L, S, W, Y, | Q | | | |
| | C, G, M, N, D Q | | | | |
| 28 | P, A, G, E, D, C, M, T, N | | | X | |
| 29 | E, Q, D | | X | | |
| 30 | A, L, Y, F, M, T, H, K, N, W, | | | | |
| | I, G, V, S, C, D, Q, R | | | | |
| 31 | A, C, V, L, W, G, F, E, T, | H | | X | |
| | D | | | | |
| 32 | M, L, V | | X | | |
| 33 | E, D, N | | | | |
| 34 | L, I, D, A, R, P, E, Q, S, | | | | |
| | G, F, T, N, C | | | | |
| 35 | I, L, V, T, M, S | H | | X | |
| 36 | L, I, G, Y, S, W, D, T, E, Q, C | | X | | |
| 37 | K, C, G, I, M, L, Q, S, V, | F | | | |
| | T, N, H , P, E, Y, W, A, | | | | |
| | R | | | | |
| 38 | C, E, Q, A, G, I, L, N, S, | W | | X | |
| | M, D, R, F, H, V, Y | | | | |
| 39 | A, S, G, N | | X | | |
| 40 | Q, D, T, H, E, P, E, Y, W, | I | X | | |
| | M, V, N, A | | | | |
| 41 | N, E, W, F, P, G, V, S, M, A, | | X | | X |
| | C, K, Y, T, R, D, H, Q, L | | | | |
| 42 | I, L, P, N, A, T, V, M, S, W, | | X | | X |
| | D, Y, F | | | | |
| 43 | D, T, S, V, P, A, M, E, I, F, | | | | |
| | W, G, R, N, Q, H, Y, K, C, L | | | | |
| 44 | E, V, T, S, F, Y, L, N, A, I, | | | | |
| | W, Q, R, P, G, M, D, H, C | | | | |
| 45 | E, V, I, M, L, R, Q, D, N, P, | | | | |
| | S, H, G, Y, C, T, W, F | | | | |
| 46 | A, W, R, S, P, Y, H, K, E, M, | | | | |
| | G, C, D, V, L, N, I, F, Q, T | | | | |
| 47 | P, V, L, I, F, A, G, T, C, N, S | | | | |
| 48 | G, M, N, P, K, E, L, W, A, | I | | | |
| | D, R, F, H, Y, C, S | | | | |
| 49 | L, V, T, P, K, H, E, I, G, D, | | | | |
| | N, F, S, C, Y, W, A, R, M, Q | | | | |
| 50 | V, M, L, C, A, Q, S, R, E, | P | | X | |
| | G, W | | | | |
| 51 | L, P, I, V, C, S, Y, N, W, K, | | | | |
| | M, T | | | | |
| 52 | N, T, P, Q, M, K, H, V, G, R, | | | | |
| | A, I, L, Y, D, C, W | | | | |
| 53 | K, F, G, R, D, T, C, M, V, H, | | | | |
| | N, P, Q, S, I, E, Y, A | | | | |
| 54 | A, E, L, S, T, Q, I, D, V, G, | | | X | |
| | N, Y, C, H, M | | | | |
| 55 | A, E, D, W, K, H, V, S, Q, P, | | | | |
| | M, I, C, Y, R, F | | | | |
| 56 | E, T, P, Q, I, W, M, A, H, K, | | | | |
| | C, Y, F, R, S, V, G, L, D, N | | | | |
| 57 | A, S, P, L, D, C, R, I, N, G, | | | X | |
| | T, K, V, W, H, Y, Q | | | | |
| 58 | W, L, V, D, A, E, G, Y, H, I, | | | | |
| | P, S, F | | | | |
| 59 | M, L, N, P, T, S, I, F, Y, A, | | | | |
| | R, G, H, Q, V, E | | | | |
| 60 | K, N, W, F, S, L, E, I, V, | Q | | | |
| | C, H, Y, P, G, A, R, D | | | | |
| 61 | S, C, W, P, T, F, K, N, D, Y, | | | X | |
| | G, I, V, E, L, M, Q, A, R | | | | |

TABLE 13MM
InsulinR_mb7
DPFTAMELVARVFELALEKNDEELQKEAETLMEIVIKAAQNNDEEAVKEVIKRAKELLSKS
(SEQ ID NO: 791)

| | | | | |
|---|---|---|---|---|
| 1 | D, E, G, F, S, P, Q, Y, N, M, K | | | |
| 2 | P, G, N, A | X | | |
| 3 | F, W | X | | |
| 4 | T, E, N, S, Q, I, E, A, H, K, | | | |
| | Y, V, D, L, R, M, W, C | | | |
| 5 | A, G, C, M, L, S, T, Q, F | | X | |
| 6 | L, M | X | | |
| 7 | E, P, G, D, N | X | | |
| 8 | G, L, E, Q, D, M, W, H, F, V, | | | |
| | C, N, I | | | |
| 9 | V, T, C, A, F, I, N, Q, Y, S, | | X | |
| | G, L, M | | | |
| 10 | A, G, S, R | X | | |
| 11 | A, V, W, T, S, N, L, C, I, R, | | | |
| | Y, D, Q, K, F, P, H, G, E | | | |
| 12 | G, N, S, V, I, M, Q, Y, W, C, | | X | |
| | F, L, T, H, A | | | |
| 13 | F, I, Y, L, W, V, S, M | X | | |
| 14 | E, D | X | | |
| 15 | L, W, F, K, Q, Y, C, R, H, S, | | | |
| | T, V, A, G, D, M, E, N, P, I | | | |
| 16 | G, N, A, Q, C, E, S, D, M | | X | |
| 17 | I, H, N, Y, L, M, V, F, E, S, A, | X | | |
| | D, Q, G | | | |
| 18 | E, F, L, P, Q, Y, M, H, A, V, G, | X | | |
| | D, S | | | |
| 19 | K, C, D, G, H, N, I, T, F, M, W, | | | X |
| | A, P, V, E, L, Y, R | | | |
| 20 | N, Q, Y, I, F, E, K, H, G, P, S, | | | X |
| | W, T, R, L, D, M, V | | | |
| 21 | N, G, D, A, H, W, I, C, L, Y, Q, | | | X |
| | R, E, M, T, S, P, V | | | |
| 22 | E, V, F, L, P, T, D, M, W, R, C, | | | |
| | Y, S, N, Q, H, A, G, I | | | |
| 23 | A, R, Y, M, E, D, I, V, G, Q, S, | | | |
| | T, N, H, W, P, K, L, F | | | |
| 24 | L, W, E, P, T, N, I, A, D, H, S, | | | |
| | M, Y, G, K, C, R | | | |
| 25 | D, L, Q, Y, T, I, H, N, M, E, V, | | | |
| | S, G, K, P | | | |
| 26 | A, V, P, Y, K, D, E, C, I, H, Q, | | | |
| | W, G, L, N, T, S, M | | | |
| 27 | A, V, Y, K, E, G, D, W, I, P, H, | | | |
| | L, M, N, F, T, R, Q, S | | | |
| 28 | A, D, P, G, S, V, E, L, N, M, Q, | | X | |
| | I, Y, H | | | |
| 29 | D, E, N, G | X | | |
| 30 | A, S, P, F, T, M, G, I, Y, C, | | | |
| | H, V, W, L, D, K, Q, N, E, R | | | |
| 31 | G, L, W, E, F, H, I, Q, C, A, | | X | |
| | N, K, M, S, V, D | | | |
| 32 | M, V, I, L, W | X | | |
| 33 | D, E, N, S | | | |
| 34 | I, E, Y, M, S, V, N, W, D, K, P, | | | |
| | F, R, A, C, H, Q, T | | | |
| 35 | V, I, M, L, E, C, F, D, N, H | | X | |
| 36 | N, T, I, S, G, A, V, M, D, L, Q | X | | |
| 37 | K, R, G, F, Q, L, Y, T, S, M, N, | | | |
| | A, E, D, W, H, I, P, V | | | |
| 38 | C, S, N, L, E, A, H, M, Y, Q, T, | | X | |
| | D, G, F, I, V, R | | | |
| 39 | G, S, A | X | | |
| 40 | Q, V, T, N, Y, H, P, M, E, S, | X | | |
| | D, A, K, F, L, I , G, W | | | |
| 41 | A, V, T, S, H, N, E, F, L, D, P, | X | | X |
| | Y,I , K, G, M, Q, C | | | |
| 42 | F, I, P, N, M, L, W, Y, S, D, V, | X | | X |
| | E, H, G | | | |
| 43 | D, V, K, Q, R, T, Y, I, M, G, N, | | | X |
| | E, H, S, P, C, A, L, F | | | |
| 44 | A, V, T, R, Q, M, E, I, H, F, P, | | | |
| | L, W, G, D, Y, S, N, K, C | | | |
| 45 | A, V, W, T, R, Q, N, L, H, F, K, | | | |
| | E, M, G, S, P, D, C, I, Y | | | |
| 46 | A, E, W, R, S, P, H, K, Y, C, G, | | | |
| | M, D, V, L, N, I, F, Q | | | |
| 47 | A, G, V, S, C, M, T, H, L, I, P | | | |
| 48 | K, V, T, G, I, R, M, P, F, E, | | | |
| | A, S, L, D, W, C, N, Y, H, Q | | | |
| 49 | E, R, K, P, A, I, D, V, N, T, | | | |
| | L, F, S, G, C, H, Q, W, M, Y | | | |
| 50 | D, L, P, V, A, C, Q, H, M, N, | | X | |
| | S, T, Y, G | | | |
| 51 | N, I, G, S, V, W, D, H, L, A, C, | | | |
| | P, F, Y, Q, E, R, K, T | | | |
| 52 | A, T, R, P, F, K, Q, D, C, G, | | | |
| | L, Y, N, S, M, V, E, H, I, W | | | |
| 53 | R, T, E, D, P, I, H, K, V, N, M, | | | |
| | S, W, A, Q, F, C, G, L | | | |
| 54 | A, E, D, H, P, Y, R, M, W, G, V, | | X | |
| | N, Q, I, T, K, C, L | | | |
| 55 | N, V, S, L, H, K, F, G, D, P, T, | | | |
| | W, M, I, Y, R, C, Q, A | | | |
| 56 | A, R, S, P, L, M, E, F, G, K, Q, | | | |
| | I, T, Y, N, W, V, D | | | |
| 57 | C, Q, L, R, K, W, M, A, F, T, E, | | X | |
| | H, S, Y, V | | | |
| 58 | V, L, S, I, M, N, K, F, H, Q, Y, | | | |
| | C, W, E, P | | | |
| 59 | L, R, P, Q, K, S, D, H, I, F, G, | | | |
| | N, W, M, C, Y, T | | | |
| 60 | K, R, S, Y, N, T, D, Q, E, P, I, | | | |
| | H, A, F, W, M, V, C, L, G | | | |
| 61 | L, V, P, N, I, S, E, Y, H, C, D, | | X | |
| | R, A, T, W, Q, K, G, F | | | |

TABLE 13NN
InsulinR_mb8
DPFTAMELVARVFELAVEKNDEELAKEAETIMEIVMKAAENNDEEAVKEVIKRAKELLSKS
(SEQ ID NO: 792)

| | | | | |
|---|---|---|---|---|
| 1 | A, V, T, Q, Y, L, I, D, G, P, F, | | | |
| | H, M, K, E, S, W, R | | | |
| 2 | P, I, G, A | X | | |
| 3 | W, F | X | | |
| 4 | A, V, W, S, N, M, L, E, K, T, Q, | | | |
| | C, P, R, Y, F, H, D | | | |
| 5 | A, T, M, C, G, F, I | | X | |
| 6 | M, I | X | | |
| 7 | H, S, E, N, A, D, M, T, L, F, G, | X | | |
| | P | | | |
| 8 | G, M, N, S, R, Y, L, K, E, Q, C, | | | |
| | A, D, H, F, T | | | |
| 9 | A, V, I, L, S, F, Y, C, M, G, W, | | X | |
| | T | | | |
| 10 | G, A, S, W, C | X | | |
| 11 | R, D, H, K, M, S, N, Q, W, C, E, | | | |
| | A, Y, P, F, T, L, V | | | |
| 12 | A, C, F, I, S, T, W, V, G, Y, M, | | X | |
| | N, L, Q | | | |
| 13 | C, Y, F, W, T, M, A, D, V, L, I, | X | | |
| | H | | | |
| 14 | E, T, D, A, G, Q, Y, S, I | X | | |
| 15 | A, R, Q, N, Y, H, L, G, D, C, I, | | | |
| | K, F, E, T, S, W | | | |
| 16 | C, W, T, S, Y, A, G, E, L, V, Q, | | X | |
| | F, N, K, R, D, I, P | | | |
| 17 | A, I, H, M, L, P, W, Y, V, T, Q, | X | | |
| | E, C, N, G, D | | | |
| 18 | N, W, P, Y, L, H, E, D, C, I, R, | | | |
| | G, F, A, V, M, T, Q, S | | | |
| 19 | A, T, S, P, K, D, F, I, H, Y, R, | | | X |
| | N, V, G, C, L, W, Q, M | | | |
| 20 | N, E, P, Y, H, F, Q, A, M, I, D, | | | X |
| | W, R, K, G, S, T | | | |
| 21 | L, V, S, P, D, G, F, K, R, T, Y, | | | X |
| | W, N, I, C, H, M, Q, A, E | | | |
| 22 | A, V, G, N, D, E, L, R, Q, T, K, | | | |
| | P, S, H, Y, C, M | | | |
| 23 | L, V, R, Q, N, Y, E, H, T, K, I, | | | |
| | W, M, D, S, A, F, G | | | |
| 24 | L, C, W, S, F, A, D, K, E, I, M, | | | |
| | T, N, G, V, Q, H | | | |
| 25 | C, W, R, S, P, Q, Y, L, A, H, I, | | | |
| | F, E, M, N, V, T, D, K | | | |
| 26 | E, L, S, K, H, R, V, N, C, G, A, | | | |
| | Y, T, M | | | |
| 27 | A, T, R, S, Q, M, N, I, F, E, W, | | | |
| | L, H, K, D, G, P, V, Y | | | |
| 28 | F, I, A, Q, S, L, N, V, G, D, M, | | X | |
| | Y, C, R, T | | | |
| 29 | N, E, G, C, T, Y, L | X | | |
| 30 | A, W, Q, N, H, M, D, T, E, C, V, | | | |
| | I, K, P, R, S, F, L, G, Y | | | |
| 31 | E, F, Q, R, Y, L, D, I, C, G, A, | | X | |
| | V, S, H, N, K | | | |
| 32 | M, I, V, W, L, Y | X | | |
| 33 | D, E, M, T, A, H, L, G, I , K, | X | | |
| | Q, Y, F | | | |
| 34 | I, C, E, D, W, Q, H, K, M, A, P, | | | |
| | V, N, S, Y, L, R, F, T, G | | | |
| 35 | E, V, F, I, T, Q, L, M, H | | X | |
| 36 | G, L, S, T, V, M, I, N, W, D, A, | X | | |
| | Q | | | |
| 37 | C, M, T, K, S, E, R, V, G, W, F, | | | |
| | D, H, Q, A, L | | | |
| 38 | C, N, H, L, D, A, K, M, Q, S, T, | | X | |
| | R, I, Y, V, P, F, E | | | |
| 39 | N, A, S, G | X | | |
| 40 | D, T, E, N, P, Y, G, Q | X | | |
| 41 | N, V, R, D, G, P, L, H, M, Q, I, | X | | X |
| | F, S, T, K, Y, C, E, A, W | | | |
| 42 | N, D, F, I, H, V, Y, P, W, R, E, | | | X |
| | G, Q, L, S, T, A | | | |
| 43 | A, T, R, S, Q, N, M, L, H, I, C, | | | X |
| | D, G, W, V, K, E, F, Y, P | | | |
| 44 | L, V, W, R, S, Q, N, Y, E, G, D, | | | |
| | M, H, F, P, T, C, A, K, I | | | |
| 45 | A, V, W, R, S, P, Y, M, L, F, G, | | | |
| | K, E, D, Q, N, I, H, C | | | |
| 46 | C, R, P, N, K, M, F, D, E, I, | | | |
| | A, T, H, W, G, V, S, L | | | |
| 47 | C, L, P, V, S, M, T, H, G, F | | | |
| 48 | A, S, N, K, I, Q, G, M, C, D, Y, | | | |
| | H, T, P, V, F, E, W, L | | | |
| 49 | A, V, W, P, Q, K, E, I, F, G, M, | | | |
| | N, S, H, Y, L, C, R, D, T | | | |
| 50 | M, P, Q, L, I, V, D, G, E, C, S, | | X | |
| | F, Y, W, A, N, T, H | | | |
| 51 | A, T, P, N, I, F, V, D, W, S, R, | | | |
| | H, M, Y, K, G, L, C, E | | | |
| 52 | N, V, T, Q, Y, M, K, E, C, L, A, | | | |
| | R, H, P, W, E, D, S | | | |
| 53 | R, C, E, G, V, T, K, S, P, I, Y, | | | |
| | A, W, M, L, N, F, H | | | |
| 54 | A, F, G, L, E, Y, Q, C, K, T, V, | | X | |
| | R, I, H, N, M, S | | | |
| 55 | K, T, R, G, I, Q, Y, F, P, N, L, | | | |
| | D, A, S, M, W, C, V, E, H | | | |
| 56 | M, S, P, V, I, E, R, L, N, A, C, | | | |
| | G, K, D, H, T, F, W | | | |
| 57 | A, T, R, N, Y, L, D, E, F, H, K, | | X | |
| | M, P, W, V, S, C, I, G, Q | | | |
| 58 | M, W, R, P, N, K, L, D, I, E, Q, | | | |
| | S, F, A, G, C, Y, T, V, H | | | |
| 59 | S, W, E, P, G, Q, I , K, M, R, F, | | | |
| | H, L, N, D, C, T, A, Y | | | |
| 60 | M, P, S , R, K, Y, I, N, V, H, | | | |
| | W, G, F, L, Q | | | |
| 61 | A, R, Q, Y, M, K, S, G, H, N, V, | | X | |
| | L, F, C, D, P, E, W, T | | | |

TABLE 13OO
InsulinR_mb9
DPFTAMELVARVFELAVEKNDEELAKEAETLMEIVTKAAQNNDEEAVKEVIKRAKELLSKS
(SEQ ID NO: 793)

| | | | | |
|---|---|---|---|---|
| 1 | V, D, M, Y, P, F, Q, R, E, L, T | | | |
| 2 | P, G, I, A | X | | |
| 3 | F, W | X | | |
| 4 | A, S, N, Y, K, H, M, F, E, T, | | | |
| | V, R, W, C, Q, P, G, I, D, L | | | |
| 5 | A, M, T, L | | X | |
| 6 | L, M | X | | |
| 7 | Q, E, P, D, T, Y, A, N, S, G, | X | | |
| | V, K | | | |
| 8 | H, V, L, K, F, A, I, T, Q, G, | | | |
| | W, S, Y | | | |
| 9 | V, L, A, Y, I, F, M, W, S | | X | |
| 10 | A, F, W, S, G, L | X | | |
| 11 | R, E, D, W, T, V, Y, A, G, N, | | | |
| | L, K, C, F, H, M, I, Q, S | | | |
| 12 | C, V, Q, I, N, T, F, A, G, Y, | | X | |
| | S | | | |
| 13 | M, W, F, Y, C, D, N, T, A | X | | |
| 14 | E, D, T | X | | |
| 15 | L, C, G, I, K, V, S, R, W, D, | | | |
| | F, T, H, E, N, Q, M, Y, A | | | |
| 16 | H, A, F, E, L, C, I, Q, S, K, | | X | |
| | G, W, D, V, M, N | | | |
| 17 | F, M, L, W, V, Y, N, T, E, S, | X | | |
| | R, G, A, I, P | | | |
| 18 | E, V, Y, M, L, F, W, Q, S, P, | | | |
| | D, N, I, R, G, T, C | | | |
| 19 | K, R, P, Q, Y, L, E, V, A, W, | | | X |
| | G, D, S, F, N, M, I, T | | | |
| 20 | N, E, D, G, F, V, P, Y, A, M, | | | X |
| | R, L, S, H, T, I, K | | | |
| 21 | A, V, W, S, N, M, D, G, E, L, | | | X |
| | R, F, T, H, Y, I, K, Q, C | | | |
| 22 | E, Y, D, F, A, I, Q, G, S, W, | | | |
| | M, R, T, N, H, K, C | | | |
| 23 | E, W, T, G, S, Q, H, K, M, Y, | | | |
| | A, P, N, I, L, C, D, R, F, V | | | |
| 24 | A, V, W, Q, N, K, L, C, Y, I, | | | |
| | G, M, D, S, H, T, P, F | | | |
| 25 | L, V, T, S, M, A, G, D, I, E, | | | |
| | P, N, H, R, C, Q, Y, F | | | |
| 26 | A, V, R, P, M, H, K, L, D, E, | | | |
| | I, W, C, T, N, Y, S, G, F | | | |
| 27 | A, R, P, L, M, E, F, D, K, S, | | | |
| | G, N, W, Q, V, H, T | | | |
| 28 | L, A, V, C, D, N, P, I, G, W, | | X | |
| | M, T, R, K | | | |
| 29 | E, F, D, N, G, I, Y | X | | |
| 30 | A, I, H, K, L, N, Q, P, S, T, | | | |
| | W, D, R, F, C, G | | | |
| 31 | C, F, N, S, V, L, G, H, E, Y, | | X | |
| | Q, W, T, D, M | | | |
| 32 | M, W, F, L, I | X | | |
| 33 | A, N, S, T, E, W, L, C, M, H, | | | |
| | Y, V, Q, I, K | | | |
| 34 | N, T, R, S, P, Q, I, H, G, D, | | | |
| | E, M, V, L, W, Y, K, C | | | |
| 35 | V, T, L, F, M, I, C | | X | |
| 36 | T, A, G, I, V, W, S, N, M | X | | |
| 37 | K, E, I, T, Y, Q, C, R, L, D, | | | |
| | V, W, N, F, H, M, P, S, A | | | |
| 38 | C, I, L, A, N, T, V, S, K, H, | | X | |
| | G, R | | | |
| 39 | A, G, S | X | | |
| 40 | A, V, G, Q, E, D, W, I, Y, R, | X | | |
| | K, P, L, F, S, T, H | | | |
| 41 | N, C, E, I, W, M, T, R, S, K, | | | X |
| | V, A, F, H, Q, L, P, Y | | | |
| 42 | M, T, S, L, H, N, F, W, V, Y, | | | X |
| | G, C, Q, P, D, I, E, K | | | |
| 43 | A, S, P, N, V, M, L, H, G, K, | | | X |
| | D, C, F, T, I, Q, W | | | |
| 44 | A, R, S, N, M, K, L, F, G, C, | | | |
| | H, E, T, Q, P, W, I, D, V, Y | | | |
| 45 | A, V, W, T, R, Y, L, E, D, K, | | | |
| | G, H, I, F, N, P, C, Q, S | | | |
| 46 | C, W, S, Y, K, H, A, D, E, V, | | | |
| | M, G, I, Q, L, N, R, P, T, F | | | |
| 47 | C, I, V, L, A, P, N, W, M, T, Q | | | |
| 48 | K, W, G, H, Y, P, F, C, S, I, | | | |
| | M, R, A, D, E, Q, V, N | | | |
| 49 | L, V, R, Q, N, M, E, I, G, C, | | | |
| | E, D, T, P, W, H, A, Y | | | |
| 50 | I, L, V, A, T, S, M, P, G, N, | | X | |
| | Q, C, D | | | |
| 51 | M, P, I, H, D, E, R, W, Q, A, | | | |
| | L, Y, V | | | |
| 52 | A, C, L, N, T, V, K, G, I, P, | | | |
| | R, H, Q, M, S, W | | | |
| 53 | A, T, S, N, H, R, F, G, D, E, | | | |
| | Q, V, I, Y, M, L, W, P, K | | | |
| 54 | V, A, T, C, H, Y, S | | X | |
| 55 | M, W, R, L, Y, H, K, G, C, D, | | | |
| | N, E, I, F, Q, V, A, S | | | |
| 56 | L, V, W, R, S, N, M, E, H, C, | | | |
| | E, D, Y, K, T, A, G, Q, P, I | | | |
| 57 | A, K, N, D, L, T, C, S, V, Y, | | X | |
| | Q, H, R, W, M, P, I, F, G | | | |
| 58 | A, E, F, P, S, L, I, V, H, G, | | | |
| | K, R, Q, M, D, W | | | |
| 59 | S, V, Q, I, E, R, P, L, Y, A, | | | |
| | H, K, M, D, W, N, F, G, C | | | |
| 60 | K, T, D, H, Y, S, V, G, N, F, | | | |
| | L, I, R, A, P, C, M, E | | | |
| 61 | S, D, V, W, R, Q, Y, N, E, L, | | | |
| | E, M, T, H, I, P, G, A | | | |

TABLE 13PP
InsulinR_mb10
DDHAQAQLVKKLLKLWQDQGDDFYALLAVEAAANERVKKYLKKYYPEVYTLLEQVKRTLEKKRS
(SEQ ID NO: 794)

| | | | | | |
|---|---|---|---|---|---|
| 1 | D, T, I, E, H, P, M, Y, V, N, | | X | | |
| | A, L, Q, S, F, W | | | | |
| 2 | Q, Y, D, H, I, L, T, E, M, N, | | | | |
| | F, V, W, G, R, C, A, P | | | | |
| 3 | L, T, K, V, Q, D, N, I, Y, | S | | | |
| | F, M, H, A, W, E, R, P, | | | | |
| | C | | | | |
| 4 | I, E, H, T, F, N, D, M, A, | P, S | X | | |
| | V, Y, K, L, Q, G, W | | | | |
| 5 | E, S, D, A, N, T, Q, G | | X | | |
| 6 | F, T, Y, I, S, A, V, L, G, | M | | | |
| | W, C, P, R, D | | | | |
| 7 | E, D, N, F, G, K, Q, S, V, | A | | | |
| | W, H , R, T | | | | |
| 8 | E, Q, D, V, Y, L, T, S, F, M | | X | | |
| 9 | A, T, V, I, Y, Q | S | | X | |
| 10 | M, P, A, F, W, N, L, Y, K, | E, S, H | | | |
| | Q, D, G | | | | |
| 11 | E, D, S, G, M, K, N, T | Q, P, R, H, Y, A | | | |
| 12 | M, L, I, V | | X | | |
| 13 | F, L, V, I, Y, N, T | | | X | |
| 14 | W, N, V, D, K, S, G, R, L, | E | | | |
| | Y, C, T, A, Q | | | | |
| 15 | K, D, F, T, M, Y, W, N, E, | Q | | | |
| | L, I, A, H, G, S, P | | | | |
| 16 | F, S, A, W, Y | | | X | |
| 17 | K, V, T, E, A, Q, I, L, Y, | M, G, R | | | |
| | S, C, H, W, N, F | | | | |
| 18 | Y, F, S, W, A, K, T, V, E, | | | | |
| | D, R, Q, H, I | M, N | | | |
| 19 | S, F, L, G, M, A, N, I, Q, | D, R, Y, K, W, H | | | |
| | V | | | | |
| 20 | K, S, L, N, M, H, Y, F, G, | T | | | X |
| | Q, D, A, E, V, R, C | | | | |
| 21 | D, S | | X | | |
| 22 | S, Y, Q, F, I, P, N, H, G, | L, T, W, A, V, E | X | | |
| | M, D, R, K | | | | |
| 23 | F | | X | | |
| 24 | Y | | X | | |
| 25 | F, R, W, Y, H, L, K, M | I, Q, V, S, A, C | | | |
| 26 | G, Y, F, M, H, L, S | A | X | | |
| 27 | L, M, I | | | X | |
| 28 | G, A, S | | | X | |
| 29 | K, Q, F, Y, A, W, S, T, R, | | | | |
| | I, H, C, V, G, L, M, D, N | | | | |
| 30 | E, T, N, A | | X | | |
| 31 | A, S, G, M, T, C, P | | X | | |
| 32 | A, G, Y, W, C, T, M, V, Q | | | X | |
| 33 | Q, D, E, T, G, F, L, A, H, | P | | | |
| | W, M, V, R, I, S, N | | | | |
| 34 | V, D, Q, N, P, H, L, S, I, | E, G, Y | | | X |
| | T, M, R | | | | |
| 35 | T, V, M, R, K, F, A, Y, E, | D, L | | | |
| | H, S, N, W, P, G, I | | | | |
| 36 | G, H, Q, V, A, K | W, Y, D, S, P, I, T, R, N, E, L, | | | |
| | | M, F | | | |
| 37 | T, H, M, E, G, Q, S, L, F, N, | | | X | |
| | A, V, C, D, I, W, Y, R, P | | | | |
| 38 | C, F, R, M, K, S, N, Y, I, | L, E, P | | | |
| | G, W, D, Q | | | | |
| 39 | S, H, M, I, R | D, C, A, E, W, N, F, Y, K, L, V, T, | | | |
| | | Q | | | |
| 40 | F, L, N, S, C, A, R, W, E, H, | | | X | |
| | V, Y, M, G, K, D, I, T, Q | | | | |
| 41 | V, T, I, L, C, M, D | | | X | |
| 42 | M, F, Q, H, I, G, E, S, K, | D | | | |
| | Y, R, V, N, L, T, C | | | | |
| 43 | E, R, A, G, L, V, I, K, N, | W | | | |
| | H, T, F, D, Q, M, C | | | | |
| 44 | A, R, G, L, H, Y, T, W, V, F, | | | | |
| | K, I, C, S, E, M, N, Q, D | | | | |
| 45 | L, A, W, Q, F, N, S, Y, G, | M, K | | | X |
| | C, I, T, R, H, D, P | | | | |
| 46 | W, N, P, M, S, I, C, R, H, | D, Y, F | | | |
| | A, Q, G, K | | | | |
| 47 | T, R, D, N, H, Y, I, F, S, | Q | | | |
| | K, E, L, A, V, G, M, C | | | | |
| 48 | Q, E, N, Y, H, C, V, G, A, | I, L, K | | | |
| | W, P, S, T | | | | |
| 49 | D, N, M, Y, L, C, V, F, T, Q, | | | | |
| | S, R, E, I, P, A, W, K, H | | | | |
| 50 | R, W, I, Q, S, N, V, M, H, | F, A | | | |
| | T, K, E, C, D, L, G | | | | |
| 51 | G, A, Y, V, P, R, L, E, M, | C, I, D, S | | | |
| | T, H, Q, F, W | | | | |
| 52 | Y, S, W, F, E, L, A, M, Q, | V, T, C | | X | |
| | G | | | | |
| 53 | P, T, E, N, I, H, R, G, L, | D | | | |
| | Q, Y, W, C, M, E, V, S, | | | | |
| | A, K | | | | |
| 54 | E, I, H, V, G, T, R, M, P, | A, N, Y | | | |
| | K, Q, L, S, F, W, D | | | | |
| 55 | T, P, F, Q, M | Y, R, L, H, K, N, I, W, V, C | | X | |
| 56 | P, F, H, N | W, V, T, E, Y, R, C, S, K, I, L, | | | |
| | | Q, G, D | | | |
| 57 | A, M, Y, F, H, I, R, S, V, | P, K, W, G | | | |
| | L, D, C | | | | |
| 58 | P, M | N, K, V, R, G, F, T, H, D, S, Q, | | | |
| | | I, W | | | |
| 59 | H, R, S, W, V, T, N | P, Q, C, K, D, E, F, M, A, L, Y, I, | | | |
| | | G | | | |
| 60 | C, F, I, M, L, W, Y, N, V, | D, K, P, E | | | |
| | H, T, R, S, A, Q, G | | | | |
| 61 | Q, T, Y, N, E, W, P, V, K, | L | | | |
| | G, D, I, S, R, M, H, C | | | | |
| 62 | Q, P, W, T, R, K, F, G, V, | S, E, N | | | |
| | A, Y, M | | | | |
| 63 | S, K, E, M, W, F, Y, R, L, | H, T, A, N | | | |
| | V, D, Q, I | | | | |
| 64 | P, H, T, G, M, W, V, L, D, R, | | | | |
| | F, S, Y, I, A, N, Q | | | | |

TABLE 13QQ
PDGER_mb1
SEIEEVLELIRRYDPEVVKELEKVLRALKRSGVSPEKILKYLLIVAHFLGSPLAVRLLFRLMTKR
(SEQ ID NO: 1049)

| | | | | | |
|---|---|---|---|---|---|
| 1 | F, I, H, K, V, R, T, P, E, M, | | | | |
| | G, Y, L, W, N, Q, D, S, A, C | | | | |
| 2 | A, P, D, H, K, Q, S, G, R, Y, | | | | |
| | N, E, T, M | | | | |
| 3 | L, A, V, N, M, T, I, F | | | | |
| 4 | D, M, K, R, P, W, I, F, G, Q, | | | | |
| | H, S, A, Y, T, L, V, N, E | | | | |
| 5 | C, W, Y, V, A, I, K, H, L, D, | | | | |
| | P, G, F, N, M, T, Q, S, R, E | | | | |
| 6 | E, M, N, I, C, H, L, Y, Q, S, | | | X | |
| | T, G, W, V, A, R | | | | |
| 7 | F, M, I, L, K | | | | |
| 8 | C, Q, Y, T, I , A, S, H, K, W, | | | | |
| | M, D, G, F, V, N, L, R, E | | | | |
| 9 | G, T, R, M, D, H, V, Q, A, | C | | | |
| | I, W, E, N, F, S, Y, K, | | | | |
| | L | | | | |
| 10 | V, M, L, I | | | X | |
| 11 | Y, H, D, S, G, K, Q, E, A, | L, T | | | |
| | N, M, W, R | | | | |
| 12 | Q, W, D, H, S, G, I, L, P, K, | | | | |
| | F, M, Y, E, N, T, A, V, R, C | | | | |
| 13 | F, T, H, A, C, N, D, W, K, Q, | | | X | X |
| | R, V, S, E, G, M, Y, I, P | | | | |
| 14 | P, E | R, S, Q, N, D | | | X |
| 15 | H, R, F, E, D, Y, K, A, G, | L | | | |
| | Q, S, N, T, P, V | | | | |
| 16 | M, N, S, A, I, D, Y, H, K, P, | | | | |
| | L, Q, R, G, V, T, E, F, C | | | | |
| 17 | P, C, H, K, T, A, S, R, N, V, I | | | X | |
| 18 | H, S, C, T, W, Q, K, G, A, Y, | | | | |
| | M, E, N, E, V, I | | | | |
| 19 | V, E, R, H, Y, F, S, G, N, | | | | |
| | L, W, M, A, C, T, Q, I, D, P, K | | | | |
| 20 | L, K, D, V, I, P, T, M, E, | S, C, F, H | | | |
| | A, R, Y | | | | |
| 21 | F, L, M | | | X | |
| 22 | C, D, Y, K, M, I, S, W, Q, | | | | |
| | T, G, H, V, R, F, N, A, L, E | | | | |
| 23 | E, D, T, N, L, I, V, M, A, | H, R, F, Y, W, P, G, C, K | | | |
| | S, Q | | | | |
| 24 | P, A, V, H | | | | |
| 25 | V, R, K, E, D, S, Y | T, N, M, H, C, Q, A, W, F, G, I, L | | | |
| 26 | Y, N, E, D, M, H, W, Q, C, L, | | X | | |
| | T, G, F, S, I, K, A, V, R | | | | |
| 27 | L, I, T, V, R, A | S, Q, P | X | | |
| 28 | M, L, F | | X | | |
| 29 | N, Y, M, E, H, D, G, V, S, | C | | | |
| | Q, R, I, A, T, L, W, K, | | | | |
| | F | | | | |
| 30 | T, K, N, H, R, Q, G, S | A, L | X | | |
| 31 | H, K, Y, R, L, V, M, F, G | I, N, P, A, C, Q, T, S | X | | X |
| 32 | R, Q, K, L, N, F, H, S, M, T, | | | | X |
| | A, D, W, Y, E, G, I, C | | | | |
| 33 | M, A, W | T, F, L, N, S, K, I, C, R, G, E, Q, | | | X |
| | | V, H | | | |
| 34 | K, R | Y, N, C, I, F, L, P, W, T, E, D, V, | | | X |
| | | G, M, Q, A, H, S | | | |
| 35 | I, N, E, L, V, H, Y, Q, F, | C | | | |
| | M, G, S, A, R, D, K, T, | | | | |
| | P, W | | | | |
| 36 | P, L, Y, A, I, M, G, S, H, D, | | | | |
| | Q, V, T, R, N, F, E, C, K | | | | |
| 37 | K, R | | X | | |
| 38 | A, K, Y, C, S, T, M, G, V, | E, H, W | | | |
| | Q, R,I , N | | | | |
| 39 | V, T, I, C | N, E, K, Y, M, S, Q, G, A, D, H, L, | | | |
| | | R | | | |
| 40 | G, K, R, Y | | X | | |
| 41 | F, Y, T, E | | X | | |
| 42 | M, C, Q, L | | | | |
| 43 | F | I, Y, L, T | X | | |
| 44 | L, V, I, K | | X | | |
| 45 | I, V | | | | |
| 46 | S, A, M, F | | | X | |
| 47 | Y | H, R | X | | |
| 48 | Y | F, R, H, Q, K | X | | |
| 49 | K | H, N, C, I, Q, S, R, V, M, E, A, L | | X | X |
| 50 | R, K, G | | | | X |
| 51 | S | | | X | X |
| 52 | E, H, N, D, Y | Q, K, T, R, G, P, S | X | | |
| 53 | F, V, R, P, L, H | K, N, I, W, M, S, Y, Q, A, E, T, | | | |
| | | D, C | | | |
| 54 | S, A | | | X | |
| 55 | I | V | X | | |
| 56 | T, D, S, Q, N, H, E, K, | | X | | |
| | A, R, L, G | | | | |
| 57 | N, D, A, I, S, M, Q, T, V, | E, F, H, W, C, K | | X | |
| | Y, L | | | | |
| 58 | I, F, L, V, S, Q | | | X | |
| 59 | F, A | | X | | |
| 60 | F, I, T, M, K, H, A, E, W, Q, | | X | | |
| | S, N, D, L, G, R, Y | | | | |
| 61 | H, Q, Y, A, M, T, V, S, | | | | |
| | W, E, I, F, L, P | | | | |
| 62 | V, I, L, M | | X | | |
| 63 | F, V | Y, M, N, I, A, H, C, L, | | | |
| | | W, T, S | X | | |
| 64 | D, T, N, Q, F, I, G, V, M, R, | | | | |
| | H, A, L, Y, S, E, W, K | | | | |
| 65 | G, N, P, T, A, D, H, Y, I, F, | | | | |
| | L, E, Q, S, V, M, K, R, W | | | | |

TABLE 13RR
PDGER mb2
SLEELERLIRKLVKEGDPAARRALLVLESLKRRGVSPEEIVRRLIAFLFILGNPELIRLAIRLFL
(SEQ ID NO: 1050)

| | | | |
|---|---|---|---|
| 1 | I ,A, V, P, Q | N, T, K, S, G, H, R, M, W, C | |
| 2 | V, W, I, Y, T, Q | L, F, M, K, S, R, C, A | |
| 3 | T, S, K, Y, H, A, W, R, L, | I, D | |
| | M, V, Q, F, G, P, N, C, | | |
| | E | | |
| 4 | Q, A, R, K | I , G, T, S, E, V, M, L, Y, D, N, H | |
| 5 | I, L, A, M, R | T | X |
| 6 | E, N, T, G, Q, R | S, H, K | |
| 7 | A, Q, L, K, H | I, N, R, M, G, T, V, E, W, S, F | |
| 8 | Y | A, L, K, V, M, N, R, I, F, T, S, Q | |
| 9 | M | E, I, L, W, F | X |
| 10 | W, R, L, K, M, D | T, F, I | |
| 11 | R, K, E, S | N, Q, I, W | |
| 12 | F, M, T, V, A, W, Y, H, S, | L, C, I, E, G | |
| | N, K, Q | | |
| 13 | T, I, R, N | K, W, V, A, E, M, L, F, Y, G | |

| | | | | | |
|---|---|---|---|---|---|
| 14 | W | R, K, H, A, C, T, G, Y, Q, F, I | | | |
| 15 | T, S, Q, A, K, Y, L, D, M, | C, F, R, V, I, W, G | | | X |
| | N, E, H | | | | |
| 16 | D, R, H, G, K | Q, V, A | | | X |
| 17 | V, I | Q, D, E, N, W, S, C, A | | | X |
| 18 | T, P, Q, W | K, A, H, N, R, G, V, F | | | |
| 19 | Q, Y, G, R, K, S | E, L, A, M, H, N, F, P, V | | | |
| 20 | E, V, I, L, P, T, A | S, M, K, H, G, D, W, N | | X | |
| 21 | P, W | R, K, F, G, V, Y, Q | X | | |
| 22 | L, R | E, D | X | | |
| 23 | A, S, N | | | X | |
| 24 | W, Y, Q, F | L, C, T, M, A, E, V, H, R, S | | | |
| 25 | L, G, H, N | S | X | | |
| 26 | V, Q, P, K | W, I, N | X | | |
| 27 | C, W, Y, H, L, G | M, F, A, V, N | | | |
| 28 | Q, R, S, M, K, T, Y, V, L, | E, A, H, F, I | | | |
| | N, D | | | | |
| 29 | M, T | L, S, F, I, R | X | | |
| 30 | D, T, A, G, S, M, N | L, E, R, V, F | X | | |
| 31 | R, K, F | Q, T, H, W, N, L | | | |
| 32 | R, A, N | T, M, W, P | X | | |
| 33 | Y, W, R | C, M | X | | X |
| 34 | P, A | K, R, G, D, W, S, I, F | | | X |
| 35 | E, C, I, D, Q, T | L, N, M, V, K, A, G, S | | | X |
| 36 | M | A, L, S, T, E, D, H, K, V, I, R, | | | X |
| | | Y, Q, P, W | | | |
| 37 | R, Y, T, H, Q, K, W, A, N, | E, P, C, F | | | |
| | M, I, G, V, D, L | | | | |
| 38 | A, P, N, Q, D, S | E, T, K, H, L, I, M, W | | | |
| 39 | E, N, A, L | H | | | |
| 40 | G, E, K, R, H, Q, S, L, A, | M, V, C, T, I | | | |
| | Y, N, F | | | | |
| 41 | S, D, M, Q, E, L, N, F, T, | V, C, A, G, H, Y | | X | |
| | I | | | | |
| 42 | R, N | M, C, F, E, T | X | | |
| 43 | K, R | I, Y | X | | |
| 44 | V, L, T | A, E | | X | |
| 45 | C, T, F, S | I, V, A, M, G, L, P, D, | X | | |
| | | N | | | |
| 46 | S, I, T | A, H, Y, C | X | | |
| 47 | Y | F, Q, S, T, G | X | | |
| 48 | L, V, Y, C | I, Q, K | | X | |
| 49 | F | M, V, D | X | | |
| 50 | Q, G | I, T, M, F | X | | |
| 51 | I, V | L, W, Q | | X | |
| 52 | N, G, K, E | C, M | | | X |
| 53 | H, Q, K | N, W | | X | X |
| 54 | D, N, M, K, C | H, P, T, Q, S, E, R, G, A, V | | | |
| 55 | E, Q, I, V, D, T, M, R | A, S, G | | | |
| 56 | M, W, E, S, F | L, H, V, T, I, C, K, R | | X | |
| 57 | I, H | P, W, Y, T | X | | |
| 58 | N, H, K, R, T, P, Q | S | S | | |
| 59 | Q, P, C, S, G, A, T, Y, V, | D, M, L, R, I | | | |
| | H, E, N, F, K | | | | |
| 60 | S, A, I | W, G, Q | | X | |
| 61 | I, L, Q | C | X | | |
| 62 | K, R | H, Q, Y, | X | | |
| 63 | I, Q | L, T, A, S, R, G, N | | X | |
| 64 | T, Y, W, M | F, A, I, S, V | X | | |
| 65 | M, C, I, Y, L | F, A, S, T, V, W, E, K | X | | |

TABLE 13SS
PDGFR_mb3
DILEKAKKVAREILDPEVVRIFELAVEVIKKKGANPSSILIAVLRLLRRRGVDPELLREVERRAR
(SEQ ID NO: 1051)

| | | | | | |
|---|---|---|---|---|---|
| 1 | Q, K, L, W, I, N, S, V, H, | M, F, Y | | | |
| | A, R, D, G, P, E, T, C | | | | |
| 2 | M, I, L, V, F | Y, T, S, K, Q, R, A, C | | | |
| 3 | L, T, M, I, A | | | | |
| 4 | T, S, G, F, H, E, L, K, Y | Q, M, D, I, W, N, V, C, R, A | | | |
| 5 | E, N, R, I, K, Q, G, A | Y, V, M, T, S, C, W | | | |
| 6 | A, G | S | | X | |
| 7 | L, I, M, V, K, T | R, H, C, G, A, Q | | | |
| 8 | M, R, F, V, A, K, Y | H, I, Q, W, N, G, C, T, D, L, S, E | | | |
| 9 | A, T, C, V, G, Q, I, S, L | R, F, Y, M, N, H, W | | | |
| 10 | A, V, S, C, G | I, M | | X | |
| 11 | H, R, G | L, Y, E, A, I, C, S, Q | | | |
| 12 | N, T, H, S, Q, K, L, R, A, | I, V, D, C | | | |
| | E, M, G | | | | |
| 13 | V, T, I, M, L, R, K, Y | S, Q, C | | X | |
| 14 | F, L, M | | | X | X |
| 15 | A, S, R, V, P, G, Q, E, M, | F, Y, H, I, C | | | |
| | L, T, K, D, N | | | | X |
| 16 | F, S, R, P, N, G, V, Q, Y, | W, T, M, H, A, K, L, I, | | | |
| | D | E, C | | | |
| 17 | V, K, M, Q, P, T, Y, L, A, | I | | | |
| | R, N, G, F, D, E, S, W, | | | | |
| | H | | | | |
| 18 | S, E, H, C, F, K, A, Q, M, | W, I | | | |
| | G, D, P, N, R, Y, T, L, | | | | |
| | V | | | | |
| 19 | Y, R, N, H, G, C, Q, K, S, | E | | | |
| | T, M, A, V, I, D, W, L, | | | | |
| | E | | | | |
| 20 | H, F, W, R | V, Y, M, K, L, I, P, G | X | | |
| 21 | L, F, P, I | M, V | X | | |
| 22 | W, M, F | L | | X | |
| 23 | F, L, C, M, S, K, R, G, I, | Q, H, T, Y, V, D, P | | | |
| | E, W, N, A | | | | |
| 24 | W, K, R, L, H, G, V | F, M, T, S, N, I, Y, A | X | | |
| 25 | H, I, L, V, F, W, K, G, C, | Y, T, R, N | X | | |
| | A, M | | | | |
| 26 | A, S, C, V, T, M | I, L | | | |
| 27 | H, M, T, I, R, K, G, N, Q, | D, C | | | |
| | V, A, F, L, S, Y, W, E | | | | |
| 28 | G, A, K, F, S, H, W, Y, V, | R | X | | |
| | T, M | | | | |
| 29 | L, A | | X | | |
| 30 | R, K, L | Q, S, T, V, G, F | | | |
| 31 | N, R, K, Q | L, Y | X | | |
| 32 | R, K, M, H, N | I, F, T, V, L, S, W, Y, | X | | |
| | | G | | | |
| 33 | Q, A, R, K, F, V, M, I, S, | Y, P | | | X |
| | L, G, W, T, H | | | | |
| 34 | L, F, T, Y, H, M, W, V, I, | R, K, C, Q | X | | X |
| | A, G | | | | |
| 35 | K, S, R, T, Q, Y, N, H | P, A, G, M, W, I | X | | X |
| 36 | H, K, A, P, R, S, N | G, Q, T, D, M, E, Y | | | |
| 37 | V, M, G, I, Q, T, E, L, A, | H, Y, D | X | | |
| | K, R, F, W, N, S, P | | | | |
| 38 | G, A, S, P, T, H, R, Q, M, | V | X | | |
| | I, F, N | | | | |
| 39 | Q, A, I, V, C, G | K, S, N, T | | | |
| 40 | L, M, I, F | V, T | | | |
| 41 | I, V, L, N | | X | | |
| 42 | Y, I, W, F, T, V, M, A, Q, | P, G, L | X | | |
| | H | | | | |
| 43 | C, T, G, V, E | I, L, S | | X | |
| 44 | L | | | | |
| 45 | A, I, L, K, R, M, F, Y, W | V | X | | |
| 46 | M, I, F, L, D | W, V | X | | |
| 47 | I, C, L, A, V, M | R, K | | X | |
| 48 | Y, W, F, N, A, R, H | L, K, G | X | | |
| 49 | F, W, R | S, Y, T, A, M, H, K | X | | |
| 50 | E, D | H, R, K, Q, T, Y, M | X | | |
| 51 | L, N, G, C, A | H, R, S, K, Q | | | X |
| 52 | T, V, E, A, C, Q, S | I, F, L, G | | X | X |
| 53 | Q, H, R, P, S, M, K, D | L, Y, G, A, N, E, T, V | | | X |
| 54 | K, L, R, A, I, Y, V, Q, G, | E, H, C, D | | | |
| | T, M, S, P | | | | |
| 55 | Q, I, N, G, L, W, K, Y, M, | C, P | | | |
| | D, A, H, R, V, S, T, F, | | | | |
| | E | | | | |
| 56 | K, S, Q, R, A, V, L, P, H | N, I, C, T, M, E, D, Y, F | | | |
| 57 | L, I | | | X | |
| 58 | S, M, Y, F, Q, K, H, I, W, | L, C, N, G, V | | | |
| | R, T | | | | |
| 59 | M, Y, V, K, E, T, A, N, Q, | I, C, F, W | | | |
| | S, R, L, H, D | | | | |
| 60 | I, V | M, P, L, R, C | | X | |
| 61 | W, Y, F, H | I, L, C | X | | |
| 62 | M, A, T, W, Y, F, Q, H, E, | G | | | |
| | K, N, V, R, I, D, S, C, | | | | |
| | L | | | | |
| 63 | Q, R, M | S, K, G, A, I, V, T, Y, H | | | |
| 64 | A, S, W | T, G, C | | X | |
| 65 | Q, M, N, K, Y, W, R, A | H, V, I, F | X | | |

TABLE 13TT
PDGER_mb4
DEREELIELILRNLDDPEKVHELLKRLLELAEELGDRRLYRYVMLIFWALRRNPERAERLLRKLK
(SEQ ID NO: 1052)

| | | | | | |
|---|---|---|---|---|---|
| 1 | T, S, C, P, K, M, N, I, Q, L, G, | | | | |
| | F, V, A, W, R, H, Y, E, D | | | | |
| 2 | T, P, A, Q, D, H, V, S, R, C, G, | | | | |
| | M, F, N, W, L, Y, I, K, E | | | | |
| 3 | K, R | V, L, I, Y, D, Q, M, H, P, N, T, G | | | |
| 4 | N, S, D, P, G, K, C, Q, R, | M, I, F, Y, W, L, E | | | |
| | V, H, A, T | | | | |
| 5 | R, A, T, P, S, Y, G, L, K, | I, H, N, W, C, M, F, Q, D, E | | | |
| | V | | | | |
| 6 | V, T | C, Y, Q, W, F, I, D, M, A, E, S, | | X | |
| | | H, K, G, P, R, N, L | | | |
| 7 | K, S, Q, C, G, R | E, A, M, L, T, Y, N, P, V, H, D, | | | |
| | | F, I | | | |
| 8 | R, K, Q, F, V, M, A, N, T, | D, L, P, I, Y, C, G, E | | | |
| | H, W, S | | | | |
| 9 | K, M, H, R, G, Q, C, A, E, S, D, | | | | |
| | I, W, N, P, Y, T, V, L | | | | |
| 10 | V, K, G, Q, N, W, E, D, P, L, | | | X | |
| | H, I, Y, T, R | | | | |
| 11 | P, K, D, H, N, S, R, Q, A, | I, V, C, Y, W, F, L | | | |
| | G, T, M, E | | | | |
| 12 | K, G, I, T, F, D, E, A, C, M, | | | | |
| | P, V, Q, H, W, N, S, R | | | | |
| 13 | Y, E, D, C, K, W, S, F, Q, M, | | | | X |
| | T, H, P, V, A, R, N, I, L, G | | | | |
| 14 | Q, R, E, A, D, V, S, P, K, | M, N, L | | | X |
| | T, Y, I, H, G, C | | | | |
| 15 | F, A, V, S, G, L, C, R, E, | N, M, Y, W, K, I, P, H, T | | | X |
| | Q, D | | | | |
| 16 | V, Q, W, N, K, I, E, R, G, | D | | | X |
| | F, H, S, A, L, P, Y, C, | | | | |
| | M, T | | | | |
| 17 | A, T, L, G, N, R, S, P | C, K, Q, E, D, M, H, I, Y | | | |
| 18 | V, Q, A, S, T, K, I, F, R, L, | | | | |
| | Y, M, N, P, G, W, H, C, E | | | | |
| 19 | Q, M, C, E, A, S, R, H, G, N, | | | | |
| | I, P, T, K, D, V, Y, L | | | | |
| 20 | I, K, W, L, R, M, F, Y | E, Q, T, H, V | | | |
| 21 | I, V, P | K, N, Q, E, L, T, F, W, | X | | |
| | | Y, G, S, M, A, H | | | |
| 22 | T, A, H, K, N, S, I, Y, W | V, L, C, D, G, R, P, M, Q, F, E | | | |
| 23 | F, R, G, N | C, H, T, S, Y, M, I, D, A, K, W, E, | | X | |
| | | Q, V, P, L, | | | |
| 24 | F, G | I, M, L | | | |
| 25 | T, K, N | R | X | | |
| 26 | K, Y, D, V, N, Q, A, P, H, W, | | | | |
| | G, T, L, C, E, S, I, R, M | | | | |
| 27 | F, I, V, L, S | Y, C | | X | |
| 28 | V, L | | X | | |
| 29 | D, P, N, I, M, V, C, W, A | E | X | | |
| 30 | Y, K, N, T, E, A, C, D, Q, | I, F, H, L | | | |
| | S, R, W, G, V | | | | |
| 31 | V, S, T | G, C, A | | X | |
| 32 | D | N, E | X | | |
| 33 | D | C, H, N, I, M, T, S, A, | X | | |
| | | G, E | | | |
| 34 | F, Y, E, T, C, I, M, H, R, | S, W, L | | | |
| | N, Q, K, V, A, G | | | | |
| 35 | E, D, T, H, W, A, C | N, P, I, S, K, Q, V, L, | | | X |
| | | Y, F, M, G | | | |
| 36 | I, N, S, D | H, Q, E | | | X |
| 37 | T, K | G, M, Q, S, H, I, A, D, | X | | |
| | | V, P, R, L | | | |
| 38 | M, D, N, K, A, P, V, G, Y, | F, I, L, W, E | X | | |
| | Q, S, T, H, R | | | | |
| 39 | Q | N, R, M, I, C, G, L | | | |
| 40 | Y, L | | X | | |
| 41 | G | A, S, Q, R | X | | |
| 42 | H, Q, A, D, G, N, S , R | M, E, I , V, W, T, L, F, | | | |
| | | K, C, Y | | | |
| 43 | I, C | G, Y, L, T, V | | X | |
| 44 | M | | X | | |
| 45 | E, S, H, M, A, Y, G, N, Q, | T, L, F | X | | |
| 46 | Y, F | T, K, R, W, V, C, I | | X | |
| 47 | V, I | A, K, E, L, T, F | X | | |
| 48 | Y | F, H, W | X | | |
| 49 | N, W, C, F, M, Y, H, L, V, | T, S, I, E, Q, R, G | | | |
| | A | | | | |
| 50 | K, Q, E, R | F, G, M, I, D, T, S, Y, L | | X | |
| 51 | H, F, Q, S, K, V, M, Y, N, P, | | X | | |
| | I, L, R, G, T, A | | | | |
| 52 | K, N, T, F, Y, H, M, Q | A, L, S, W, E, V, G, D, I, | | | X |
| | | C, R | | | |
| 53 | E, K, M, H, Q, D, F, Y, A | R, T, G, I , S, L, V, W,N | | | X |
| 54 | F, M, N, V, K, W, A, I, T, Q, | | | | |
| | H, S, Y, R, P, G, D, E | | | | |
| 55 | K, P, V, N, Y, H, F, L, G, Q, | | | | |
| | D, I, W, C, S, A, M, T, E, R | | | | |
| 56 | L, M | K, W, G, Y, N, I, Q, V, | X | | |
| | | H, F, S, T, A, R | | | |
| 57 | T, Y, V, G, A | F, I, S, L | | | |
| 58 | N, Y, M, D, K, S, Q, T, R | F, W, I, A, H, L, C, P, V, G, | | | |
| | | E | | | |
| 59 | L, Q, V, T, I, N | M, A, K, S, P, F, C, H, W, | | | |
| | | E, D, Y, G, R | | | |
| 60 | M | A, F, Q, I, L | X | | |
| 61 | T, I, M, L, G | V, F | | X | |
| 62 | A | M, W, G, V, S, N, F, K, E, L, | | | |
| | | D, T, Q, H, I, Y, R | | | |
| 63 | N, G, H, A, M | L, W, F, I, S, Q, C, T, | X | | |
| | | Y, R, K | | | |
| 64 | F, W, M, L, I, Q | | | X | |
| 65 | P, G | R, C, T, A, F, H, D, M, V, Q, | | | |
| | | N, L, I, E, W, S, Y, K | | | |

TABLE 13UU
PDGFR_mb5
PELIEEALELLKEGDPKKVFRVLARLVQLLREKGDPRYRLVLLILAYVRFGNPEAARQLLRIVLK
(SEQ ID NO: 1053)

| | | | | | |
|---|---|---|---|---|---|
| 1 | V, C, G, Q, H, A, E, F, Y, K, L, | | | | |
| | I, R, W, M, P, T, S | | | | |
| 2 | H, K, I, D, P, G, N, W, V, Y, C, | | | | |
| | S, L, M, T, Q, A, F, R, E | | | | |
| 3 | T, S, H, E, Y, R, N, K, A, I, F, | | | | |
| | G, V, Q, W, M, L, P, C | | | | |
| 4 | A, V, N, F, P, Y, W, C, E, G, R, | | | | |
| | Q, T, L, S, K, M, I, D, H | | | | |
| 5 | M, R, N, L, H, K, Y, W, P, A, F, | | | | |
| | D, C, T, I, Q, V, G, S, E | | | | |
| 6 | K, H, F, D, N, I, S, C, A, G, V, | | | | |
| | W, Y, L, T, R, P, Q, M, E | | | | |
| 7 | Y, G, T, C, S, A, Q, H, D | | | X | |
| 8 | A, C, N, W, Y, D, S, K, M, R, H, | | | | |
| | E, I, V, Q, G, F, T, L | | | | |
| 9 | R, K, Q, W, T | S, L, Y, C, V, P, M, D, A, F, G, | | | |
| | | H, N, I, E | | | |
| 10 | Y, A, G | F, R, H, W, D, K, T, N, Q, I, V, | | X | |
| | | C, M, S, L | | | |
| 11 | A, Y, W, S, I, M, V, T, Q, F, | | | X | |
| | L, C, K, H | | | | |
| 12 | Y, A, Q, C, F, T, I, M, V, W, S, | | | | |
| | G, E, H, R, D, L, N, P, K | | | | |
| 13 | R, I, K, Y, Q, E, D, W, C, | G, H, E | | | |
| | T, P, V, N, L, S, M, A | | | | |
| 14 | N, E, Q, R, T, F, W, Y, H, K, S, | | | | X |
| | D, M, P, V, C, G, A, I | | | | |
| 15 | H, K, R, N, P, Q, L, C, V, F, A, | | | | X |
| | I, G, M, S, T, E, Y, D | | | | |
| 16 | K, T, N, V, H, I, S | Q, F, M, A, L, Y, R, E, C, G, W, | | | |
| | | D, P | | | |
| 17 | A, S , V, M, G, Q, H, T, P, R, | | X | | |
| | I, N, Y, K, L, D, E, F, W | | | | |
| 18 | G, P | D, N, S, H, L, E, M, Y, Q, R, V, | | | |
| | | A, K | | | |
| 19 | A | P, Y, K, T, C, M, S, F, W, G, I, | | X | |
| | | L, N, V | | | |
| 20 | C, H, K, R, I, G, T, S, P, M, W, | | X | | |
| | N, L, Y, Q, V, E, A, F | | | | |
| 21 | Y, M, E, Q, D, A, N, L, K, H, I, | | X | | |
| | F, V, S, T, G, P, R, W | | | | |
| 22 | P, Y, S, H, A, I, N, Q, M | L, C, R, K, G, T, V | | X | |
| 23 | T, I, V, M, A, G, C, L, S, F | | | X | |
| 24 | E, W, Y, N, T, V, M, Q, G, H, | | X | | |
| | L, K, R, S, A, I, P, F, C | | | | |

| | | | | | |
|---|---|---|---|---|---|
| 25 | P | K, L, D, E, H, A, M, N, | X | | |
| | | Q, Y, T, F, V, S, G, | | | |
| | | R | | | |
| 26 | Y, F, I, H, N, S, M, R, A, G, | | | X | |
| | Q, T, W, C, K, E, V, L | | | | |
| 27 | H, F | I, M, W, C, N, Q, A, T, | X | | |
| | | Y, L, R, V | | | |
| 28 | N, K | G, P, T, F, D, H, I, Y, | X | | |
| | | V, L, E, W, S, M, R, | | | |
| | | A, Q | | | |
| 29 | M, G, H, C, D, F, T, N, Q, P, | | | X | |
| | E, W, K, R, A, I, S, V, Y, L | | | | |
| 30 | Y | F, M, W, I, A, C, K, T, | | X | |
| | | Q, H, V, L | | | |
| 31 | E, D, H, S, N, Y, A, V, T, | C, I, F, G, L, K, M, P, | X | | |
| | Q | W, R | | | |
| 32 | T, D, S, K, W, C, R, F, M, | P, H, N, Q, A, E | X | | |
| | Y, L, V, G, I | | | | |
| 33 | S, F, N, C, L, M, E, W, V, T, Q, | | | | |
| | P, A, D, G, R, H, Y, K, I | | | | |
| 34 | C, V, L, Q, D, T, H, K, R, | | | | X |
| | P, M, S, Y, A, E, N, G, F | | | | |
| 35 | S, Y, E, N, H, D, Q | | | | X |
| 36 | K, L, T, V, Y, Q, D, G, I, R, | | | | |
| | N, H, M, A, E, W, S, P, F | | | | |
| 37 | K, E, S, L, M, T, A, Y, N, G, Q, | | | | |
| | H, R, V, I, F, D | | | | |
| 38 | L, I, V, E | C, M, F, D, A, H, T, Q, S, Y | | | |
| 39 | K, R, L | | X | | |
| 40 | H, V, W, T, Y, R, A, N, I | S, Q, F, K, P, D, E, M, G, L | | | |
| 41 | G, L, C, T, A, M, I, F, S, V, Y, | | | X | |
| | W | | | | |
| 42 | I | V, M, Y, F, P, L | X | | |
| 43 | R, Q, S, W, F, H, T, P, N, | | X | | |
| | Y, I, M, K, V, L | | | | |
| 44 | V, T, A, S, C | P, M, L, G, Y, I | | X | |
| 45 | A, G, V, I | S, T, F, W, P, Y, M, C, | X | | |
| | | L | | | |
| 46 | M, G, Y, A, S, F, W, E, V | | X | | |
| 47 | F, H, R, N, Q, Y, L, M | | X | | |
| 48 | I, T, Y, M, A, F, L, R, Q, | | | X | |
| | V, W, H | | | | |
| 49 | K | H, R | X | | |
| 50 | Y, I, S, K, L, H, Q, G, T, R, | | X | | |
| | V, M, N, W, A, F | | | | |
| 51 | H, Q, N, K, R, G, D, S, A, M | | | | X |
| 52 | P | Q, K, D, H, V, S, T, M, | X | | X |
| | | A, L, W, E, R, Y, N | | | |
| 53 | R, W, A, M, K, G, Y, N, E, Q, | | | | |
| | D, S, F, L, I, H, T, V, P | | | | |
| 54 | K, T, L, V, P, W, N, F, Y, S, G, | | | | |
| | M, I, H, D, R, A, Q, E, C | | | | |
| 55 | G, Y | M, V, I, H, K, R, Q, F, N, L, T, | | | |
| | | S, P, A | | | |
| 56 | Q, T, V, S, A | | | X | |
| 57 | F, I, H, V, N, E, L, Q, D, G, | | | | |
| | T, S, K, M, A, R, Y, C | | | | |
| 58 | N, M, T, V, R, E, G, L, K, S, H, | | X | | |
| | A, Y, D, Q, I, F | | | | |
| 59 | A, G, V, M, I, Q, L, F | | X | | |
| 60 | Q, G, T, R, A, I, S, M, E, L, E, | | | X | |
| | K, V, H | | | | |
| 61 | W, I, N, F, S, C, G, D, Y, A, E, | | | | |
| | P, M, L, Q, V, T, K, H, R | | | | |
| 62 | S, G, M, L, R, D, K, T, E, Y, N, | | X | | |
| | F, H, W, Q, V, A, I, P | | | | |
| 63 | L, M, P, F, I | R, E, H, D, T, N, W, S, G, C, Q, | | X | |
| | | K, A, Y, V | | | |
| 64 | K, Q, F, A, P, R, T, E, D, G, V, | | | | |
| | H, W, C, I, M, Y, S, N, L | | | | |
| 65 | F, Y, H, I, M, L, Q, P, V, N, E, | | | | |
| | D, G, S, T, A, R, K, W | | | | |

TABLE 13VV
PDGFR_mb6
DELREILREALEKGDPELVKELLELLARLADETGDPKLRIVIAYVWFAKKKNDPRLLKTALQVLK
(SEQ ID NO: 1054)

| | | | | | |
|---|---|---|---|---|---|
| 1 | K, R, V | H, A, Y, P, M, F, N, T, Q, E, W, | | | |
| | | S, I, G, L, D | | | |
| 2 | K, R, Q, H | N, Y, V, I, P, F, M, T, L, G, D, | | | |
| | | A, S, E, W | | | |
| 3 | I, V, H, P, M, T, G, F, W, D, | | | | |
| | N, Q, S, R, A, K, E, Y, L, C | | | | |
| 4 | A, K, S, N, I, P, Q, E, M | L, H, Y, F, T, G, W, V, R, D | | | |
| 5 | R, K, Q, H, N, T, S, V | I, A, M, C, W, D, L, F, G, Y, P, | | | |
| | | E | | | |
| 6 | K, R, H, L, M, Q, A, T | S, N, E, F, Y, C, W, D, P, V, I, | | | |
| | | G | | | |
| 7 | A, I, V, G, T, F, C, M, L, | S, W, Y | | X | |
| 8 | M, I, V, K | A, T, C, E, W, Q, F, H, N, G, S, | | | |
| | | Y, L, R | | | |
| 9 | R, K, S, A, M, V, H | Q, D, C, L, T, N, G, I, E, P | | | |
| 10 | K | G, R, M, Q, T, S, L, V, E, N, Y, C, | | X | |
| | | H, I, A, F, D, W | | | |
| 11 | I, Y, A | M, C, S, Q, V, N, H, W, T, G, F, L, | | | |
| | | D, E | | | |
| 12 | N, T, S, D, I, H, A, L, V, M, | | | | |
| | F, W, Q, Y, K, G, R, E, P | | | | |
| 13 | T, N, Q, S, R, A, K, H, V, G, | | | | |
| | I, L | | | | |
| 14 | K, N, E, S, H, W, D, P, R, F, | | | | |
| | Q, Y, T, G, A | | | | |
| 15 | K, E, I, M, W, L, S, Q, F, | H, A, Y, R, T, D, N, V, P | | | |
| 16 | W, L, M, I, V | N, K, H, G, Q, Y, E, S, R, A, F, | | | |
| | | T, D, P | | | |
| 17 | K, S, R, N , G, T , M, A, H, | Q, C, P, D, I, Y, V, W, F, E | | | |
| | L | | | | |
| 18 | H, N, K, Q, S, R, A | G, V, I, Y, C, F, M, P, D, E, T, | | | |
| | | L | | | |
| 19 | G, S, P, N, T, Y, M, W, A, | Q, H, I, E, R, V | | X | |
| | L, D, F, K, C | | | | |
| 20 | F, W, Y, M, I, P, V, G, N, | D, C, Q, H, R, K, E | X | | |
| | A, L, S, T | | | | |
| 21 | H, K, R, P, N, G, Q, Y | T, S, L, D, W, C, I, V, A, F, M, E | | | |
| 22 | A, P, C, G, S, Y, M, K, T, | E, R, V, I, W, F, D, L | | X | |
| | H, Q, N | | | | |
| 23 | W, M, F, I, L, Y | | | | |
| 24 | Y, F, I, K, M, L, V, Q, P, | C, D, E | X | | |
| | R, T, W, A, N, H, S, G | | | | |
| 25 | P, G, R, D, K, S, Q, N | A, M, H, E, T, C, I, Y, V, W, F, L | | | |
| 26 | V,T, S, C, M, I, A, L, F | | | X | |
| 27 | H | V, Y, R, K, I, P, A, L, | X | | |
| | | W, S, G | | | |
| 28 | E, A, P, V, Q, H, K, N, T, S, | | X | | |
| | R, D, L, M, C, G | | | | |
| 29 | E, M, T, N, A, H, Q, S, K, | | | | |
| | V, Y, W, C, I, F, R, L | | | | |
| 30 | H, Y, F, W, V | N, S, M, C, L, E, R, Q, I, A, T, G | | | |
| 31 | G, A | H, S, R, N, K, Y, E, Q, | X | | |
| | | W, P, D, V, F, T, M | | | |
| 32 | K, M, H, T, G, S, N, A, L, | Y | X | | |
| | I, Q, D, R, V, E | | | | |
| 33 | E, R, H, N, W, D, G, S, A, | | | | |
| | T, K, Q, Y, C, P | | | | |
| 34 | D, E | V, P, H, T, M, Q, S, A, K, R, Y, | | | X |
| | | N, G, F, W, L, I | | | |
| 35 | D, N, E | | | | X |
| 36 | D, R, K, P, Q, F | H | X | | |
| 37 | N, Q, R, H, V, T, Y, M, I, | | | | |
| | A, L, F, S, W, K | | | | |
| 38 | Y, M, I, W, F, L, V | | | X | |
| 39 | K, R, D | | | | |
| 40 | L | T, V, M, I | X | | |
| 41 | I, A, V, C, L | | | | |
| 42 | A, L, M, V, T, F, I | | X | | |
| 43 | L, I | K, T, Y, F, M, V, A, S | X | | |
| 44 | Y | | X | | |
| 45 | W, I, C, Y, M, V, L, T | | | X | |
| 46 | L, M | I, V, Y, W, F | X | | |
| 47 | Y, W, F, C | | X | | |
| 48 | W, G, S, A | | | X | |
| 49 | W | Y, R, Q, F, V, I, H, M, | X | | |
| | | L, S, K, N, C | | | |
| 50 | R, K, L, E | | | | |
| 51 | F | N, R, K | X | | X |
| 52 | S, Q, H, G, A, R, N, K | | | | X |
| 53 | D | | | | X |
| 54 | L, K, M, V, Y, W, F, I, R, P, | | | | |
| | A, S, T, N, Q | | | | |
| 55 | R | | X | | |
| 56 | L | | X | | |
| 57 | L, M, W | | | X | |
| 58 | H, A, R, K, Q, V, N, Y, W, M | | X | | |
| 59 | M, T, I, S, A | | X | | |
| 60 | A | | | X | |
| 61 | Y, F, W, M, L, I | | | | |
| 62 | D, S, L, G, I, N, M, T, E, | F | X | | |
| | H, V, W, A, R, Q, Y | | | | |
| 63 | L, I | M, F, Y, V, W | X | | |
| 64 | F, W, Y, M, L, I | | | X | |
| 65 | W, E, A, G, N, H, I, V, D, Y, | | | | |
| | F, R, L, T, C, S, Q, M, K | | | | |

TABLE 13WW
PDGER_mb7
DDERLATLAFRALIKRAGVKNLDVKVTNGKVRVTITGRDQASFKALQLVFALARRLGLQVQIDTR
Optimized SEQ ID NO: 1055

| | | | | | |
|---|---|---|---|---|---|
| 1 | V, A, L, R, H, T, I, N, Q, Y, M, | | | | |
| | S, W, F, P, G, E, K, D | | | | |
| 2 | T, Y, S, L, I , P, F, Q, K, H, | | X | | |
| | N, M, E, V, W, G, R, A, D | | | | |
| 3 | S, Y, F, V, H, D, I, N, Q, A, L, | | | | |
| | M, W, E, T, P, G | | | | |
| 4 | L, V, E, S, D, T, W, Y, A, M, H, | | X | | |
| | G, K, F, Q, R, N, P, I | | | | |
| 5 | H | Q, F, W, R, T, M, G, V, | X | | |
| | | L, N, I, K | | | |
| 6 | Y, W, Q, I, T, H, A, N, F, K, V, | | | X | |
| | S, R | | | | |
| 7 | T, Q, E | V, N, A, K, I, D, Y, W, F, R, H, | | | |
| | | S, G, L, M, C, P | | | |
| 8 | E, Y, H, I, K, S, Q, V, N, R, M, | | X | | |
| | A, L, D, T, F | | | | |
| 9 | A, L, M, V, P | | X | | |
| 10 | F, H | | | X | |
| 11 | E, P, M, Y, N, L, A, S, D, Q, V, | | | | |
| | F, T, G, H, W, K, R, I, C | | | | |
| 12 | A | S, I, Y, M, T, F, V, Q | X | | |
| 13 | L, M, I | | X | | |
| 14 | L, V, M, I | | X | | |
| 15 | Q, A, R, T, K, S, H, N, I, W, V, | | X | | |
| | M | | | | |
| 16 | V, K, R, T | Q, W | X | | |
| 17 | T, F, H, R, W, L, I, M, S, Y, | | | X | |
| | A, Q, V, K | | | | |
| 18 | G, K, N, R, W | | | | X |
| 19 | M, H, E, F, K, I, S, R, L, A, | | | X | X |
| | N, V, Y, Q, T | | | | |
| 20 | K, R | H, Q, T, N, V, I, E, D, G, M, L, | | | X |
| | | P, A, S | | | |
| 21 | W, F, M, Q, A, G, Y, E, H, L, | | | | X |
| | C, S, R, K, N, D, T, P | | | | |
| 22 | W | G, L, M, I, V, C, A, F | | X | |
| 23 | Y, T, I, E, L, H, N, G, A, K, | | | | |
| | Q, R, S, P, D, M, V, W, F | | | | |
| 24 | L, V, I, F, W | | | X | |
| 25 | F, E, D, I, H, S, A, Q, W, N, | | | | |
| | T, V, R, M, K, Y, L, C | | | | |
| 26 | I, V, L | | | X | |
| 27 | S, M, I, N, K, Y, R, A, F, Q, | | | | |
| | E, V, H, D, L, C, G, T, W | | | | |
| 28 | M, D, Q, R, T, A, H, S, G, K, | | | | X |
| | E, P, N, Y | | | | |
| 29 | H, Q, N, K, R, G, S, C | | | | X |
| 30 | T, N, A, S, Y, F, H, M, W, C, | | | | |
| | V, I, L, Q, R, K | | | | |
| 31 | V, A, C | | | X | |
| 32 | M, V, L, W, N, H, E, T, Q, A, | | | | |
| | K, Y, S, F, I, R | | | | |
| 33 | I, F, V, L, C, S | | | X | |
| 34 | Y, S, I, M, D, L, N, Q, W, H, | | | | |
| | G, P, V, A, F, R, K, T, E | | | | |
| 35 | G, F, V, S, M, C, Y, T, K, W, | | | X | |
| | D, E, H, N, Q, P, R, A, L, I | | | | |
| 36 | Q, S, N, C, I, A, M, H, K, G, | | | | X |
| | P, W, E, L, V, R, D, T, Y, F | | | | |
| 37 | K, T, A, E, C, H, N, S, Q, D, | | | | X |
| | P, R, G, Y, M | | | | |
| 38 | S, I, Q, M, N, V, F, P, A, Y, | | X | | X |
| | K, L, R, H, T, G, W, C | | | | |
| 39 | H, M, R, K, Q, S, G, F, W, N, | | | | X |
| | E, Y, C, T, D, L, A, V, I, P | | | | |
| 40 | D, G, I, T, C, F, Y, S, P, M, | | X | | |
| | V, H, W, N, A, L, Q, K, R, E | | | | |
| 41 | K, R, M, I, Y, N, G, F, T, H, | | | | |
| | W, S, C, Q, A, L, V, P | | | | |
| 42 | R, M, A, E, Y, F, H, C, N, Q, | | | | |
| | G, T, P, W, K, S, V, L | | | | |
| 43 | F, Y | W, H, P, T, E, I, G, M, | X | | |
| | | N, A, L, R, V, Q, D, | | | |
| | | K, S | | | |
| 44 | R, K, N, Y, H, M, T, Q, S | V, I, E, W, L, P, D, G, | X | | |
| | | A, E | | | |
| 45 | C, P, L, T, M, G, V, A, I, K, | | | X | |
| | Q, S, W | | | | |
| 46 | W, P, F, T, H, V, K, Q, S, M, | | X | | |
| | C, A, L, R, Y, I , G | | | | |
| 47 | R, P, K, Y, V, H, M, F, I, N, | | X | | |
| | W, T, A, S, L, Q, G | | | | |
| 48 | T, Y, K, M, R, L, Q, W | | X | | |
| 49 | L, I, V, F | M, C | | X | |
| 50 | K, Y, R, F, H, W | | X | | |
| 51 | I, H | A | X | | |
| 52 | L, F | | X | | |
| 53 | G, A | | | | |
| 54 | E, A, N, H, R, K, T, Y, F, S, D | | X | | |
| 55 | R | | X | | |
| 56 | F, M, L, E, P | | X | | |
| 57 | H, N, D, S, A, Q, R, E, K, G, | | | | X |
| | M, L, C | | | | |
| 58 | K, Y, R, M, F, L | | | X | X |
| 59 | T, E, A, K, N, M, L, P, H, V, | | | | |
| | D, I, F, S, W, R, Y, Q, G | | | | |
| 60 | T, I, C, M, L, A, P, V, F | | | X | |
| 61 | T, G, D, L, N, M, F, W, E, S, | | | | |
| | A, K, H, Y, I, R, V, Q | | | | |
| 62 | L, V, F, M, I, E | | | X | |
| 63 | W, T, N, E, I, S, K, L, Q, H, | | | | |
| | F, A, G, V, Y, C, D, R, M | | | | |
| 64 | K, R, S, N, A, C, H, L, P, W, | | | | |
| | Y, Q, M, I, G, V, T, F | | | | |
| 65 | C, N, L, W, V, M, S, G, Y, I, | | | | |
| | K, Q, T, A, H, F, P, R, E, D | | | | |

TABLE 13XX
PDGFR_mb8
SELAEEILELIEKGDPRVRKLILELLKELKERGDEKRFKLLVRVWFLLRSGNPELAKQLLKKIKK
(SEQ ID NO: 1057)

| | | | | | |
|---|---|---|---|---|---|
| 1 | F, V, N, H, G, W, T, K, Y, Q, | | | | |
| | R, L, S, I, M, A, E, P, C, D | | | | |
| 2 | S, H, D, Y, C, A, V, W, L, I, | | | | |
| | F, E, M, R, Q, K, N, P, T, G | | | | |
| 3 | C, A, Y, F, M, P, W, G, I, T, N, | | | | |
| | K, S, R, Q, L, V, H, D | | | | |
| 4 | M, P, S, F, I, L, E, V, T, Y, W, | | | X | |
| | N, C, H, G, A, K, Q, R, D | | | | |
| 5 | W, P, L, C, F, I, Y, V, M, G, | | | | |
| | N, D, K, S, R, A, E, H, Q, T | | | | |
| 6 | V, Y, G, C, R, P, A, N, D, K, | | | | |
| | S, M, Q, E, W, L, H, I, T, F | | | | |
| 7 | C, V, L, F, M, W, Y, I, A, E | | | X | |
| 8 | M, G, Q, V, H, A, F, Y, W, I, | | | | |
| | R, C, S, T, L, K, N, E | | | | |
| 9 | I, P, G, H, V, F, Y, A, T, Q, | | | | |
| | L, M, K, D, C, E, W, R, N, S | | | | |
| 10 | Y, W, G, K, Q, R, L, M, H, F, T | | | | |
| 11 | F, V, M, L, I, Y, W | | | X | |
| 12 | Y, H, I, C, G, D, A, N, V, T, | | | | |
| | R, L, Q, M, K, S, E | | | | |
| 13 | G, C, D, S, Y, A, H, M, N, Q, | | | | X |
| | T, V, K, L, R, I, F, E | | | | |
| 14 | H, N, D, Q, E, G, R, K, S | | | | X |
| 15 | Q, E, D, V, N, H, Y, A | | | | X |
| 16 | F, D, Y, W, S, N, H, T, M, V, | | | | |
| | E, R, A, L, K, I, G, P, Q, C | | | | |
| 17 | L, Q, C, H, K, I, T, G, D, Y, | | | | |
| | M, F, N, S, R, A, W, E, V | | | | |
| 18 | I, T, G, C, S, V, L, A, W, P, H | | | | |
| 19 | Q, V, M, L, K, I, R, T, N, S | | X | | |
| 20 | T, G, F, E, S, R, I, Y, N, L, | | X | | |
| | M, H, V, A, Q, D, K, P, W, C | | | | |
| 21 | C, N, R, T, Q, F, H, K, L, M, | | | | |
| | S, V, I, W, Y, A, D, G, E | | | | |
| 22 | M, V, I, L, T, F | | | | |
| 23 | Y, T, F, H, K, E, P, M, G, S, | | X | | |
| | Q, L, I, V, A, B, C, N | | | | |
| 24 | L, S, D, A, F, Y, C, V, T, I, | | | | |
| | K, Q, P, R, W, H, E, N, M, G | | | | |
| 25 | K, H, Y, V, M, Q, W, C, G, P, | | | X | |
| | F, S, E, D, I, A, T, L, N, R | | | | |
| 26 | V, S, C, A, T, I, L, M | | X | | |
| 27 | L | V, T, Q, A, H, R, K | X | | |
| 28 | W, V, D, F, R, L, T, Q, A, I, | | | | |
| | G, C, M, S, P, E, N, H, K, Y | | | | |
| 29 | H, A, S | M, Q, W, C, N, T, I, G, V, E, | | X | |
| | | L, F, K, R | | | |
| 30 | H, R, K, Q, Y, N | | | | |
| 31 | T, S, C, H, Y, Q, R, A, D, G, | | X | | |
| | F, I, K, M, V, W, L, E, N, P | | | | |
| 32 | L, S, T, V, M, N, A, K, Q, R, | | | | X |
| | I, H, G, Y, C, F | | | | |
| 33 | A, Y, N, H, S , R, G, Q, D, K, | | | | X |
| | P, F, M | | | | |
| 34 | R, E, F, N, M, A, S, H, Y, D, | | | | X |
| | Q, W, L, K, T, I, P, G | | | | |
| 35 | P, L, T, K, V, R, N, A, I, D, | | | | |
| | G, E, M, C, W, Q, S, Y, | | | | |
| | H, F | | | | |
| 36 | M, P, N, R, A, K, S, Q, V, H, | | | | |
| | T, F, Y, I | | | | |
| 37 | H, M, A, N, R, G, T, K, Q, W, | | | | |
| | Y, S, L, P, F, E | | | | |
| 38 | G, M, V, Y, W, S, F, R, I, A, T | | X | | |
| 39 | V, I, L, H, N, Q, M, R, T, W, | | X | | |
| | K, Y, A, F | | | | |
| 40 | E, Y, D, N, P, A, I, W, G, V, | | X | | |
| | H, K, S, T, C, L, Q, R, M, F | | | | |
| 41 | I, F, Y, M, L, W, C, V | | | X | |
| 42 | I, V, M, L, A, Q | | X | | |
| 43 | T, G, K, P, R, S | | X | | |
| 44 | T, L, I, V, P, C, G | | | X | |
| 45 | F, Y, W, P | | X | | |
| 46 | F | | X | | |
| 47 | W | G, F, M, Y, H, L, D | X | | |
| 48 | I, M, V, L, H, W | | | X | |
| 49 | I, R, A | | | | |
| 50 | I, V, L | M, T, C, A, S | X | | X |
| 51 | N, G, S, R, K, H, F | | | | X |
| 52 | H, R, L, K | Y, T, I, C, A, Q, W, S, | X | | X |
| | | N, M, F, V | | | |
| 53 | V, K, N, W, A, S, Q, T, G, R, | | | | |
| | P, D, M, H, L, E, I | | | | |
| 54 | N, C, R, D, G, F, I, H, V, M, | | | | |
| | Y, L, E, S, Q, P, W, A, T, K | | | | |
| 55 | I, V, L, H | | X | | |
| 56 | V, A, I, E, M | | | X | |
| 57 | R, M, K, A, I, V, S, T, Q, L, E | | | | |
| 58 | M, W, T, E, S, L, G, F, H, Y, | | X | | |
| | R, V, Q, N, I, K, A | | | | |
| 59 | Y | H, R, A, T, V, K, Q, | X | | |
| | | I, S, L | | | |
| 60 | I, V, L, M, A, W, T | | | X | |
| 61 | G, C, A, T, V, S, F, N, L, I, | | | | |
| | M, Y, Q, K, W, H, R, E, D, P | | | | |
| 62 | N, W, S, G, P, H, K, R, A, M, Q, | | X | | |
| | C, T, L, Y, F, I, V | | | | |
| 63 | Y, E, A, H, K, P, D, G, W, | T, Q | | X | |
| | M, C, L, R, I, V, F, N | | | | |
| 64 | S, A, T, N, Q, R, I, H, L, P, | | | | |
| | C, E, D, M, V, K, G, W, Y | | | | |
| 65 | Q, F, C, P, T, N, S, A, V, H, | | | | |
| | L, G, D, M, R, I, Y, K, E | | | | |

TABLE 13YY
PDGER_mb9
DKAREFLRAAAKYGDPSAKRALKLLEELRKRGIPPEEALKIVVEFLRKAGNPRLARVVEEALKDE
(SEQ ID NO: 1058)

| | | | | | |
|---|---|---|---|---|---|
| 1 | G, R, K, E, F, M, Y, W, H, S, C, | | | | |
| | A, V, L, Q, D, T, I, N, P | | | | |
| 2 | T, K, V, I, R, E, N, Q | A | X | | |
| 3 | A, I | G | | X | |
| 4 | F, R, M, H, Y, N, T, I, C, | E, Q, V, G, A | | | |
| | W, S, L, K, P | | | | |
| 5 | R, K, D, T, P, A, M, S | L, N, E, Q, H, I, V, W, | X | | |
| | | Y, G | | | |
| 6 | F | | X | | |
| 7 | L | V | | X | |
| 8 | K, R, M, H, S, C, L | T, Q, G, A, F | X | | |
| 9 | R, A, L, Y, M, F, E, T, V, | K | X | | |
| | S, P | | | | |
| 10 | A, C | T | X | | |
| 11 | A, R, G | S, T | | | |
| 12 | K, R, Q | G | | | |
| 13 | H, Y, R, K, L, F | W | X | | |
| 14 | K, R, G, H, Q, N, E, W, A, | M, L | | | X |
| | V | | | | |
| 15 | D | | X | | X |
| 16 | Y, G, H, I, T, R, W, F, V, | K, E | X | | |
| | S, P, M, N, A, L, Q | | | | |
| 17 | W, Y, F | H, S, G, M, D, C | | X | |
| 18 | A | S | | X | |
| 19 | R, K, I, N, E | | | | |
| 20 | I, M, W, F, V, L, R, Y, T, | N, Q, A | | | |
| | S, G, C, H, P | | | | |
| 21 | M, T | Y, L, Q, H, F, I, V, A, C, N, S, E | | X | |
| 22 | F, T, Y, N, I, S, L, C, V, | Q, K, G | | | |
| | W, M, R, H, A | | | | |
| 23 | W, Y, I, F, T, M, R, V, K, | A, N, S, C, Q | | | |
| | H, L | | | | |
| 24 | W | K, A, R, V, F, H, Q, L, G, Y, N, | | | |
| | | D, T, E, C, I, S | | | |
| 25 | D, N, L, F, V, I, T, M, Y, | C, E, S | | | |
| | A, Q, R | | | | |
| 26 | N, G, F, Q, Y, V, L, H, A, | C, P | | | |
| | T, M, S, E, I, W, D, R | | | | |
| 27 | I, V, L | A, M, T, G, H, N, Y, Q, F, E, S, | | | |
| | | P, R, D, W | | | |
| 28 | I, L, F | V, C, M, W, Y | | X | |
| 29 | R, V, E, I, K, C, M, D, H, | A, T, S, N, G, L, Y | | | |
| | Q | | | | |
| 30 | K, R, C | H, P, V, L, W, G, M | | | |
| 31 | H, R, M, A, L, F, S | V, I, W, C, P, N, T, G, Y | | | |
| 32 | R, G, K, Y, F, H, C, W, M, | D, E, P | | | X |
| | A, L, S, N, Q, T, V, I | | | | |
| 33 | Y, F, W, I, C, H, L, V, T | R, K, Q | | | X |
| 34 | R, N, L, H, P, S, D, F, E, | C | | | X |
| | K, Q, Y, W, A, M, I, T, | | | | |
| | G, V | | | | |
| 35 | Q, P, G, L, A, K, S, T, R | N, M, W, C, H | | | |
| 36 | M, Y, V, F, S, W, C, A, E, | H | | | |
| | K, Q, G, L, I, N, T, R, | | | | |
| | D, P | | | | |
| 37 | C, V, H, M, Y, Q, S, I, G, W, | | | | |
| | K, D, P, R, A, T, L, E, N, F | | | | |
| 38 | S, H, A, D, G, E, C, Q | T | | | |
| 39 | G, M, P, T, L, E, A, S, I, | N, F, H, Q | | X | |
| | D, V, C | | | | |
| 40 | I, S, D, K, E, N, Q, A, C, | G, W, F | | | |
| | M, L, H, Y, V, T, R | | | | |
| 41 | I, W, F | L, M, R, Y, V, K, E, C, G | | | |
| 42 | M, L, T, F, V, N, S, A, C, | E, Y, P | | X | |
| | D, G, I, H | | | | |
| 43 | I, P, V, L, M, E, H, A, G | C, F | | | |
| 44 | W, F, A, H, T, Q, V, L, I, | D, P, C | | | |
| | G, E, K, Y, M, N , R, S | | | | |
| 45 | I, R, V, K, W, E, C, L, N, D, | | | | |
| | T, F, M, S, Y, G, H, A, Q | | | | |
| 46 | E, N, T, Q, M, F, I, S | V, W, C, Y, H, A, G, L, D | | X | |
| 47 | K, A, R, G, S, L, P, H | I, T, W, C, N | | | |
| 48 | I, F, T, S, Q, P, Y | C, R, W, N, M, G, K, H, V, A, | | | |
| | | L, E, D | | | |
| 49 | T, F, W, I | C, P, V, H, L, R, M, Y, N, S, A, | | X | X |
| | | G, K, D, Q, E | | | |
| 50 | I, W, V | L, M, E, F, T, C, Y, D, | X | | X |
| | | S, G, R, H, A, P, K | | | |
| 51 | I, W, F, G, Y, N, P, D | H, Q, M, A, T | | | X |
| 52 | P, K | R, C, S, E, F, D | | | |
| 53 | R | K | X | | |
| 54 | M, I, L, Q, V, N, P | | X | | |
| 55 | F, A, G, S, Y, H, W, V, T, | N, I, K | | | |
| | E | | | | |
| 56 | R | N, K, G, D, C, M, A | X | | |
| 57 | V, L | C, I | X | | |
| 58 | A, T, V, L, S, F, I, N | Y, C, E | | X | |
| 59 | D, G, S, E, N, C, F, Y | H, T, A, M, Q, L, R, W, K | | | |
| 60 | E | D | X | | |
| 61 | F, M, I | V, A, L, W, R | | X | |
| 62 | W, S, G, A | H, M, T, C, F, R, L, I, V, Q, Y, | | X | |
| | | N, K | | | |
| 63 | D, T | E, G, L, C, F, M, S, W, H, Q, P, | | | |
| | | R, V, A, K, Y, N, I | | | |
| 64 | N, D, Y, G, V, E | S, H, P, T | X | | |
| 65 | S, N, H, K, Q, E, R, P, V, | W | | | |
| | G, I, T, L, A, F, D, M, | | | | |

Y, C

TABLE 13ZZ
PDGFR_mb10
SLIEEILKEVRKKNPRVVYILERLLRRLKEDGVPPEKILRILIAAAKRLGDPEASELLRRALKRK
(SEQ ID NO: 1059)

| | | | | | |
|---|---|---|---|---|---|
| 1 | H, P, R, M, I, L, A, K, C, | W | | | |
| | V, F, E, D, G, Y, N, Q, | | | | |
| | T, S | | | | |
| 2 | R, Q, K, E, S, Y, T, A | N, C, D, H, W, M, G, I, P, V, E, | | | |
| | | L | | | |
| 3 | P, I, N, H, Q, T, V, S, F, K, | | | | |
| | D, M, G, E, W, A, C, R, Y, I | | | | |
| 4 | H, N, Y, T, G, F, W | M, R, S, V, A, Q, P, I, D, L, C, | | | |
| | | K, E | | | |
| 5 | N, R, A, Q, F, M, H, K, S | I, T, P, L, V, D, W, C, G, E | | | |
| 6 | Q, W, F, Y, M, R, K, V, G, | E, N, D, T | | X | |
| | P, C, S, H, A, I, L | | | | |
| 7 | N, F, D, P, G, I, H | A, T, K, M, S, Q, W, L | | | |
| 8 | R, M, W, E, H, N, A, G, T, | | | | |
| | S, V, F, I, D, Y, P, Q, L, C, K | | | | |
| 9 | Y, H, R, K, N, Q, A, T, D, | C, I, W, M, E | | | |
| | F, G, P, S, V, L | | | | |
| 10 | Y, F, L, D, K, W, C, M, N, | H, G, I, E, P, R | | X | |
| | Q, S, T, A, V | | | | |
| 11 | Q, F, G, D, A, H, E, N, M, | | | | |
| | P, K, T, W, L, I, S, Y, V, C, R | | | | |
| 12 | C | A, R, H, Q, T, I, G, V, N, S, D, | | | |
| | | Y, E, P, M, L, F, K | | | |
| 13 | H, C, R, W, V, I, S, E, M, | | | | X |
| | T, Q, Y, P, L, F, A, G, N, K | | | | |
| 14 | E, Y, V, Q, I, G, S, A, R, | D, H, M, T, P, C | | | X |
| | K, N | | | | |
| 15 | M, C, K, H, L, R | W, D, Q, F, S, T, V, E, | X | | |
| | | G, A, N, Y, I, P | | | |
| 16 | A, T, L, M, Y, W, K, C, H, D, E, | | X | | |
| | V, P, Q, I, N, G, S, F, R | | | | |
| 17 | H, A, Y, T, F, I, N | L, W, K, S, R, Q, D, M, G, V | | X | |
| 18 | R, M, Y, W | F, H, G, K, A, S, C, D, P, T, Q, | | | |
| | | E, N, I, L, V | | | |
| 19 | L, R, G, S, I, M, Q, N, A, | E, C, T, K, W, D, H, Y | X | | |
| | V, F, P | | | | |
| 20 | P, A, T, S | Y, V, F, M, Q, L, W, R, | X | | |
| | | G, H, I | | | |
| 21 | I | M, Y, V, L | | X | |
| 22 | N, Q, H, L, R, V, M, A, K, | W, T, I, C, P, D, F, G, E | | | |
| | S, Y | | | | |
| 23 | D, H, Q, K | A, M, T, I, V, N, G, L, | X | | |
| | | S, F, Y, E, P, R | | | |
| 24 | I, F, M, V, L | | X | | |
| 25 | I, V, H, W, F, T, M, N, Q, | | | | |
| | C, K, R, Y, S, L | | | | |
| 26 | M, L, C, Q, I, A, G, K, S, | | | | |
| | W, H, F, V, P, N, Y, T, R | | | | |
| 27 | K, Q, W, D, E, R, N | | X | | |
| 28 | M, C, V, I, T, L | | X | | |
| 29 | P ,R, K | F, D, S, Y, V, N, C, E, A, T, I, | | | |
| | | G, L, W, M, H, Q | | | |
| 30 | V, I, M, L, A | N, T, F, Q, S, R, H, G, W, C, K, | | | |
| | | D, Y, E | | | |
| 31 | F, T, W, Q, H, I, E, N, S, G, | | X | | |
| | D, A, L, Y | | | | |
| 32 | K, E, A, D, L, H, I, C, R | T, V, N, Y, F, S, M, W, G | | | X |
| 33 | H, L, I, W, F, C, Q | Y, D, S, T, E, A, M, N, V | | | X |
| 34 | A, Q, M, F, K, D, G, L, S, | | | | X |
| | T, V, H, E, R, N, Y, I, P, C | | | | |
| 35 | W, H, P | I, Q, L, C, A, Y, F, V, | | | |
| | | M, E, G, N, S, D, R, K, T | | | |
| 36 | M, F, T, N, W, H, L, I, C, | | | | |
| | A, Q, V, G, S, Y, D, P, E, K | | | | |
| 37 | N, M, T, Y | D, R, L, I, H, Q, V, F, | X | | |
| | | E, A, G, S, W, K | | | |
| 38 | Q, H, W, Y, N, R, F, T, M | A, V, L, E, G, K, C, S, I | | | |
| 39 | P, A, V, R | Q, K, T, H, E, G, I, C, | | X | |
| | | D, M, W, N, Y, F, S, L | | | |
| 40 | Q, L, H, P, A, N, M, F, I, | G, Y, W, D, T | X | | |
| | V, S, E, K, R | | | | |
| 41 | M, G, V, F, Y, A, I, L | | X | | |
| 42 | M | C, S, V, Y, F, L | | X | |
| 43 | G, R, S, A, K, V, Y | P, L, W, E, H, F, M, C, | X | | |
| | | N, T, I | | | |
| 44 | G, M | Q, L, I, F, V, S, W, H, | X | | |
| | | Y, A | | | |
| 45 | A, G | | X | | |
| 46 | I, Y, T, N, C, W, S, Q, V, | | | X | |
| | G, D, E, A, M, H | | | | |
| 47 | D, E, Q, M, N, L, G, V, I, | | X | | |
| | A, S, H, T, R, Y, K, W | | | | |
| 48 | K, F | Y, W, H, R | X | | |
| 49 | I, M, F, V, L | | X | | |
| 50 | A, T, Q, N, L, G | I, H, W, R, E, K, S, F, | | | X |
| | | D, M, Y, C | | | |
| 51 | H, Y, I, A, T, W, V, S, Q, | G, C, E, D | | X | X |
| | F, N, L, M | | | | |
| 52 | W, Y, V, M | H, T, A, L, E, D, N, Q, | | | |
| | | S, G, C, R, P | | | |
| 53 | K, N, V, L, R, I, S, M, G, | T, D, C, E | | | |
| | W, Q, A, F, Y, H | | | | |
| 54 | Y, L, S, A, Q | E, G, H, V, F, W, C | | X | |
| 55 | Y, M, G, F, L, R, V, T, W, | Q, H, P, D, I, N, A, C | | | |
| | S, K | | | | |
| 56 | N, S, R, F, W, I, Q, M, K | A, V, C, H, D, Y, T, E | | | |
| 57 | P, H, S, W, T, Y, E, F, N, | M, K, R, D, A, Q, I, C, | | | |
| | V, G | L | | | |
| 58 | G, I, F, P, A, D, Q, C, M, | W | | | X |
| | L | | | | |
| 59 | N, A, S, H, M, Y, T, G, D, | I, V, C, R | | X | |
| | Q, E, W, K, L | | | | |
| 60 | E, M, G, L, F, I | K, T, S, N, D, H, P, Y, V, A, Q, W, | | | |
| | | C, R | | | |
| 61 | N, Q, S, I, L, F, P, M, Y, | T, R, E, W | | | X |
| | V, K, G, C, A | | | | |
| 62 | I, F, G, K, V, D, E, W, T, | Y, S, H, P, N, Q, C, A | | | |
| | M, R, L | | | | |
| 63 | M, V, A, Q, G, Y, L, S, I, | F, H, R, D, W, C, P, K | | | |
| | E, T | | | | |
| 64 | P, Y, L, Q, W, N, V, T, I, S, | | | | |
| | G, F, C, K, E, M, D, A, H, R | | | | |
| 65 | F, D, T, C, M, I, H, R, W, | Y, G, N, K | | | X |
| | L, S, V, A, Q, E, P | | | | |

TABLE 13AAA
TGFb_mb1
HCTIEVVGVDPEKVEAIAAAYGAEVCEKDGKFEIHLDDPHSAESAAVAISVLTNRPVRLQC
(SEQ ID NO: 1347)

| 1 | C, L, R, D, K, H, Y, Q, W, | T, P, E, S | | | |
|---|---|---|---|---|---|
| | I, F | | | | |
| 2 | C, E, V, D, L, R | A, Y | | X | |
| 3 | I, E, G, N, V, F, Q, C, P, | T, D, S, Y | | | |
| | K, H | | | | |
| 4 | T, I, P | G, S, M | | X | |
| 5 | F, G, N, S, Q, P | E, T, L, M, C | | | |
| 6 | C, F, T | N, A, S, V, E, K, R | | X | |
| 7 | P ,K, G | N, C, V, D, L, A, E, S, W, Q, M, Y | | | |
| 8 | N, V | G, C, E, L, I, W | | | X |
| 9 | E, V, K | S, F, A, Y, L, R | | X | X |
| 10 | E, K | D, Y, A, W, C, S, G, Q, | X | | X |
| | | P | | | |
| 11 | E, S, F | P, G, R, H, V, K, I | | | |
| 12 | F, D, H, E, V, R, N, L, Q | W, P, T | X | | |
| 13 | I, K, L, R | T, H, A, S, D, E, N, F | | X | |
| 14 | I, L, V, Y | A, Q, M, K, P, G, F, R | | X | |
| 15 | G, F, C | Q, E, N, D, R, W | X | | |
| 16 | V, I | A, W, C, Q, K, H, M, G, | X | | |
| | | L | | | |
| 17 | E, T | I, P, R, Q, L, K, N, C, | X | | |
| | | Y | | | |
| 18 | C, I, K | A, Q, G, T, M, V | | | |
| 19 | Q, F, A, E, C | N, S, D, T, V, L | | | |
| 20 | T, N, C | A, M, W, E, S, H, I, V, | X | | |
| | | G | | | |
| 21 | Q | S, V, F, P, C, R, L, N, Y, K | | X | |
| 22 | E, G, V, K, F | Y, A, S, L, T, C, N | | | X |
| 23 | G, T, A, P, K, R | S, I, H, L, E, F, C, N | | X | X |
| 24 | W | E, G, P, I, K, H, L, A, V | | | |
| 25 | D, N, S, V, K, Q | E, T, R, H, F, A, I, P, M, G | | X | |
| 26 | D, L, H, Q, F, R, Y, K, V | I, P, N, C, G, S, E, T, W | | | |
| 27 | R, E, D, L | S, H, I, V, A, P, G, Q | | | |
| 28 | K, S, G, Q, N, F | D, H, P, C, E, I, Y, T, R | | | |
| 29 | G, T, D, P, V, R | H, S, N, Y, E, A, I | | | X |
| 30 | F, A, G, W, L | M, D, S, Q, C, N, K, V, I | | | X |
| 31 | K, A, G, I, P, F, V, N, R | Y, M, S, L, H, T | | | |
| 32 | F | T, L, I, V, S, K, Y, C, R, N, Q, D, | | X | |
| | | H | | | |
| 33 | A, S, F, K, Y, T, V | E, C, M, I, Q | | | |
| 34 | I, R, F, M, L, T | Y, Q, H, W, G, N, D, P | | X | |
| 35 | G, R, T, V | H, K, D, L, C, S, N, M, A, I | | | |
| 36 | L, C, I, R | G, A, Q, D, P, Y, N, M | | X | |
| 37 | D, K, G, E | R, H, S, Q, Y | | | X |
| 38 | D, G | Q, C, I, N, E | | | X |
| 39 | Y, F, P | D, N, T, V, A, L, S | | | |
| 40 | W, V | H, E, N, L, Y, K, G, Q, | X | | |
| | | D, P | | | |
| 41 | Y, K, M, V, N | S, T, G, A, D, Q, C | | | |
| 42 | P, G, A, F | L, N, V, I, M, E, K, Y, C | | | |
| 43 | Y, F | L, V, E, H, D, S, G, K, | X | | |
| | | Q, A, I, P, N | | | |
| 44 | K, G, S | I, W, E, P, M, Q, N | X | | |
| 45 | A, N, E, M | R, W, T, P, S | | X | |
| 46 | Y, A, T, L, M | P, W, E, G, I, S, V, C, K, Q, D, N | | | |
| 47 | A, C, R, T, N | I, W, G, P, M, V | X | | |
| 48 | A, G | S, P, T, I, N, E | X | | |
| 49 | I, N | F, Q, D, A, R, C | | X | |
| 50 | H, S, G, E, Y | V, R, I, N, D, T, C, K, F | | | |
| 51 | M, S, F, V, A, I | L, D, P, W | X | | |
| 52 | L, G, E, F | I, Y, S, C, A | X | | |
| 53 | C, G, M, T | A, L, P, D, S, H, E, I, R, K | | X | |
| 54 | N, T | G, P, Y, K, I , F, V, C, | X | | |
| | | L | | | |
| 55 | R, K | Q, V, N, C, H, F, E, G, Y, A, P | | X | X |
| 56 | D | P, R, G, N, I, S, C, L, W, M, F, Y | | | X |
| 57 | P, C, S | G, V, D, T, E, I, W | | X | |
| 58 | D, G, P, H, R, N | S, E, W, Q, A, Y, T, L, V, M | | | |
| 59 | E, S, A, D | Q, H, E, T, R, L, K, M, I, G, W | | | |
| 60 | T, M, Q, R, H | P, W, G, Y, F, D, K, C, A, V, L, I | | | |
| 61 | E, C, G, F | M, W, N, A, Q, P | | | |

TABLE 13BBB
Tie2_mb1
PEEFVREALRRLIPDPRLQKIVEEALELAKRLGIDPFEVLQILDFLLIYLGDPEEAAKILEELVR
(SEQ ID NO: 1449)

| | | | | | |
|---|---|---|---|---|---|
| 1 | W, M, I, P, D, Y, K, H, V, | R, T | X | | |
| | L, N, Q, A, E, G, F, S, | | | | |
| | C | | | | |
| 2 | W, Y, C, I, L, H, V, E, F, | N, T, P, Q | | | |
| | M, A, S, D | | | | |
| 3 | M, L, W, I, A, V, C, F, E, | G, R, K | | | |
| | Q, P, D, H, N, T, Y, S | | | | |
| 4 | Y, F, C | | X | | |
| 5 | V, C, T, I | | X | | |
| 6 | C, R, M, N | T, K, H, Y, Q, A, L, V | | | |
| 7 | C, L, F, M, W, A, E, Q, V, | H, Y, S, D, T | | | |
| | I | | | | |
| 8 | V, C, I, W, Y, A, M, T, L | S, Q | X | | |
| 9 | C, L, M | A, F | | X | |
| 10 | S, C, R, Y, W, F, L, N, M, | H, D | | | |
| | G, Q, K, T, A | | | | |
| 11 | C, W, M, F, I, L, V, R, Y, | Q | X | | |
| | H, S, A, T, N, K | | | | |
| 12 | C, F, L, I, W, Y | Q, M | | X | |
| 13 | H, Y, F, W, A, C, N, S, T, | | | X | X |
| | G, R, Q, D, E, M, I, L, K, P, V | | | | |
| 14 | C, N, H, K, S, F, D, T, Q, R, | | | | X |
| | Y, M, I, A, L, W, G, P, E, V | | | | |
| 15 | H, T, W, N, S, G, I, Q, C, A, | | | | X |
| | P, V, L, K, Y, M, F, R, D, E | | | | |
| 16 | G, D, A, P, R, F, K, W, M, | L | | | |
| | N, T, I, S, E, H, C, Y, | | | | |
| | V, Q | | | | |
| 17 | H, R, M, P, S, Q, C, G, K, N, | | | | |
| | V, T, F, D, E, L, A, Y, W, I | | | | |
| 18 | H, F, W, G, Y, L, N, C, R, S, | | | | |
| | M, D, A, Q | | | | |
| 19 | W, E, G, K, T, E, D, A, Q, | I | | | |
| | M, N, L, Y, S, H, C | | | | |
| 20 | P, Y, C, Q, W, K, H, R, D, L, | | | | |
| | N, T, I, S, G, E, F, A, M, V | | | | |
| 21 | P, A, I, V, G, S, T, C | | | | |
| 22 | S, W, A, G, I, P, F, M, V, | Y | | X | |
| | T, L, C | | | | |
| 23 | D, E, N, Q, H, C, K, G, S, | T, A, V, I | | | |
| | R | | | | |
| 24 | M, E, P, Q, K, A, S, D, T, | G | | | |
| | H | | | | |
| 25 | M, A, Y, C, L | V | | X | |
| 26 | W, L, Q, C, V, I, M, Y, F | T | | | |
| 27 | C, S, R, D, Q, L, E, F, A, V, | | | | |
| | I, G, K, M, N, Y, T, W, H | | | | |
| 28 | L, Y, F, V, M, C | | | | |
| 29 | G, A, S | | | X | |
| 30 | F, N, Y, K, H, D, R, S, A, | M, I, L, W, V | | | |
| | C, Q, G, E, T | | | | |
| 31 | R, M, I, L, F, N, V, K, D, C, | | | | |
| | H, S, Q, A, T, G, P, Y, W | | | | |
| 32 | L, E, Y, D, M, W, C, T, H, F | | | | X |
| 33 | V, D, A, K, H, G, Q, E, S, L, | | | | X |
| | C, N, R, I, M, T, Y, F | | | | |
| 34 | Y, I, E, V, H, F, T | N, A, Q | | | X |
| 35 | C, D, E, H | | X | | X |
| 36 | L, C, W, P, M, Q, Y, T, A, | G, H, E, V, I | | | |
| | D, S | | | | |
| 37 | Y, C, F, G, V, M, N, L, W, A, | | X | | |
| | D, Q, T, H, I | | | | |
| 38 | E, D, N, Y | C | X | | |
| 39 | A, V, G, L, F, T, S, C, Y | | | X | |
| 40 | F, W, Y, L, C, V, M | | X | | |
| 41 | E, T, Q, H, N, Y, D, W | S, C | X | | |
| 42 | I, W, F, V, Y | | | | |
| 43 | I, V, T, C, A, L, M, S, F | | | X | |
| 44 | D, R | | X | | |
| 45 | L, M, I, V, F, W, T, C | E | X | | |
| 46 | F, L, M | | | X | |
| 47 | C, V, F, M, W, L, I, Y, T | N | | | |
| 48 | C, I, F | M | X | | |
| 49 | C, S, L, W, V, Y, F, I, A, M, G | | X | | |
| 50 | W, V, F, I, T, M, R, K, S, A, C, | | | | |
| | L, Y, Q, G, N | | | | |
| 51 | P, C, H, T, S, K, G, Q, N, | E | | | X |
| | A | | | | |
| 52 | C, K, E, D, G, T, S, P | V, A, R, Q | | | X |
| 53 | G, S, T, N, C, R, Y, Q, K, F, P, | | | | |
| | W, H, A, E, D, M, V | | | | |
| 54 | C, I, K, M, H, T, R, Y, Q, | A, P | | | |
| | N, V, W, F, D, E, G, L, | | | | |
| | S | | | | |
| 55 | D, E, S, V, T, C, P, I, A, Q | | | | |
| 56 | V, L, F, I, T, W, M, C, A | | | X | |
| 57 | M, A, C, G, I, T, S, L | V | | | |
| 58 | K, P, Q, N, A, G, T, H, C, S, D, | | | | |
| | R, E, M, I, L | | | | |
| 59 | P, I, Y, V, L | M, C, T, Q, W | | | |
| 60 | M, W, F, L | V, I | | X | |
| 61 | E, G, Q, D, A, S | | | | |
| 62 | K, R, N, A, L, P, Q, V, G, T, S, | | | | |
| | M, C, E, D, H, I | | | | |
| 63 | T, E, L, G, A, F, S, V, C, M, Q | | | | |
| 64 | M, L, C, S, T, V, I | | | X | |
| 65 | H, P, R, G, M, K, F, A, E, | T | | | |
| | N, S, L, Y, C, D, V, I, | | | | |
| | W, Q | | | | |

TABLE 13CCC
Tie2_mb2
SIDEELERLLEEAGVDPELIDDLYAVIYQLYIRGLSDKDVLRFLLENLERDGTPLRRVVEELLKR
SEQ ID NO: 1451

| | | | | | |
|---|---|---|---|---|---|
| 1 | D, F, C, T, N, Y, W, G, S, P, M, | | | | |
| | H, V, L, E, A, I | | | | |
| 2 | W, C, P, I, M, F, V, L, G, Y, E, | | | X | |
| | A, D, S, T, H | | | | |
| 3 | D, W, F, E, C, P, Y, N, T, | A | | | |
| | M, H, V, L, S, G, Q, I | | | | |
| | D, W, | | | | |
| 4 | P, C, D, E, F, Y, S, L, W | I | | | |
| 5 | V, C, F, I, Y, T, M, W, A, H, P, | | | | |
| | E, L, G, R, N, D, K, S, Q | | | | |
| 6 | S, T, C, V, M, L, I, G, P, A, E, | | | X | |
| | W, Q, H, N | | | | |
| 7 | P, F, E, L, W, Y, D, M, I, H | | | | |
| 8 | Y, W, P, F, A, G, M, C, E, V, L, | | | | |
| | D, I, Q, N, H, S, T, R, K | | | | |
| 9 | E, L, D, C, P, W, N, S, Y, A, M, | | | | |
| | F, I, Q, R, T, K, V, H | | | | |
| 10 | N, E, H, Q, P, C, D, K, L, | M | | X | |
| | G, T, R, Y, A, I, V | | | | |
| 11 | W, E, P, D, Y, Q, F, V, T, M, S, | | | | |
| | A, H, L, I, N, R, G, K | | | | |
| 12 | E, D, Q, K, G, S, R, N, P | | | | |
| 13 | D, P, E, A, H, Q, R, Y, K, T, N, | | | | X |
| | G, S, L, V, M, C, W | | | | |
| 14 | E, R, N, H, S, M, V, G, C, D, L, | | | | X |
| | K, Y, F, P, A, I, Q, T, W | | | | |
| 15 | V, C, T, I, E | | | X | |
| 16 | C, D, E, H, M, S, L, K, T, Q, F, | | | | X |
| | N, I, A, V, G, P, Y | | | | |
| 17 | E, D, C, Y, A, F, W, V, N, G, M, | | | | |
| | T, Q, S, P, I, L, H, K, R | | | | |
| 18 | C, E, D, M, I, S, N, L, A, P, V, | | | | |
| | G, T, Q, F, H | | | | |
| 19 | D, E, L, T, I , Q, C, N, A, H, S, | | | | |
| | V, P, M, Y, K | | | | |
| 20 | I, Q, V, S, E, M, R, C, F, W, T, | | | | |
| | G, Y, H, A | | | | |
| 21 | D, E, C, F, Y, A, Q, W, M, S, H, | | X | | |
| | N, G, L, T | | | | |
| 22 | D, Q, N, S, E, G, A, C, K, M, R | | X | | |
| 23 | V, L, T, C, M, N, A, S, I | | | X | |
| 24 | Y, V, E, T, Q, I, D, H, M, L, F, | | | | |
| | N, W, S, G, A, K | | | | |
| 25 | E, Q, D, N, G, A, C, M, H, V, S, | | X | | |
| | L, T, W, I, Y | | | | |
| 26 | V | | X | | |
| 27 | I, V, E, Y | | | X | |
| 28 | Y, N, F, C, A, E, W, G, I, | M | X | | |
| | D, V, H, P, L, S, Q, T | | | | |
| 29 | E, Q, D | | X | | |
| 30 | L | | X | | |
| 31 | Y, M, A, E, F, R, T, Q, I, S, L, | | | | |
| | V, W, C, H, K | | | | |
| 32 | I, M, L, V, C | | X | | X |
| 33 | I, L, V, R, T, M | | X | | X |
| 34 | G | | | | X |
| 35 | L, M | | | | X |
| 36 | G, R, H, W, N, A, Y, S, K, Q, F, | | | | X |
| | V, M, T, C, P, L, E, I, D | | | | X |
| 37 | D, N, C, E, F, W, G, H, Y, | M | | | |
| | R, S, T, L, K, Q, A, V, | | | | |
| | I, P | | | | |
| 38 | C, I, M, L, F, W, P, Y, V, E, K, | | | | |
| | D, Q, R, T, S, A, G, N, H | | | | |
| 39 | Q, A, G, S, N, E, T, C, D, M, H, | | | | |
| | R, K, Y, W | | | | |
| 40 | I, V, Q, W, T, M, C | | | | |
| 41 | L, C, F, M, I | | | | |
| 42 | Y, R, L, C, I, H, Q, V, K, G, S, | | | | |
| | M, A, F | | | | |
| 43 | Y, L, F, V, I, H | | X | | |
| 44 | C, I, V, L, T, M | | | X | |
| 45 | D, C, L, T, S, A, V, I, N, M, H, | | | | |
| | E, Y, K, R, Q, G, F | | | | |
| 46 | E, V, A, I, Q, D, C, T | | | | |
| 47 | N, H, Y, E, F, W, Q, T, S, M, V, | | X | | |
| | G, A, C, L | | | | |
| 48 | L, R, I | | | X | |
| 49 | P, E, D, A, V | | | | |
| 50 | D, E, V, Q, K, R, L, I, P, | N | X | | |
| | H, M, A, S, F, Y, T, W, | | | | |
| | C | | | | |
| 51 | D, E, N | | X | | |
| 52 | M, E, Y, L, F, D, Q, G, N, H, C, | | | | X |
| | K, A, S, R, W, V, T | | | | |
| 53 | T, S, G, A, C, N, L, H, K, P, Q, | | | | X |
| | D, M, E, F, R | | | | |
| 54 | P, M, I, D, A, V, Q, E, G, N, S, | | | | |
| | L, T, H | | | | |
| 55 | M, W, L, T, V, C, F, I, Y | | | | |
| 56 | I, Y, F, R, T, W, V, Q, M, A, C, | | | | |
| | L, P, S, N | | | | |
| 57 | P, R, T, L, M, S, E, G, D, N, A, | | | | |
| | F, H, V, K, Y, I, W, Q, C | | | | |
| 58 | Y, V, H, C, T, W, P, F, L, I, M, | | | X | |
| | S, N, A, Q, G, E | | | | |
| 59 | C, V, I, L, M, T | | | X | |
| 60 | F, W, E, A, Y, C, Q, S, N, | V | | | |
| | R, T, G, H, M, K, D, L | | | | |
| 61 | D, N, E, H, Q, S, M, C, R, F, K, | | | | |
| | A, T, G, W, Y, V, I | | | | |
| 62 | H, Y, L, E, C, T, Q, S, E, V, A, | | | X | |
| | R, M, K | | | | |
| 63 | H, N, L, W, A, S, Y, Q, F, T, V, | | | | |
| | E, M, D, C | | | | |
| 64 | F, W, T, I, L, A, C, M, V, Y, E, | | | | |
| | D, K, S, R, N, H, Q, P, G | | | | |
| 65 | C, A, R, D, V, Q, K, T, L, S, N, | | | | |
| | H, G, I, Y, E, M, F | | | | |

TABLE 13DDD
Tie2_mb3
SVEEFLKKLVEEFGVPPEEVERALEIIARLLNVPPEAALFFLIAIILDYNVPPEEAVEIVRRNVT
(SEQ ID NO: 1452)

| | | | | | |
|---|---|---|---|---|---|
| 1 | W, F, E, Y, D, C, G, M, N, | Q, K, A | X | | |
| | S, H, L, R, P, T, V | | | | |
| 2 | D, E, N, L, T, Q, H, M, V, C, | | X | | |
| | A, P, S, F, I, Y | | | | |
| 3 | D, E, S, R, H, K, T, N, P, | Q, A, I, L, V, G | | | |
| 4 | E, P, R, M, S, Y, A, G, H, D, F, | | | | |
| | Q, C, V, W, K, N, T, L, I | | | | |
| 5 | E, Q, D, T, V, F, I | C, L, W, Y, N, R | X | | |
| 6 | I, V, R, N, L, D | S, K, W, C, T, Q, Y, A, E, P, H | | | |
| 7 | G, A, D, E, V, C, S, T, I, | H, R | | | |
| | N, E, M, Q, Y, P, L, K | | | | |
| 8 | C, D, H, Q, A, N, L, V, E, | W | | | |
| | T, M, S, I, Y, G, E, K, | | | | |
| | R, P | | | | |
| 9 | F, M, I, V, A, S, C, E, G, | T | | X | |
| | W, Q, L, H, Y | | | | |
| 10 | Y, H, N, S, F, A, E, C, W, | Q, P, V, I, L, K, G | | | |
| | T | | | | |
| 11 | D, C, E, T, R, N, M, H, A, S, | | | | |
| | Y, Q, K, L, G, W, F, V | | | | |
| 12 | E, T, H, M, Q | | | | |
| 13 | L, I, G, V, A, C, T, F | Y, E, M, W | | X | |
| 14 | E, D, Q, H, T, G, V, M, R, S, | | | | X |
| | A, N, C, L, F, Y | | | | |
| 15 | C, A, I, M, N, V, G, Q | E, W, S, L, T | | X | X |
| 16 | D, C, I, E, N, V, A, Q, P, | F, Y, K, M, H, R, G | | | X |
| | S | | | | |
| 17 | Y, H, M, I, L, R, F, K, Q, | P, N, D, G, S, C, W | | | |
| | E, V, A, T | | | | |
| 18 | W, I, E, V, F, S, L, N, M, D, | | | | |
| | Y, Q, H, G, A, K, C, T, R | | | | |
| 19 | D, Q, E, P, V, Y, T, C | | | | |
| 20 | Y, L, I, H, T, V, W, G, C | A, P, M | | | |
| 21 | C, R, E, D, G, W, Q, L, Y, M, | | | | |
| | K, T, A, I, H, N | | | | |
| 22 | F, C, Y, A, E, Q, L, M, H, | P, V, K | | | |
| | I, S, T, D, G, N, W, R | | | | |
| 23 | D, L, W, N, Y, Q, A, M, F | S | | X | |
| 24 | L, M, H | | | | |
| 25 | C, Y, W, M, F, H, L, V, D, | R, N, Q, A, G, S, T | | | |
| | I, E | | | | |
| 26 | Y, L, W, H, F, A, G, C, M, | D, Q, E | | | |
| | V, S, I | | | | |
| 27 | V, C, I, L, M, T, S | | | X | |
| 28 | C, M, A, S, Q, I, E, V, T | | | | |
| 29 | C, E, N, H, D, A | T, R, Q, M, G, F, V, Y, L, W, S, K | | | |
| 30 | F, H, Y, V, T, C, W, M, Q, | A, N | | X | |
| | L, I | | | | |
| 31 | Y, M, W, C | L, H, S, F, T | X | | X |
| 32 | C, N, E, S, H, G, K, M, Q, L | | | | X |
| 33 | C, V, T, I, W, S, A, K | | X | | X |
| 34 | N, P, R, E, T, Q, V, H, S, | | | | X |
| | K, G, A, D, L | | | | |
| 35 | M, P, N, S, H, R, Y, Q, A, C, | | | | |
| | V, T, L, G, E, K, I, D, W | | | | |
| 36 | N, E, S, Q, D, P, H, C, A, | | X | | |
| | T, G, Y, E, V, K | | | | |
| 37 | I, V, T, E, D, Q, A | L, M, C, N, H, G, S, R, | X | | |
| | | K | | | |
| 38 | A, S, G, C | | | X | |
| 39 | M, I, E, A, W, V, S, H, L, | Q, Y, F, C, G, P | | | |
| | R, T | | | | |
| 40 | W, Y, F, C | I | X | | |
| 41 | Y, P, I, E, C, V, L, M, F | S, D, A | X | | |
| 42 | Y, M, L | F | | X | |
| 43 | L, M, T, I | V, C | X | | |
| 44 | C, M, L, I, T, V | A, S, N | X | | |
| 45 | Y, M, S, A, L, I, V | | | X | |
| 46 | F, L, T, I | S, M, A, C | | X | |
| 47 | M, I, V, R, T, Q, C, W, L | A, F, K, S | X | | |
| 48 | D, T, E | | X | | |
| 49 | Y, R | | X | | |
| 50 | D, E, S, K, N, G | | | | X |
| 51 | V, I, C, M, A, L | | | | X |
| 52 | E, D, Y, V, Q, C, P, H, A, I, L, | | | | X |
| | F, M | | | | |
| 53 | W, P, C, Y, G, A, V, T | | | X | |
| 54 | P, E, I, K, H, N, D, T, Q, V, C, | | | | |
| | L, F, A, S, R, G | | | | |
| 55 | D, N, L, C, E, R, M, V, K, Q, | | | | |
| | S, G, T, A, P, Y, H, I, F | | | | |
| 56 | Y, I, V, A, F, L, Q, M, N, T, S, | | | X | |
| | H, C, E | | | | |
| 57 | N, T, Q, G, V, A, Y, I, K, H, | | | | |
| | F, L, S | | | | |
| 58 | D, E, I, P, V, Q, N, S, C, T, | | | | |
| | G, H, L, Y, K, W, A, M, R, F | | | | |
| 59 | E, D, L, A, V, P, S, Q, T, | W | | | |
| | G, M, Y, N, R, I, H, F | | | | |
| 60 | D, N, P, T, G, S, A, V, E, | W | X | | |
| | M, Q, E | | | | |
| 61 | Q, S, N, A, T, E, P, G, H, D, V, | | | | |
| | R, W, K, M, Y | | | | |
| 62 | D, E, P, T, R, I, F, V, K, L, M, | | | | |
| | Y, G, H, S, A, W | | | | |
| 63 | P, E, A, G, N, T, R, S, H, Q, C, | | X | | |
| | V, K, M | | | | |
| 64 | R, G, P, E, K, D, N, V, S, A, Q, | | | | |
| | H, Y, M, I, L, C | | | | |
| 65 | N, S, Q, E, I, T, V, K, D, R, H, | | X | | |
| | G, M, Y, F, L, A, W, P, C | | | | |

TABLE 13EEE
Tie2_mb4
DESAAEQIARLLIQGDPAAKQTVRAFVQAAKRGNPAAKRALEVIRKVLRKLGNPEVVKKVKKLIK
(SEQ ID NO: 1453)

| | | | | | |
|---|---|---|---|---|---|
| 1 | L, M, T, V, Y, P, C, D, A, I, N, Q, | | | | |
| | G, H, R, E, K, S, F, W | | | | |
| 2 | C, V, F, D, G, E, N, P, Y, S, T, M, | | | | |
| | H, K, R, L, A, I, Q | | | | |
| 3 | K, Q, I, A, W, C, F, S, G, Y, T, L, | | X | | |
| | P, R, M, D, E, V, N, H | | | | |
| 4 | R, W, V, T, N, I, S, G, P, H, Q, A, | | X | | |
| | C, L, M, Y, F, D, K, E | | | | |
| 5 | M, H, P, A, V, S, E, N, L, F, G, C, | | | X | |
| | I, Y, W, T | | | | |
| 6 | K, I, V, Q, S, M, E, N, G, Y, A, | | | | |
| | F, R, L, H, W, T | | | | |
| 7 | M, F, C, L, V, Q, D, E, K, S, W, | | X | | |
| | I, N, Y, T, A, R, G, H | | | | |
| 8 | I, V, W, L, M | | X | | |
| 9 | A, G | | | X | |
| 10 | K, V, F, L, R, M, W, T, Y, G, C, | | | | |
| | H, I, A, S, Q, N, E | | | | |
| 11 | G, N, S, R, K, L, C, D, M, Q, W, | | X | | |
| | F, A, Y, H, E | | | | |
| 12 | F, L, M | | | X | |
| 13 | W, F, Y, E, D, S, L, A, I, T, N, V, | | | | |
| | M, C, Q, H, K, R | | | | |
| 14 | S, I, Y, M, W, Q, K, H, L, F, T, | | | | |
| | A, D, R, V, E, N, C, G | | | | |
| 15 | W, N, G, D, Q, H, S, K, R, Y, C, | | | | X |
| | A, F, P, E | | | | |
| 16 | H, D, V, Y, N, S, Q, K, A, | E, M, R | | | X |
| | I, L, F | | | | |
| 17 | N, L, T, W, V, Q, P, D, I, K, R, E, | | | | |
| | Y, M, H, F, A, S, G, C | | | | |
| 18 | R, H, W, V, C, A, N, F, Y, Q, K, S, | | X | | |
| | M, I, G, E, T, L, D | | | | |
| 19 | A, T, V | | | X | |
| 20 | K, I, V, R, M, W | | | | |
| 21 | W, Y, F, R, N, Q, H, M, I, L, S, | | X | | |
| | C, K, V, A, G, T | | | | |
| 22 | I, V, R, C, A, T, M, Y, L, H, E, | | X | | |
| | K, Q, S, W, N | | | | |
| 23 | V, L, I | | | X | |
| 24 | Y, F, W, H, R, K, A | | | | |
| 25 | K, Y, M, V, F, T, A, L, W, S, R, | | X | | |
| | Q, G, N, I, C, H | | | | |
| 26 | Y, L, F, W, A, M, I, C | | X | | |
| 27 | V, W, F, Y | | | | |
| 28 | F, Q, H, W, V, R, Y, K, M, I, N, | | X | | |
| | T, G, L, S, A | | | | |
| 29 | E, A, L, W, Y, M, S, R, K, Q, F, | | X | | |
| | H, C, N | | | | |
| 30 | A | | | | |
| 31 | K, R | | | | |
| 32 | R, C, S, A, K, G, N | | | | |
| 33 | G, K | | | | X |
| 34 | N, H, C, D, S, A, F, Y, V, Q, W, K | | X | | X |
| 35 | V, T, R, P, K, M, C, H, G, L, N, | | | | |
| | F, S, Y, Q, I, W, E, A | | | | |
| 36 | G, H, A, C, S, K, Q, V, R, T, N, E, | | X | | |
| | L, D, I, Y, F, W, M | | | | |
| 37 | A, C, S | | X | | |
| 38 | K, R | | | | |
| 39 | M, R, A, K, W, C, Q, Y, S, H, N, | | | | |
| | E, L, G, T, F, V, D, I | | | | |
| 40 | Q, F, K, R, S, A, C, T, M, Y, L, | | | | |
| | V, I, E, N, W, H | | | | |
| 41 | L | | | | |
| 42 | K, E, S, H, W, N | | | | |
| 43 | S, R, G, D, C, K, N, Q, V, H, W, | | | | |
| | A, T, M, L, E, I, Y, F | | | | |
| 44 | L, I, V | | | X | |
| 45 | H, Y, R, A | | | | |
| 46 | D, M, G, Q, K, T, N, L, V, S, A, | | | | |
| | R, I, H, W, Y, E | | | | |
| 47 | Q, E, V, T, W, C, K, Y, S, H, N, | | | X | |
| | I, A, M, G, L, R, F | | | | |
| 48 | L, F, M, I | | | X | |
| 49 | R, G, Q, K, W, V, H, A, N, M, S, | | | | |
| | Y, I, T, D, F, L | | | | |
| 50 | G, S, E, K, D, R, H, A, I, Y, L, | | | | |
| | M, N, Q, W, V, F, T, C | | | | |
| 51 | N, D, R, V, Q, S, F, G, E, | C | | X | X |
| | A, H, L, K, T, I, M, W, | | | | |
| | P, Y | | | | |
| 52 | D, E, T, N, G, H, S, P, C, | F | | | X |
| | Q, Y, A, I, M | | | | |
| 53 | P, N, D, E, S, K, T, C, G, Q, R, A | | | | X |
| 54 | G, P, D, H, K, Q, N, R, C, M, A, | | | | |
| | E, S, V, T, L, Y | | | | |
| 55 | E, D, C, K, R, A, Q, W, T, V, S, N | | | | |
| 56 | K, H, T, Y, R, V, Q, N, G, S, I, | | | X | |
| | W, C, M, D, P, A, E | | | | |
| 57 | N, R, S, V, P, Q, A, D, T, E, K, | | | | |
| | G, H, C, Y | | | | |
| 58 | K, R, G, H, Q, S, T, N, P | | | | |
| 59 | K, G, R, H, V, S, T, N, I, A, B | | | | |
| 60 | V, G, T, Y, D, P, E, C, W, I, A, | | | X | |
| | M, K, S, L, Q, H, R, F | | | | |
| 61 | K, R, P, A, T, H, V, Q, Y, N, S, | | | | |
| | G, F, C, I | | | | |
| 62 | C, T, K, N, Q, H, P, V, M, S, L, | | | | |
| | G, A, Y, I, F | | | | |
| 63 | L, E, Y, M, F, C, W, I, A, V, Q, | | | | |
| | K, G, T, D, P | | | | |
| 64 | Y, M, I, C, W, L, F, V, E, G, A, | | | | |
| | T, D, N, K, Q, R | | | | |
| 65 | R, K, F, N, I, Q, V, S, L, T, W, | | | | |
| | M, H, Y, P, G, E, A | | | | |

TABLE 13FFF
TrkA_mb1
RDEIKERIFKAVVRAIVTGNPEQLKEAKKLLEKLKKLGRLDQDAKKFEKAIRQVEKRLRS
SEQ ID NO: 1457

| | | | | | |
|---|---|---|---|---|---|
| 1 | A, Q, S, G, E, R, D | V | | | |
| 2 | H, E, Y, D, F | | | | |
| 3 | E, I, D, C, S | | | | |
| 4 | I, V, L | Y | | X | |
| 5 | I, N, V | M, T, K | | | |
| 6 | E, R | | X | | |
| 7 | R, K, Q, A, C, E | | | | |
| 8 | M, I | | | X | |
| 9 | K, F | | X | | |
| 10 | W | R, K, H | X | | |
| 11 | V, I, Q, T | A | | X | |
| 12 | V | I | X | | |
| 13 | | | X | | |
| 14 | R | | X | | |
| 15 | A, S | | | X | |
| 16 | I | K, V, R | X | | |
| 17 | C,V | | X | | |
| 18 | K | T | | | |
| 19 | H, K | R, S, A, N, Y, G | | | X |
| 20 | N, R, K, Q | T, C, M, E, Y, D, I, W | | | X |
| 21 | A, P, V, K, C, L, I | | | | |
| 22 | N, G, S, E, C, D, R, T | | | | |
| 23 | R, K, W, N, H, F, Q, Y | I, P, C, V, M, L | | X | |
| 24 | L | | | | |
| 25 | T, G, N, I, R, V, K, C, E, Q, | | | | |
| | S, H | | | | |
| 26 | D, C, I, G, E, V, Y | | | | |
| 27 | A, P | | | X | |
| 28 | E, D, R, M, H, T, K, A, W, S | | | | |
| 29 | K | | | | |
| 30 | I, F, Y, C, T, V, N, L, M, | S | | X | |
| | H | | | | |
| 31 | V, I | L | | | |
| 32 | F, E, N, D, Y, T, C, V, M | | | | |
| 33 | C, K, S, R, T, D, Q, M, F | | | | |
| 34 | M, L, I | | | X | |
| 35 | A, H, F, S, K, R, Y, G | I | | | |
| 36 | R, Q, H, S, K, L, F, M, C, | T, E | | | |
| | D, I, G, V | | | | |
| 37 | T, Y, F, L, Q, I | C | | | |
| 38 | P | G, A, S | | | X |
| 39 | C, P, G, D, A, R, S, H, N, | V | | | X |
| | T, M, K, Q | | | | |
| 40 | W, Y, F, P, I | V, N, A, M, Q, T, L, E, H, C, S, G | | | X |
| 41 | D | | | | X |
| 42 | T, M, L, H, Q, R, A, I | Y | | | |
| 43 | A, G, E, D, Q, P | S | X | | |
| 44 | E, Y, T | V, G, I, A, C, F | | X | |
| 45 | Y, K, M, R, H, L | I | | | |
| 46 | K | Q, A | X | | |
| 47 | F | L | X | | |
| 48 | E, W, V | | | | |
| 49 | K, R | | | | |
| 50 | A, M, H | | X | | |
| 51 | I, C | V, T | | | |
| 52 | Y, F, H | G, I, S, W, R, V, K | | | |
| 53 | K, R, S, N, G, L, Q, V, T, | F | X | | |
| | E, A, M, D, I, H | | | | |
| 54 | V, C, Q | | X | | |
| 55 | A, G, E, S, Q | | | | |
| 56 | K, R | | | | |
| 57 | R, K | Q | | | |
| 58 | L | | | | X |
| 59 | R, Q, S | | | | |
| 60 | K, C, A, G, S, H, Q | N, R | | | |

TABLE 13GGG
TrkA_mb2
DEIEKRLRTALEELVRADKNNDEEKVRKAWQKAVQIVIEANDDNVSKLASKIYRELAKRVKS
(SEQ ID NO: 1458)

| | | | | | |
|---|---|---|---|---|---|
| 1 | I, T, N, P, C, H, E, F, V, Q, G, | | | | |
| | L, Y , R, D, S, A, M, W, K | | | | |
| 2 | S, C, D, W, P, G, Q, T, H, K, E, R, | | | | |
| | M, I, L, F, A, V, N, Y | | | | |
| 3 | Q, A, M, Y, C, K, V, R, F, H, L, | | | | |
| | N, W, T, I, S, G, E | | | | |
| 4 | I, Y, D, V, K, H, T, R, G, A, S, | | | | |
| | Q, N, E, L, P, F | | | | |
| 5 | C, E, H, P, D, F, I, T, W, N, A, | | | | |
| | V, Q, S, L, Y, G, K, M, R | | | | |
| 6 | P, Y, H, V, C, M, W, A, E, L, I, | | | | |
| | F, T, K, D, R, G, S, Q, N | | | | |
| 7 | L, I | | | X | |
| 8 | K, W, S, Y, V, Q, H, T, E, R, I, | | | | |
| | C, M, A, N, L, G, E, D, P | | | | |
| 9 | V, I, C, W, E, D, A, Y, R, T, N, | | | | |
| | M, P, F, S, G, K, H, L, Q | | | | |
| 10 | N, Q, V, E, Y, S, C, I, H, P, W, | | | X | |
| | A, T, F, L, R, G, M, K | | | | |
| 11 | M | V, I, L | X | | |
| 12 | W, G, T, S, M | F, C, R, L, K, D, H, V, A, E, N | | | |
| 13 | M, Q, A, Y, W, G, D, F, L, T, K, | | | | |
| | H, C, R, E, N, V, S | | | | |
| 14 | L, M, Y | | X | | |
| 15 | I, L, T, C, V, A, H, Y, D, N, R | | | X | |
| 16 | W, Y, C, D, I, A, G, E, K, Q, H, | | | | |
| | L, M, V, F, R, S, N, T | | | | |
| 17 | G, Y, S, W, A | H | | X | |
| 18 | L, N, I, R, V, H | K, E, G, T, F, M, W, Q, | X | | |
| | | S, A, Y, D | | | |
| 19 | F, Y, T, D, S, E, L, G, A, V, Q, | | | | |
| | I, W, K, H, C, N, R, M | | | | |
| 20 | C, H, D, F, M, E, Q, L, W, S, T, | | | | X |
| | V, A, G, Y, R, N, I, K, P | | | | |
| 21 | K, R, H, Y, T, E, C, D, V, L, F, | | | | X |
| | A, M, Q, N, S, G | | | | |
| 22 | E, M, F, Y, V, C, H, L, I, R, A, | | | | X |
| | S, K, N, W, D, Q | | | | |
| 23 | Q, G, Y, L, A, N, V, F, I, S, D, | | | | |
| | T, M, K, P, R, E, H, W | | | | |
| 24 | Y, C, F, G, M, R, Q, H, K, L, E, | | | | |
| | W, D, N, S, V, I, A, P, T | | | | |
| 25 | D, E, P, L, A, G, H, Y, C, M, Q, | | | | |
| | V, R, T, I, K, N, S, F, W | | | | |
| 26 | L | R, N, A, S, G, E, Y, Q, K, V | | | |
| 27 | W, R | A, T, K, D, H, E, G, N, S | | | |
| 28 | M, L, Y, T, I, E, H, V, N, F, Q, | | | | |
| | R, W, D, P, A, C, G, S, K | | | | |
| 29 | Q, W, S, N, Y, M, G, C, H, F, A, | | | X | |
| | T, K, L, V, E, I | | | | |
| 30 | F, W, K, S, G, Y, A, C, R, M, L | | X | | |
| 31 | N, T | W, I, R, V, E, K, M, D, | X | | |
| | | S, Q, A, H | | | |
| 32 | W, N, M, G, Y, E, F, D, V, C, L, | | | | |
| | H, T, K, I, A, S, R, Q | | | | |
| 33 | T, V | N, S, A | X | | |
| 34 | I, V, L | | X | | |
| 35 | K, L, M, R, I, Y, F, H, T, W | S, C, E, G, N, A, Q | X | | |
| 36 | V, C, M, Y, L, T, I, A, W, F | | | X | |
| 37 | I, V, L, N | | X | | |
| 38 | I | | X | | |
| 39 | R, M, H, K, F, Y, Q, W, L, | V, C, D, E | | | |
| | A, S, N, T, G, I | | | | |
| 40 | T, Q, G, C, S, A, E, V, N, M, L, | | | X | |
| | I, P, W | | | | |
| 41 | A, G, K, R, H, D, Q, S, N, C, M, | | X | | |
| | T, Y, L, W, I | | | | |
| 42 | N, S, D, E, H, V, I | | | | X |
| 43 | S, A, P, G, H, E | T, F, W, Y, V, N, L, K, | X | | |
| | | R, Q, M, D | | | |
| 44 | K, R, C, H, I, W, E, A, G, D, M, | | | | |
| | F, Y, V, L, Q, N, T, S | | | | |
| 45 | A, T, I, V, F, Y, M, L, G, R | | | | |
| 46 | N, A, S | | X | | |
| 47 | H, N | D, S, G, I, V, E, P, F, Q, T, Y, | | | |
| | | M, A, K | | | |
| 48 | M, L, I, V | | | | |
| 49 | C, A, T | | X | | |
| 50 | T, S, G, C, A, F, Y, P | | X | | |
| 51 | D, R, Y, E, N, M, C, G, T, L, V, | | | | |
| | S, A, Q, K, H, W, I | | | | |
| 52 | L, V, I, W, T, M, Y, C, A, F, N | | | X | |
| 53 | W, Y, H | | X | | |
| 54 | K, N, R, G, H, L, C, S, P | | X | | |
| 55 | N, A, T, D, Q, S, H, M, E, Y, G, | | | | |
| | F, L, K, V, I | | | | |
| 56 | E, A, T, M, V, L, I, S, F, N, Q, | | | X | |
| | H, P | | | | |
| 57 | D, N, L, T, M, H, V, R, K, Q, E, | | | | |
| | I, Y, A, S, F, G, P | | | | |
| 58 | F, P, T, Y, G, N, Q, W, R, L, S, | | | | |
| | V, H, D, I, M, E, K, A, C | | | | |
| 59 | K, H, N, I, L, D, E, Q, F, P, W, | | | | |
| | G, V, T, Y, A, M, R, S, C | | | | |
| 60 | Y, I, R, A, P, N, M, G, D, K, S, | | | X | |
| | W, T, Q, H, V, E, F, L | | | | |
| 61 | I, Y, D, S, T, P, G, A, F, W, L, | | | | |
| | E, R, K, Q, H, N, M, V | | | | |
| 62 | R, L, E, F, I, M, H, W, T, Q, K, | | | | |
| | A, G, N, S, Y, P, C, V, D | | | | |

TABLE 13HHH
VirB8_mb1
NAEEITEKATLVGIEAWLLAKDEEQKKKVRTLNRQVKKLLQQNDLDQAKRVLDQLKSVLEDLKS
(SEQ ID NO: 1468)

| | | | | | |
|---|---|---|---|---|---|
| 1 | G, A, Q, K, E, N, S, R, V, | C, W | | | |
| | D, I, P, F, T, L, H, M, | | | | |
| | Y | | | | |
| 2 | Q, H, A, K, S, P, R, T, G, | F, I | X | | |
| | E, N, V, D, Y, M | | | | |
| 3 | I, V, M, L, D, P, E, A, Q, | T, H, W, G | X | | |
| | S, N, E | | | | |
| 4 | E, D, A, Q, V, W, Y, S, M, | I, L, N, F | X | | |
| | T | | | | |
| 5 | I, L, T, V | M | | X | |
| 6 | T, S, A, Q, M, V, H, Y, L, | E, R, F, G, D | X | | |
| | W, N, I | | | | |
| 7 | E, T | G | X | | |
| 8 | Q, A, S, D, G, K, N, H, R, | C, T, M | | | |
| | E | | | | |
| 9 | D, S, A, T, M, C, G, V, P | | | X | |
| 10 | T, V, P | I | X | | |
| 11 | Q, L, F, I, M, R, P | | X | | |
| 12 | T, W, V, N, M, I, Y, F, A | L, S | | X | |
| 13 | G, S, A | E, V | X | | |
| 14 | N, I, L, S | | X | | |
| 15 | E, D | A | X | | |
| 16 | E, A, S, C | | | X | |
| 17 | G, R, L, W, C, Y, N | | X | | |
| 18 | L, V, K, I, R, M | F | X | | |
| 19 | L, M, T, E, I | | | | X |
| 20 | V, A, E, T, S, G | | | | X |
| 21 | P, D, E, K, T, S, Q, V, A, | N, H, M | | | X |
| | R, G | | | | |
| 22 | D, Y, N, A, T, E | S | | | X |
| 23 | Q, E, A, D, N, P, H, T, L, | M, C | | | |
| | S, V, I, K, Y, R, F, G, | | | | |
| | W | | | | |
| 24 | E, K, N, G, R, S, Q, D, T, | L, M, | | | |
| | A, P, H, Y, V, I | | | | |
| 25 | Q, E, D, T, M, G, H, K, N, | Y, R | | | |
| | S, A, V, I, C, L | | | | |
| 26 | K, R, N | | | | |
| 27 | E, G, M, T, S, A, H, N, Q, | W | | | |
| | I, K, L, V, R, D, F, C, | | | | |
| | Y | | | | |
| 28 | E, Y, H, Q, N, T, R, K, D, | W | | | |
| | M, S, L, F, I, A, G, V, | | | | |
| | C | | | | |
| 29 | E, L, I, V, C | | X | | |
| 30 | Q, T, V, A, I, R, K, S | C, M, L, H, E, G | X | | |
| 31 | D, G, N, T, H, S, P, R, Q, | W | | | |
| | M, E, V, C, I, F, A, Y, | | | | |
| | K, L | | | | |
| 32 | L, K, M, Y, H, Q | C, S, I | | X | |
| 33 | N, S, M, A | T, V, D, G | X | | |
| 34 | G, T, I, H, A, S, M, R, V | Q, K, Y, F, L, W, N, E | X | | |
| 35 | D, Q, E, N, H, T, K, R | S, M, L | | | |
| 36 | G, I, V, C, L, T, N, M, A, F | | | X | |
| 37 | N, L, T, R, S, K | P, G, H, Q | X | | |
| 38 | M, I, V, T, Q, C, L, N, E, Y, A, W, | | | | |
| | H, S, D, E, K, R, G | | | | |
| 39 | D, E, A, S, H, N, F, C, Y, | R, W | | X | |
| | K, M, Q, L, T, G | | | | |
| 40 | I, L, V, T, W, C | F, M, A | | X | |
| 41 | E, D, A, Q, M, S, N, T, H, | F | | | |
| | Y, L, V, G, I, K, W, R | | | | |
| 42 | V, Q, E, I, N, A, T, D, M, | | | | X |
| | K, S, L, R, H, C, Y, G, F, W | | | | |
| 43 | G, H, N, K, Q, D, S, E, Y, | I, F | | | X |
| | R, A, C | | | | |
| 44 | V, M, Q, D, E, L, N, K, R, H, | | | | |
| | A, Y, C, S, G | | | | |
| 45 | L, C, F, N, M, Y, W, T, I, V, | | | | |
| | H, S, A, P, Q, E, R, D, K, G | | | | |
| 46 | A, E, Q, M, V, D, K, S, T, | C,W, P | | | |
| | I, N, L, R, H, G, Y, F | | | | |
| 47 | G, K, Q, R, S, A, N, M, E, | D, V, I | | | |
| | T, H, L, C | | | | |
| 48 | E, S, T, A | | | X | |
| 49 | A, S, N, L, R, I, K, T, Q, | D, C | | | |
| | M, Y, E, V, W, G, F, H | | | | |
| 50 | K, A, R, L, I, D, Q, E, T, | W | | | |
| | N, V, S, H, M, Y, G, C, | | | | |
| | F | | | | |
| 51 | F, D, V, T, A, K, C, I, M, S, G, | | | | X |
| | L, Y, H, Q | | | | |
| 52 | L, V, T, M | | | | |
| 53 | D, M, I, V, A, L, S, E, N, F, Q, H, | | | | |
| | C, T, R, K, G, Y, W | | | | |
| 54 | Q, E, T, N, H, K, R, V, D, | G | | | |
| | S, I, A, L, M, C | | | | |
| 55 | L, M | F | | | X |
| 56 | E, T, S, A, V, Y, G, M, Q, | L | | | |
| | H, R, C, K, N, F, I, W, | | | | |
| | D | | | | |
| 57 | F, W, Q, T, D, M, G, H, S, N, V, | | | | |
| | E, Y, L, A, R, C, I, K | | | | |
| 58 | V, L, I, G, M, T | F | | | X |
| 59 | L | Y, H, W, C | | | |
| 60 | Q, D, E, N, S, A, H, Y, M, | K, V, I, G, T, W, L, F, C | | | |
| | R | | | | |
| 61 | D, E, N, G, Q, S, H, K, T, | F, Y, C, V, I | | | |
| | M, W, A, R | | | | |
| 62 | P, I, L, V, M, Q, K | | | | X |
| 63 | R, K, Q, H | | | | |
| 64 | E, M, K, N, Y, R, S, Q, D, | P | | | |
| | G, L, F, T, V, H, A, I, | | | | |
| | C, W | | | | |

In one embodiment, amino acid substitutions relative to the reference polypeptide are conservative amino acid substitutions. As used herein, “conservative amino acid substitution” means a given amino acid can be replaced by a residue having similar physiochemical characteristics, e.g., substituting one aliphatic residue for another (such as Ile, Val, Leu. or Ala for one another), or substitution of one polar residue for another (such as between Lys and Arg, Glu and Asp, or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity. e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gin (Q): (3) acidic: Asp (D), Glu (E); (4) basic: Lys (K), Arg (R). His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the percent identity of the polypeptide to the reference polypeptide does not include any functional domains added fused to the polypeptides (whether N-terminal, C-terminal, or internal), and wherein the 1, 2, 3, 4, or 5 N-terminal and/or C-terminal amino acid residues may be present or absent when considering the percent identity.

In another embodiment, 1, 2, 3, 4, or 5 N-terminal and/or C-terminal amino acid residues are not included when determining the percent identity of the polypeptide relative to the reference polypeptide. In another embodiment, all residues are included when determining the percent identity relative to the reference polypeptide.

In another embodiment, the disclosure provides fusion proteins comprising the polypeptide of any embodiment disclosed herein fused to a functional polypeptide. Any suitable functional polypeptide may be used, including but not limited to a therapeutic polypeptide, diagnostic polypeptide, targeting polypeptide, scaffold polypeptide, or polypeptide that confers stability on the fusion protein. Such fusion proteins may be used, for example, to target the functional polypeptide to the target of the polypeptides of the disclosure in or on cells. In one embodiment, the fusion protein comprises two or more copies of the polypeptide of any embodiment of the target binding polypeptides of the disclosure. In one such embodiment, the two or more copies of the polypeptide are identical. In the embodiments, the two or more copies of the polypeptide are not all identical. In all these embodiments, the fusion protein components may be directly adjacent in the fusion protein, or may be separated by an amino acid linker of any suitable length and amino acid composition.

In another embodiment, the disclosure provides scaffolds comprising 2, 3, 4, 5, 6, 7, 8, 9, 10, 11, 12, 13, 14, 15, 16, 17, 18, 19, 20, or more copies of the polypeptide or fusion protein of any embodiment disclosed herein. Any suitable scaffold can be used, including but not limited to designed polypeptide scaffolds, virus-like particles, beads, etc.

In another aspect the disclosure provides nucleic acids encoding the polypeptide or fusion protein of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e.: episomal or chromosomally integrated), non-naturally occurring polypeptides, fusion protein, or compositions disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the present disclosure provides pharmaceutical compositions, comprising one or more polypeptides, fusion proteins, compositions, nucleic acids, expression vectors, and/or host cells of the disclosure and a pharmaceutically acceptable carrier. The pharmaceutical compositions of the disclosure can be used, for example, in the methods of the disclosure described below. The pharmaceutical composition may comprise in addition to the polypeptide of the disclosure (a) a lyoprotectant; (b) a surfactant; (c) a bulking agent; (d) a tonicity adjusting agent; (e) a stabilizer; (f) a preservative and/or (g) a buffer.

In another aspect, the disclosure provides uses and methods for use of the polypeptides, fusion proteins, scaffolds, nucleic acids, expression vectors, host cells, and/or pharmaceutical compositions of the disclosure for any suitable use as disclosed herein. In one non-limiting embodiment, the polypeptides, fusion proteins, scaffolds, nucleic acids, expression vectors, host cells, and/or pharmaceutical compositions are used as a targeting moiety, to direct a “payload” to the target to which the polypeptide binds. In one embodiment, the payload may be a functional domain as described herein, and the polypeptide may be provided as a fusion protein with a polypeptide functional domain. The payload may include, but is not limited to, a detectable moiety (fluorescent protein, luminescent compound or protein, radioactive isotope, etc.), a therapeutic functional domain, and/or a diagnostic functional domain.

In another non-limiting embodiment, the polypeptides, fusion proteins, scaffolds, nucleic acids, expression vectors, host cells, and/or pharmaceutical compositions are used as a therapeutic moiety. In non-limiting embodiments, the methods may comprise treating a tumor or infection, such as a viral infection. The protein targets fall into two classes: (1) human cell surface or extracellular proteins involved in signaling, for which binders have utility as therapeutics for treating a tumor (Tropomyosin receptor kinase A (TrkA)15, Fibroblast growth factor receptor 2 (FGFR2)16, Epidermal growth factor receptor (EGFR)17, Platelet-derived growth factor receptor (PDGFR)18, Insulin receptor (Insulin®)19, Insulin-like growth factor 1 receptor (IGFIR)20, Angiopoietin-1 receptor (Tie2)21, Interleukin-7 receptor alpha (IL-7Rα)22, CD3 delta chain (CD3δ)23, Transforming growth factor beta (TGF-β)24); and (2) pathogen surface proteins for which binding proteins have therapeutic utility in treating infections (Influenza A H3 hemagglutinin (H3)25 (H3_mb series of proteins disclosed herein), VirB8-like protein from Rickettsia typhi (VirB8)26, and the SARS-CoV-2 coronavirus spike protein (LCB series of proteins).

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The subject may be any subject that has a relevant disorder. In one embodiment, the subject is a mammal, including but not limited to humans, dogs, cats, horses, cattle, etc. In one embodiment, the subject is a human subject.

In another aspect, the disclosure provides methods for designing protein binding proteins from target structural information alone comprising any steps or combination of steps as described in the examples that follow.

### EXAMPLES

### Abstract

The design of proteins that bind to a specific site on the surface of a target protein using no information other than the three-dimensional structure of the target remains an outstanding challenge. We describe a general solution to this problem, which starts with a broad exploration of the very large space of possible binding modes and interactions, and then intensifies the search in the most promising regions. We demonstrate its very broad applicability by de novo design of binding proteins to 12 diverse protein targets with very different shapes and surface properties. Biophysical characterization shows that the binders, which are all smaller than 65 amino acids, are hyperstable and bind their targets with nanomolar to picomolar affinities. Crystal structures of four of the binder-target complexes were solved, and all four are very close to the corresponding computational design models (data not shown). Experimental data on nearly half a million computational designs and hundreds of thousands of point mutants provide detailed feedback on the strengths and limitations of the method and of our current understanding of protein-protein interactions, and should guide improvement of both. Our approach now enables targeted design of binders to sites of interest on a wide variety of proteins for therapeutic and diagnostic applications.

### Design Method

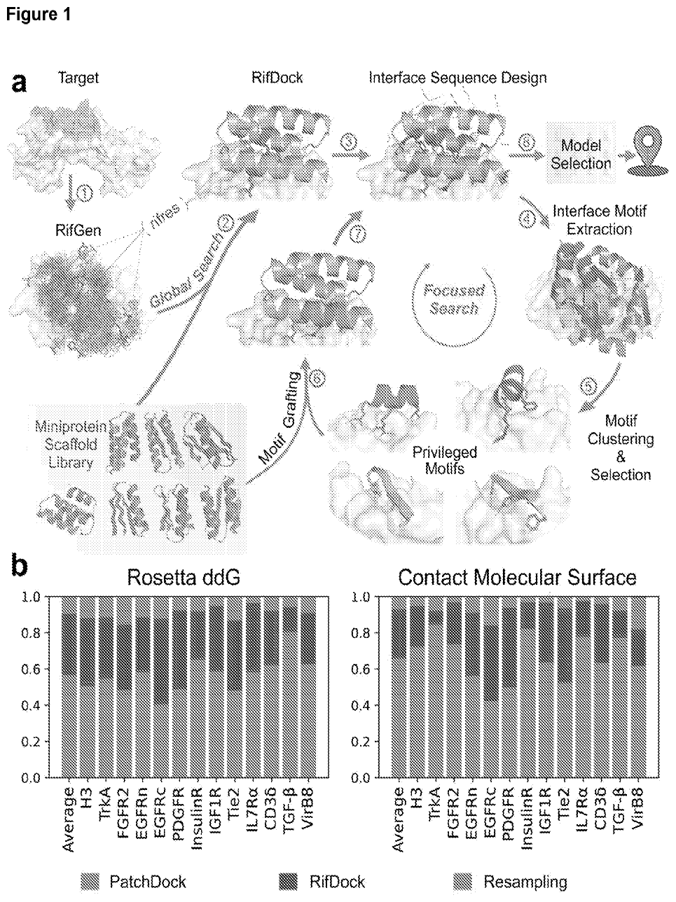

We sought to develop a general approach to design of high affinity binders to arbitrary protein targets that addresses two major challenges. First, in the general case, there are no clear sidechain interactions or secondary structure packing arrangements that can mediate strong interactions with the target; instead there are a very large number of individually very weak possible interactions. Second, the number of ways of choosing from these numerous weak interactions to incorporate into a single binding protein is combinatorially large, and any given protein backbone is unlikely to be able to simultaneously present sidechains that can encompass any preselected subset of these interactions. To motivate our approach, consider the simple analogy of a very difficult climbing wall with only a few good footholds or handholds distant from each other. Previous “hotspot” based approaches correspond to focusing on routes involving these footholds/handholds, but this greatly limits the possibilities and there may be no way to connect them into a successful route. An alternative is to first, identify all possible handholds and footholds, no matter how poor, second, have thousands of climbers select subsets of these, and try to climb the wall, third, identify those routes that were most promising, and fourth, have a second group of climbers explore them in detail. Following this analogy, we devised a multi-step approach to overcome the above two challenges by 1) enumerating a large and comprehensive set of disembodied sidechain interactions with the target surface, 2) identifying from large in silico libraries of protein backbones those that can host many of these sidechains without clashing with the target, 3) identifying recurrent backbone motifs in these structures, and 4) generating and placing against the target a second round of scaffolds containing these interacting motifs (FIG. 1a). Steps 1 and 2 search the space very widely, while steps 3 and 4 intensify search in the most promising regions. We describe and motivate each step in the following paragraphs.

We began by docking disembodied amino acids against the target protein, and storing the backbone coordinates and target binding energies of the typically billions of amino acids making favorable hydrogen bonding or non-polar interactions in a 6-dimensional spatial hash table for rapid lookup (FIG. 1a; see methods). This “rotamer interaction field” (RIF) enables rapid approximation of the target interaction energy achievable by a protein scaffold docked against a target based on its backbone coordinates alone (with no need for time consuming sidechain sampling)—for each dock, the target interaction energies of each of the matching amino acids in the hash table are summed. A related approach was used for small molecule binder design{Dou, 2018 #7}; since protein targets are so much bigger, and non-polar interactions are the primary driving force for protein-protein association, we focused the RIF generation process on non-polar sites in specific surface regions of interest: for example in the case of inhibitor design, interaction sites with biological partners. The RIF approach improves upon previous discrete interaction-sampling approaches {Fleishman, 2011 #3} by reducing algorithmic complexity from O(N) or O(N²) to O(1) with respect to the number of sidechain-target interactions considered, allowing for billions, rather than thousands, of potential interfaces to be considered.

For docking against the rotamer interaction field, it is desirable to have a very large set of protein scaffold options, as the chance that any one scaffold can house many interactions is small. The structure models of these scaffolds must be quite accurate so that the positioning is correct. Using fragment assembly {Koga, 2012 #14}, piecewise fragment assembly {Linsky, 2021 #16}, and helical extension {Maguire, 2021 #15}, we designed a large set of miniproteins ranging in length from 50 to 65 amino acids containing larger hydrophobic cores than previous miniprotein scaffold libraries {Chevalier, 2017 #1}, which makes the protein more stable and more tolerant to introduction of the designed binding surfaces. 84,690 scaffolds spanning 5 different topologies with structural metrics predictive of folding were encoded in large oligonucleotide arrays and 34,507 were found to be stable using a high-throughput proteolysis based protein stability assay {Rocklin, 2017 #8}. We experimented with several approaches for docking these stable scaffolds against the target structure rotamer interaction field, balancing overall shape complementarity with maximizing specific rotamer interactions. The most robust results were obtained using direct low resolution shape matching {Schneidman-Duhovny, 2005 #9} followed by grid based refinement of the rigid body orientation in the RIF (RIFDock™). This resulted in better Rosetta™ binding energies (ddGs) and packing after sequence design (contact molecular surface, see below) than shape matching alone with PatchDock™ (FIG. 1b), and more extensive non polar interaction with the target than hierarchical search without PatchDock™ shape matching {Dou, 2018 #7}.

Because of the loss in resolution in the hashing used to build the RIF, and the necessarily approximate accounting for interactions between sidechains (see methods), we found that evaluation of the RIF solutions is considerably enhanced by full combinatorial optimization using the Rosetta™ forcefield, allowing the target sidechains to repack and the scaffold backbone to relax. Full combinatorial sequence optimization is quite CPU intensive, however, and to enable rapid screening through millions of alternative backbone placements, we developed a rapid pre-screening method using Rosetta™ to identify promising RIF docks. We found that including only hydrophobic amino acids, using a reduced set of rotamers than in standard Rosetta™ design calculations, and a more rapidly computable energy function sped design more than 10-fold while retaining a strong correlation with results after full sequence design (next paragraph); this pre-screen (referred to as the “Predictor” below) substantially improved the binding energies and shape complementarity of the final designs as far more RIF solutions could be processed.

We observed that application of standard Rosetta™ design to the set of filtered docks in some cases resulted in models with buried unsatisfied polar groups and other suboptimal properties. To overcome these limitations, we developed a combinatorial sequence design protocol that maximizes shape and chemical complementarity with the target while avoiding buried polar atoms. Sequence compatibility with the scaffold monomer structure was increased using a structure based sequence profile (Brunette, 2020 #17), the cross-interface interactions were upweighted during the Monte Carlo-based sequence design stage to maximize the contacts between the binder and the target (see method for the ProteinProteinInterfaceUpweighter), and rotamers containing buried unsatisfiable polar atoms were eliminated prior to packing and buried unsatisfied polar atoms penalized by a pair-wise decomposable pseudo-energy term {Coventry, 2021 #18}. This protocol yielded amino acid sequences more strongly predicted to fold to the designed structure and to bind the target than standard Rosetta™ interface design.

In the course of developing the overall binder design pipeline, we noticed upon inspection that even designs with favorable Rosetta™ binding free energies, large changes in Solvent Accessible Surface Area (SASA) upon binding, and high shape complementarity (SC) often lacked dense packing and interactions involving several secondary structural elements. We developed a quantitative measure of packing quality in much closer accord with visual assessment—the contact molecular surface (see methods)—which balances interface complementarity and size in a manner that explicitly penalizes poor packing. We used this metric to help select designs at both the rapid Predictor stage and after full sequence optimization (see Methods).

The space sampled by the search over structure and sequence space is enormous: tens of thousands of possible protein backbones×nearly one billion possible disembodied sidechain interactions with target×10¹⁶ interface sequences per scaffold placement. Sampling of spaces of this size is necessarily incomplete, and many of the designs at this stage contained buried unsatisfied polar atoms (only rotamers that cannot make hydrogen bonds in any context are excluded at the packing stage) and cavities. To generate improved designs, we intensified the search around the best of the designed interfaces. We developed a resampling protocol which first extracts all the secondary structural motifs making good contacts with the target protein from the first “broad search” designs, clusters these motifs based on their backbone coordinates and rigid body placements, and then selects the binding motif in each cluster with the best per-position weighted Rosetta™ binding energy; around 2,000 motifs were selected for each target. These motifs, which are privileged because they contain a much greater density of favorable side chain interactions with the target than the rest of the designs, were then used to guide another round of docking and design. Scaffolds from the library were superimposed on the privileged motifs, the favorable-interacting motif residues transferred to the scaffold, and the remainder of the scaffold sequence optimized to make further interactions with the target, allowing backbone flexibility to increase shape complementarity with the target (FIG. 1a). Interface metrics for the designs based on the resampling protocol were considerably improved relative to those of the designs from the broad searching stage (FIG. 1b). The designs with the most favorable protein folding and protein interface metrics from both the broad searching and resampling stages were selected for experimental validation.

### Experimental Testing

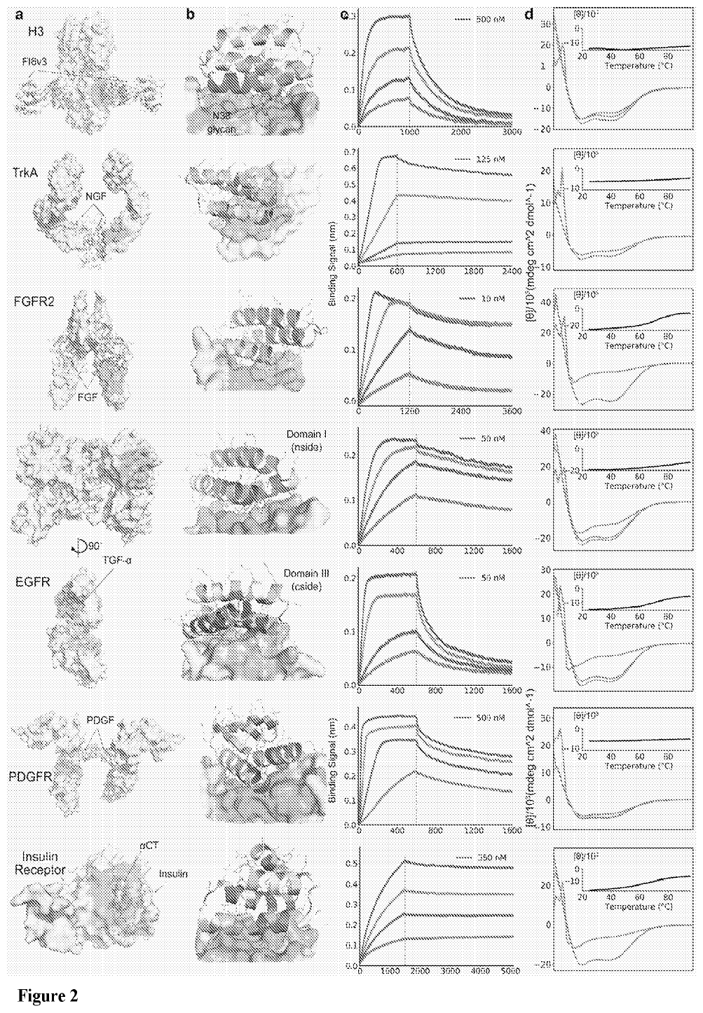

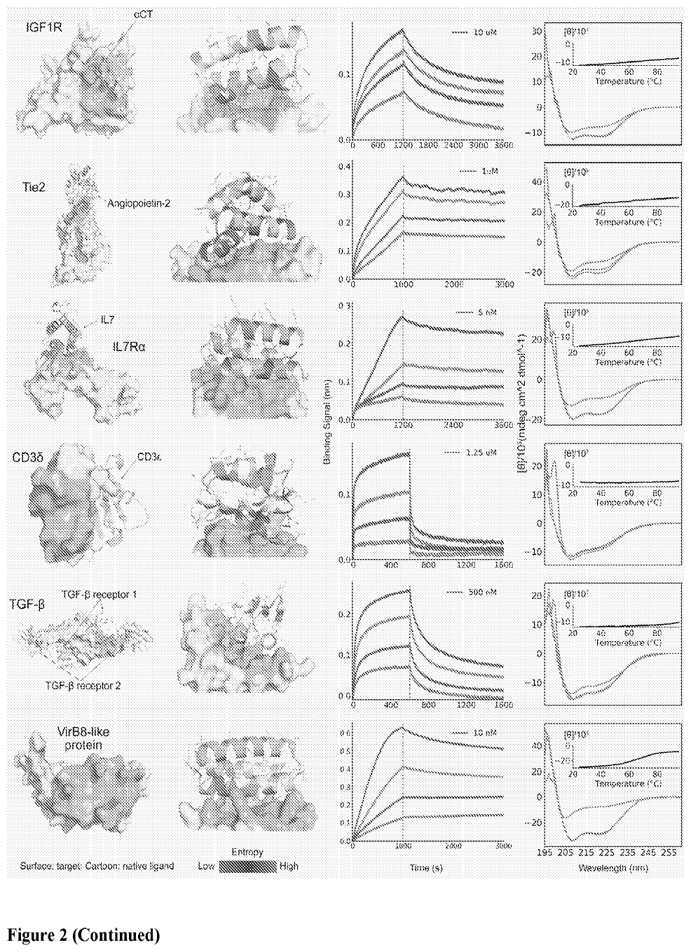

Previous protein binder design approaches have been tested on only one or two targets, which limits assessment of their generality. To robustly test our new binder design pipeline, we selected thirteen native proteins of considerable current interest spanning a wide range of shapes and biological functions. These proteins fall into two classes: first, human cell surface or extracellular proteins involved in signaling, for which binders could have utility as probes of biological mechanism and potentially as therapeutics (Tropomyosin receptor kinase A (TrkA) {Wiesmann, 1999 #30}, Fibroblast growth factor receptor 2 (FGFR2) {Plotnikov, 2000 #31}. Epidermal growth factor receptor (EGFR) {Garrett, 2002 #21}, Platelet-derived growth factor receptor (PDGFR) {Shim, 2010 #22}. Insulin receptor (Insulin®) {Croll, 2016 #23}, Insulin-like growth factor 1 receptor (IGF1R) {Xu, 2018 #24}, Angiopoietin-1 receptor (Tie2) {Barton, 2006 #25}, Interleukin-7 receptor alpha (IL-7Rα) {McElroy, 2009 #26}, CD3 delta chain (CD38) {Amett, 2004 #27}, Transforming growth factor beta (TGF-β) {Radaev, 2010 #28}); and second, pathogen surface proteins for which binding proteins could have therapeutic utility (Influenza A H3 hemagglutinin (H3) {Ekiert, 2012 #20}, VirB8-like protein from Rickettsia typhi (VirB8) {Gillespie, 2015 #29}, and the SARS-CoV-2 coronavirus spike protein) (FIG. 2a). For each target, we selected one or two regions to direct binders against for maximal biological utility and for potential downstream therapeutic potential. These regions span a wide range of surface properties, with diverse shape and chemical characteristics (FIG. 2a).

Using the above protocol, we designed 15,000-100,000 binders for each of thirteen target sites on the twelve native proteins (see Methods; we chose two sites on the EGF receptor). Synthetic oligonucleotides (230 bp) encoding the 50-65 residue designs were cloned into a yeast surface expression vector, the designs were displayed on the surface of yeast, and those which bind their target enriched by several rounds of fluorescence-activated cell sorting using fluorescently labelled target proteins. The starting and enriched populations were deep sequenced, and the fraction of each design after each sort was determined by comparing the frequency of the design in the parent and child pools. From multiple sorts at different target protein concentrations, we determined, as a proxy for binding Kd's, the midpoint concentration (SC₅₀) in the binding transitions for each design in the library (Table 14 and Methods).

TABLE 14
Number of binders against the 12 targets as estimated from FACS
sorting. SC₅₀ (Sorting Concentration₅₀) refers to the target concentration
where 50% of expressing yeast cells for a given design are collected.
The “SC₅₀ < 4 μM” column was produced by looking
for binders that saw >20% collection frequency during a 1 μM w/o
avidity sort (see Method). When a 1 μM sort was not performed,
500 nM and 11% were used instead. A similar procedure was used
to estimate the 400 nM column. Some binders saturate their binding
signal at 20% collection frequency (likely expression problems),
for this reason, the H3 data were estimated at 800 nM to avoid
needing a threshold higher than 20%. Additionally, binders with
very low counts were discarded to guard against doubly-transformed
yeast (See Methods).

| | | | Total Designs |
|---|---|---|---|
| Target | SC₅₀ < 4 μM | SC₅₀ < 400 nM | Tested |
| H3 | 50 (0.08%) | 21* (0.04%) | 60000 |
| TrkA | 10 (0.07%) | 3 (0.02%) | 15000 |
| FGFR2 | 604 (1.00%) | 196 (0.33%) | 60000 |
| EGFR | 15 (0.01%) | 12 (0.01%) | 100000 |
| PDGFR | 284 (0.28%) | 0 (0.00%) | 100000 |
| InsulinR | 259 (0.43%) | 2 (0.00%) | 60000 |
| IGF1R | 45 (0.30%) | 1 (0.01%) | 15000 |
| Tie2 | 5 (0.01%) | 0 (0.00%) | 100000 |
| IL-7Rα | 22 (0.14%) | 7 (0.05%) | 15000 |
| CD3^{a} | 1 (0.00%) | 0 (0.00%) | 60000 |
| TGF-β | 100 (0.67%) | 12 (0.08%) | 15000 |
| VirB8 | 72 (0.48%) | 10 (0.07%) | 15000 |
| SARS-Cov-2 RBD | 18 (0.02%) | 9 (0.01%) | 100000 |

*Number of binders with SC₅₀ < 800 nM estimated from 200 nM sort.
^{a}SSM sorts used to estimate the number of binders.

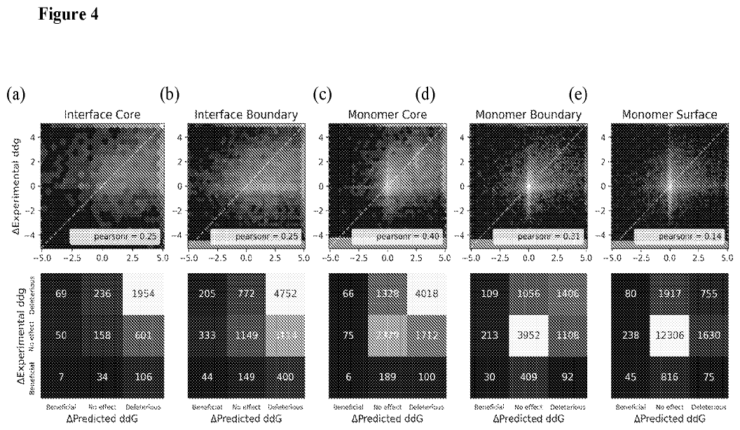

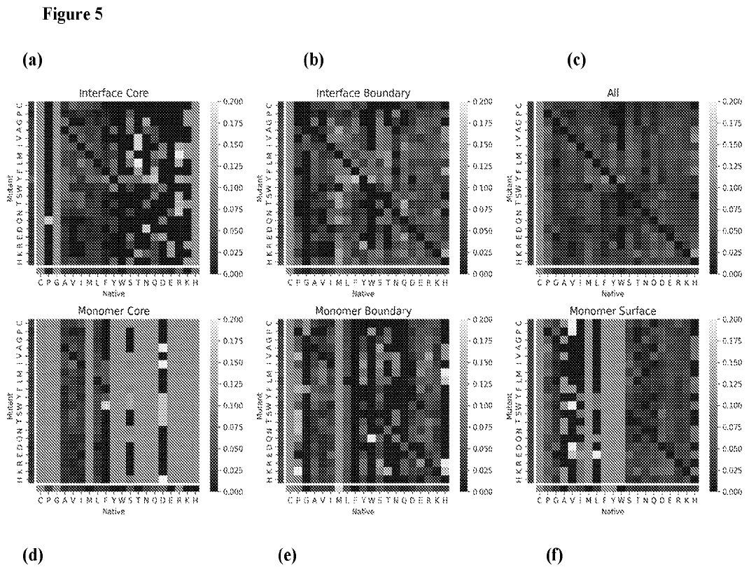

To assess whether the top enriched designs for each target fold and bind as in the corresponding computational design models, and to investigate the sequence dependence of folding and binding, we generated high resolution footprints of the binding surface by sorting site saturation mutagenesis libraries (SSMs) in which every residue was substituted with each of the 20 amino acids one at a time. For the majority, but not all, enriched designs, substitutions at the binding interface and in the protein core were less tolerated than substitutions at non-interface surface positions (FIG. 2b), and all the cysteines were highly conserved in designs containing disulfides. The effects of each mutation on both binding energy and monomer stability were estimated using Rosetta™ design calculations, and a reasonable correlation was found between the predicted and experimentally determined effect of mutations (FIG. 4). In almost all cases, a small number of substitutions were found to increase apparent binding affinity, and we generated libraries combining 5-15 of these combinatorially and sorted for binding under increasingly stringent (lower target concentrations) conditions. Many of these affinity-enhancing substitutions were mutations to tyrosine (FIG. 5), consistent with the high relative frequency of tyrosine in natural protein interfaces{Birtalan, 2008 #42}. The set of affinity increasing substitutions provide valuable information for improving the approach as these substitutions ideally would have been identified in the computational sequence design calculations (see discussion below).

We expressed the highest affinity combinatorially-optimized binders for each target in E. coli for more detailed structural and functional characterization. All of the designs were in the soluble fraction, and could be readily purified by nickel-NTA chromatography. All had circular dichroism spectra consistent with the design model, and most (9 out of 13) were stable at 95° C. (FIG. 2d). The binding affinities for the targets were assessed by biolayer interferometry, and found to range from 300 μM to 900 nM (FIG. 2c and Table 15). The sequence mapping data report on the residues on the design critical for binding, but only weakly on the region of the target bound. We investigated this using a combination of binding competition experiments, biological assays, and structural characterization of the complexes. For the nine targets for which these were available, this characterization suggested binding modes consistent with the design models, as described in the following paragraphs.

TABLE 15
Topologies, initial amino acid sequences, final optimized amino acid sequences
and physicochemical properties of the de novo miniprotein binders for all 12 targets.

| | | | Original | Optimized | # | Octet | |
|---|---|---|---|---|---|---|---|
| Target | Binder | Topology | sequence | sequence | mutations | kD | TM |
| H3 | H3_mb | HHH | SEREEFLEWLLR | SQHEKFLEWMLR | 8 | 320 nM | >95° C. |
| | | | KIEEAIKRGNKI | KIEEAIKRGNKI | | | |
| | | | SAEFLIGLAKQF | SAEFLINLAKNF | | | |
| | | | LHVINDDEIRRR | IHVLGDDEIRRR | | | |
| | | | LERLERLLH | LERLERQLH | | | |
| | | | (SEQ ID NO: | (SEQ ID | | | |
| | | | 644) | NO: 645) | | | |
| TrkA | TrkA_mb | HHH | RDELKERIEKTI | RDEIKERIFKAV | 12 | 1.4 nM | >95° C. |
| | | | VRAVVTGDPELL | VRAIVTGNPEQL | | | |
| | | | KEAKKLLEKLKK | KEAKKLLEKLKK | | | |
| | | | LGRLDQSAKQLE | LGRLDQDAKKFE | | | |
| | | | KAVREVEKQLRS | KAIRQVEKRLRS | | | |
| | | | (SEQ ID | (SEQ ID | | | |
| | | | NO: 1456) | NO: 1457) | | | |
| FGER2 | FGFR2_mb | HHH | DPSKELDKVYRT | DRRKEMDKVYRT | 12 | 543 pM | 71.1 ºC |
| | | | AFKRITSIPDKE | AFKRITSTPDKE | | | |
| | | | KQKEVVKEATEL | KRKEVVKEATEQ | | | |
| | | | LRRIAKDEEEKK | LRRIAKDEEEKK | | | |
| | | | LASLISLELKTL | KAAYMILFLKTL | | | |
| | | | S (SEQ ID | G (SEQ ID | | | |
| | | | NO: 22) | NO: 144) | | | |
| EGER | EGFRn_mb | HHH | DHWEEVERWALE | DHWEEVERWALE | 5 | 1.7 nM | >95° C. |
| | | | LLQEATEQNDPT | HLQEATQQNDPQ | | | |
| | | | KAKKILEEAHKL | KAKKILEEAHKW | | | |
| | | | LRRELSEEEARA | LRRELSEEEARA | | | |
| | | | VVRYLKQLVDRE | VVRWLKQLVDRE | | | |
| | | | LS (SEQ ID | LS (SEQ ID | | | |
| | | | NO: 6) | NO: 143) | | | |
| EGER | EGFRc_mb | HHH | SPDEAKKLLQEA | SLDEAKKLLQEA | 10 | 6.8 nM | 71.2° C. |
| | | | EKLARKQNDRME | EKLARKLNDRME | | | |
| | | | LAYVEFLKHVLE | LAYVEFLKHILE | | | |
| | | | NAKRINDKRAVE | TAKKQNDKRTIE | | | |
| | | | SVRELARDALEE | SVRDMARDALEE | | | |
| | | | LQS (SEQ ID | LQS (SEQ ID | | | |
| | | | NO: 3) | NO: 142) | | | |
| PDGFR | PDGFR_mb | HEEHE | DDERLARLAFRV | DDERLATLAFRA | 7 | 53 nM | >95° C. |
| | | | LIKRAGVPDFDV | LIKRAGVKNLDV | | | |
| | | | KVTNGKVRVTIT | KVTNGKVRVTIT | | | |
| | | | GRDQASQEALQL | GRDQASFKALQL | | | |
| | | | VEALARREGLQV | VFALARRLGLQV | | | |
| | | | QIDTR | QIDTR | | | |
| | | | (SEQ ID NO: | (SEQ ID | | | |
| | | | MMG) | NO: 1056) | | | |
| Insulin | Insulin | HHHH | DEELLELVYEAV | DEELMELVYEAV | 10 | 270 nM | 65 °C. |
| R | Rmb | | EKNDPRLLFEAW | EKNDPELLFEAW | | | |
| | | | MILASLLDKTGD | MELASLLDETGD | | | |
| | | | PKIEELLRLLQL | PKIEEALGLLQQ | | | |
| | | | VDRGDPDARRRI | VDGGNPDAGRRI | | | |
| | | | KELEK (SEQ | KELFK (SEQ | | | |
| | | | ID NO: 788) | ID NO: 789) | | | |
| IGF1R | IGF1R_mb | HHH | STRNAEFIVLLA | STRNAEFIMLLL | 11 | 860 nM | >95° C. |
| | | | ELCAKSQNDPSL | ELCVKSKNDPQV | | | |
| | | | QEYVKKVKKIVE | QEYVKKVKKQVE | | | |
| | | | SLLSNGDEKSAE | RLVGNGDEKKAE | | | |
| | | | EVARKALEYCAD | EVARKALEYCAD | | | |
| | | | G (SEQ ID | G (SEQ ID | | | |
| | | | NO: 712) | NO: 713) | | | |
| Tie2 | Tie2_mb | HHHH | SLVEELERLLEE | SIDEELERLLEE | 11 | 584 nM | >95° C. |
| | | | AGVDPELIEKLS | AGVDPELIDDLY | | | |
| | | | AVILQLLIRGLD | AVIYQLYIRGLS | | | |
| | | | PKDVLRFLLEML | DKDVLRELLENL | | | |
| | | | ERDGNPLRRVVE | ERDGTPLRRVVE | | | |
| | | | ELLKR (SEQ | ELLKR (SEQ | | | |
| | | | ID NO: 1450) | ID NO: 1451) | | | |
| IL-7Rα | IL- | HHH | SVKKKVRKVEKK | SVIEKERKLEKQ | 13 | 312 pM | >95° C. |
| | 7Rα_mb | | ARKAGDELAVLL | ARKQGDEVLVML | | | |
| | | | ARRVLEALEKGL | ARMVLEYLEKGW | | | |
| | | | VSEEDADESADR | VSEEDADESADR | | | |
| | | | IEEALKK (SEQ | IEEVLKK (SEQ | | | |
| | | | ID NO: 760) | ID NO: 761) | | | |
| CD3δ | CD3δ_mb | EHEEHE | NHIACEIHNPEA | NHIACEIHNPEA | 5 | 612 nM | >95° C. |
| | | | AKEIAKVANVRR | AKEIAKVANVRR | | | |
| | | | VFVIKQPGNRYF | VYFIKQPGNRYF | | | |
| | | | VLLKDADPEGVK | VLLKNADPEGVK | | | |
| | | | KVASKYNARCVI | KVRSKYNVRCVI | | | |
| | | | RE (SEQ ID | RE (SEQ ID | | | |
| | | | NO: 1) | NO: 141) | | | |
| TGF-β | TGF-β_mb | EHEEHE | TCTIEVVGVDPE | HCTIEVVGVDPE | 10 | 270 nM | >95° C. |
| | | | AVEAIAAAFGAE | KVEAIAAAYGAE | | | |
| | | | VREKDGKLEIHL | VCEKDGKFEIHL | | | |
| | | | DDPHGAESAAAA | DDPHSAESAAVA | | | |
| | | | ISVLANVRVRLQ | ISVLINRPVRLQ | | | |
| | | | C (SEQ ID | C (SEQ ID | | | |
| | | | NO: 1346) | NO: 1347) | | | |
| VirB8 | VirB8_mb | HHH | NAEEILEKATLI | NAEEITEKATLV | 14 | 500 pM | 66.2 |
| | | | AIEAWMLAKDEE | GIEAWLLAKDEE | | | |
| | | | VKKLVRTLARQV | QKKKVRTLNRQV | | | |
| | | | RKLLSNNDDDSA | KKLLQQNDLDQA | | | |
| | | | KSVLDTLKSVLE | KRVLDQLKSVLE | | | |
| | | | DLKS (SEQ ID | DLKS (SEQ ID | | | |
| | | | NO: 1467) | NO: 1468) | | | |

### Host Protein Targets Involved in Signaling

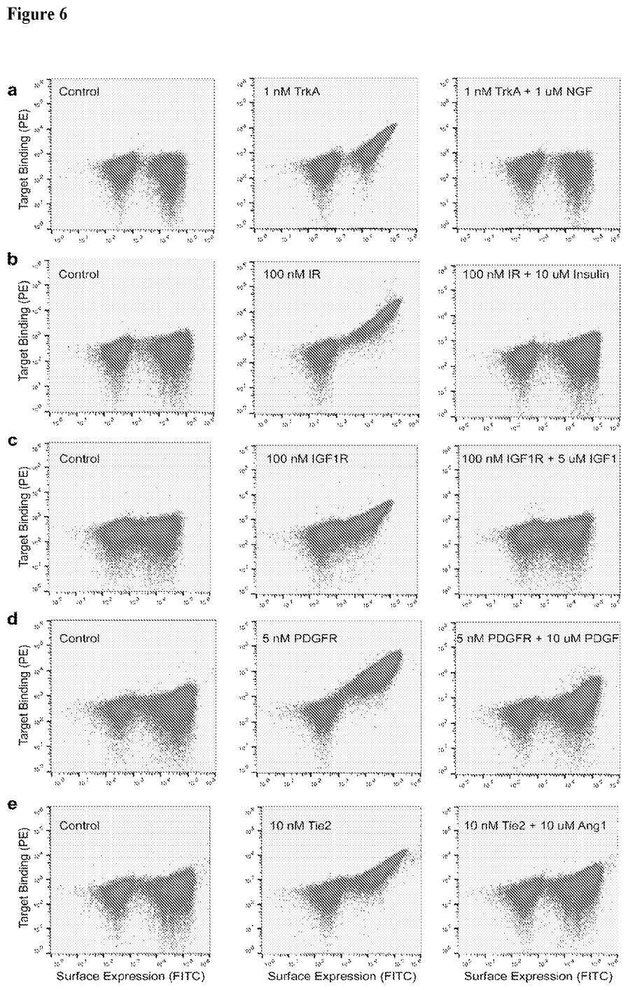

The receptor tyrosine kinases TrkA, FGFR2, PDGFR. EGFR, InsulinR. IGF1R and Tie2 are key regulators of cellular processes and are involved in the development and progression of many types of cancer{Lemmon, 2010 #32}. We designed binders targeting the native ligand binding sites for PDGFR, EGFR (on both domain I and domain III, the binders are then referred as EGFRn_mb and EGFRc_mb respectively), InsulinR. IGFIR and Tie2, and targeting surface regions proximal to the native ligand binding sites for TrkA and FGFR2 (FIG. 2a and see methods for criteria). We obtained binders to all eight targeting sites; the binding affinities of the optimized designs ranged from −1 nM or better for TrkA and FGFR2, to 860 nM for IGFIR. Competition experiments with nerve growth factor (NGF), Platelet Derived Growth Factor-BB (PDGF-BB), insulin, insulin growth factor-1 (IGF-1) and Angiopoietin 1 (Ang1) on yeast suggest that the binders for TrkA, PDGFR, InsulinR, IGF1R and Tie2 bind to their targeted sites (FIG. 6), and hence that the binding mode is consistent with the computational design models. The receptor tyrosine kinase binders as monomers are all expected to be antagonists. The effect of signaling through TrkA, FGFR2 and EGFR of the cognate binders on cells in culture was tested, and strong inhibition of signaling by the native agonists was observed in all three cases (Data not shown), including picomolar affinity binder for IL-7Rα targeting the IL-7 binding site.

### Pathogen Target Proteins

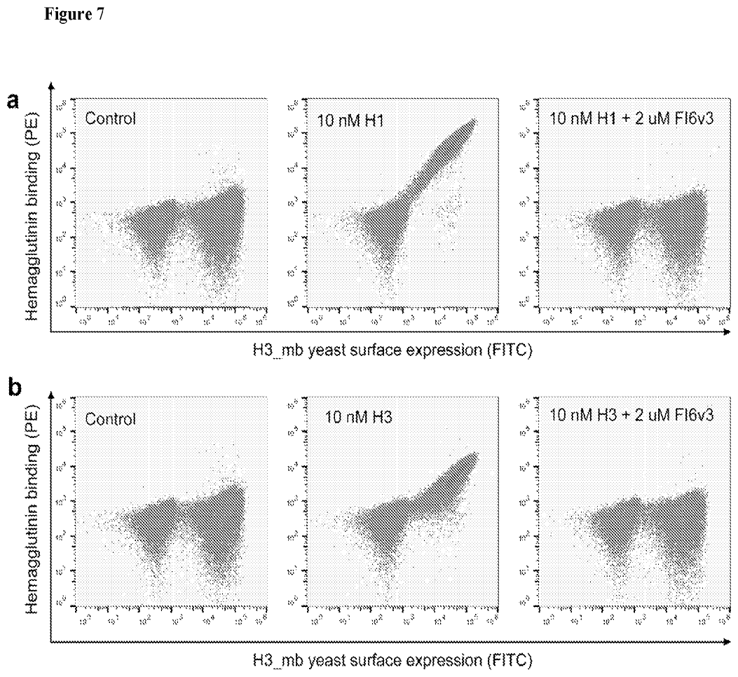

Hemagglutinin (HA) is the main target for influenza A virus vaccine and drug development, and it can be genetically classified into two main subgroups, group 1 and group 2{Webster, 1992 #83; Nobusawa, 1991 #84}. The HA stem region is an attractive therapeutic epitope, as it is highly conserved across all the influenza A subtypes and targeting this region can block the low pH-induced conformational rearrangements associated with membrane fusion, which is vital to the virus infection {Bullough, 1994 #85; Ekiert, 2009 #82}. Protein {Fleishman, 2011 #3; Chevalier, 2017 #1}, peptide {Kadam, 2017 #33} and small molecule inhibitors {van Dongen, 2019 #34} have been designed to bind to the stem region of group 1 HA to neutralize the influenza A viruses, but none of them is able to recognize the group 2 HA. Neutralizing antibodies targeting the stem region of group 2 HA have been identified through screening of large B-cell libraries after vaccination or infection, and some of them showed broad specificity and neutralized both group 1 and group 2 influenza A viruses {Corti, 2011 #35; Joyce, 2016 #86}. However, rational design of group 2 HA stem region binders remains a longstanding challenge, let alone the de novo designed pan-specific HA stem region binders which can bind both group 1 HA and group 2 HA. The challenge is mainly due to three differences between the group 1 HA and the group 2 HA: the group 2 HA stem region contains more polar residues and is more hydrophilic; in group 2 HA, Trp21 adopts a configuration roughly perpendicular to the surface of the targeting groove, which makes the targeting groove much shallower and less hydrophobic; the group 2 HA is glycosylated at Asn38, and the carbohydrate side chains covers the hydrophobic groove and protected the HA stem region from binding by antibodies or designed binders. We applied our new method to design binders to H3 HA (A/Hong Kong/1/1968), the main pandemic subtype of group 2 influenza virus, and obtained a binder with an affinity of 320 nM to the wild type H3 (FIG. 2) and 28 nM to the deglycosylated H3 variant (N38D) (FIG. 7a); the reduction in affinity is likely due to the entropy loss of the glycan upon binding and/or the steric clash with the glycan. The binder also binds to H1 HA (A/Puerto Rico/8/1934) which belongs to the main pandemic subtype of group 1 influenza virus (FIG. 7b); the binding with both H1 and H3 is competed by the stem region binding neutralizing antibody FI6v3{Corti, 2011 #35} on the yeast surface (FIG. 7c,d), suggesting that the binder binds the hemagglutinin at the targeted site. We also designed binders to the prokaryotic pathogen protein VirB8 which belongs to the type IV secretion system of Rickettsia typhi, which is the causative agent of murine typhus {Gillespie, 2015 #29}. We selected the surface region composed of the second and the third helices of VirB8, and obtained binders with 500 pM affinity (FIG. 2).

With the outbreak of the SARS-CoV-2 coronavirus pandemic we applied our method to design miniproteins targeting the receptor binding domain of the SARS-CoV-2 Spike protein near the ACE2 binding site to block receptor engagement. Due to the pressing need for coronavirus therapeutics, we recently described the results of these efforts {Cao, 2020 #2} ahead of those described in this manuscript; As in the case of FGFR2, IL-7Rα and VirB8, the method yielded picomolar binders, which are among the most potent compounds known to inhibit the virus in cell culture (IC₅₀ 0.15 ng/ml) and subsequent animal experiments have shown that they provide potent protection against the virus in vivo {Case, 2021 #43}. The modular nature of the miniprotein binders enables their rapid integration into designed diagnostic biosensors which we have demonstrated for both influenza and SARS-CoV-2 binders {Quijano-Rubio, 2021 #69}.

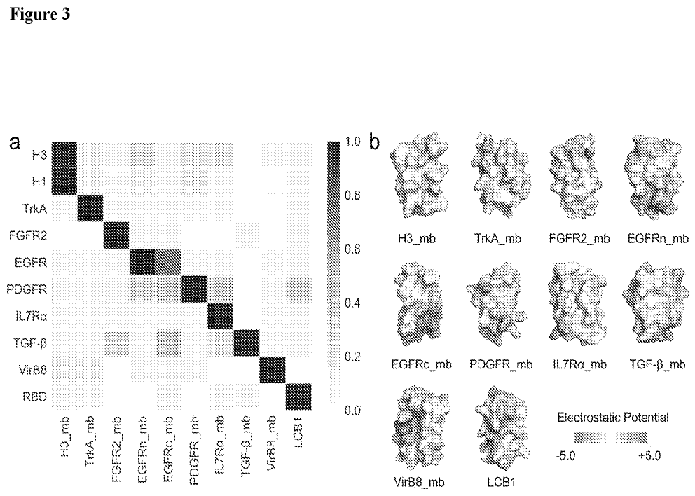

The designed binding proteins are all very small proteins (<65 amino acids), and many are 3-helix bundles. To evaluate their target specificity, we tested the highest affinity binder to each target for binding to all other targets. There was very little cross reactivity (FIG. 3a), likely due to the diverse surface shape and electrostatic properties of the designed binders (FIG. 3b). Consistent with previous observations with affibodies {Frejd, 2017 #10}, this suggests that a wide variety of binding specificities can be encoded in simple helical bundles; in our approach, scaffolds are customized for each target, so the specificity arises both from the set of sidechains at the binding interface, and the overall shape of the interface itself. To probe the dependence of scaffold design accuracy on design success, large numbers of scaffolds were superimposed onto 11 interface helical binding motifs for the IL-7Rα target, and sequence design was carried out as described above. There was a strong correlation between the extent of binding and the RMSD to the binding motif, suggesting that designed backbones must be quite accurate to achieve binding.

Structural validation There is a clear reorientation of the oligosaccharide at Asn38 compared with the unbound HK68/H3 structure, which has been seen in HK68/H3 structures bound with stem region neutralizing antibodies {Corti, 2011 #35; Joyce, 2016 #86}, and this explains the different binding affinities of the H3 binder with the wild type H3 HA (A/Hong Kong/1/1968) and the deglycosylated H3 variant (N38D) in the BLI assay (FIG. 2 and FIG. 7). High resolution sequence footprinting (FIG. 2b & Table 16) and competition results suggest that the interfaces involve both the designed residues and the intended regions on the target. The very close agreement between the experimentally determined structures and the original design models suggests that the substitutions required to achieve high affinity play relatively subtle roles in tuning interface energetics; the overall structure of the complex, including the structure of the monomer binders and the detailed target binding mode, are determined by the computational design procedure.

TABLE 16
SSM Fingerprint Scores. Shown here are the SSM fingerprint scores
for the 12 characterized binders as well as the 2 Cryo-EM verified
SARS-CoV-2 binders. Using LCB1's P-Entropy column as the reference
for verification, all but CD3δ_mb and IGF1R_mb pass this
validation metric in both columns. Values in red are below the threshold
p-value of 0.005. Possible explanations for the failures are that
the IGF1R design model was lost (user error) and had to be
reconstructed via prediction. The CD3δ binder is weak and the
target protein is sticky. K/O mutations may still be able to
bind via alternate binding configurations

| Binder | P-Rosetta ™ | P-Entropy |
|---|---|---|
| H3_mb | 1e−16 | 5e−62 |
| TrkA_mb | 6e−08 | 6e−14 |
| FGFR2_mb | 2e−08 | 5e−05 |
| EGFRn_mb | 4e−17 | 1e−31 |
| EGFRc_mb | 4e−12 | 3e−08 |
| PDGFR_mb | 5e−10 | 3e−04 |
| InsulinR_mb | 9e−07 | 7e−13 |
| IGF1R_mb | 7e−01 | 6e−22 |
| Tie2_mb | 3e−03 | 4e−11 |
| IL-7Rα_mb | 4e−16 | 2e−10 |
| CD3δ_mb | 1e−01 | 5e−02 |
| TGF-β_mb | 2e−04 | 6e−05 |
| VirB8_mb | 1e−24 | 2e−36 |
| LCB1 | 2e−03 | 5e−03 |
| LCB3 | 5e−04 | 3e−21 |

### Conclusions

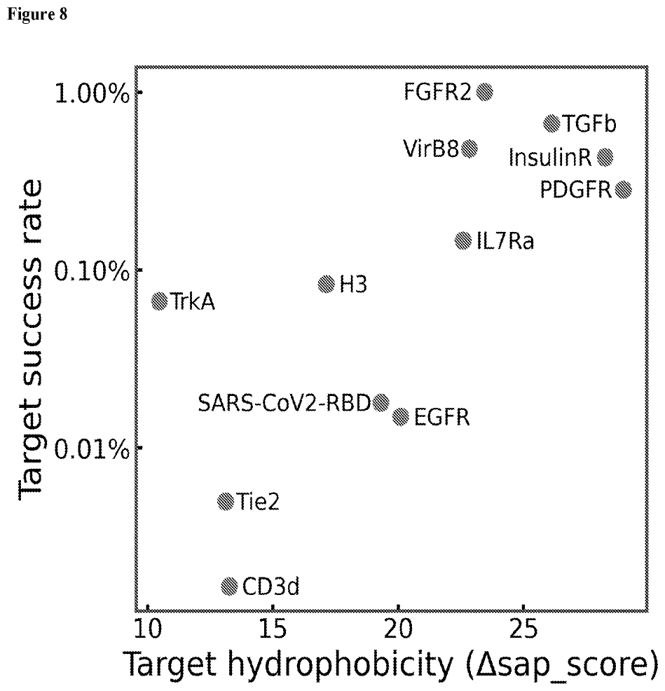

Our de novo binder design method and the large data set (810,000 binder designs and 240,000 single mutants) generated here provide a starting point for investigating the fundamental physical chemistry of protein-protein interactions, and for developing and assessing computational models of protein-protein interactions. Across all targets, there was a strong correlation between success rate and the hydrophobicity of the targeted region (FIG. 8), and designs observed experimentally to bind their targets tended to have stronger predicted binding energy, and larger contact molecular surfaces. As found previously for design of protein stability {Rocklin, 2017 #8}, iterative design-build-test cycles in which the design method is updated at each iteration to incorporate feedback from the previous design round should lead to systematic improvement in the design methodology and success rate.

Our success in designing nM affinity binders for 14 target sites demonstrates that binding proteins can be designed de novo using only information on the structure of the target protein, without need for prior information on binding hotspots or fragments from structures of complexes with binding partners. The success also suggests that our design pipeline provides a quite general solution to the de novo protein interface design problem that goes far beyond previously described methods. This work is a major step forward towards the longer range goal of direct computational design of high affinity binders starting from structural information alone. We expect the binders created here, and new ones created with the method moving forward, will find wide utility as signaling pathway antagonists as monomeric proteins and as tunable agonists when rigidly scaffolded in multimeric formats, and in diagnostics and therapeutics for pathogenic disease. More generally, the ability to rapidly and robustly design high affinity binders to arbitrary protein targets could transform the many areas of biotechnology and medicine that rely on affinity reagents.

### Methods

### Broad Search Stage

The crystal structures of HA (PDB: 4FNK) {Ekiert, 2012 #20}, EGFR (PDB: 1MOX, 4UV7) {Garrett, 2002 #21; Lim, 2016 #36}, PDGFR (PDB: 3MJG) {Shim, 2010 #22}, IR (PDB: 4ZXB) {Croll, 2016 #23}, IGFIR (PDB: 5U8R) {Xu, 2018 #24}, Tie2 (PDB: 2GY7) {Barton, 2006 #25}, IL-7Rα (PDB: 3DI3) {McElroy, 2009 #26}, CD3 (PDB: 1XIW) {Arnett, 2004 #27}, TGF-β (PDB: 3KFD) {Radaev, 2010 #28} and VirB8 (PDB: 403V) {Gillespie, 2015 #29} were refined in the Rosetta™ energy field constrained by experimental diffraction data. The crystal structures of TrkA (PDB: 1WWW) {Wiesmann, 1999 #30} and FGFR2 (PDB: 1EV2) {Plotnikov, 2000 #31} were refined with the Rosetta™ FastRelax protocol with coordinate constraints. The targeting chain or the selected targeting region were extracted and used as the starting point for docking and design. To run PatchDock™ {Schneidman-Duhovny, 2005 #9}, the scaffolds were mutated to poly-valine first and default parameters were used to generate the raw docks. Rifdock™ was used to generate the rotamer interacting field by docking billions of individual disembodied amino acids to the selected targeting regions {Dou, 2018 #7}. In detail, hydrophobic sidechain R-groups are docked against the target using a branch-and-bound search to quickly identify favorable interactions with the target, and polar sidechain R-groups are enumeratively sampled around every target bond donor or acceptor. To identify backbone placements from which these interactions can be made, side chain rotamer conformations are grown backwards for all R-group placements, and their backbone coordinates stored in a 6-dimensional spatial hash table for rapid lookup. For the hierarchical searching protocol, the miniprotein scaffold library (50-65 residues in length) was docked into the field of the inverse rotamers using a branch-and-bound searching algorithm from low resolution spatial grids to high resolution spatial grids. For the PatchDock™+Rifdock™ protocols, the PatchDock™ outputs were used as seeds for the initial positioning of the scaffolds and the docks were further refined in the finest resolution rotamer interaction field. These docked conformations were further optimized to generate shape and chemically complementary interfaces using the Rosetta™ FastDesign protocol, activating between side-chain rotamer optimization and gradient-descent-based energy minimization. Serval improvements were added to the sequence design protocol to generate better sequences for both folding and binding. These include a better repulsive energy ramping strategy {Maguire, 2021 #15}, upweighting cross-interface energies, a pseudo-energy term penalizing buried unsatisfied polar atoms {Coventry, 2021 #18} and a sequence profile constraint based on native protein fragments {Brunette, 2020 #17}. Computational metrics of the final design models were calculated using Rosetta™, which includes ddg, shape complementary and interface buried solvent accessible surface area, contact molecular surface, etc, for design selection. All the script and flag files to run the programs are in the Supplementary file.

### Focused Search Stage

The binding energy and interface metrics for all the continuous secondary structure motifs (helix, strand and loop) were calculated for the designs generated in the broad search stage. The motifs with good interaction (based on binding energy and other interface metrics, like SASA, contact molecular surface) with the target were extracted and aligned using the target structure as the reference. All the motifs were then clustered based on an energy based-TMalign™ like clustering algorithm. Briefly, all the motifs were sorted based on the interaction energy with the target, and the lowest energy motif in the unclustered pool was selected as the center of the first cluster. A similar score between this motif and every motif remaining in the unclustered pool was calculated based on the TMalign™ algorithm {Zhang, 2005 #37} without any further superimposition. Those motifs within a threshold similar score (default 0.7) from the current cluster center were removed from the unclustered pool and added to the new cluster. The lowest-energy motif remaining in the unclustered pool was selected as the center of the next cluster, and the second step was repeated. This process continued for subsequent clusters until no motifs remained in the unclustered pool. The best motif from each cluster was then selected based on the per-position weighted Rosetta™ binding energy, using the average energy across all the aligned motifs at each position as the weight. Around 2,000 best motifs were selected and the scaffold library was superimposed onto these motifs using the MotifGraft mover {Silva, 2016 #38}. Interface sequences were future optimized and computational metrics were computed for the final optimized designs as described in the broad search stage. CPU-time requirements to produce 100,000 designed binders to be tested experimentally were typically around 100.000 CPU-hours (usually at least 10× as many binders were computationally designed than were ordered).

### Rapid Rosetta™ Packing Evaluation (the Predictor)

A severe speed mismatch exists between the docking methods (RifDock™ and Focused search) and the subsequent full sequence design step. While the docking methods can typically produce outputs every 1 to 3 seconds, the full sequence design can take upwards of 4 minutes. To remedy this situation, a step was designed to take about 20 seconds that would be more predictive than metrics evaluated on raw docks, but faster than the full sequence design.

A stripped down version of the Rosetta™ beta_nov16 score function was used to design only with hydrophobic amino acids. Specifically, fa_elec, lkball[iso,bridge,bridge_unclp], and the_intra_terms were disabled as these proved to be the slowest energy methods by profiling. All that remained were Lennard-Jones, implicit solvation, and backbone-dependent one-body energies (fa_dun, p_aa_pp, rama_prepro). Additionally, flags were used to limit the number of rotamers built at each position (See Supplementary Information).

After the rapid design step, the designs are minimized twice: once with a low-repulsive score function and again with a normal-repulsive score function. Metrics of interest were then evaluated including like Rosetta™ ddG, Contact Molecular Surface, and Contact Molecular Surface to critical hydrophobic residues.

Using the fact that these predicted metrics correlate with the values after full sequence design, a Maximum Likelihood Estimator (functional form similar to logistic regression) was used to give each predicted design a likelihood that it should be selected to move forward. A subset of the docks to be evaluated are subjected to the full sequence design, and their final metric values calculated. With a “goal threshold” for each filter, each fully-designed output can be marked as “pass” or “fail” for each metric independently. Then, by binning the fully-designed outputs by their values from the rapid trajectory and plotting the fraction of designs that pass the “goal threshold”, the probability that each predicted design passes each filter can be calculated (sigmoids are fitted to smooth the distribution). From here, the probability of passing each filter may be multiplied together to arrive at the final probability of passing all filters. This final probability can then be used to rank the designs and pick the best designs to move forward to full sequence optimization. The rapid design protocol here is used merely to rank the designs, not to optimize them; the raw, non-rapid-designed docks are the structures carried forward.

### Contact Molecular Surface

Solvent-accessible surface area (SASA) is a measure of the exposure of amino acids to the solvent and it is typically calculated by methods involving in-silico rolling of a spherical probe, which approximates a water molecule (radius 1.4 Å), around a full-atom protein model. Delta-SASA upon protein-protein binding has been widely used to analyze native protein interactions. Unlike the crystal structures of the native protein complexes, design models for the de novo interactions are usually imperfectly packed, and contain many holes or cavities. If the sizes of the holes or cavities in the interface are smaller than the rolling probe, the SASA can not capture those holes and cavities and the real contacts are usually overestimated by the delta-SASA metric. The contact molecular surface was developed to capture the flaws of the de novo designed interactions. Firstly, the molecule surfaces of the binder and the target were calculated by the triangularization algorithm in the Rosetta™ shape complementary filter. For each triangle, the distance to the closest triangle on the other side was calculated and used to downweight the area of the triangle by the equation: A′=A*exp(−0.5*distance²) Then all the down-weighted areas were summed up to get the contact molecular surface. In this way, the real contacts between the target and the binder are penalized by the cavities and holes in the interface. The contact molecular surface was implemented as the ContactMolecularSurface filter in the Rosetta™ macromolecular modelling suite.

Upweight Protein interface Interactions

Rosetta™ sequence design starts from generating an interaction graph by calculating the energies between all designable rotamer pairs {Leaver-Fay, 2011 #39}. The best rotamer combinations are searched using a Monte Carlo Simulated Annealing protocol by optimizing the total energy of the protein (monomer/complex). To obtain more contacts between the binder and the target protein, we can upweight the energies of all the cross interface rotamer pairs by a defined factor. In this way, the Monte Carlo protocol will be biased to find solutions with better cross interface interactions. The upweight protein interface interaction protocol was implemented as the ProteinProteinInterfaceUpweighter task operation in the Rosetta™ macromolecular modelling suite.

### DNA Library Preparation

All protein sequences were padded to 65aa by adding a (GGGS)n (SEQ ID NO: 1574) linker at the C terminal of the designs, to avoid the biased amplification of short DNA fragments during PCR reactions. The protein sequences were reversed translated and optimized using DNAworks2.0 {Hoover, 2002 #1} with the S. cerevisiae codon frequency table. Oligo pool encoding the de novo designs and the point mutant library were ordered from Agilent Technologies. Combinatorial libraries were ordered as IDT (Integrated DNA Technologies) ultramers with the final DNA diversity ranging from 1e6 to 1e7.

All libraries were amplified using Kapa HiFi Polymerase (Kapa Biosystems) with a qPCR machine (BioRAD CFX96). In detail, the libraries were firstly amplified in a 25 ul reaction, and PCR reaction was terminated when the reaction reached half maximum yield to avoid over amplification. The PCR product was loaded to a DNA agarose gel. The band with the expected size was cut out and DNA fragments were extracted using QlAquick™ kits (Qiagen, Inc.). Then, the DNA product was re-amplified as before to generate enough DNA for yeast transformation. The final PCR product was cleaned up with a QlAquick™ Clean up kit (Qiagen, Inc.). For the yeast transformation, 2-3 μg of linearized modified pETcon™ vector (pETcon3) and 6 μg of insert were transformed into EBY100 yeast strain using the protocol as described in {Benatuil, 2010 #12}.

DNA libraries for deep sequencing were prepared using the same PCR protocol, except the first step started from yeast plasmid prepared from 5×10⁷ to 1×10⁸ cells by Zymoprep™ (Zymo Research). Illumina adapters and 6-bp pool-specific barcodes were added in the second qPCR step. Gel extraction was used to get the final DNA product for sequencing. All different sorting pools were sequenced using Illumina NextSeq™ sequencing.

### Target Protein Preparation

Influenza A hemagglutinin (HA) ectodomain was expressed using a baculovirus expression system as described previously {Stevens, 2004 #62; Ekiert, 2012 #63}. Briefly, each HA was fused with gp67 signal peptide at the N-terminus and to a BirA biotinylation site, thrombin cleavage site, trimerization domain and His-tag at the C-terminus. Expressed HAs were purified using metal affinity chromatography using Ni-NTA resin. For binding studies, each HA was biotinylated with BirA and purified by gel filtration using S200 16/90 column on ÄKTA protein purification system (GE Healthcare). The biotinylation reactions contained 100 mM Tris (pH 8.5), 10 mM magnesium acetate, 10 mM ATP, 50 μM biotin and <50 mM NaCl, and were incubated at 37° C. for 1 hr.

For TrkA, the DNA encoding human TrkA ECD (residues 36-382) was cloned into pAcBAP, a derivative of pAcGP67-A modified to include a C-terminal biotin acceptor peptide (BAP) tag (GLNDIFEAQKIEWHE) (SEQ ID NO:1571) followed by a 6×HIS tag for affinity purification. It was then transfected into Trichoplusia ni (High Five) cells (Invitrogen) using the BaculoGold™ baculovirus expression system (BD Biosciences) for secretion and purified from the clarified supernatant via Ni-NTA followed by size exclusion chromatography with a Superdex™-200 column in sterile Phosphate Buffer Saline (PBS) (Cat. 20012-027; Gibco). The ectodomains of FGFR2 (residues 147-366, Uniprot ID P21802), EGFR (residues ID 25-525, Uniprot ID P00552), PDGFR (residues 33-314, Uniprot ID P09619), InsulinR (residues ID 28-953, Uniprot ID P06213), IGF1R (residues 31-930, Uniprot ID P08069), Tie2 (residues 23-445, Uniprot ID Q02763), IL-7Rα (residues 37-231, Uniprot ID P16871) were expressed in mammalian cells with a IgK Signal peptide (METDTLLLWVLLLWVPGSTG) (SEQ ID NO:1572) at the N-terminus and a C-terminal tag (GSENLYFQGSHHHI-HHHGSGLNDIFEAQKIEWHE) (SEQ ID NO:1573) which contains a TEV cleavage site, a 6-His-tag and an AviTag. VirB8 was expressed in E. coli with a C-terminal AviTag as previously described {Gillespie, 2015 #19}. The proteins were purified by Ni-NTA, and polished with size exclusion chromatography. Then, the AviTag-proteins were biotinylated with the BirA biotin-protein ligase bulk reaction kit (Avidity) following the manufacturer's protocol and the excessive biotin was removed through size exclusion chromatography. Biotinylated CD3 protein was bought from Abcam (Cat #ab205994). TGF-β was bought from Acro Biosystems (Cat #TG1-H8217). IGF1 was bought from Sigma (Cat #407251-100 ug). Insulin was bought from Abcam (Cat #ab123768). The caged Ang1-Fc protein was prepared as described previously {Divine, 2021 #77}, and was kindly provided by George Ueda.

### Yeast Surface Display

S. cerevisiae EBY100 strain cultures were grown in C-Trp-Ura media supplemented with 2% (w/v) glucose. For induction of expression, yeast cells were centrifuged at 6,000×g for 1 min and resuspended in SGCAA media supplemented with 0.2% (w/v) glucose at the cell density of 1×10\hat{ }7 cells per ml and induced at 30° C. for 16-24 h. Cells were washed with PBSF (PBS with 1% (w/v) BSA) and labelled with biotinylated targets using two labeling methods, with-avidity and without-avidity labeling. For the with-avidity method, the cells were incubated with biotinylated target, together with anti-c-Myc fluorescein isothiocyanate (FITC, Miltenyi Biotech) and streptavidin-phycoerythrin (SAPE, ThermoFisher). The concentration of SAPE in the with-avidity method was used at % concentration of the biotinylated targets. For the without-avidity method, the cells were firstly incubated with biotinylated targets, washed, secondarily labelled with SAPE and FITC. All the original libraries of de novo designs were sorted using the with-avidity method for the first few rounds of screening to fish out weak binder candidates, followed by several without-avidity sorts with different concentrations of targets. For SSM libraries, two rounds of without-avidity sorts were applied and in the third round of screening, the libraries were titrated with a series of decreasing concentrations of targets to enrich mutants with beneficial mutations. The combinatorial libraries were sorted to convergence by decreasing the target concentration with each subsequent sort and collecting only the top 0.1% of the binding population. The final sorting pools of the combinatorial libraries were plated on C-trp-ura plates and the sequences of individual clones were determined by Sanger sequencing. The competition sort was done following the without-avidity protocols with a very minor modification. Briefly, the biotinylated target proteins (H1, H3, TrkA, InsulinR, IGFIR, PDGFR and Tie2) were first incubated with an excessive amount of competitors (FI6v3. FI6v3, NGF, insulin, IGF1, PDGF and caged Ang1-Fc) respectively for 10 mins, and the mixture was used for labeling the cells. The non-specificity reagent was prepared using the protocol as described in {Xu, 2013 #13}. For non-specificity sort, the cells were firstly washed with PBSF and incubated with the non-specificity reagent at the concentration of 100 μg/ml for 30 mins. The cells were then washed and secondarily labelled with SAPE and FITC for cell sorting. The cells were then labeled with RBD using the above mentioned protocol.

### Miniprotein Expression

Genes encoding the designed protein sequences were synthesized and cloned into modified pET-29b(+) E. coli plasmid expression vectors (GenScript™, N-terminal 8 His-tag followed by a TEV cleavage site). For all designed proteins, the sequence of the N-terminal tag is MSHHHHHHHHSENLYFQSGGG (SEQ ID NO: 1560 (unless otherwise noted), which is followed immediately by the sequence of the designed protein. For proteins expressed with the maltose binding protein (MBP) tag, the corresponding genes were subcloned into a modified pET-29b(+) E. coli plasmid, which has a N-terminal 6 His-tag and a MBP tag. Plasmids were transformed into chemically competent E. coli Lemo21 cells (NEB). For the designs for TrkA, FGFR2, EGFR. IR, IGF1R, Tie2, IL-7Rα, TGF-β and the MBP tagged miniproteins, protein expression was performed using the Studier autoinduction media supplemented with antibiotic, and cultures were grown overnight. For designs for HA, PDGFR and CD3δ, the E. coli cells were grown in LB media at 37° C. until the cell density reached 0.6 OD600. Then, IPTG was added to the final concentration of 500 mM and the cells were grown overnight at 22° C. for expression. The cells were harvested by spinning at 4,000×g for 10 min and then resuspended in lysis buffer (300 mM NaCl, 30 mM Tris-HCL, pH 8.0, with 0.25% CHAPS for cell assay samples) with DNAse and protease inhibitor tablets. The cells were lysed with a sonicator for 4 minutes total (2 minutes on time, 10 sec on-10 sec off) with an amplitude of 80%. Then the soluble fraction was clarified by centrifugation at 20,000×g for 30 min. The soluble fraction was purified by Immobilized Metal Affinity Chromatography (Qiagen) followed by FPLC size-exclusion chromatography (Superdex™ 75 10/300 GL, GE Healthcare). All protein samples were characterized with SDS-PAGE with the purity higher than 95%. Protein concentrations were determined by absorbance at 280 nm measured using a NanoDrop™ spectrophotometer (Thermo Scientific) using predicted extinction coefficients.

### Circular Dichroism

Far-ultraviolet CD measurements were carried out with an JASCO-1500 equipped with a temperature-controlled multi-cell holder. Wavelength scans were measured from 260 to 190 nm at 25, 95° C. and again at 25° C. after fast refolding (˜5 min). Temperature melts monitored dichroism signal at 222 nm in steps of 2° C./minute with 30s of equilibration time. Wavelength scans and temperature melts were performed using 0.3 mg/ml protein in PBS buffer (20 mM NaPO4, 150 mM NaCl, pH 7.4) with a 1 mm path-length cuvette. Melting temperatures were determined fitting the data with a sigmoid curve equation. 9 out of the 13 designs retained more than half of the mean residue ellipticity values, which indicated the Tm values are greater than 95° C. Tm values of the other designs were determined as the inflection point of the fitted function.

### Biolayer Interferometry

Biolayer interferometry binding data were collected on an Octet RED96™ (ForteBio) and processed using the instrument's integrated software. For minibinder binding assays, biotinylated targets were loaded onto streptavidin-coated biosensors (SA ForteBio) at 50 nM in binding buffer (10 mM HEPES (pH 7.4), 150 mM NaCl, 3 mM EDTA, 0.05% surfactant P20, 1% BSA) for 360 s. Analyte proteins were diluted from concentrated stocks into the binding buffer. After baseline measurement in the binding buffer alone, the binding kinetics were monitored by dipping the biosensors in wells containing the target protein at the indicated concentration (association step) and then dipping the sensors back into baseline/buffer (dissociation). The binding affinities of Tie2- and IGF1R-mini binders were low, and MBP tagged proteins were used for the binding assay to amplify the binding signal. The binding assay for the Insulin receptor (IR) designs were conducted with Amine Reactive Second-Generation (AR2G ForteBio) Biosensors with the recommended protocol. In brief, the miniproteins were immobilized onto the AR2G tips and the InsulinR were used as the analyte with the indicated concentrations.

For the cross-reactivity assay, each target protein was loaded onto SA tips at the concentration of 50 nM for 325s. The tips were dipped into the miniprotein wells for 300s (association) and then dipped into the blank buffer wells for 600s (dissociation). The maximum raw bio-layer Interferometry signal binding was used as the indicator of binding strength. The maximum signal among all the miniprotein binders for a specific target was used to normalize the data for heatmap plotting.

Apparent SC₅₀ Estimation from FACS and NGS

The Pear™ program {Zhang, 2014 #61} was used to assemble the fastq files from the Next Generation Sequencing runs. Translated, assembled reads were matched against the ordered designs to determine the number of counts for each design in each pool.

The critical assumption to the fitting here is to pretend that the yeast cells displaying a particular design will follow a modified version of the standard KD equation relating fraction bound to concentration:

$${fraction\_collected}_{i} = \frac{concentration}{( concentration + {SC}_{50 , i} )} \qquad EQ - KD$$

where fraction_collectedᵢ is the fraction of the yeast cells displaying design i that were collected, concentration is the target concentration for sorting, and SC_{50,i} is the apparent SC₅₀ of the design (the concentration where 50% of the cells would be collected).

The next assumption is that all designs have the same expression level on yeast surface and that 100% of yeast cells express well enough to be collected in the “expression” gate.

These two assumptions, while probably false, allow fitting the data with only one free parameter per design and no global free parameters. The correct version of EQ-KD for this experiment likely has a different shape and slope from a perfect sigmoid, the net effect of correcting this would be that all SC₅₀ are scaled by a constant factor (which would not affect the relative comparisons made here). It can be shown by analyzing the data that different designs result in different expression levels on yeast (one can examine the fraction_collectedᵢ for strong binders at concentrations where binding should be saturated). The net result is that experimentally, EQ-KD is multiplied by a constant between 0 and 1 for each design. This constant seems to range from 0.2 to 0.7. As such, when fitting the data, fraction_collectedᵢ values above 0.2 are considered saturating. However, because the 0.2 mark may represent 90% collection for poorly-expressing designs and 30% collection for strongly-expressing designs, the resulting SC₅₀ fits may vary by up to 5-fold. The alternative is to try to estimate an expression level: however, this becomes increasingly difficult with weaker binders that never saturate the experiment.

Apparent SC₅₀ Estimation from FACS and NGS: Point Estimates

The following equation may be used to determine the fraction_collectedᵢ for a single design in a single sort:

$$EQ - FRAC$$

$${fraction\_collected}_{i} = \frac{proportion\_child {\_pool}_{i}}{proportion\_parent {\_pool}_{i}} \times facs\_collection \_fraction$$

where fraction_collectedᵢ is the proportion of cells carrying design i that were collected during the sort, proportion_child_poolᵢ is the proportion of the total NGS counts for design i from the pool that was collected, proportion_parent_poolᵢ is the proportion of the total NGS counts for design i from the pool that was the input for the sorter, and facs_collection_fraction was the fraction of the yeast cells collected during the specific sort (a number extracted from the FACS machine itself).

This point-estimate method is best suited for asking the question: which designs have SC₅₀<SC_{50,0?} by determining the expected fraction_collectedᵢ for a given sorting concentration and SC_{50,0}. The sorting concentration and SC_{50,0} should be selected such that EQ-KD results in an expected fraction_collectedᵢ less than 0.2 in order to circumvent the expression issues mentioned above. Then, any designs with fraction_collectedᵢ greater than the cutoff may say their SC₅₀ is less than SC_{50,0}. Designs with low numbers of counts are suspect, see the Doubly-Transformed Yeast Cells section. For this analysis, any designs with fewer than max_possible_passenger_cells cells were eliminated.

This method may be applied to avidity sorts, however, the resulting SC₅₀ would be the SC₅₀ during avidity experiments. It is unclear to the authors what the precise mathematical effect of avidity is and as such we do not compare avidity SC_{50S} with non-avidity SC_{50S}.

Apparent SC₅₀ Estimation from FACS and NGS: Doubly-Transformed Yeast Cells

Doubly-transformed yeast cells represent a major source of error in these experiments. While rare, a yeast cell that contains two plasmids, one of a strong binder and one of a non-binder, will carry the non-binder plasmid through the sorting process. The net result is that the non-binder will end up with counts that track the strong binder, however, at a greatly reduced absolute number. (Rare is a relative term here. While the odds of any two specific plasmids being in one cell is low, in the entire pool of yeast, doubly transformed cells seem to be quite common.)

We chose to address this issue by making the following assumption: non-binders that take advantage of a doubly-transformed yeast cell do so from precisely one double-transformation event. In other words, we assume that the same non-binding plasmid did not get doubly transformed into two separate strong-binding yeast. This assumption allows us to estimate the largest number of cells we would expect to see from a doubly-transformed plasmid:

$$EQ - MAX$$

$$max\_possible \_passenger \_cells = \frac{{cells\_collected}_{i\_ max}}{cells\_sorted \_R {1}_{i\_ max}} \times cell\_copies \_before \_first \_sort$$

where max_possible_passenger_cells is the highest number of cells that we would expect a non-binding plasmid to occupy, cells_collected_{i_max} is the number of cells collected in this round for the design with the most number of cells collected, cells_sorted_Rl_{i_max} is the number of cells sorted for design i_max (the same design from cells_collected_{i_max}), and cell_copies_before_first_sort is the number of copies of each cell that occurred before the first sort (2^{#cell_divisions}) The number of cells_collectedᵢ may be approximated by multiplying the number of cells the FACS machine collected by the proportion of the pool that design i represents. The number of cells_sortedᵢ may be estimated by either dividing the cells_collectedᵢ by the facs_collection_fraction or by multiplying the number of cells fed to the FACS machine by the proportion of design i in that pool.

With this number in hand, one can set a floor for the number of cells that one would expect to see. Any design with fewer than this number of cells cannot be considered for calculations because it is unclear whether or not that cell is part of a doubly-transformed yeast cell. On the whole, this method reduces false-positive binders, but also removes true-positive binders that did not transform well. It is wise to simply drop designs from the downstream calculations that did not transform well.

Apparent SC₅₀ Estimation from FACS and NGS: Full Estimate

Estimation of an upper and lower bound on the SC₅₀ from the data may be performed by looking at an arbitrary number of sorting experiments. Taking a P(SC₅₀=SC_{50,0}|data) and performing Bayesian analysis, one arrives at a confidence interval for the actual SC₅₀ value. This analysis may be performed at every sort and the resulting distributions combined to produce a robust estimate.

Each sort may be modeled as a binomial distribution where: p=fraction_collected from EQ-KD using concentration=sorting_concentration and SC₅₀=SC_{50,0}; n=cells_sortedᵢ; and x=cells_collectedᵢ. By performing this analysis at a range of SC_{50,0} values and examining the probability this could happen by the binomial distribution, one arrives at P(SC₅₀=SC_{50,0}|data). Specifically for this analysis, the cdf of the binomial was used with the null hypothesis that SC₅₀=SC_{50,0}.

Care should be taken for the valid range of p. As stated previously, it is wise to cap the expected value of p to 0.2 to account for expression levels and to floor the value such that n*p does not fall below max__possible_passenger_cells. In our implementation, if x falls into a range that has been clipped, a probability of 1 is returned.

### SSM Validation: Relax Protocol

In order to remove artifacts from design and to discover the best orientation for each SSM mutation, all binders were relaxed using the Rosetta™ beta_nov16 score function before calculations began (30 replicates using 5 repeats of cartesian FastRelax™ taking the best scoring model). Relaxation of point mutants then used the standard cartesian FastRelax procedure and allowed all residues within 10 Å of the mutation to relax. The backbone coordinates of those residues on the binder were allowed to relax while the target was held constant. The best of 3 (as evaluated by Rosetta™ energy) was chosen as the representative model.

### SSM Validation: Entropy Score

In order to validate that the designed binder was folded into the correct shape and was using its designed interface to bind to the target, the entropy of the interface, monomer core, and monomer surface were examined. For each position on the binder, the sequence entropy (Shannon entropy) of each position was calculated using the observed frequencies of each amino acid in the Next Generation Sequencing data. The specific pool that was chosen for this analysis was the pool with concentration closest to 10-fold lower than the calculated SC₅₀ of the parent.

After the per-position sequence entropy was calculated, the average per-position entropy of the SASA-hidden positions contacting the target (interface core), the SASA-hidden positions not contacting the target (monomer core), and the fully exposed positions not contacting the target (monomer surface) were calculated. A simple subtraction was performed according to EQ-ENTROPY:

$$EQ - ENTROPY$$

$$intermediate\_entropy \_score = {S}_{monomer \_ core} + {S}_{interface \_ core} - {S}_{monomer \_ surface}$$

$$where {S}_{region} is the average entropy of that region .$$

Finally, the probability that the score could have come from totally random data was computed by performing the above calculation on the actual data, and then performing the same calculation 100 times, but randomly mismatching the observed counts among all SSM point mutations. In this way, the experimental noise is kept constant among the 100 decoy datasets. The final step to arrive at a p-value was to calculate the mean and standard deviation of the 100 decoy intermediate_entropy_scores and to find the p-value with the Normal CDF function of the binder's intermediate_entropy_score.

### SSM Validation: Rosetta™ Accuracy Score

In order to further assess the accuracy of the design model, the correlation between the predicted effect on binding by Rosetta™ was compared with the experimental data. The effect from Rosetta™ can be broken into two components: monomer stabilization/destabilization and interface stabilization/destabilization. The effect on the monomer energy will affect the fraction of the proteins that are folded in solution. This fraction of folded proteins will then worsen the affinity because only the folded proteins are able to bind. The effect on the monomer stability was estimated by taking the difference in Rosetta™ energy between the native relaxed dock and the mutant relaxed dock and looking only at the change in Rosetta™ score of the docked protein (excluding energies arising from cross-interface edges). The effect on the target energy was calculated the same was and was considered to directly affect the binding energy. The binding energy was calculated by taking the difference in Rosetta™ score between the docked and undocked conformations (but with no repacking or minimization in the unbound form).

The effect on the P(fold_monomer) was estimated by first determining the predicted ΔG_{fold} of the native protein.

$$P ( fold\_monomer ) = exp ( \frac{Δ {G}_{fold} + Δ {G}_{mutant \_ effect}}{kT} ) \qquad EQ - PFOLD$$

$$Δ {ddG}_{monomer \_ effect} = kT ln ( \frac{{P ( fold\_monomer )}_{native}}{{P ( fold\_monomer )}_{mutant}} ) \qquad EQ - ddg\_monomer$$

where k is the Boltzmann constant and T is temperature which was set to 300 K for this calculation.

Using EQ-ddg_monomer and EQ-PFOLD, the predicted ΔG_{fold} for the native design was estimated by performing a least-squares fit of all mutations that did not occur in residues at the interface. A rudimentary confidence interval was created by allowing all ΔG_{fold} values that resulted in a root mean squared error of within 0.25 kcal/mol of the best ΔG_{fold} value. Typical confidence intervals spanned 3 kcal/mol.

$$EQ - DDG\_SUM$$

$$Δ {ddG}_{Rosetta} = Δ {ddG}_{monomer \_ effect} + Δ {ddG}_{interface \_ effect} + Δ {ddG}_{target \_ effect}$$

With the ΔG_{fold} in hand, the predicted effect on the binding energy could be computed according to EQ-DDG_SUM. The values of ΔG_{fold} inside the confidence range for ΔG_{fold} that produced the largest and smallest ΔddG_{Rosetta} were used to produce a confidence interval for ΔddGa_{Rosetta}.

The per-position accuracy was assessed by determining whether the confidence interval for ΔddG_{Rosetta} was compatible with the confidence interval for the SC₅₀ from the experimental data. A buffer of 1 kcal/mol was allowed.

With the per-position accuracies in hand, the overall percentage of mutations that Rosetta™ was able to explain in the monomer_core and interface_core was assessed. This produced an overall Rosetta™ accuracy score.

In the same way as the Entropy score, 100 decoys with randomly shuffled SC₅₀ values were subjected to the same procedure. The mean and standard deviation of the decoys was determined and the p-value for the Rosetta™ score was determined using the Normal CDF function.

1. A polypeptide comprising an amino acid sequence at least 35% identical to the amino acid sequence selected from the group consisting of SEQ ID NO:1-1559 and 1561-1570, not including any functional domains added fused to the polypeptides (whether N-terminal, C-terminal, or internal), and wherein the 1, 2, 3, 4, or 5 N-terminal and/or C-terminal amino acid residues may be present or absent when considering the percent identity.

2. The polypeptide of claim 1, wherein substitutions relative to the reference polypeptide are selected from the residues listed as “best” or “tolerable” at each position immediately below the reference polypeptide listed in Tables 13A-13HHH.

3. The polypeptide of claim 1, wherein substitutions relative to the reference polypeptide are selected from the residues listed as “best” or “tolerable” at each position immediately below the reference polypeptide listed in Tables 13A-13HHH.

4. The polypeptide of claim 1, wherein 1, 2, 3, 4, 5, 6, 7, 8, 9, 10, 11, 12, 13, 14, 15, or all interface residues are as defined in the reference polypeptide listed in Tables 13A-13HHH.

5. The polypeptide of claim 1, wherein protein core residues listed in Tables 13A-13HHH are substituted relative to the reference polypeptide only with conservative amino acid substitutions.

6. The polypeptide of claim 1, wherein insertion of amino acid residues relative to the reference polypeptide occurs at a residue indicated in the column “loop/insertion” column of Tables 13A-13HHH.

7. The polypeptide of claim 1, wherein 1, 2, 3, 4, or 5 N-terminal and/or C-terminal amino acid residues are not included when determining the percent identity relative to the reference polypeptide.

8. The polypeptide of claim 1, wherein all residues are included when determining the percent identity relative to the reference polypeptide.

9. A fusion protein comprising the polypeptide of claim 1 fused to a functional polypeptide.

10. A fusion protein comprising two or more copies of the polypeptide of claim 1.

11. The fusion protein of claim 10 wherein the two or more copies of the polypeptide are identical.

12. The fusion protein of claim 10 wherein the two or more copies of the polypeptide are not all identical.

13. A scaffold comprising 2 or more copies of the polypeptide or fusion protein of claim 1.

14. A nucleic acid encoding the polypeptide of claim 1.

15. An expression vector comprising the nucleic acid of claim 14 operatively linked to a suitable control sequence.

16. A host cell comprising the expression vector of claim 15.

17. A pharmaceutical composition comprising:
(a) the polypeptide claim 1, ; and
(b) a pharmaceutically acceptable carrier.

18. A method for using; the polypeptide claim 1 for any suitable use as disclosed herein.

19. The method of claim 19, wherein the use comprises treating a tumor or infection.

20. (canceled)
